SEQUENCE LISTING (182 sequences)

SEQ ID NO: 1 - DNA, 1494 bp, Homo sapiens
atggggtccctagaaatggtgccaatgggcgcgggtccccctagccccggcggggatccggatgggtacgatggcggaaacaactcccaatatccatctgcttctggctcttctgggaacacccccaccccaccgaacgatgaggaacgtgaatctaatgaagagcccccaccgccttatgaggacccatattggggcaatggcgaccgtcactcggactatcaaccactaggaacccaagatcaaagtctgtacttgggattgcaacacgacgggaatgacgggctccctccccctccctactctccacgggatgactcatctcaacacatatacgaagaagcgggcagaggaagtatgaatccagtatgcctgcctgtaattgttgcgccctacctcttttggctggcggctattgccgcctcgtgtttcacggcctcagttagtaccgttgtgaccgccaccggcttggccctctcacttctactcttggcagcagtggccagctcatatgccgctgcacaaaggaaactgctgacaccggtgacagtgcttactgcggttgtcactttctttgcaatttgcctaacatggaggattgaggacccaccttttaattctcttctgtttgcattgctggccgcagctggcggactacaaggcatttacgttctggtgatgcttgtgctcctgatactagcgtacagaaggagatggcgccgtttgactgtttgtggcggcatcatgtttttggcatgtgtacttgtcctcatcgtcgacgctgttttgcagctgagtcccctccttggagctgtaactgtggtttccatgacgctgctgctactggctttcgtcctctggctctcttcgccagggggcctaggtactcttggtgcagcccttttaacattggcagcagctctggcactgctagcgtcactgattttgggcacacttaacttgactacaatgttccttctcatgctcctatggacacttgtggttctcctgatttgctcttcgtgctcttcatgtccactgagcaagatccttctggcacgactgttcctatatgctctcgcactcttgttgctagcctccgcgctaatcgctggtggcagtattttgcaaacaaacttcaagagtttaagcagcactgaatttatacccaatttgttctgcatgttattactgattgtcgctggcatactcttcattcttgctatcctgaccgaatggggcagtggaaatagaacatacggtccagtttttatgtgcctcggtggcctgctcaccatggtagccggcgctgtgtggctgacggtgatgtctaacacgcttttgtctgcctggattcttacagcaggattcctgattttcctcattggctttgccctctttggggtcattagatgctgccgctactgctgctactactgccttacactggaaagtgaggagcgcccaccgaccccatatcgcaacactgtataa

SEQ ID NO: 2 - protein, 497 aa, Homo sapiens
MGSLEMVPMGAGPPSPGGDPDGYDGGNNSQYPSASGSSGNTPTPPNDEERESNEEPPPPYEDPYWGNGDRHSDYQPLGTQDQSLYLGLQHDGNDGLPPPPYSPRDDSSQHIYEEAGRGSMNPVCLPVIVAPYLFWLAAIAASCFTASVSTVVTATGLALSLLLLAAVASSYAAAQRKLLTPVTVLTAVVTFFAICLTWRIEDPPFNSLLFALLAAAGGLQGIYVLVMLVLLILAYRRRWRRLTVCGGIMFLACVLVLIVDAVLQLSPLLGAVTVVSMTLLLLAFVLWLSSPGGLGTLGAALLTLAAALALLASLILGTLNLTTMFLLMLLWTLVVLLICSSCSSCPLSKILLARLFLYALALLLLASALIAGGSILQTNFKSLSSTEFIPNLFCMLLLIVAGILFILAILTEWGSGNRTYGPVFMCLGGLLTMVAGAVWLTVMSNTLLSAWILTAGFLIFLIGFALFGVIRCCRYCCYYCLTLESEERPPTPYRNTV

SEQ ID NO: 3 - DNA, 1137 bp, Homo sapiens
atgaatccagtatgcctgcctgtaattgttgcgccctacctcttttggctggcggctattgccgcctcgtgtttcacggcctcagttagtaccgttgtgaccgccaccggcttggccctctcacttctactcttggcagcagtggccagctcatatgccgctgcacaaaggaaactgctgacaccggtgacagtgcttactgcggttgtcactttctttgcaatttgcctaacatggaggattgaggacccaccttttaattctcttctgtttgcattgctggccgcagctggcggactacaaggcatttacgttctggtgatgcttgtgctcctgatactagcgtacagaaggagatggcgccgtttgactgtttgtggcggcatcatgtttttggcatgtgtacttgtcctcatcgtcgacgctgttttgcagctgagtcccctccttggagctgtaactgtggtttccatgacgctgctgctactggctttcgtcctctggctctcttcgccagggggcctaggtactcttggtgcagcccttttaacattggcagcagctctggcactgctagcgtcactgattttgggcacacttaacttgactacaatgttccttctcatgctcctatggacacttgtggttctcctgatttgctcttcgtgctcttcatgtccactgagcaagatccttctggcacgactgttcctatatgctctcgcactcttgttgctagcctccgcgctaatcgctggtggcagtattttgcaaacaaacttcaagagtttaagcagcactgaatttatacccaatttgttctgcatgttattactgattgtcgctggcatactcttcattcttgctatcctgaccgaatggggcagtggaaatagaacatacggtccagtttttatgtgcctcggtggcctgctcaccatggtagccggcgctgtgtggctgacggtgatgtctaacacgcttttgtctgcctggattcttacagcaggattcctgattttcctcattggctttgccctctttggggtcattagatgctgccgctactgctgctactactgccttacactggaaagtgaggagcgcccaccgaccccatatcgcaacactgtataa

SEQ ID NO: 4 - protein, 378 aa, Homo sapiens
MNPVCLPVIVAPYLFWLAAIAASCFTASVSTVVTATGLALSLLLLAAVASSYAAAQRKLLTPVTVLTAVVTFFAICLTWRIEDPPFNSLLFALLAAAGGLQGIYVLVMLVLLILAYRRRWRRLTVCGGIMFLACVLVLIVDAVLQLSPLLGAVTVVSMTLLLLAFVLWLSSPGGLGTLGAALLTLAAALALLASLILGTLNLTTMFLLMLLWTLVVLLICSSCSSCPLSKILLARLFLYALALLLLASALIAGGSILQTNFKSLSSTEFIPNLFCMLLLIVAGILFILAILTEWGSGNRTYGPVFMCLGGLLTMVAGAVWLTVMSNTLLSAWILTAGFLIFLIGFALFGVIRCCRYCCYYCLTLESEERPPTPYRNTV

SEQ ID NO: 5 - DNA, 303 bp, Homo sapiens
atgccttggcatgacgtcaactttacttttatttcagttctggtgatgcttgtgctcctgatactagcgtacagaaggagatggcgccgtttgactgtttgtggcggcatcatgtttttggcatgtgtacttgtcctcatcgtcgacgctgttttgcagctgagtcccctccttggagctgtaactgtggtttccatgacgctgctgctactggctttcgtcctctggctctcttcgccagggggcctaggtactcttggtgcagcccttttaacattggcagcaggtaagccacacgtgtga

SEQ ID NO: 6 - protein, 100 aa, Homo sapiens
MPWHDVNFTFISVLVMLVLLILAYRRRWRRLTVCGGIMFLACVLVLIVDAVLQLSPLLGAVTVVSMTLLLLAFVLWLSSPGGLGTLGAALLTLAAGKPHV

SEQ ID NO: 7 - DNA, 135 bp, Homo sapiens
atgacttacatgggtttggcttttgtaggctttgccctctttggggtcattagatgctgccgctactgctgctactactgccttacactggaaagtgaggagcgcccaccgaccccatatcgcaacactgtataa

SEQ ID NO: 8 - protein, 44 aa, Homo sapiens
MTYMGLAFVGFALFGVIRCCRYCCYYCLTLESEERPPTPYRNTV

SEQ ID NO: 9 - DNA, 3957 bp, Homo sapiens
atggaagagaggggcagggaaacgcaaatgccggttgcccggtatgggggcccgtttattatggtaaggctcttcgggcaagatggagaggcaaacatacaggaggaaaggctatatgagctactctctgacccacgctccgcgctcggcctagacccggggcccctgattgctgagaacctgctgctagtggcgctgcgtggcaccaacaacgatcccaggcctcagcgtcaggagagggccagagaactggccctcgttggcattctactaggaaacggcgagcagggtgaacacttgggcacggagagtgccctggaggcctcaggcaacaactatgtgtatgcctacggaccagactggatggcaaggccttccacatggtccgcggaaatccagcaattcctgcgactcctgggcgccacgtacgtgcttcgcgtggagatgggcaggcagtttggcttcgaggtgcatagaagccggccctccttccgtcagttccaggccatcaatcaccttgtcctgtttgacaacgcccttcgcaagtacgattccggccaggtggcggcgggcttccagagggcccttctggtggccgggccagagaccgctgacacgaggccggacctccgcaagctgaatgagtgggtgtttggtggcagggctgctggtggcagacagctggccgacgagctaaagatcgtgtccgcgctgcgagacacttactcgggccacttggtccttcagcccacggagacccttgacacatggaaggtgttgagcagggacacacgaaccgctcatagtttggagcacggattcattcatgccgcggggaccatccaggccaactgcccacagctgtttatgagacgccagcaccccggcctctttcccttcgttaatgcaatagcatcatcgctgggctggtactaccagaccgccaccggccccggagcagatgccagggcggcggcccggcgccaacaggcctttcagaccagggcggcggctgaatgccatgccaaaagcggggtgccggtcgtggccggcttctacaggaccatcaacgccacgctcaagggaggagagggcctacagcccactatgtttaacggggagctgggggccatcaagcaccaggcacttgacactgtgaggtatgactacggccactatctcataatgttggggccattccagccatggagcggactgacggcccctccgtgcccctacgccgaaagttcatgggcacaggcggccgtgcagacggccctcgagctgttctcggccctgtacccggccccgtgcatctcgggctacgcgcgccccccgggccccagtgctgtgatcgagcatctggggtccctagttccaaaggggggtctgctgttgtttctgtctcacctaccggatgatgttaaggacgggctcggagaaatggggccggccagggccacgggacctggaatgcagcagtttgtcagcagctacttcctcaaccccgcctgttccaacgtcttcattacagtgaggcagcgaggggagaagatcaacggccgtaccgtcctccaagcgctcggacgcgcatgcgatatggcaggctgccagcactatgtgctgggctccacggttcccctcggtggactcaactttgtcaacgacctggcgtccccggtttccaccgccgagatgatggatgatttctctcccttcttcaccgtggagtttcccccgattcaagaggagggcgcaagttctccggtacccttagatgtggacgagagcatggacatctctccgtcttacgagttgccctggctctcgctggagtcatgcctcacaagcatcctgtcacaccccaccgtgggaagcaaggagcacttggtcaggcacacggacagggtcagcggaggacgcgtggcacagcagcccggggtaggtcccctggacctgccgctggcggactacgccttcgttgcccacagtcaggtctggaccaggcccggtggggctcctcccttgccctatcgtacctgggatcgaatgacagagaagctgcttgtctccgcaaaacccggcggagagaacgttaaggtttcaggtaccgtgattacattgggagaacaggggtacaaagtgtcgttggatctgagggagggaaccaggctggcaatggctgaggcgctgctgaacgcagcatgtgccccaatcttggatccggaagacgtcttgctcaccctgcatctacacctggatccgcgccgggcagacaactcggccgtgatggaggctatgacggcggcgagtgactacgcgcgtggcctgggcgtgaagctgacctttggctcggcctcctgccccgagaccggctcgtccgcctccaacttcatgaccgtggtggcctctgtctccgccccaggggaattctcgggtcctctgatcacgccagtgcttcagaagacgggcagtctcctgattgcggtgcgttgcggggatggcaagatccagggagggtcgctgtttgagcagctctttagcgacgtggccacgaccccacgggcacccgaggcgttgtctctgaagaatctcttccgggcagtccagcagctggtcaagagcggcatcgtgctgtcagggcatgacatcagcgacgggggcctggtgacctgcctggtggagatggccctggccgggcagcggggagtgaccatcactatgccggtggcctccgactacctcccggagatgtttgcagagcaccccggcctggtgtttgaggtggaggagcgcagcgtgggtgaggtgctgcagaccctgcgctccatgaacatgtacccggcagtcctcggtcgagtgggcgagcaaggtccagatcaaatgtttgaggtgcagcacggcccagagacggtgttgcgccagtcgctgcgcctgctgctgggaacctggtcatcctttgccagcgagcagtacgagtgcctgcgaccagatcggattaaccggtccatgcacgtgtccgactacggctataacgaagcactggcagtctccccgttgacaggaaagaatctcagcccacgccggttggtgacagagcctgacccacgatgtcaggtggccgtgctatgcgccccgggcaccaggggccatgaaagcctcctggcggccttcacgaatgccggatgcctgtgccgacgggtgttctttcgcgaggttagggacaacacgttcctcgacaagtacgtgggtctggccatcggaggagttcatggggccagggactctgccctggcaggccgtgccaccgtggcgctgattaatcgtttccccgccctgcgtgacgctattctaaagttcctcaacaggccagatacgttctcggtggccttgggggagctgggggtgcaagttttggctggcctgggggccgtggggtcaacagataatccacccgcccctggcgtggaagttaatgtccagagatcacctctgattctggcccccaacgcctctggcatgtttgagtcccgctggctgaacattagcatcccggcgaccaccagctctgtcatgctgcgtggcctccggggctgcgtcctgccttgttgggtgcaaggctcgtgcctgggcctgcaatttactaacctcgggatgccatatgttttgcagaatgcccaccagatcgcctgccacttccacagcaatggcacggatgcctggcgctttgctatgaattatccaagaaaccccacggagcagggcaacattgcagggctctgttcacgcgatggtcgtcatctggctctcctgtgtgacccctcactttgtacagacttttggcaatgggagcacattccccccgcctttgggcaccccacggggtgctccccctggacacttatgtttcaagcagctcacctatggtcactcaggcacggtcgcccctccgagtga

SEQ ID NO: 10 - protein, 1318 aa, Homo sapiens
MEERGRETQMPVARYGGPFIMVRLFGQDGEANIQEERLYELLSDPRSALGLDPGPLIAENLLLVALRGTNNDPRPQRQERARELALVGILLGNGEQGEHLGTESALEASGNNYVYAYGPDWMARPSTWSAEIQQFLRLLGATYVLRVEMGRQFGFEVHRSRPSFRQFQAINHLVLFDNALRKYDSGQVAAGFQRALLVAGPETADTRPDLRKLNEWVFGGRAAGGRQLADELKIVSALRDTYSGHLVLQPTETLDTWKVLSRDTRTAHSLEHGFIHAAGTIQANCPQLFMRRQHPGLFPFVNAIASSLGWYYQTATGPGADARAAARRQQAFQTRAAAECHAKSGVPVVAGFYRTINATLKGGEGLQPTMFNGELGAIKHQALDTVRYDYGHYLIMLGPFQPWSGLTAPPCPYAESSWAQAAVQTALELFSALYPAPCISGYARPPGPSAVIEHLGSLVPKGGLLLFLSHLPDDVKDGLGEMGPARATGPGMQQFVSSYFLNPACSNVFITVRQRGEKINGRTVLQALGRACDMAGCQHYVLGSTVPLGGLNFVNDLASPVSTAEMMDDFSPFFTVEFPPIQEEGASSPVPLDVDESMDISPSYELPWLSLESCLTSILSHPTVGSKEHLVRHTDRVSGGRVAQQPGVGPLDLPLADYAFVAHSQVWTRPGGAPPLPYRTWDRMTEKLLVSAKPGGENVKVSGTVITLGEQGYKVSLDLREGTRLAMAEALLNAACAPILDPEDVLLTLHLHLDPRRADNSAVMEAMTAASDYARGLGVKLTFGSASCPETGSSASNFMTVVASVSAPGEFSGPLITPVLQKTGSLLIAVRCGDGKIQGGSLFEQLFSDVATTPRAPEALSLKNLFRAVQQLVKSGIVLSGHDISDGGLVTCLVEMALAGQRGVTITMPVASDYLPEMFAEHPGLVFEVEERSVGEVLQTLRSMNMYPAVLGRVGEQGPDQMFEVQHGPETVLRQSLRLLLGTWSSFASEQYECLRPDRINRSMHVSDYGYNEALAVSPLTGKNLSPRRLVTEPDPRCQVAVLCAPGTRGHESLLAAFTNAGCLCRRVFFREVRDNTFLDKYVGLAIGGVHGARDSALAGRATVALINRFPALRDAILKFLNRPDTFSVALGELGVQVLAGLGAVGSTDNPPAPGVEVNVQRSPLILAPNASGMFESRWLNISIPATTSSVMLRGLRGCVLPCWVQGSCLGLQFTNLGMPYVLQNAHQIACHFHSNGTDAWRFAMNYPRNPTEQGNIAGLCSRDGRHLALLCDPSLCTDFWQWEHIPPAFGHPTGCSPWTLMFQAAHLWSLRHGRPSE

SEQ ID NO: 11 - DNA, 513 bp, Homo sapiens
atggagcgaaggttagtggtcactctgcagtgcctggtgctgctttacctggcacctgagtgtggaggtacagaccaatgtgacaattttccccaaatgttgagggacctaagagatgccttcagtcgtgttaaaacctttttccagacaaaggacgaggtagataaccttttgctcaaggagtctctgctagaggactttaagggctaccttggatgccaggccctgtcagaaatgatccaattctacctggaggaagtcatgccacaggctgaaaaccaggaccctgaagccaaagaccatgtcaattctttgggtgaaaatctaaagaccctacggctccgcctgcgcaggtgccacaggttcctgccgtgtgagaacaagagtaaagctgtggaacagataaaaaatgcctttaacaagctgcaggaaaaaggaatttacaaagccatgagtgaatttgacatttttattaactacatagaagcatacatgacaattaaagccaggtga

SEQ ID NO: 12 - protein, 170 aa, Homo sapiens
MERRLVVTLQCLVLLYLAPECGGTDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 13 - DNA, 1152 bp, Homo sapiens
gtctgggaggcagagggtcggcctaggcccggggaagtggagggggatcgcccgggtctctgttggcagagtccgggcgatcctctgagaccctccgggcccggacggtcgccctcagccccccagacagaccccagggtctccaggcagggtccggcatcttcaggggcagcaggctcaccaccacaggccccccagacccgggtctcggccagccgagccgaccggccccgcgcctggcgcctcctcggggccagccgccggggttggttctgcccctctctctgtccttcagaggaaccagggacctcgggcaccccagagcccctcgggcccgcctccaggcgccctcctggtctccgctcccctctgagccccgttaaacccaaagaatgtctgaggggagccaccctcggggcccaggccccagagtccagaggtcaggggcacctcagggtgcctccccgggtcccaggccagccggagggaccccggcagcccgggcggccccagaggccggttcctcgccccttccccgggcttcagagcccaggatgtcccccagaagggaccctaggcgtcccctctcctcccctccaggcccgagcctctccctcgcggagaggggcctctttgggccctcaagtccagccccaccgagacccgagtggcccggatccccccaccggcccttctctctgtccccctgctcctctccaaccttcgctccaccctagaccccagcttctggcctccccgggtccaccaggccagccggagggaccccggcagcccgggcgagtcgccttccctctcccctggcctctccttcccgcctcccacccgagccccctcagcttgcctccccaccgggtccatcaggccggccggagggaccccggcggcccggtgtcagtcccccctgcagccgcccagtctctgcctccaggcaagggcgccagcttttctccccccagcctgaggcccagtctcctgtgcactgtctgtaaagtccagcctcccacgcccgtccacggctcccgggcccagcctcgtccacccctccccacggtggacaggccctctgtccacccgggccatccccgcccccctgtgtccaccccagtcccgtccaggggggactttatgtga

SEQ ID NO: 14 - protein, 383 aa, Homo sapiens
VWEAEGRPRPGEVEGDRPGLCWQSPGDPLRPSGPGRSPSAPQTDPRVSRQGPASSGAAGSPPQAPQTRVSASRADRPRAWRLLGASRRGWFCPSLCPSEEPGTSGTPEPLGPASRRPPGLRSPLSPVKPKECLRGATLGAQAPESRGQGHLRVPPRVPGQPEGPRQPGRPQRPVPRPFPGLQSPGCPPEGTLGVPSPPLQARASPSRRGASLGPQVQPHRDPSGPDPPTGPSLCPPAPLQPSLHPRPQLLASPGPPGQPEGPRQPGRVAFPLPWPLLPASHPSPLSLPPHRVHQAGRRDPGGPVSVPPAAAQSLPPGKGASFSPPSLRPSLLCTVCKVQPPTPVHGSRAQPRPPLPTVDRPSVHPGHPRPPVSTPVPSRGDFM

SEQ ID NO: 15 - DNA, 1152 bp, Homo sapiens; identical to SEQ ID NO: 13

SEQ ID NO: 16 - protein, 383 aa, Homo sapiens; identical to SEQ ID NO: 14

SEQ ID NO: 17 - DNA, 168 bp, Homo sapiens
gtaacccagcactggcgtgtgacgtggtgtaaagttttgcctgaacctgtggttgggcaggtacatgccaacaaccttctaagcacccgcgcttgtgttttgctttatctgccgccatcatgcctacattctatcttgcgttacatgggggacaaacatatcatctaa

SEQ ID NO: 18 - protein, 55 aa, Homo sapiens
VTQHWRVTWCKVLPEPVVGQVHANNLLSTRACVLLYLPPSCLHSILRYMGDKHII

SEQ ID NO: 19 - DNA, 1464 bp, Homo sapiens
atgcctacattctatcttgcgttacatgggggacaaacatatcatctaattgttgacacggatagtcttggaaacccgtcactctcagtaattccctcgaatccctaccaggaacaactgtcagacactccattaattccactaacaatctttgttggggaaaacacgggggtgcccccaccactcccaccaccccccccaccaccacccccaccacccccaccacccccaccacccccaccacccccaccacctccaccaccttcaccaccacccccgcccccaccacccccaccacctcagcgcagggatgcctggacacaagagccatcacctcttgatagggatccgctaggatatgacgtcgggcatggacctctagcatctgctatgcgaatgctttggatggctaattatattgtaagacaatcacggggtgaccggggccttattttgccacaaggcccacaaacagcccctcaggccaggttggtccagccacatgtcccccctctacgcccgacagcacccaccattttgtcacctctgtcacaaccgaggcttacccctccacaaccactcatgatgccaccaaggcctacccctcctacccctctgccacctgcaacactaacggtgccaccaaggcctacccgtcctaccactctgccacccacaccactactcacggtactacaaaggcctaccgaacttcaacccacaccatcaccaccacgcatgcatctccctgtcttgcatgtgccagaccaatcaatgcaccctcttactcatcaaagcaccccaaatgatccagatagtccagaaccacggtccccgactgtattttataacattccacctatgccattacccccctcacaattgccaccaccagcagcaccagcacagccacctccaggggtcatcaacgaccaacaattacatcatctaccctcggggccaccatggtggccacccatctgcgaccccccgcaaccctctaagactcaaggccagagccggggacagagcagggggaggggcaggggcaggggcaggggcaggggcaagggcaagtccagggacaagcaacgcaagcccggtggaccttggagaccagagccaaacacctccagtcctagcatgcctgaactaagtccagtcctcggtcttcatcagggacaaggggctggggactcaccaactcctggcccatccaatgccgcccccgtttgtagaaattcacacacggcaacccctaacgtttcaccaatacatgaaccggagtcccataatagcccagaggctcccattctcttccccgatgattggtatcctccatctatagaccccgcagacttagacgaaagttgggattacatttttgagacaacagaatctcctagctcagatgaagattatgtggagggacccagtaaaagacctcgcccctccatccagtaa

SEQ ID NO: 20 - protein, 487 aa, Homo sapiens
MPTFYLALHGGQTYHLIVDTDSLGNPSLSVIPSNPYQEQLSDTPLIPLTIFVGENTGVPPPLPPPPPPPPPPPPPPPPPPPPPPPPPPSPPPPPPPPPPPQRRDAWTQEPSPLDRDPLGYDVGHGPLASAMRMLWMANYIVRQSRGDRGLILPQGPQTAPQARLVQPHVPPLRPTAPTILSPLSQPRLTPPQPLMMPPRPTPPTPLPPATLTVPPRPTRPTTLPPTPLLTVLQRPTELQPTPSPPRMHLPVLHVPDQSMHPLTHQSTPNDPDSPEPRSPTVFYNIPPMPLPPSQLPPPAAPAQPPPGVINDQQLHHLPSGPPWWPPICDPPQPSKTQGQSRGQSRGRGRGRGRGRGKGKSRDKQRKPGGPWRPEPNTSSPSMPELSPVLGLHQGQGAGDSPTPGPSNAAPVCRNSHTATPNVSPIHEPESHNSPEAPILFPDDWYPPSIDPADLDESWDYIFETTESPSSDEDYVEGPSKRPRPSIQ

SEQ ID NO: 21 - DNA, 957 bp, Homo sapiens
atggccccggtcaccccagatgccgtgaatgcacgccaacagcgaccggcagatcccgcattgcgccgcctaatgcatccgcaccacagaaactacacggcctcaaaggcctcggcgcatagcgtgaagtcggtgtccaggtgtggaaaatctcgctcagagctgggaagaatggaaagggttggcagtgtggcccgatcaatatgttcccggcacaccagacatggtgtagacagatcccatttttcactacgggacttcttcaggggaatctctgccaactttgagctgggcaaagattttctgcgtgagatgaacacccccatacatgtctcagaggccgtgtttctcccactgtcactgtgcactctctcccccgggcgctgccttcgcctgtctcccttcggccacagcctgactctggggtctcactgcgagatctgcatcaataggtcccaggttcatgtgcctcaggagtttagctccacccagctctccttcttcaacaatgtccacaagataatacccaacaagaccttctatgtgtctctgctcagcagctctcccagtgcagtaaaggctggactttcccaacccagccttctctacgcttacctggtcaccggacacttttgtggcaccatctgccccatcttcagcacaaatggaaaagggcgcctaatcatgcatctcctgctccagggcacctcccttcacatcccagagacctgcttgaaactgttatgtgaaaacataggccccacctacgagctggccgtggacctagtaggggacgccttctgtataaaggtcagccccagagacacggtatatgagaaggctgtcaatgtcgacgaagatgccatctacgaggccatcaaggacctggaatgtggggatgagctgcgcctacagatcatcaactatacccagctcattttggaaaataaacagtag

SEQ ID NO: 22 - protein, 318 aa, Homo sapiens
MAPVTPDAVNARQQRPADPALRRLMHPHHRNYTASKASAHSVKSVSRCGKSRSELGRMERVGSVARSICSRHTRHGVDRSHFSLRDFFRGISANFELGKDFLREMNTPIHVSEAVFLPLSLCTLSPGRCLRLSPFGHSLTLGSHCEICINRSQVHVPQEFSSTQLSFFNNVHKIIPNKTFYVSLLSSSPSAVKAGLSQPSLLYAYLVTGHFCGTICPIFSTNGKGRLIMHLLLQGTSLHIPETCLKLLCENIGPTYELAVDLVGDAFCIKVSPRDTVYEKAVNVDEDAIYEAIKDLECGDELRLQIINYTQLILENKQ

SEQ ID NO: 23 - DNA, 1578 bp, Homo sapiens
atggctcacaaagtcacgagcgcaaacgagccaaaccccctgaccggaaaaagactgtccagctgccccctcacgaggagtggcgtaacagaggtggcacagatcgcgggcagaactccaaagatggaggactttgtgccctggaccgtggacaatctgaaaagccagttcgaggcagtcggccttttgatggcccactcctatcttccagccaacgcggaagaaggcatagcctacccaccactcgtccacacctacgagtccctctctccagccagcacctgccgagtttgtgatctgctcgacaccctggtcaatcactcagatgcccccgtggccttctttgaggactatgccctgctctgctactactgtctaaatgccccacgagcctggatctctagcctcatcacaggcatggactttttacacatcctgataaaatacttccccatggccggggggttggattccctcttcatgccctccaggattcttgccattgacatccagcttcacttttacatttgccggtgcttcctccctgtgtcctcttctgacatgattagaaatgccaacctcggctactacaaattggagtttctgaaatccatcctcaccggccagtccccggccaacttctgcttcaagtccatgtggcccagaaccacccccacttttcttaccctccctggacccagaacctgcaaagattcccaggatgtcccaggggacgtcggcaggggcctgtacaccgccctgtgctgccacctgcctaccagaaaccgagttcagcatccatttctcagggccgaaaagggaggactctctcctgaaattacaaccaaggccgactactgtggactactattaggtacgtggcaggggacggacctgctcggggggccaggccaccatgctatcggtttaaacgcggaatacagcggcgatgagctggcggagttggccctggccatcaccagaccggaggctggcgaccattcgcagggcccctgcctcctggcccccatgtttggactaaggcacaagaatgcctcacggaccatctgcccactctgcgagagcctcggggcccacccggacgccaaggacacccttgaccgcttcaagagcctgattcttgactcgttcggcaacaacatcaaaatcctagacaggatcgtgttcctcatcaagacccaaaacacgctgctggatgtgccatgccccaggctaagggcctggctgcagatgtgcaccccgcaagacttccacaagcacctgttctgcgaccccctgtgtgccatcaaccacagcatcacgaatccgagcgtgctgtttggtcagatttacccacccagcttccaggcctttaaggcagcgcttgcagccggtcagaatctggaacagggggtctgtgactcgttaattacgcttgtgtacatatttaaatccacacaagtggccagagtgggcaaaacaatcctcgtggatgtcactaaggaactggacgtggtcctgcgcatccacggccttgacctggtacagtcctatcaaacttcccaggtctacgtgtga

SEQ ID NO: 24 - protein, 525 aa, Homo sapiens
MAHKVTSANEPNPLTGKRLSSCPLTRSGVTEVAQIAGRTPKMEDFVPWTVDNLKSQFEAVGLLMAHSYLPANAEEGIAYPPLVHTYESLSPASTCRVCDLLDTLVNHSDAPVAFFEDYALLCYYCLNAPRAWISSLITGMDFLHILIKYFPMAGGLDSLFMPSRILAIDIQLHFYICRCFLPVSSSDMIRNANLGYYKLEFLKSILTGQSPANFCFKSMWPRTTPTFLTLPGPRTCKDSQDVPGDVGRGLYTALCCHLPTRNRVQHPFLRAEKGGLSPEITTKADYCGLLLGTWQGTDLLGGPGHHAIGLNAEYSGDELAELALAITRPEAGDHSQGPCLLAPMFGLRHKNASRTICPLCESLGAHPDAKDTLDRFKSLILDSFGNNIKILDRIVFLIKTQNTLLDVPCPRLRAWLQMCTPQDFHKHLFCDPLCAINHSITNPSVLFGQIYPPSFQAFKAALAAGQNLEQGVCDSLITLVYIFKSTQVARVGKTILVDVTKELDVVLRIHGLDLVQSYQTSQVYV

SEQ ID NO: 25 - DNA, 1011 bp, Homo sapiens
atggcgagcccggaagagaggctcctagacgagctcaataacgtaattgtgtcatttctgtgtgactctgggtctctggaagtggagagatgctccggggcgcatgtgttctccaggggcagctcccaacccctctgcaccgtgaagctgcgccacggacagatttaccacctggagtttgtctacaagttcctggcctttaagctgaagaactgcaactacccctcctcgcccgtgtttgtgatatccaacaacggcctggccaccaccctgaggtgctttttgcacgagccgtcgggtctcagatcgggccagagcggcccttgcctgggtctctcaacggatgttgacctaccaaagaactccatcattatgctgggccaggatgacttcattaagttcaaaagccccctggtcttccctgctgagcttgatctcctgaaatctatggtggtctgccgggcctacatcacggaacaccggacgacgatgcagtttctggtgtttcaggccgccaacgcccagaaggcctcgcgggtcatggatatgattagtgatatgtctcagcaactgtctcggtctggtcaagtcgaggatacgggcgccagagtcacaggtggaggaggtcccaggcctggcgtcacgcactcggggtgtcttggggactcacacgttagggggcgcggtggttgggacttggataacttttcagaagctgagaccgaagacgaggcgagttacgctccttggagggacaaagactcgtggtcggaatccgaggcggcgccgtggaagaaggaactcgtgaggcaccccatccgcaggcaccggacacgcgagactcgccgtatgcgcgggagccattcacgggtggaacacgtgccccccgagacccgggagacggtggtggggggagcatggcgttattcttggcgcgccacaccttatctggcacgggtgctggctgtcacggccgtggccctgctcctgatgtttctgaggtggacctga

SEQ ID NO: 26 - protein, 336 aa, Homo sapiens
MASPEERLLDELNNVIVSFLCDSGSLEVERCSGAHVFSRGSSQPLCTVKLRHGQIYHLEFVYKFLAFKLKNCNYPSSPVFVISNNGLATTLRCFLHEPSGLRSGQSGPCLGLSTDVDLPKNSIIMLGQDDFIKFKSPLVFPAELDLLKSMVVCRAYITEHRTTMQFLVFQAANAQKASRVMDMISDMSQQLSRSGQVEDTGARVTGGGGPRPGVTHSGCLGDSHVRGRGGWDLDNFSEAETEDEASYAPWRDKDSWSESEAAPWKKELVRHPIRRHRTRETRRMRGSHSRVEHVPPETRETVVGGAWRYSWRATPYLARVLAVTAVALLLMFLRWT

SEQ ID NO: 27 - DNA, 1776 bp, Homo sapiens
atggcgttattcttggcgcgccacaccttatctggcacgggtgctggctgtcacggccgtggccctgctcctgatgtttctgaggtggacctgacgttgcaggcccttggggagcgggggttctccaggctcctggatctggggctggcctgcctggatctgagctatgtggaaatgagggaatttgtggtttggggcaggcccccagcttctgaggcggctgtggcctctacgccaggctcgcttttccgaagccactcgtccgcctactggttgtcggaggtggagaggcccgggggccttgtccgctgggccaggtcacagaccagcccctcatccctgaccctcgcgccccatcttggcccgtccctcttgtccctttcagtggtcaccggtggtgggtgtggagccgtggccttttgcaacgcctttttcctagcttattttttggttgtgcggtctgttttccccgcgttttccgatagaatagctgcctggatctgcgaccggtcccctttctgcgaaaacacccgggccgtggccaggggttaccgaggcctcgtgaagaggttcttggcattcgtgtttgagcgtagtagctatgacccccccttgttgaggcaaaactctaggcctgtggagcgctgctttgccatcaagaattatgtcccgggcctggactcacaaagctgtgtgacggtcccgagcttctcccgctgggcccagtctcacgccagcgagctcgatccccgggagattcgcgacagagttacaccagcgactgcaccttcgttcgtggctgatcatgcctcggctctattggcctccctccagaagaaggcctccgacaccccctgtgggaatcccattcagtggatgtggtaccgcctgttggtaaactcgtgcctgaggagtgcccactgtcttctgcctatacctgccgtctctgagggggggagaaagacgggcgggggcgtaggggaggagctcgtgggggccggggggccctgcctgagccgggatgttttcgtggcgatcgtaagccgcaatgttctctcgtgtctgctgaacgtgcctgccgcgggtccccgggcctacaagtgtttcagatcccacgcctccagaccggtgtctggcccggattaccctcccttggccgtgttttgcatggactgcggttactgcttgaactttggaaagcagacaggtgtaggaggcaggctcaattcctttagacccactctccagttttatccccgtgaccagaaggagaagcatgtgctgacctgccatgccagcggccgtgtgtactgctccaactgcggctctgcggcggtgggctgccagaggctggctgagccaccgagcgcccgctcgggctggcggccccgaatccgggcagtgctgccgcacaacgcggcctacgagctcgaccgtggctcccgcctcttggatgccatcatcccctgcttgggacccgaccgcacttgcatgcggccggtggtcctgcggggggtgacggtcaggcagctcctgtatttaactttgcggacagaggccagagccgtttgctccatctgtcagcaacgccaagctccagaggacgcccgcgacgagcctcacctgttctcctcctgtttagaggtagaattgccacctggtgagcggtgtgcgggctgccgtctctatcagacgcgttatggcacgccggctgcccaagcccaccctccaggggaggctggaggcggattttccagacagtcccctgcttcctaa

SEQ ID NO: 28 - protein, 591 aa, Homo sapiens
MALFLARHTLSGTGAGCHGRGPAPDVSEVDLTLQALGERGFSRLLDLGLACLDLSYVEMREFVVWGRPPASEAAVASTPGSLFRSHSSAYWLSEVERPGGLVRWARSQTSPSSLTLAPHLGPSLLSLSVVTGGGCGAVAFCNAFFLAYFLVVRSVFPAFSDRIAAWICDRSPFCENTRAVARGYRGLVKRFLAFVFERSSYDPPLLRQNSRPVERCFAIKNYVPGLDSQSCVTVPSFSRWAQSHASELDPREIRDRVTPATAPSFVADHASALLASLQKKASDTPCGNPIQWMWYRLLVNSCLRSAHCLLPIPAVSEGGRKTGGGVGEELVGAGGPCLSRDVFVAIVSRNVLSCLLNVPAAGPRAYKCFRSHASRPVSGPDYPPLAVFCMDCGYCLNFGKQTGVGGRLNSFRPTLQFYPRDQKEKHVLTCHASGRVYCSNCGSAAVGCQRLAEPPSARSGWRPRIRAVLPHNAAYELDRGSRLLDAIIPCLGPDRTCMRPVVLRGVTVRQLLYLTLRTEARAVCSICQQRQAPEDARDEPHLFSSCLEVELPPGERCAGCRLYQTRYGTPAAQAHPPGEAGGGFSRQSPAS

SEQ ID NO: 29 - DNA, 531 bp, Homo sapiens
atggcacgccggctgcccaagcccaccctccaggggaggctggaggcggattttccagacagtcccctgcttcctaaatttcaagagctgaaccagaataatctccccaatgatgtttttcgggaggctcaaagaagttacctggtatttctgacatcccagttctgctacgaagagtacgtgcagaggacttttggggtgcctcggcgccaacgcgccatagacaagaggcagagagccagtgtggctggggctggtgctcatgcacaccttggcgggtcatccgccacccccgtccagcaggctcaggccgccgcatccgctgggaccggggccttggcatcatcagcgccgtccacggccgtagcccagtccgcgaccccctctgtttcttcatctattagcagcctccgggccgcgacttcgggggcgactgccgccgcctccgccgccgcagccgtcgataccgggtcaggtggcgggggacaaccccacgacaccgccccacgcggggcacgtaagaaacagtag

SEQ ID NO: 30 - protein, 176 aa, Homo sapiens
MARRLPKPTLQGRLEADFPDSPLLPKFQELNQNNLPNDVFREAQRSYLVFLTSQFCYEEYVQRTFGVPRRQRAIDKRQRASVAGAGAHAHLGGSSATPVQQAQAAASAGTGALASSAPSTAVAQSATPSVSSSISSLRAATSGATAAASAAAAVDTGSGGGGQPHDTAPRGARKKQ

SEQ ID NO: 31 - DNA, 9450 bp, Homo sapiens
atgagtaacggcgactgggggcaaagccagcgcacccggggaaccggccccgtgcgcggaatcaggaccatggatgtgaatgcccccgggggcgggagtggaggctcggccctccgcatcctaggcacggcctcgtgcaaccaggcccactgcaagtttggccgctttgccggcatccagtgcgtcagcaactgcgtcctctacctggtcaagagcttcctggccggccgccccctgacctcccgccctgagctggacgaggtcctggacgagggggcgcggctggatgccctcatgcgccagagcggcatcctcaaggggcacgagatggcccagttgacggacgtgcccagctccgtggtcctgaggggcggtgggcgcgtgcacatataccgctcggcggagatctttggcctcgtcctattccctgcccagatcgcaaactcggcagttgttcagtccctggccgaggtcctgcacggcagttacaacggggtggcccagttcatcctctacatctgcgacatctacgcgggggccatcatcatcgagacagatggctccttctacttgtttgacccccactgccagaaggatgcggccccgggcacccccgcccatgtcagagtcagcacctatgcgcacgacatcctgcagtacgtgggggcaccaggggcccagtacacttgcgtgcatctctattttctacccgaggcctttgagacagaggacccccgcatctttatgctggaacactatggcgtgtatgacttttatgaggccaacggcagcggctttgacctggtggggcccgagcttgtgtccagtgatggggaggcggcggggacgcccggtgctgatagcagccctcccgtcatgctaccgtttgaacggcgaataataccgtataacctcaggccactgccctctcgatctttcacatccgactcattccctgccgccaggtatagtcctgcaaagaccaactccccgccgtcgtctccggcctctgcggccccggcctctgcggccccggcctctgcggccccggcctccgcggccccggcctccgcggccccggcctccgcggccccggcctccgcggccccggcctccgcggccccggcctccagccctcccctcttcatccccatccccggcctaggtcacacccccggggtccccgctccctccacgccaccaagggcgtcgagcggcgcagcgcctcagactcccaagaggaaaaagggattgggcaaagatagcccccacaagaagcccacctccggccgccgcctccctctgtcctccaccactgacacagaggatgatcagctgccacgcacccacgttccgccccatcgccccccttcagcagctcgccttccccctccagtaatccccatccctcaccaatcaccacccgcctccccgacaccccaccccgcgccagtctcaactatcgctccttcagtcactccatctcctcgcctccctttgcaaatacccatccctctgccacaggcagctccctcgaacccaaaaatccctctgaccaccccatccccttccccgactgcagcagcagcccccaccaccacgactctttctcctcctccaactcagcagcagcccccgcaatcagccgccccggctccttctccacttcttcctcagcagcagcccacgccatcagccgccccggctccttctccacttcttcctcagcagcagcccccgccatcagccgcccgggctccttctcctcttcctcctcagcagcagcccctgccatcagctaccccggctcctcctcctgctcagcagctcccaccatcagccacgacactcgaaccagaaaaaaaccatcccccggccgctgacagggctggaacggagatatccccatcacctcccttcgggcagcagccctcctttggggatgatgcatctgggggttctggcctcgtccgctacctgtctgacctagaggagcctttcctatccatgtccgactctgaagaagcggagagcgacttagcctccgatatccccaccacagaggatgaagacatgttcgaagatgaagtgttctccaacagcctggaatcgggatccagtgcccccacgtcccccatcacactggacaccgccaggagccaatactatcagaccacctttgacatagagactcctgagatggactttgtgcccctcgagagcaacattgcgagaatagccggacatacctaccaggaacaggccatcgtctacgacccggcctctaaccgggaagtgccagaggcagatgccttgagcatgatagactatcttcttgtgacagtggtcctcgagcaggggctaataaggagtagggaccgatcgtctgtgctaaatctgctggaattcttaaaggactggtcgggacatctgcaggtccccacgctggacctggagcaactgctcacttccgaacttaacatccaaaacctcgcaaacatgctatcagaaaacaagggaagagcgggagagttccataagcacctggccgcaaagctggaggcctgcctgccgtccctggccacgaaggacgcggtccgtgtagatgccggtgctaagatgctggctgaaattccccagctggccgagagcgacgatggaaaatttgacctggaggcggcccgcagacgcctgaccgacctgctctcgggcggagatcaggaggcgggggaaggaggaggagaacccgaggataattccatctacaggggcccgcacgtggacgtccccttggtcctggacgacgaaagctggaagcgcctcctatccctggcggaggccgcccgaacggccgtggccagacaacaggctggggtagatgaggaggacgtccgctttttggcgctgttgacagcgatagagtatggggcccctcccgccgcctcagtgcccccctttgtccacaacgtggccgtccggtccaagaatgcggccctgcacgtgcgcaggtgtacggcggacatcagggacaaggtggcctcggcggcctccgattatctctcctaccttgaggacccaagcctcccaacagtgatggattttgacgacctcctgacacacctgcggcacacatgccagatcatcgcctccctgcctctcctaaacatccggtacacgtccattgaatgggactatcgcgagctcctctacctgggcaccgcccttagtgatatgagcggtatcccctggcccctggagcgagttgaggaggacgaccccagcattgccccccttccagagtttgagacagtcgcgaaaaaacagaaggagctcgagaccaccagagaaaacgaaaagcgcctccgcaccatcctggacgacatcgaggcaatgttgggcctggccggtgtggcctcggccccaggcgcccccatctctcccgcctctccctctgcgacacccgccaaccacgacaaccccgaagccacgccaccgctggccgacaccgcggccctcaccatccccgtcatagaaaagtacatcgcaaatgcagggtcgatagtcggggccgcaaagaaccccacgtacatccgcctacgtgacactatacagcagattgttcgctccaaaaagtacctgatgaacatcctgaaatccatcaccttctacaccatcgataactacattgcctccttcgaggagagcatagaccacctctatcgtgacttgccggtccttgaccctgaggtgcaggatggcattgaccgcatcctggaccccatggtctcagaggccctgcacacattcgagatgggcaaccggctgacgctggagccagcgcgcctagtggccttgcagaattttgctactcacagcaccttaaaggagacggccgcggcggtgaatctgctcccaggtctactggcggtctatgatgcgacgatcacaggtcaggctccagaggatgccctgagactactctctgggctgcagaatcagctctcccaaacgctcatcccagggaagctcaagaaacgcttcctaagttacctgcagaaattgaagaataacaacaacgatcagctcagacagaaggaggtgcaggcctggcgcctggaggccgaaggctttaagcccgccaccgaggaacagctcgaggcctttctggacactgcccccaacaaagagctcaagcgacagtatgagaagaagctgcggcagcttatggagaccgggcgcaaggaaaaggagaagctccgagaacaggaagacaaggagaggcaggagcggcgagcgcgagaggcgaatgaggcctgggccagaattaggaaggcgctgggggcacgcccagagcccgctcccacttcgcccgatgactggaacaccctgctggcctccctgctgccagacaatacggactcagccgcggcagcagcggcggcagtagccagaaacacggacatcttggactccctcacacagatccttgccgctatgctattgggaataacgagggtgaggagggagagactgcgatccctgctcgtggacgatggtggtgcggcggagagaatggaggccgcggaacccggctggttcacggatatcgagaccggccctctggccagactggacgcttggcccgccacccccgcggctaccgccaaagaaggaggaggagggcgaggagcggaggaagcggccggggccctctttcgcgccaggacggcggccgatgccatccgttcggccctcgcgcagacgcgccaggccctgcagtccccggacatgaaatcagcggtggtcaacacggatctggaggccccctacgcggagtacgagcgggggctggccgggcttctggaaaaaagacgagcagccgaggctgccctgacggccatcgtgagcgagtacgtggatcggacgctacccgaagccactaatgacccaggccaggctaacctgcctcctcctccaactatcccccaggcaaccgccccgcccaggctggcctcggactcggcgctctggcctaagaagccccagctgctgacaaggcgagagcgggacgatctcctccaggccacgggggacttcttctcggagctgctgaccgaggccgaggcggccgaggtccgggcgctggaagagcaggtccgggagagccagaccctgatggcgaaggcccacgagatggcggcaagcactcggcggggctttcacacggctctggaggccgtcctctccaggtcacgcgacgaagcccccgacgatgaactccggagcctgcttccctccccgcccaaagcccctgtccaggctcccctcgaggccgccctggcccgggcagcggccgggaacggctcatggccctaccggaaatccctggcagccgccaagtggatccggggcatctgcgaggccgtgcggggtctttccgaaggagccctggccttagctgggggcgcgggtgcctggctgaacctagccgctgcggctgacggtgaaattcatgagctaacacggctcctggaagtcgagggcatggcccagaactccatggatggcatggaggagctgaggttggctctggccacgttggaccccaagcgggtcgcggggggcaaggagaccgtcgcagactggaaaaggcgcctctcccgactggaggccatcatccaggaggcccaggaagagtcccagctgcagggaacgctgcaggatctggtcacccaggccaggggccacaccgacccgcgccagctcaagatcgtggtggaggctgccaggggcctggcgcttggggcctcggctggctcccagtatgccctcctcaaggacaagctgctgcgctatgcctcggccaagcagagtttcctggctttttacgagaccgcccagcctaccgttttcgttaagcatcccttgaccaacaacctgcccctcctcatcacgatttcggcaccaccaactggatggggcaatggagccccgacccggcgggcacaatttctggcggcggcagggccggcgaaatacgccggcaccctctggctggaaacagagtctccctgcgacccccttaacccggcctacgtctccgcagacacccaggagccgctcaactacatccccgtctaccacaactttctggaatatgtcatgcccaccgtgctggagaacccagaggccttttccctgaccccagccggacgcccccaagctattggtcccccgcaggatgaccaagagaggaggagaagaaccctggccagtgtagcttccgcccgcctcagcgccgccgccgccgactcctactgggacacctggcccgacgtggagtccaacgccggggagctgcttcgggaatacgtctctgcgcccaaagccctgatggaagacctggcagacaaccccattgtggccatgacacttttagcgcacgccagtctcatcgcctctaggaatcaccccccttatccagccccggccacagaccgggaagttattctattggaacaacgggagatgatggcgctgctggtcggcacacatccggcctacgctgccgccttcctgggcgccccatcgttctacgcaggactcggcctggtctcggctctagcgagagatgggggtttgggagacctcctgtcggactcggtcctcacctacaggctggtcaggagtccagcctccgggagggggggcatgccctcaaccacccgcggcagcaacgacggggaagacgcgcgccgcctcacccgccaccgcatcgccggtccacccaccggcttcatcttcttccaggacgcctgggaggagatggatacccgagccgccctctggccgcaccccgagttcttgggcctggtccacaaccaaagtacggcccgcgcccgagcctgcatgctgctcctcgcacgccggtgcttcgcccccgaggcccttcagcagctctggcactcattgcgcccgctggagggcccggtcgcatttcaggactacctgcgcgactttgtgaagcaggcctatacacgcggagaagagctccccagggcggagggactcgaggtcccccgcgaaaccccgtcttcctacggcaccgttaccggccgggccctgagaaatcttatgccttatggcactcccattaccggtcctaagaggggctccggtgacaccatccccgtctccgtttttgaggcggccgtggcggcggccttcctcggtcggcccctaactctctttgtctcctctcaatatctgtttaacctgaagaccctgggacaggtcagggtcgttgctcccctcctctactgcgacgggcactctgagcccttccggtccctggtggagaccatctcgctgaattttctgcaggacctggacggctactccgagtctttcgagccagagatgtccatctttgcacgccaggccgtgtggctacgcgagcttctcaccgaggccagggccgccaaacctaaggaggccaggccccccaccgtggccatcctggccaacagaaaaaacatcatctggaagtgtttcacctatcggcacaatctgccggacgtgcagttctactttaacgcggccggggcctcacgatggccgaccgatgtactcaacccctccttctacgagcacgaagatcctccactgccggttggataccagcttcccccgaatccacgcaacgtccaggagctgttctccggtttccccccaagggtggggcacggactggtcagcggggacgggtttcagtcagcagataacaccccggcctcctccgaccggctccagcagttgggaggaggggagacggaccagggggaaaaggggagcactactgctgaatctgaggcctctggccctcccagcccacagagccccctcttagagaaggtggcccctggcaggcccagggactggctgtctcctacctcctccccccgggacgtgacagttaccccgggattggccgcccccatcactctcccaggcccccgattgatggcaaggccctatttcggggccgaaacgagggcttccgagagcccagaccggtctccgggaagctccccaaggccatggcccaaagattccctggagctccttccccaaccagcaccacaacagcccccctcaagtccctgggcttctgaacaagggcccatcgtctacacattgtctccacactctacaccatcgactgcctcggggtcacagaagaaacataccatccagattccagggttggtgccttcacagaagcccagttatccaccctcagccccctacaagcccggccagtcgacgggaggcatagctcccacaccatcagcggcatctttaacgacttttgggctccagccccaggacacacaagcctcctctcaggacccaccctatggccattctatcatgcaacgggagaagaaacagcaggggggccgggaagaagccgcggagattcggccctccgccacccgcctccccacggccgtcgggctgcgaccccgtgcgcccgtggtggccgccggcgcagcagcctccgctactccagcgttcgacccgggagaagcgccgtccggctttcccatcccccaggcacccgccctggggtccggcttggccgccccggcgcacaccccagttggtgcattggcaccgcgcccccaaaaaacgcaagcacaaaggccccaagatgcagctgccctgcccacccccacaattaaagcggtgggtgccaggcccgtgccaaaggccacgggggcgctggcggccggcgcccgtcctcgggggcagcccaccgcggccccgccgtcggctgcatctccgccgcgcgtgtctctccctgtcaggagcagacagcaacaatcccccgcaatcccattgcccccaatgcattccggctcggagcctggcgcgcggcccgaggtgcgcctatcccagtaccggcatgcgggtccccaaacatacaccgtgcgaaaagaagcacccccatccgccgcctcccagctgcccaaaatgccaaaatgtaaggatagcatgtattacccgccatccggtagcgcacgctatcccgcgccttttcaagcactttcgttttcgcaaagcgtggcctcacccgcgccatctagcgaccagacaacattactgtggaacactccctcagtggtcacccaatttctgtctattgaagacattatcagggaggtggtgacaggagggtcgacctccggcgacctagtggtcccctccggatccccctcctctctatccaccgccgccccggaacaggacctgcgttatagcctcacccttagtcaggctagccgagtactgtctcgcttcgtgagccagcttcgccgcaagctggagcgttccacccaccgcctcatcgcagacctcgagagactcaagtttttgtatctgtaa

SEQ ID NO: 32 - protein, 3149 aa, Homo sapiens
MSNGDWGQSQRTRGTGPVRGIRTMDVNAPGGGSGGSALRILGTASCNQAHCKFGRFAGIQCVSNCVLYLVKSFLAGRPLTSRPELDEVLDEGARLDALMRQSGILKGHEMAQLTDVPSSVVLRGGGRVHIYRSAEIFGLVLFPAQIANSAVVQSLAEVLHGSYNGVAQFILYICDIYAGAIIIETDGSFYLFDPHCQKDAAPGTPAHVRVSTYAHDILQYVGAPGAQYTCVHLYFLPEAFETEDPRIFMLEHYGVYDFYEANGSGFDLVGPELVSSDGEAAGTPGADSSPPVMLPFERRIIPYNLRPLPSRSFTSDSFPAARYSPAKTNSPPSSPASAAPASAAPASAAPASAAPASAAPASAAPASAAPASAAPASSPPLFIPIPGLGHTPGVPAPSTPPRASSGAAPQTPKRKKGLGKDSPHKKPTSGRRLPLSSTTDTEDDQLPRTHVPPHRPPSAARLPPPVIPIPHQSPPASPTPHPAPVSTIAPSVTPSPRLPLQIPIPLPQAAPSNPKIPLTTPSPSPTAAAAPTTTTLSPPPTQQQPPQSAAPAPSPLLPQQQPTPSAAPAPSPLLPQQQPPPSAARAPSPLPPQQQPLPSATPAPPPAQQLPPSATTLEPEKNHPPAADRAGTEISPSPPFGQQPSFGDDASGGSGLVRYLSDLEEPFLSMSDSEEAESDLASDIPTTEDEDMFEDEVFSNSLESGSSAPTSPITLDTARSQYYQTTFDIETPEMDFVPLESNIARIAGHTYQEQAIVYDPASNREVPEADALSMIDYLLVTVVLEQGLIRSRDRSSVLNLLEFLKDWSGHLQVPTLDLEQLLTSELNIQNLANMLSENKGRAGEFHKHLAAKLEACLPSLATKDAVRVDAGAKMLAEIPQLAESDDGKFDLEAARRRLTDLLSGGDQEAGEGGGEPEDNSIYRGPHVDVPLVLDDESWKRLLSLAEAARTAVARQQAGVDEEDVRFLALLTAIEYGAPPAASVPPFVHNVAVRSKNAALHVRRCTADIRDKVASAASDYLSYLEDPSLPTVMDFDDLLTHLRHTCQIIASLPLLNIRYTSIEWDYRELLYLGTALSDMSGIPWPLERVEEDDPSIAPLPEFETVAKKQKELETTRENEKRLRTILDDIEAMLGLAGVASAPGAPISPASPSATPANHDNPEATPPLADTAALTIPVIEKYIANAGSIVGAAKNPTYIRLRDTIQQIVRSKKYLMNILKSITFYTIDNYIASFEESIDHLYRDLPVLDPEVQDGIDRILDPMVSEALHTFEMGNRLTLEPARLVALQNFATHSTLKETAAAVNLLPGLLAVYDATITGQAPEDALRLLSGLQNQLSQTLIPGKLKKRFLSYLQKLKNNNNDQLRQKEVQAWRLEAEGFKPATEEQLEAFLDTAPNKELKRQYEKKLRQLMETGRKEKEKLREQEDKERQERRAREANEAWARIRKALGARPEPAPTSPDDWNTLLASLLPDNTDSAAAAAAAVARNTDILDSLTQILAAMLLGITRVRRERLRSLLVDDGGAAERMEAAEPGWFTDIETGPLARLDAWPATPAATAKEGGGGRGAEEAAGALFRARTAADAIRSALAQTRQALQSPDMKSAVVNTDLEAPYAEYERGLAGLLEKRRAAEAALTAIVSEYVDRTLPEATNDPGQANLPPPPTIPQATAPPRLASDSALWPKKPQLLTRRERDDLLQATGDFFSELLTEAEAAEVRALEEQVRESQTLMAKAHEMAASTRRGFHTALEAVLSRSRDEAPDDELRSLLPSPPKAPVQAPLEAALARAAAGNGSWPYRKSLAAAKWIRGICEAVRGLSEGALALAGGAGAWLNLAAAADGEIHELTRLLEVEGMAQNSMDGMEELRLALATLDPKRVAGGKETVADWKRRLSRLEAIIQEAQEESQLQGTLQDLVTQARGHTDPRQLKIVVEAARGLALGASAGSQYALLKDKLLRYASAKQSFLAFYETAQPTVFVKHPLTNNLPLLITISAPPTGWGNGAPTRRAQFLAAAGPAKYAGTLWLETESPCDPLNPAYVSADTQEPLNYIPVYHNFLEYVMPTVLENPEAFSLTPAGRPQAIGPPQDDQERRRRTLASVASARLSAAAADSYWDTWPDVESNAGELLREYVSAPKALMEDLADNPIVAMTLLAHASLIASRNHPPYPAPATDREVILLEQREMMALLVGTHPAYAAAFLGAPSFYAGLGLVSALARDGGLGDLLSDSVLTYRLVRSPASGRGGMPSTTRGSNDGEDARRLTRHRIAGPPTGFIFFQDAWEEMDTRAALWPHPEFLGLVHNQSTARARACMLLLARRCFAPEALQQLWHSLRPLEGPVAFQDYLRDFVKQAYTRGEELPRAEGLEVPRETPSSYGTVTGRALRNLMPYGTPITGPKRGSGDTIPVSVFEAAVAAAFLGRPLTLFVSSQYLFNLKTLGQVRVVAPLLYCDGHSEPFRSLVETISLNFLQDLDGYSESFEPEMSIFARQAVWLRELLTEARAAKPKEARPPTVAILANRKNIIWKCFTYRHNLPDVQFYFNAAGASRWPTDVLNPSFYEHEDPPLPVGYQLPPNPRNVQELFSGFPPRVGHGLVSGDGFQSADNTPASSDRLQQLGGGETDQGEKGSTTAESEASGPPSPQSPLLEKVAPGRPRDWLSPTSSPRDVTVTPGLAAPITLPGPRLMARPYFGAETRASESPDRSPGSSPRPWPKDSLELLPQPAPQQPPSSPWASEQGPIVYTLSPHSTPSTASGSQKKHTIQIPGLVPSQKPSYPPSAPYKPGQSTGGIAPTPSAASLTTFGLQPQDTQASSQDPPYGHSIMQREKKQQGGREEAAEIRPSATRLPTAVGLRPRAPVVAAGAAASATPAFDPGEAPSGFPIPQAPALGSGLAAPAHTPVGALAPRPQKTQAQRPQDAAALPTPTIKAVGARPVPKATGALAAGARPRGQPTAAPPSAASPPRVSLPVRSRQQQSPAIPLPPMHSGSEPGARPEVRLSQYRHAGPQTYTVRKEAPPSAASQLPKMPKCKDSMYYPPSGSARYPAPFQALSFSQSVASPAPSSDQTTLLWNTPSVVTQFLSIEDIIREVVTGGSTSGDLVVPSGSPSSLSTAAPEQDLRYSLTLSQASRVLSRFVSQLRRKLERSTHRLIADLERLKFLYL

SEQ ID NO: 33 - DNA, 3720 bp, Homo sapiens
atggcgtccgctatggagagtgacagcagcggcggtagcggaggggcagacgcccagccacccctggcagaggtggacggggggctcgcccgcgtgacgcgtcagttgctgctgtctggggacgaccccgctgcccgcctgcgcgcgctgatgcctctggagctggggatctttggactgggggatttggcccagccggtgctggtgcgcgattttttaaatactctcaccctcatgagcggccacgcttacccggcggcggtcctccgccaccacgcctactacctactccgggcggcctccttttccaggcgcagcttcggcctcggccatctggaggcggccctggacgtgctggcatcctcactgccccccacgacagcctctccggccaccgacgatccgctggacggctctcggctcatcgcagagacccgggccctggcggccgcgtaccggcgcatcatcgaagagggcagcggggaggtgctcgcggtctctggcccaacggccacctttgccttcgtggaagagctggtggccgacacttatctggcccggtgggacgccttcccgcgggaggggctgtcattctacgcttttaacgcggcgaaaacgacgctggggagatggctggtcaccgtatacgcggagaccaaccgctacccctgggcggctgcagggcagggtcagcccacggccgcggacatcaaggccatggccgtggagctggtggagcacagtgggggcggggccggcgggggagagggtgaagaaagcggtggtggtctctttcaccgccccgagtcactttccagcgtggtggcctcgctgcccctggcccggcggcgggcggtagagatcctgggcgtgtacgcggaggccagcgggggacaaacgccccccgtcgcggctgtgcccgtcctggcctttgacgccgcccggctccggcttttggagccctcaggggccctcttttacgactatgtgtacgaggccctgctctgggaccagacttacggcgtcccagactcggtcatcgaggccttcctggccgggatggcggcagagatggaggccctggccgctcgtgtgcaggaggcggcggggagccgggcatccttctctccagccgccatcgagcaggtggccacggttctgctctcggccgggctcaacgagaccgtggcgggggactacgccatgatgctggcctctgtgccccgggtgtcccgctcgcggtggaggtggctcgaggccaccgccgccctcctagagagtctatcgggctttgccctgcactttttccgactgctgcccaccgcgagccccacgagccgcttcgcccgtgtggcccgggcggcctacctgcgggcggaggccgaggccgtggacagacgagcccggcgcaccagcggcccctctacgcccgccgccgcccccgccgcgacggctgtgggtgtgggggcggcggcggacccttgggacgccgtaacccctctgcgcatcttcatcgtcccccctcccgcggccgagtatgagcaggtggcgggggacctttcatccgaactcctcaggtctctgctatgggtccgctacagccgcctgtggcaggccccggccccggctccggccctcccctgtaagccccccctactccccggcgagcagggaagaaggcagtggacggcagcggtggcggcggccccgcggacagacgtcgaggcatactgccggtccctgagggccggccaaacggcgcgcgcggatccggcctatgtccacagccccttcttcccggccgccttcatcgagttccaaatctggcccgcccttcgccgggtcctctccaacgagctgcccaaaacccgctctctggccgccctgcgctggctggtctccttcggcagcgacctggccctcccttcccccgagctgacccgggcccgccgtcctctcgagctcatatacgccaccgtctgggagatctatgacggggcccctccgatgcctggggagtccccccaggcggtcggactgcgccccctcaacttagagggggaaggcaaggccggggacgcaggagccgagggtgcagaagacgaagagggcgggggcccctgggggctgtcgtcccacgacgcagtccttaggatcatggacgccgtccgagaggtctcaggcatcatctccgagactatctcagcttcagagcgggcggcggaagcaccaccgcttgcctggcccacctccctcttttcactcctcttcaccctgagatacagcacgacggcggagtcgttgggcctggccacccgccgctttctggtctcgggtgagaccctctcggaggacatctcgcgcttgacgggggcagcttggaggctgtgctcccgcccgctcctgtacgacgcagagaccgggagggtgcagatccctctggcgacggaagaggaggaggaggcggtggtagcggtgaaggaaaagagcgtttcatcctccccccgccactactccacggacctccagacgctaaagagcgtcgtggagggcatccaggatgtgtgccgggacgccgccgccaggtgggccctggccacggccgacacggccaccctcagaaggcgcctcctggtgccggccctcagggagagccggggcatcgcggatcaccccctctgggcccacaccagcgagcccctgcgtccggatctcgaggagctcaacgagcgggtggagcacgccctggagctgggatacagcctgacgggcgccctcaggcgcagcgtcgcctaccggttccgggattacacattcgcccgcctgttccagccacccgccatcgacgccgagcgtgccgaggccatagtccggcgagacgcccgcccaccacctgtcttcatccccgcacccaggcggcttccgcaggggggagcagacaccccacctcccctcagcatggacgacatcctgtatctaggcaagagcatctgcaaggccctggtggacgtcctcgaccatcatcctgccgcgccagagaccacccctattaaaacatacacacccgccatggacctaaatccggaacagatcacagtcacccccagaagcccctcggttctcgccgcctttgctcgcacggcccgggtccagacccaccacctcgtgccggccctaaccgacgattccccctcacccgtgggacaaacgcccccgccattccgcatcctccccgccaaaaaactcgcggccattctcctgggtaacggcaggaacgcgagcaagcgccgggccagccgggacctgtcaccaccgccccacggcaggtggcgtgccgttttggactcctccccattctccttctcctcctcagacttttccgaccaggacgagggagagggaggggaggcagatctcaggggcgtgccgggaggaggaggagaaggagcatacgaggaagacagggaaaggccatcggatatcgacaccgcggcccgagcccagaaggttgagacctcctgcccccgcagacgcagtccgcggactaccccctctccttcaaggcgggcaagcggcggcggcggcccagacagaggagaggcggaggcacacacgtaccccccttatctctcggcggctgccgccgccagccgcgtaagaccccggaccagaaggggggcgacaaggcgtcctccccgccccaccgccgaagatgagtaa

SEQ ID NO: 34 - protein, 1239 aa, Homo sapiens
MASAMESDSSGGSGGADAQPPLAEVDGGLARVTRQLLLSGDDPAARLRALMPLELGIFGLGDLAQPVLVRDFLNTLTLMSGHAYPAAVLRHHAYYLLRAASFSRRSFGLGHLEAALDVLASSLPPTTASPATDDPLDGSRLIAETRALAAAYRRIIEEGSGEVLAVSGPTATFAFVEELVADTYLARWDAFPREGLSFYAFNAAKTTLGRWLVTVYAETNRYPWAAAGQGQPTAADIKAMAVELVEHSGGGAGGGEGEESGGGLFHRPESLSSVVASLPLARRRAVEILGVYAEASGGQTPPVAAVPVLAFDAARLRLLEPSGALFYDYVYEALLWDQTYGVPDSVIEAFLAGMAAEMEALAARVQEAAGSRASFSPAAIEQVATVLLSAGLNETVAGDYAMMLASVPRVSRSRWRWLEATAALLESLSGFALHFFRLLPTASPTSRFARVARAAYLRAEAEAVDRRARRTSGPSTPAAAPAATAVGVGAAADPWDAVTPLRIFIVPPPAAEYEQVAGDLSSELLRSLLWVRYSRLWQAPAPAPALPCKPPLLPGEQGRRQWTAAVAAAPRTDVEAYCRSLRAGQTARADPAYVHSPFFPAAFIEFQIWPALRRVLSNELPKTRSLAALRWLVSFGSDLALPSPELTRARRPLELIYATVWEIYDGAPPMPGESPQAVGLRPLNLEGEGKAGDAGAEGAEDEEGGGPWGLSSHDAVLRIMDAVREVSGIISETISASERAAEAPPLAWPTSLFSLLFTLRYSTTAESLGLATRRFLVSGETLSEDISRLTGAAWRLCSRPLLYDAETGRVQIPLATEEEEEAVVAVKEKSVSSSPRHYSTDLQTLKSVVEGIQDVCRDAAARWALATADTATLRRRLLVPALRESRGIADHPLWAHTSEPLRPDLEELNERVEHALELGYSLTGALRRSVAYRFRDYTFARLFQPPAIDAERAEAIVRRDARPPPVFIPAPRRLPQGGADTPPPLSMDDILYLGKSICKALVDVLDHHPAAPETTPIKTYTPAMDLNPEQITVTPRSPSVLAAFARTARVQTHHLVPALTDDSPSPVGQTPPPFRILPAKKLAAILLGNGRNASKRRASRDLSPPPHGRWRAVLDSSPFSFSSSDFSDQDEGEGGEADLRGVPGGGGEGAYEEDRERPSDIDTAARAQKVETSCPRRRSPRTTPSPSRRASGGGGPDRGEAEAHTYPPYLSAAAAASRVRPRTRRGATRRPPRPTAEDE

SEQ ID NO: 35 - DNA, 1095 bp, Homo sapiens
atgaaggtccaggggtccgtcgatcgccgccgtctgcaacgccgaatcgcggggctgctgccccctccggcccggcgtctaaatatttcccgggggtccgaattcacgcgggacgttcgtgggctggttgaggaacacgcgcaggcctcctcgctgagtgcggcggccgtctggcgcgcagggctgctggccccgggggaggtggcggtcgccgggggtggcagtggaggggggagcttcagctggtctgggtggcggccgccagtctttggggactttctgatacacgccagctccttcaacaacgccgaggccactggaacgccccttttccaattcaagcagagtgacccgttctcgggcgtcgacgcggtattcactcctctctccctgtttatcctaatgaatcacggccggggtgtagccgcccgggtcgaggcaggtgggggcctgacgcggatggccaacctgctgtacgacagccccgcaaccctggctgacctggtcccggactttgggcggctggtggccgaccgccgcttccacaacttcatcacccctgtgggccccctggtggagaatataaagagcacctatctgaataaaatcaccacggtggtccacgggcctgtggtcagcaaggccatccctcgcagcaccgtcaaggtgacggtgccccaggaggcctttgtggatctggacgcgtggctctccggcggcgccgggggtggcggtggagtatgcttcgtcggggggctgggcctgcagccgtgccccgccgatgcgcgcctctatgtcgctctgacctatgaggaagccgggccgcggtttacgtttttccagtcgtcccgcggccactgtcagatcatgaatatcttaagaatttattactcaccatccatcatgcaccgctatgctgtggtccagcccctacatatagaggagctaaccttcggggcggttgcctgtctggggacatttagtgctactgacggttggaggaggtctgccttcaattaccgtggctctagcctccccgtggtggagattgacagcttttattccaacgtctctgactgggaggtgattctctag

SEQ ID NO: 36 - protein, 364 aa, Homo sapiens
MKVQGSVDRRRLQRRIAGLLPPPARRLNISRGSEFTRDVRGLVEEHAQASSLSAAAVWRAGLLAPGEVAVAGGGSGGGSFSWSGWRPPVFGDFLIHASSFNNAEATGTPLFQFKQSDPFSGVDAVFTPLSLFILMNHGRGVAARVEAGGGLTRMANLLYDSPATLADLVPDFGRLVADRRFHNFITPVGPLVENIKSTYLNKITTVVHGPVVSKAIPRSTVKVTVPQEAFVDLDAWLSGGAGGGGGVCFVGGLGLQPCPADARLYVALTYEEAGPRFTFFQSSRGHCQIMNILRIYYSPSIMHRYAVVQPLHIEELTFGAVACLGTFSATDGWRRSAFNYRGSSLPVVEIDSFYSNVSDWEVIL

SEQ ID NO: 37 - DNA, 2481 bp, Homo sapiens
atggcaacgaccagtcatgtcgagcatgagctcctctccaaattgattgatgagttaaaggtcaaggccaactcagaccccgaggctgatgtcctggccgggcgcctgctccaccgccttaaggccgagtcagttacacacacagtagccgaatatctggaggtcttctctgacaaattctacgatgaggaattcttccagatgcaccgggatgagctggagacccgagtctctgctttcgcgcagagcccggcctacgagcgcatcgtctccagcggctacctgtcggccctgcgctactatgacacctatctgtatgtggggcgcagcgggaagcaggagagtgtgcagcacttttacatgcggttagccggcttctgtgcctcaaccacctgcctctacgcgggtctcagggcagccctgcagcgggccaggccggagattgagagtgacatggaggtgtttgattactactttgagcacctaacctcccagacggtgtgctgctccacgccctttatgcgctttgccggggtggaaaactccactctggccagctgcatcctcaccacccccgacctcagctccgagtgggacgtgacccaggccctctataggcacctggggcgctacctctttcagcgagccggggtgggtgtaggggtgacgggggctggccaggatgggaaacacatcagcctcctgatgaggatgatcaacagccacgtggagtaccacaactatggctgcaagaggccggtcagtgtggcggcctacatggagccctggcacagccagattttcaagtttttggaaacgaagctgccggagaaccacgagaggtgcccgggcatctttacggggctctttgtccccgagctcttcttcaagctttttagggacacgccctggtcggactggtacctgtttgaccccaaggacgccggggacctggagaggctctacggggaggagtttgagcgcgagtactatcggctggtgacagcgggcaagttttgtgggcgggtctccatcaagtccctgatgttctctatcgtcaactgcgccgtcaaggccggcagccccttcatccttttgaaggaggcctgcaacgcccacttttggcgcgacctgcagggcgaggccatgaacgccgccaacctgtgcgccgaggtgctgcagccctcgaggaagtctgtggccacctgcaatctggccaacatctgcctcccgcgctgcctggtgaatgcgcctctggcggtgcgggcacagcgggccgacacgcagggggatgaactcctgctggccctccctcgactctcagtcaccctacctggagagggggcagtcggtgatggattctcgctagcccgcctcagagatgccacccagtgtgccacctttgtggtggcctgctccattcttcagggatcccccacttatgattccagggatatggcctccatgggcctcggggtgcagggcctggccgatgtctttgcggacctgggctggcagtacactgaccctccctctcgctcgttaaacaaggaaatattcgaacatatgtactttacggccctctgcaccagtagtctgattggacttcacaccaggaagatttttccgggtttcaaacagagcaagtatgccggggggtggtttcactggcacgattgggcaggaacagacctttctattcccagggaaatttggtctcgcctctctgaacgcattgtgagggatgggcttttcaattcacagtttatcgccctgatgcccacctcaggctgtgcccaggtgacgggctgttcggacgccttctaccccttctatgccaatgcgtccaccaaggtcaccaacaaggaggaggcccttaggccaaaccggtctttttggcgtcatgtgcgtctggatgacagggaagctttgaatcttgtcgggggccgtgtctcctgcctcccggaggctctgcggcagcgctacctgcgtttccaaacggcctttgattacaaccaggaggacctgattcagatgtcccgggacagggccccctttgtggaccagagccaatctcacagcctgtttttgcgtgaggaagatgccgcgcgggccagcacgctagccaacctactggtgcgcagctacgagctgggcctgaagactatcatgtactattgtcgcattgagaaggccgccgatctgggggtgatggagtgtaaggccagcgcggctctgtcggtgccgcgggaggaacagaatgagcggagtcccgctgagcagatgccgcctcgtcccatggaaccggcgcaggttgcggggccggttgacatcatgagcaagggcccaggggagggaccaggtgggtggtgtgtgcccgggggattggaagtgtgctataagtaccgtcagctcttctcagaggatgatctgttggagactgacggttttactgaacgagcctgtgaatcttgccaataa

SEQ ID NO: 38 - protein, 826 aa, Homo sapiens
MATTSHVEHELLSKLIDELKVKANSDPEADVLAGRLLHRLKAESVTHTVAEYLEVFSDKFYDEEFFQMHRDELETRVSAFAQSPAYERIVSSGYLSALRYYDTYLYVGRSGKQESVQHFYMRLAGFCASTTCLYAGLRAALQRARPEIESDMEVFDYYFEHLTSQTVCCSTPFMRFAGVENSTLASCILTTPDLSSEWDVTQALYRHLGRYLFQRAGVGVGVTGAGQDGKHISLLMRMINSHVEYHNYGCKRPVSVAAYMEPWHSQIFKFLETKLPENHERCPGIFTGLFVPELFFKLFRDTPWSDWYLFDPKDAGDLERLYGEEFEREYYRLVTAGKFCGRVSIKSLMFSIVNCAVKAGSPFILLKEACNAHFWRDLQGEAMNAANLCAEVLQPSRKSVATCNLANICLPRCLVNAPLAVRAQRADTQGDELLLALPRLSVTLPGEGAVGDGFSLARLRDATQCATFVVACSILQGSPTYDSRDMASMGLGVQGLADVFADLGWQYTDPPSRSLNKEIFEHMYFTALCTSSLIGLHTRKIFPGFKQSKYAGGWFHWHDWAGTDLSIPREIWSRLSERIVRDGLFNSQFIALMPTSGCAQVTGCSDAFYPFYANASTKVTNKEEALRPNRSFWRHVRLDDREALNLVGGRVSCLPEALRQRYLRFQTAFDYNQEDLIQMSRDRAPFVDQSQSHSLFLREEDAARASTLANLLVRSYELGLKTIMYYCRIEKAADLGVMECKASAALSVPREEQNERSPAEQMPPRPMEPAQVAGPVDIMSKGPGEGPGGWCVPGGLEVCYKYRQLFSEDDLLETDGFTERACESCQ

SEQ ID NO: 39 - DNA, 909 bp, Homo sapiens
atgtccaagttgttgtacgtgcgtgatcatgagggctttgcctgcctaacggtcgaaacccaccgcaaccgctggttcgcggctcacattgtcctcaccaaggactgcgggtgtctcaagctactcaatgagagggacttggagttttacaagttcctctttacgttcctggccatggccgagaagcttgtgaactttaacattgatgaactggtcaccagcttcgagagccacgacattgatcactactacaccgagcagaaggccatggagaacgtccacggggagacttatgctaacattttaaacatgctctttgatggggacagggcggcgatgaacgcctacgcagaggccatcatggccgacgaggccctgcaagccaagatttcctggctccgtgacaaggtggcggccgccgtcaccctgccggagaagattcttgtgttcctgctgattgaaggcatcttcttcattagctccttctacagcatagccctgctgcgggtccggggcctaatgcctggcatctgcctggccaataactacataagtagggatgagctgctccacacccgcgctgcctccctgttatacaatagcatgacagccaaggctgaccgaccaagggccacctggatccaggagctgtttcgcactgcggtggaggtagagactgccttcatcgaggctcgtggagagggggttaccttggtggatgtgcgagccataaagcagtttctggaggccacggccgatcgcatcctgggtgacattggtcaggctcccttgtatggcacaccaccccccaaggactgcccgctcacctacatgactagcatcaagcaaactaatttctttgagcaagagagttccgattacaccatgctggtggtagatgacctttga

SEQ ID NO: 40 - protein, 302 aa, Homo sapiens
MSKLLYVRDHEGFACLTVETHRNRWFAAHIVLTKDCGCLKLLNERDLEFYKFLFTFLAMAEKLVNFNIDELVTSFESHDIDHYYTEQKAMENVHGETYANILNMLFDGDRAAMNAYAEAIMADEALQAKISWLRDKVAAAVTLPEKILVFLLIEGIFFISSFYSIALLRVRGLMPGICLANNYISRDELLHTRAASLLYNSMTAKADRPRATWIQELFRTAVEVETAFIEARGEGVTLVDVRAIKQFLEATADRILGDIGQAPLYGTPPPKDCPLTYMTSIKQTNFFEQESSDYTMLVVDDL

SEQ ID NO: 41 - DNA, 1215 bp, Homo sapiens
atggaaaccactcagactctccgctttaagaccaaggccctagccgtcctgtccaagtgctatgaccatgcccagactcatctcaagggaggagtgctgcaggtaaaccttctgtctgtaaactatggaggcccccggctggccgccgtggccaacgcaggcacggccgggctaatcagcttcgaggtctcccctgacgctgtggccgagtggcagaatcaccagagcccagaggaggccccggccgccgtgtcatttagaaaccttgcctacgggcgcacctgtgtcctgggcaaggagctgtttggctcggctgtggagcaggcttccctgcaattttacaagcggccacaagggggttcccggcctgaatttgttaagctcactatggaatatgatgataaggtgtccaagagccaccacacctgcgccctgatgccctatatgcccccggccagcgacaggctgaggaacgagcagatgattgggcaggtgctgttgatgcccaagacggcttcctcgttgcagaagtgggcacgccagcaaggctcaggcggcgttaaggtgacactcaatccggatctctacgtcaccacgtatacttctggggaggcctgcctcaccctagactacaagcctctgagtgtggggccatacgaggccttcactggccctgtggccaaggctcaggacgtgggggccgttgaggcccacgttgtctgctcggtagcagcggactcgctggcggcggcgcttagcctctgccgcattccggccgttagcgtgccaatcttgaggttttacaggtctggcatcatagctgtggtggccggcctgctgacgtcagcgggggacctgccgttggatcttagtgttattttatttaaccacgcctccgaagaggcggccgccagtacggcctctgagccagaagataaaagtccccgggtgcaaccactgggcacaggactccaacaacgccccagacatacggtcagtccatctccttcacctccgccacctcctaggacccctacttgggagagtccggcaaggccagagacaccctcgcctgccattcccagccactccagcaacaccgcactggagaggcctctggctgttcagctcgcgaggaaaaggacatcgtcggaggccaggcagaagcagaagcaccccaagaaagtgaagcaggcctttaaccccctcatttaa

SEQ ID NO: 42 - protein, 404 aa, Homo sapiens
METTQTLRFKTKALAVLSKCYDHAQTHLKGGVLQVNLLSVNYGGPRLAAVANAGTAGLISFEVSPDAVAEWQNHQSPEEAPAAVSFRNLAYGRTCVLGKELFGSAVEQASLQFYKRPQGGSRPEFVKLTMEYDDKVSKSHHTCALMPYMPPASDRLRNEQMIGQVLLMPKTASSLQKWARQQGSGGVKVTLNPDLYVTTYTSGEACLTLDYKPLSVGPYEAFTGPVAKAQDVGAVEAHVVCSVAADSLAAALSLCRIPAVSVPILRFYRSGIIAVVAGLLTSAGDLPLDLSVILFNHASEEAAASTASEPEDKSPRVQPLGTGLQQRPRHTVSPSPSPPPPPRTPTWESPARPETPSPAIPSHSSNTALERPLAVQLARKRTSSEARQKQKHPKKVKQAFNPLI

SEQ ID NO: 43 - DNA, 1074 bp, Homo sapiens
atgttctcgtgcaagcagcacctgtccctgggggcctgtgtcttctgtctcggcctcctggccagcacccccttcatttggtgctttgtctttgccaacctgctctctctggagatcttctcaccgtggcagacacacgtgtacaggcttggattcccgacggcatgcctaatggccgtcctctggacgctggtacccgccaagcacgcggtgagggccgtcactccagccatcatgctgaatattgccagcgccttgatcttcttctccctcagagtctactcgaccagcacgtgggtttctgccccctgtctctttctggccaacctgcctctcttatgcctgtggccccggctggccatcgagattgtttacatctgcccggctatacaccaaaggttctttgaacttgggttgctcttggcctgcaccatctttgccctgtccgtggtctccagggccctggaggtgtcggctgtcttcatgtctccatttttcatctttctggctttgggctctggaagcctggccggtgctcggcgtaaccagatttacacctcgggtctcgagcggagacgcagcattttctgcgcccggggagatcattcggtggcatccctgaaggagaccctccataaatgcccgtgggatctgctggccatctctgccttgaccgttcttgtcgtctgtgtgatgattgtgttgcatgtgcacgcagaggtgttctttggactctctagatacctgcccctctttctctgtggggcgatggcctccggggggctgtacctgggccattccagcatcattgcatgtgtcatggccaccctctgcaccctgacatctgttgtggtatatttcctccatgaaacccttggacccctgggcaagaccgtgctgtttatctcaatctttgtctattactttagcggggtagcggccctgagcgcagctatgcgctacaagcttaagaagtttgtgaacggacccctggtccatctccgtgtggtatacatgtgctgttttgtctttactttttgtgaatatctgttggtgacattcattaaatcctaa

SEQ ID NO: 44 - protein, 357 aa, Homo sapiens
MFSCKQHLSLGACVFCLGLLASTPFIWCFVFANLLSLEIFSPWQTHVYRLGFPTACLMAVLWTLVPAKHAVRAVTPAIMLNIASALIFFSLRVYSTSTWVSAPCLFLANLPLLCLWPRLAIEIVYICPAIHQRFFELGLLLACTIFALSVVSRALEVSAVFMSPFFIFLALGSGSLAGARRNQIYTSGLERRRSIFCARGDHSVASLKETLHKCPWDLLAISALTVLVVCVMIVLHVHAEVFFGLSRYLPLFLCGAMASGGLYLGHSSIIACVMATLCTLTSVVVYFLHETLGPLGKTVLFISIFVYYFSGVAALSAAMRYKLKKFVNGPLVHLRVVYMCCFVFTFCEYLLVTFIKS

SEQ ID NO: 45 - DNA, 1317 bp, Homo sapiens
atggagggcagcgaagaacactccacagatggagagatttcatcctcagaggaggaggatgaagatccaactccggcccacgccatacctgcacggccctccagcgtggtcataacccctacctcggcatcgtttgtgattcccagaaagaagtgggacctacaggacaagacagtcacattgcatcgctcacccctgtgcagggacgaggacgagaaggaggagactggcaactcctcttacaccagaggccacaaaaggcgacgcggagaggtccatggctgcaccgatgaaagttatggcaagcgccgacacctgcccccgggagcaagagcgcccagagccccaagggcccccagggtgcctagagcaccgaggtctccaagagctccccgaagcaacagagcaaccagaggtccccggtcagaatctcgaggggccggcaggagcacaaggaagcaggcgaggcaagaacgcagccagaggcccctgccgaacaaaccgtggtttgacatgagtctggttaagcctgtctccaagattacatttgtcaccttgcccagccccctggcctctctgaccctagagcccatccaagacccgttcctacagtcgatgctggcggtggccgcccatccagagattggagcctggcagaaagtgcaacccagacacgagctgcgcaggagctacaagacactacgtgagtttttcaccaagtcaaccaacaaggacacatggctggatgcacgcatgcaggcgatccagaacgcggggctctgcaccctggtggccatgctagaagagaccatcttttggctccaggagatcacctaccacggcgacctgcccctagctcccgcggaagacatcctcctggcctgcgccatgagtcttagcaaggtgatcctgaccaagctcaaagagctggcaccctgcttccttcctaacacgcgagactacaactttgtgaagcaactcttctacatcacctgtgccacggcccgtcaaaacaaggtggtggagaccctgagcagctcatatgtgaagcagcccctctgtctcttggcagcatatgcggcagtagccccagcctacattaacgccaactgcagacggagacacgatgaagttgaattcctgggccactacatcaagaattacaaccctggcacgctaagctcccttttgacagaggccgtggagactcacacacgtgactgccgaagtgcatcatgcagccgacttgtcagggccattctctctccgggcactgggtcactaggactgttttttgttcctggattaaatcaataa

SEQ ID NO: 46 - protein, 438 aa, Homo sapiens
MEGSEEHSTDGEISSSEEEDEDPTPAHAIPARPSSVVITPTSASFVIPRKKWDLQDKTVTLHRSPLCRDEDEKEETGNSSYTRGHKRRRGEVHGCTDESYGKRRHLPPGARAPRAPRAPRVPRAPRSPRAPRSNRATRGPRSESRGAGRSTRKQARQERSQRPLPNKPWFDMSLVKPVSKITFVTLPSPLASLTLEPIQDPFLQSMLAVAAHPEIGAWQKVQPRHELRRSYKTLREFFTKSTNKDTWLDARMQAIQNAGLCTLVAMLEETIFWLQEITYHGDLPLAPAEDILLACAMSLSKVILTKLKELAPCFLPNTRDYNFVKQLFYITCATARQNKVVETLSSSYVKQPLCLLAAYAAVAPAYINANCRRRHDEVEFLGHYIKNYNPGTLSSLLTEAVETHTRDCRSASCSRLVRAILSPGTGSLGLFFVPGLNQ

SEQ ID NO: 47 - DNA, 120 bp, Homo sapiens
atggttccttctcagagactctcccgaactagcagcatttcctccaacgaggatcccgcaggtaagaagctacaccggccagtggccggggccgtggagccgggggcatccggtgcctga

SEQ ID NO: 48 - protein, 39 aa, Homo sapiens
MVPSQRLSRTSSISSNEDPAGKKLHRPVAGAVEPGASGA

SEQ ID NO: 49 - DNA, 2625 bp, Homo sapiens
atgtccgcccccgtcgtcatcaaggcccttgtggcctcaaacactgacattgcggaggccatcctcgatgccatcctctcgcggcctgacgagggcttccgcctgttttgtttgtgccacaacgcctcgcccctccaccacgtggcgggttctctcgtggaactgcagcttcacctgcccaagaagcggctgacctcacagagccgctgcggcctggttctgacgctgcacctgccggcggaggaggccttccccttcctgcgcggcctcacgccgctcaccgcggaccgtctatccacctacctagatcgcgcgggggccctgcgctcgctcacgcccctcgtggaactgctcacactgagcgctaagaaacagccacagggggatgccagaggccgcgtggcgtggctgcgtcccaagatcgtgggctgtctgcgccgcatctaccgcgttaacatctcggccaggtggttcattagcacctttggctcccacgaggcccagttcgtgctagtcacggctgcttactatttttggggcattccttgtacgattgagaccctggcgcacctcacggagctatttacttccgagtctggtcagagcctggccgccgtcacgtccctggccgagctgggggaggtctttggctcctcggcatgggcggagcagaccgaggcttttgcccactttgcacacgagaaactgcggcgggactcgcgagagatccgcgctgtggcaaggacgatagacgcctacagggggcgccttcccctggcctcggccgatctcgtgcgctacgtctatttggcccacgcacagtgcttcaacgagggaacctttaagcgatactctcagttaacgagcatgggagaaattgggtgtctgccttcggggggcgtcgtgctcccctcgctcctggacaggggctttgcggagcacatgcgcacctacttcacccgggagacttacctggctgagcatgtgcgtgtccagcagctgaaaatccgcatggagcccccggccccatacacgtgggaccccgaccccgatgacggactcatgcgggcctgggccgggctcagtgtggacgtggcccgggagctggtggagctcgcgcgctggcacgcggatgagggtcccacatacccccccacgcttcaagggtttctatgcttggccggccaggccacctgccggggccagtggaatcccaaggaacaattcctcccacccaccgtccttcgaagggtgcagcggctaccggtcttcctctgccattttgcagacaggcactactttgtaatgacagccgctgaccccttctcatcccacctggcggaggtcgtctccaccccgaccaactgccgcctcccagacacgtgtctcaccagggccctctcctacaccccagtgtactattcacagaacagcctgagcgagcagctctttgtctcccggcatgaatactttaatccccggcttccggtctgcaacctggtcttggacctggatctaaagatcaagggggccccctggtcgctggaggaaatctatgacctgtgccggaccgtgcggcgtgaggtactgcgcctcatgcgccgcctgggtccagtgtccagggcccacccagtctattttttcaaatcagcttgtccacccgccgacccggataatatggaagatgtgctccccttttgcatatgcacgggaaaactgggctttcgcgtcatcacccccctacctagaggccatgctattgtgggaacaagcgcagtacaagggtttgtgtctgtgctgcagaagctcatgggcctaacggcctgcctgcgccgcatgcgtcacaagatcaaagagattggggccccgctttttgacagcggcgtgtatcacgccgggcggtgcatccggctgccgcatacctacaaggtggacaggggcggtggtcttagccggcagctgcgcctctttgtctgtcatccggaagaggaagacaagcacagctatgttaagaatgccctcaacattcaaaacctcttacatcactcactgcacgtgggctggccggcccccaaaaccttctgctaccacatcgcggatgatgggcgtgactatctaatccagaggacccgcgagaccctgccccccaccgtggagaatgtctgcgccatgatagagggacatctgggcctggatctcgtcgcctgggtcagctcctgcatctggccctcgctcatgagcaccctggcaacagctgtgccagaagacaaattcccccagtttctccatgtcacgtttgagcaaaccgggccaaacttagttcaggtgtgccatgcccggggcaggaactttgcgtgcctgaggcatacccacagggccagctccaagaatgtgagggtgtttctggtactctactacacatcacaggccatcacggtcaccttcatgagtcagtgcttcgccggtcgctgtggggccaatcaaccgaccgcccatttctccatcagcgtgcccgcctccagaatcataaatagggctgaggccagtcaagacagcactacatcccagctagcccgtcgtagagacagacaagatggttccttctcagagactctcccgaactag

SEQ ID NO: 50 - protein, 874 aa, Homo sapiens
MSAPVVIKALVASNTDIAEAILDAILSRPDEGFRLFCLCHNASPLHHVAGSLVELQLHLPKKRLTSQSRCGLVLTLHLPAEEAFPFLRGLTPLTADRLSTYLDRAGALRSLTPLVELLTLSAKKQPQGDARGRVAWLRPKIVGCLRRIYRVNISARWFISTFGSHEAQFVLVTAAYYFWGIPCTIETLAHLTELFTSESGQSLAAVTSLAELGEVFGSSAWAEQTEAFAHFAHEKLRRDSREIRAVARTIDAYRGRLPLASADLVRYVYLAHAQCFNEGTFKRYSQLTSMGEIGCLPSGGVVLPSLLDRGFAEHMRTYFTRETYLAEHVRVQQLKIRMEPPAPYTWDPDPDDGLMRAWAGLSVDVARELVELARWHADEGPTYPPTLQGFLCLAGQATCRGQWNPKEQFLPPTVLRRVQRLPVFLCHFADRHYFVMTAADPFSSHLAEVVSTPTNCRLPDTCLTRALSYTPVYYSQNSLSEQLFVSRHEYFNPRLPVCNLVLDLDLKIKGAPWSLEEIYDLCRTVRREVLRLMRRLGPVSRAHPVYFFKSACPPADPDNMEDVLPFCICTGKLGFRVITPLPRGHAIVGTSAVQGFVSVLQKLMGLTACLRRMRHKIKEIGAPLFDSGVYHAGRCIRLPHTYKVDRGGGLSRQLRLFVCHPEEEDKHSYVKNALNIQNLLHHSLHVGWPAPKTFCYHIADDGRDYLIQRTRETLPPTVENVCAMIEGHLGLDLVAWVSSCIWPSLMSTLATAVPEDKFPQFLHVTFEQTGPNLVQVCHARGRNFACLRHTHRASSKNVRVFLVLYYTSQAITVTFMSQCFAGRCGANQPTAHFSISVPASRIINRAEASQDSTTSQLARRRDRQDGSFSETLPN

SEQ ID NO: 51 - DNA, 657 bp, Homo sapiens
atggccttctatctcccagactggtcgtgctgcgggctctggctctttggccggcccaggaatagatacagccagctccctgaggagccggagacctttgagtgcccggaccgctggcgagccgagatagatctgggcctgccccctggtgtgcaggtgggagatttgctaagaaatgagcagacgatgggctcactgagacaggtttatttgctcgctgttcaagccaatagcatcacggatcacctgaagcgctttgacgccgtccgcgtccctgagagctgtcgtggggtggtggaggcccaggtggccaagcttgaggccgtgcgctcagtcatctggaataccatgatctctctggctgtaagcggcatcgagatggacgagaatgggctcaaggccctgctggacaaacaggctggcgacagcctggccctgatggagatggagaaggtggccacggcgctcaagatggacgagaccggtgcctgggcgcaagagatctcggccgttgtctcatcggtgaccgccccctcagcctcggcccctttcatcaactccgcctttgagcccgaggtgcccacccccgtccttgcaccgcctcccgtggtgcggcagccggagcactctgggcccacggagctcgcgttaacgtag

SEQ ID NO: 52 - protein, 218 aa, Homo sapiens
MAFYLPDWSCCGLWLFGRPRNRYSQLPEEPETFECPDRWRAEIDLGLPPGVQVGDLLRNEQTMGSLRQVYLLAVQANSITDHLKRFDAVRVPESCRGVVEAQVAKLEAVRSVIWNTMISLAVSGIEMDENGLKALLDKQAGDSLALMEMEKVATALKMDETGAWAQEISAVVSSVTAPSASAPFINSAFEPEVPTPVLAPPPVVRQPEHSGPTELALT

SEQ ID NO: 53 - DNA, 837 bp, Homo sapiens
atggaggcctgtccacacatacgctacgccttccagaatgacaagctgttgctccagcaggctagtgtagggcggctcaccttggtcaacaagaccaccatcctgctgcgcccgatgaagaccacaactgtggacctaggcctctatgcccgcccacccgagggtcatgggctcatgctgtggggcagcacctcccgtccggtcacgtctcatgttggcatcatcgatcccggctacacgggggaactccggctaatcctccagaatcagcggcgctacaactccacgctgcgtccatcggagctcaaaatccacctggctgccttcagatatgccaccccccagatggaggaggacaagggtcccatcaaccacccccagtaccccggggacgtgggcctggacgtctctttgccaaaggacctggccctcttcccccatcagaccgtctcagtgacactcaccgtgccccccccttctatccctcaccacaggccgacaatctttggcaggtcgggcctggccatgcagggtattctagtgaagccctgcaggtggcgccggggtggggtggacgtcagcctgaccaactttagtgaccagaccgtgttccttaacaagtaccggcgcttctgtcagcttgtttaccttcacaagcaccacctcacctccttctacagcccccacagtgacgcgggggtccttggccccagatctctctttaggtgggccagctgcaccttcgaggaggtgccgagcctggccatgggtgatagtgggctgagcgaggcgctcgaggggagacaggggagggggtttggatcctcgggtcaatga

SEQ ID NO: 54 - protein, 278 aa, Homo sapiens
MEACPHIRYAFQNDKLLLQQASVGRLTLVNKTTILLRPMKTTTVDLGLYARPPEGHGLMLWGSTSRPVTSHVGIIDPGYTGELRLILQNQRRYNSTLRPSELKIHLAAFRYATPQMEEDKGPINHPQYPGDVGLDVSLPKDLALFPHQTVSVTLTVPPPSIPHHRPTIFGRSGLAMQGILVKPCRWRRGGVDVSLTNFSDQTVFLNKYRRFCQLVYLHKHHLTSFYSPHSDAGVLGPRSLFRWASCTFEEVPSLAMGDSGLSEALEGRQGRGFGSSGQ

SEQ ID NO: 55 - DNA, 309 bp, Homo sapiens
atggggaaggtcctaagaaagccgtttgcaaaggctgtgccactgctcttcctcgccgccacctggcttctgaccggggtgctgccggccggcgcttccagtcccacaaacgcggcggcggcttccctgactgaagcccaggaccagttctactcctacacatgtaatgcggacacattctcgccttctttgaccagctttgcctccatctgggcacttctgacgcttgtcttagtcattatagcctcagccatctacctgatgtacgtctgctttaacaagtttgtgaacacgctgctgacggattag

SEQ ID NO: 56 - protein, 102 aa, Homo sapiens
MGKVLRKPFAKAVPLLFLAATWLLTGVLPAGASSPTNAAAASLTEAQDQFYSYTCNADTFSPSLTSFASIWALLTLVLVIIASAIYLMYVCFNKFVNTLLTD

SEQ ID NO: 57 - DNA, 489 bp, Homo sapiens
atgtcagctccacgcaaagtcagattgccttctgttaaggctgttgacatgagcatggaagacatggccgcccgcctggctcgcctggagtctgagaataaggctctgaagcaacaggtcctcagagggggtgcctgtgcctcgtctacctctgttccttctgctccagtgcctccgcctgagccgcttacagctcgacagcgagaggtaatgattacgcaggccacgggccgtttggcgtctcaggctatgaagaagattgaagacaaggttcggaaatctgttgacggtgtaactacccgcaatgaaatggaaaatatattgcaaaatctgaccctccgcattcaagtatctatgttgggtgcaaaaggccaacccagccctggtgagggaacacgaccacgagaatcaaacgaccccaacgccacccgacgtgcccgctcccgctcccggggacgtgaagcaaagaaagtgcaaatttctgattaa

SEQ ID NO: 58 - protein, 162 aa, Homo sapiens
MSAPRKVRLPSVKAVDMSMEDMAARLARLESENKALKQQVLRGGACASSTSVPSAPVPPPEPLTARQREVMITQATGRLASQAMKKIEDKVRKSVDGVTTRNEMENILQNLTLRIQVSMLGAKGQPSPGEGTRPRESNDPNATRRARSRSRGREAKKVQISD

SEQ ID NO: 59 - DNA, 2724 bp, Homo sapiens
atggaggcagccttgcttgtgtgtcagtacaccatccagagcctgatccatctcacgggtgaagatcctggttttttcaatgttgagattccggaattcccattttaccccacatgcaatgtttgcacggcagatgtcaatgtaactatcaatttcgatgtcgggggcaaaaagcatcaacttgatcttgactttggccagctgacaccccatacgaaggctgtctaccaacctcgaggtgcatttggtggctcagaaaatgccaccaatctctttctactggagctccttggtgcaggagaattggctctaactatgcggtctaagaagcttccaattaacgtcaccaccggagaggagcaacaagtaagcctggaatctgtagatgtctactttcaagatgtgtttggaaccatgtggtgccaccatgcagaaatgcaaaaccccgtgtacctgataccagaaacagtgccatacataaagtgggataactgtaattctaccaatataacggcagtagtgagggcacaggggctggatgtcacgctacccttaagtttgccaacgtcagctcaagactcgaatttcagcgtaaaaacagaaatgctcggtaatgagatagatattgagtgtattatggaggatggcgaaatttcacaagttctgcccggagacaacaaatttaacatcacctgcagtggatacgagagccatgttcccagcggcggaattctcacatcaacgagtcccgtggccaccccaatacctggtacagggtatgcatacagcctgcgtctgacaccacgtccagtgtcacgatttcttggcaataacagtatcctgtacgtgttttactctgggaatggaccgaaggcgagcgggggagattactgcattcagtccaacattgtgttctctgatgagattccagcttcacaggacatgccgacaaacaccacagacatcacatatgtgggtgacaatgctacctattcagtgccaatggtcacttctgaggacgcaaactcgccaaatgttacagtgactgccttttgggcctggccaaacaacactgaaactgactttaagtgcaaatggactctcacctcggggacaccttcgggttgtgaaaatatttctggtgcatttgcgagcaatcggacatttgacattactgtctcgggtcttggcacggcccccaagacactcattatcacacgaacggctaccaatgccaccacaacaacccacaaggttatattctccaaggcacccgagagcaccaccacctcccctaccttgaatacaactggatttgctgatcccaatacaacgacaggtctacccagctctactcacgtgcctaccaacctcaccgcacctgcaagcacaggccccactgtatccaccgcggatgtcaccagcccaacaccagccggcacaacgtcaggcgcatcaccggtgacaccaagtccatctccatgggacaacggcacagaaagtaaggcccccgacatgaccagctccacctcaccagtgactaccccaaccccaaatgccaccagccccaccccagcagtgactaccccaaccccaaatgccaccagccccaccccagcagtgactaccccaaccccaaatgccaccagccccaccttgggaaaaacaagtcctacctcagcagtgactaccccaaccccaaatgccaccagccccaccttgggaaaaacaagccccacctcagcagtgactaccccaaccccaaatgccaccagccccaccttgggaaaaacaagccccacctcagcagtgactaccccaaccccaaatgccaccggccctactgtgggagaaacaagtccacaggcaaatgccaccaaccacaccttaggaggaacaagtcccaccccagtagttaccagccaaccaaaaaatgcaaccagtgctgttaccacaggccaacataacataacttcaagttcaacctcttccatgtcactgagacccagttcaaacccagagacactcagcccctccaccagtgacaattcaacgtcacatatgcctttactaacctccgctcacccaacaggtggtgaaaatataacacaggtgacaccagcctctatcagcacacatcatgtgtccaccagttcgccagcaccccgcccaggcaccaccagccaagcgtcaggccctggaaacagttccacatccacaaaaccgggggaggttaatgtcaccaaaggcacgcccccccaaaatgcaacgtcgccccaggcccccagtggccaaaagacggcggttcccacggtcacctcaacaggtggaaaggccaattctaccaccggtggaaagcacaccacaggacatggagcccggacaagtacagagcccaccacagattacggcggtgattcaactacgccaagaccgagatacaatgcgaccacctatctacctcccagcacttctagcaaactgcggccccgctggacttttacgagcccaccggttaccacagcccaagccaccgtgccagtcccgccaacgtcccagcccagattctcaaacctctccatgctagtactgcagtgggcctctctggctgtgctgacccttctgctgctgctggtcatggcggactgcgcctttaggcgtaacttgtctacatcccatacctacaccaccccaccatatgatgacgccgagacctatgtataa

SEQ ID NO: 60 - protein, 907 aa, Homo sapiens
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATSPTLGKTSPTSAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 61 - DNA, 447 bp, Homo sapiens
atgtgtccaccagttcgccagcaccccgcccaggcaccaccagccaagcgtcaggccctggaaacagttccacatccacaaaaccgggggaggttaatgtcaccaaaggcacgcccccccaaaatgcaacgtcgccccaggcccccagtggccaaaagacggcggttcccacggtcacctcaacaggtggaaaggccaattctaccaccggtggaaagcacaccacaggacatggagcccggacaagtacagagcccaccacagattacggcggtgattcaactacgccaagaccgagatacaatgcgaccacctatctacctcccagcacttctagcaaactgcggccccgctggacttttacgagcccaccggttaccacagcccaagccaccgtgccagtcccgccaacgtcccagcccagattctcaaacctctccatgctag

SEQ ID NO: 62 - protein, 148 aa, Homo sapiens
MCPPVRQHPAQAPPAKRQALETVPHPQNRGRLMSPKARPPKMQRRPRPPVAKRRRFPRSPQQVERPILPPVESTPQDMEPGQVQSPPQITAVIQLRQDRDTMRPPIYLPALLANCGPAGLLRAHRLPQPKPPCQSRQRPSPDSQTSPC

SEQ ID NO: 63 - DNA, 2835 bp, Homo sapiens
atggacaaggacaggccgggtcccccggccctggatgacaacatggaagaagaagtcccatctacctcggttgtgcaggaacaggtatcggcgggagattgggaaaatgtcctcatagagttatcagatagcagctcagaaaaggaagcagaagatgcccacctggagccggcccagaagggtacgaagagaaagcgggtcgatcatgatgccggtgggtcagctccagcacgacccatgctcccaccccagccggatctccctgggcgagaagccattctccgcaggtttccactagatctaagaacacttcttcaagcgattggagccgcggctacgcgcatcgacacacgagccatagaccagtttttcggatcccagatttcaaataccgagatgtacataatgtatgccatggccattcgacaggccattagagatcgtcggagaaatccagcttctcgtagagatcaggccaaatggagactgcaaaccctggccgccggatggcctatgggttaccaggcatacagcagctggatgtacagctacaccgatcaccagacgactcccacattcgtacatctccaggcgacacttgggtgcacaggtggccgtaggtgtcacgtgaccttttccgccggcacctttaagctgccgcgatgtacccccggggatcgccagtggttgtatgttcagagctccgtgggtaacattgtacagagctgtaatccccgctacagtattttctttgactatatggctatacacaggagcctcacgaaaatctgggaggaagttttaacacctgaccagcgtgtttcatttatggaattcctaggatttttgcagagaacggatttgtcctatatcaagagctttgtcagcgatgccctgggcaccactagtatccaaacaccgtggatcgatgacaatcctagcacggagacggcacaggcttggaatgccggctttctccggggtcgtgcgtatgggatagacttgcttagaactgaaggggaacatgtcgaaggtgctaccggtgaaacgcgagaagaaagtgaggacacggagagcgatggagatgatgaagatcttccttgtatagtgtccagaggtggacctaaggtcaaacgaccccctatatttataagacgtctgcacaggttgctgctgatgagagcgggcaaacgaacagaacagggcaaggaggtactggaaaaggcccgtgggagcacttatggcacacctaggccgcctgttccgaaaccaagaccagaggtcccacaaagcgacgagacagctaccagtcacgggtcggcgcaagtcccagaacccccaaccattcacctagcagctcagggaatggcatacccattacatgaacaacacggcatggccccgtgtccggtagcacaggccccacctacgcccttgccccctgtatctccaggggatcaactcccaggtgtttttagcgacgggcgagtggcgtgtgcaccagtacccgccccggctgggcctattgtccggccctgggagccatccctgacacaggctgcggggcaggcctttgcacccgttagaccacaacacatgccagtagaacccgtccctgtcccgacagtggcacttgagcgaccagtttaccccaagccagttcgtccggcacctcctaagattgctatgcagggccccggggaaacttctggcattagacgcgcgcgggagcgttggaggcccgcaccttggacgccaaatccaccccgttctcccagtcagatgtccgtgcgtgaccgtctggctcgtttgcgtgctgaggcacaggtcaaacaggctagtgttgaggtgcagcccccccagttgacccaagtatcccctcagcaaccaatggaggggccgttggtaccagagcagcagatgttccctggtgccccctttagccaggttgctgatgtggtccgggcacctggggtaccggcgatgcagccacagtactttgacctccccttaattcaacccattagccagggggcacccgtggccccgttgagggctagtatgggcccggtacctccggtaccggcaacacagccacagtattttgacatccccttaactgaacccattaaccagggggcatccgcggcccattttctccctcagcaaccgatggaggggccgttggtacctgagcagtggatgttcccaggtgccgccctgagccagagtgttaggccaggggtagcgcagtcacaatattttgacctccccttaactcaacccattaaccatggggcacccgcagcccatttcctccatcagccaccaatggaggggccgtgggtacccgagcagtggatgttccaaggtgccccccctagccaaggcactgacgtggtccaacatcagctggatgctttggggtatacactccatggtcttaaccatcccggggttcccgtgtctcctgccgttaaccaatatcatctcagccaggctgcctttgggttacctattgatgaggatgagagtggcgaggggtccgatacctccgagccgtgtgaagctcttgatttgtcaatccatggcaggccctgccctcaggcccccgagtggcctgttcaagaggagggtggccaggatgccaccgaggttcttgatttgtcaatccatggcaggccccgccctcggacccccgagtggcctgttcaaggggaaggtggccaaaatgtcacaggccctgaaactagaagggtggtggtgtcagctgttgttcacatgtgtcaggatgacgagtttccggatctacaagatcctccagatgaggcctaa

SEQ ID NO: 64 - protein, 944 aa, Homo sapiens
MDKDRPGPPALDDNMEEEVPSTSVVQEQVSAGDWENVLIELSDSSSEKEAEDAHLEPAQKGTKRKRVDHDAGGSAPARPMLPPQPDLPGREAILRRFPLDLRTLLQAIGAAATRIDTRAIDQFFGSQISNTEMYIMYAMAIRQAIRDRRRNPASRRDQAKWRLQTLAAGWPMGYQAYSSWMYSYTDHQTTPTFVHLQATLGCTGGRRCHVTFSAGTFKLPRCTPGDRQWLYVQSSVGNIVQSCNPRYSIFFDYMAIHRSLTKIWEEVLTPDQRVSFMEFLGFLQRTDLSYIKSFVSDALGTTSIQTPWIDDNPSTETAQAWNAGFLRGRAYGIDLLRTEGEHVEGATGETREESEDTESDGDDEDLPCIVSRGGPKVKRPPIFIRRLHRLLLMRAGKRTEQGKEVLEKARGSTYGTPRPPVPKPRPEVPQSDETATSHGSAQVPEPPTIHLAAQGMAYPLHEQHGMAPCPVAQAPPTPLPPVSPGDQLPGVFSDGRVACAPVPAPAGPIVRPWEPSLTQAAGQAFAPVRPQHMPVEPVPVPTVALERPVYPKPVRPAPPKIAMQGPGETSGIRRARERWRPAPWTPNPPRSPSQMSVRDRLARLRAEAQVKQASVEVQPPQLTQVSPQQPMEGPLVPEQQMFPGAPFSQVADVVRAPGVPAMQPQYFDLPLIQPISQGAPVAPLRASMGPVPPVPATQPQYFDIPLTEPINQGASAAHFLPQQPMEGPLVPEQWMFPGAALSQSVRPGVAQSQYFDLPLTQPINHGAPAAHFLHQPPMEGPWVPEQWMFQGAPPSQGTDVVQHQLDALGYTLHGLNHPGVPVSPAVNQYHLSQAAFGLPIDEDESGEGSDTSEPCEALDLSIHGRPCPQAPEWPVQEEGGQDATEVLDLSIHGRPRPRTPEWPVQGEGGQNVTGPETRRVVVSAVVHMCQDDEFPDLQDPPDEA

SEQ ID NO: 65 - DNA, 105 bp, Homo sapiens
gtttcagcgcatcgacacacgagccatagaccagtttttcggatcccagatttcaaataccgagatgtacataatgtatgccatggccattcgacaggccattag

SEQ ID NO: 66 - protein, 34 aa, Homo sapiens
VSAHRHTSHRPVFRIPDFKYRDVHNVCHGHSTGH

SEQ ID NO: 67 - DNA, 2895 bp, Homo sapiens
atgaagaaagcgtggctcagcagagcacagcaagccgatgccgggggggcatctggctccgaggacccaccagattatggagatcaaggtaatgtgacacaggtgggatctgagcctatttcacctgagattggcccctttgaactctctgcggccagtgaggatgatcctcaatctgggccagtggaagagaatttagatgccgctgcaagagaggaagaggaacctcatgagcaggagcacaatggtggtgacgatcccttggatgtccatactcgccagcctagatttgtggatgtgaacccaacgcaggctccagtgatccaactagtccacgctgtctatgattccatgttggtaagaggcacctagaacatttccagatgtttcgcttggattttttggccagtcttaattgattgtcattggtttcagcaatcggacctccggcccctaggcagtttattccttgagcaaaacctgaacatcgaagaatttatatggatgtgcatgaccgtgcgtcacagatgtcaggccatcagaaaaaaaccattaccaattgttaagcagaggcgttggaagctcctgtcatcttgcagatcctggcgtatgggttaccgcacgcataacctcaaagtaaacagttttgagtcagggggggacaatgtccacccggtccttgtgactgctacgctaggatgtgatgagggcacgcggcatgcaacaacgtacagtgctggcattgtacagataccacgaatatcagaccaaaaccaaaagatcgaaacagccttcctgatggcacgtcgtgctaggtcactttcggcagaaagatatactttgttctttgatttagtatcctccggaaacaccctgtatgctatatggattgggctgggcacgaaaaaccgagtttcatttattgagtttgtaggatggttatgcaagaaggaccacactcatatacgcgaatggttccgccagtgcaccgggagacccaaagcagccaagccatggttaagagcgcatcctgtcgccattccttatgatgatccgttaacaaacgaggagattgatctggcctatgcccgcgggcaggccatgaatattgaggctcctagactgccagatgatcctataattgttgaggatgacgacgaaagtgaggaaattgaagctgaaagcgacgaggaggaagacaagagtggaatggaatctcttaaaaatataccgcaaacactgccgtacaatccaacagtatacggcaggcccgcggtgtttgaccgaaagtcagatgcaaaatcaaccaaaaaatgcagggccatagtaactgactttagtgtaatcaaggccattgaagaggaacacagaaagaagaaggcagccagaacagagcagccaagagccacgcctgaatcccaggcccccacagtggtcctccagcgaccacccacgcagcaagagcctggccccgtcggcccactgagtgtccaggctcggctggagccatggcaacctttgcctgggccccaagtgacagcagttctacttcacgaagaatccatgcagggtgtccaagtacatggttcgatgctagaccttcttgaaaaagacgatgaagtcatggagcagagggttatggcaaccctactgccaccagtaccacaacagccccgggctggcagaagaggcccttgtgtcttcaccggtgacctaggcatagagagtgatgagcccgcttcgacagagccggttcatgatcagctactgcctgccccaggacctgaccctcttgaaatccaaccactaacgtcccccaccacgtctcaacttagcagttcggcaccaagctgcgcacaaactccatggccggtggttcagccaagtcagactccagatgacccaacgaaacagtcccggccaccggaaacagctgccccacgccagtggccaatgcccctgcgacctatccctatgcgccccttgcggatgcagccaatcccatttaatcatccagtgggacccactccccatcagacacctcaagtggaaataacaccatataagcccacttgggctcagatagggcacattccatatcagcctacaccaacgggtcctgctaccatgctgttgcgccagtgggcacccgccaccatgcagacaccaccgagagcgcccactcccatgtcaccacctgaggtgccacccgttccccggcagaggcctcggggggcgcccactcccacgccacctcctcaggtgccgcccgttccccggcagaggcctcggggggcgcccactcccacgccacctcctcaggtgctgcccactcccatgcagctggcactaagggctcctgctggtcagcaggggccgacaaagcaaattttgcgccaattgttaacggggggcgtcaagaaagggagaccatcacttaagttacaggccgcccttgagcgtcaagccgctgcgggctggcagccttcaccagggtccggcaccagtgacaagattgtgcaggcgcctattttctacccacccgttttgcagcccatacaggttatggggcaagggggttccccaacggccatggccgcctcagcggtgacacaggcacccacggaatataccagggaaaggaggggagtggggcctatgcctcccaccgatattccgccgtctaaacgagcgaagatcgaggcctatacagagcccgagatgccgcacgggggggcctcgcactctcccgtcgttatcttggagaatgtcggccaggggcaacagcagactctggagtgcggaggaactgctaaacaggaaagggacatgttggggctgggggacattgcagtttcttccccttcctcttctgaaacatcgaacgatgagtga

SEQ ID NO: 68 - protein, 938 aa, Homo sapiens
MKKAWLSRAQQADAGGASGSEDPPDYGDQGNVTQVGSEPISPEIGPFELSAASEDDPQSGPVEENLDAAAREEEEPHEQEHNGGDDPLDVHTRQPRFVDVNPTQAPVIQLVHAVYDSMLQSDLRPLGSLFLEQNLNIEEFIWMCMTVRHRCQAIRKKPLPIVKQRRWKLLSSCRSWRMGYRTHNLKVNSFESGGDNVHPVLVTATLGCDEGTRHATTYSAGIVQIPRISDQNQKIETAFLMARRARSLSAERYTLFFDLVSSGNTLYAIWIGLGTKNRVSFIEFVGWLCKKDHTHIREWFRQCTGRPKAAKPWLRAHPVAIPYDDPLTNEEIDLAYARGQAMNIEAPRLPDDPIIVEDDDESEEIEAESDEEEDKSGMESLKNIPQTLPYNPTVYGRPAVFDRKSDAKSTKKCRAIVTDFSVIKAIEEEHRKKKAARTEQPRATPESQAPTVVLQRPPTQQEPGPVGPLSVQARLEPWQPLPGPQVTAVLLHEESMQGVQVHGSMLDLLEKDDEVMEQRVMATLLPPVPQQPRAGRRGPCVFTGDLGIESDEPASTEPVHDQLLPAPGPDPLEIQPLTSPTTSQLSSSAPSCAQTPWPVVQPSQTPDDPTKQSRPPETAAPRQWPMPLRPIPMRPLRMQPIPFNHPVGPTPHQTPQVEITPYKPTWAQIGHIPYQPTPTGPATMLLRQWAPATMQTPPRAPTPMSPPEVPPVPRQRPRGAPTPTPPPQVPPVPRQRPRGAPTPTPPPQVLPTPMQLALRAPAGQQGPTKQILRQLLTGGVKKGRPSLKLQAALERQAAAGWQPSPGSGTSDKIVQAPIFYPPVLQPIQVMGQGGSPTAMAASAVTQAPTEYTRERRGVGPMPPTDIPPSKRAKIEAYTEPEMPHGGASHSPVVILENVGQGQQQTLECGGTAKQERDMLGLGDIAVSSPSSSETSNDE

SEQ ID NO: 69 - DNA, 3053 bp, Homo sapiens
atggaatcatttgaaggacagggggactctagacagtcacccgacaatgagcggggagataatgtacagactaccggcgagcatgatcaggaccctgggccggggcctccatccagtggggcttctgagagattggtaccagaagagtcatactcaagagatcagcaaccttgggggcaaagcaggggtgatgaaaacagaggctggatgcagcgcatcaggcgaaggcggagaagacgggctgccttgtccggccatcttttagacacggaagacaatgtgccgccatggttgcctccacacgacatcacaccatataccgcaaggaatatcagggatgctgcctgccgggctgtcaaggtgagtatgcctctaactgggttcatgggggccatctaaggcccacgtgtgacccatgtttccattaattttagcaatcgcacctgcaagcgctatcaaacctgatactcgatagtgggttagacacacaacacatcttgtgcttcgtgatggcagccaggcagcgtcttcaggacattcgacgtggacccttggttgcggagggcggtgtgggttggcgacattggcttctaacatctcccagccaatcctggcccatgggatatcgtacagcaacactacgcacattaactcccgtgcctaacagggttggggctgacagcatcatgttaactgccacatttggatgccaaaatgcggcacgaactctaaacaccttctctgccaccgtgtggacaccaccccatgctggaccaagagagcaagaaagatacgctcgggaagccgaggtacgcttccttcgtggtaaatggcagaggcggtaccgaagaatctatgatttgatagaactgtgtggctctctgcaccacatctggcaaaacttgctccagaccgaggagaaccttttagatttcgtgcgtttcatgggtgtcatgtccagctgcaataatccagctgtgaattactggtttcacaagacaatcggaaactttaagccatattacccgtggaatgcaccacctaatgaaaatccatatcacgcgcggagaggcataaaagaacacgtaatccagaacgcatttcgaaaggcacaaatacagggtttatcaatgttagcaacgggaggtgaacccagaggtgatgctactagtgaaacgagcagtgatgaggacaccggtagacagggttcggacgtggagctagagtcctcggacgatgagctgccatatatcgatcccaatatggagccggttcagcagaggcccgtcatgtttgtgagccgtgtgcctgcaaagaaaccgaggaaactgccttggcccacgcccaagacgcacccagtgaagcgcacaaatgttaagacctctgatagatctgataaggcagaagcacaaagcacccctgaaaggccgggcccttccgaacaatcatcagtgaccgtggagcccgcccacccgaccccggtggagatgccaatggtgattctccatcaaccacctccagtgcccaaaccggttccagtcaagcctacgccaccgccttcccgtaggagaaggggagcgtgtgttgtgtacgacgatgatgtcatagaggtgattgatgttgaaaccaccgaagattcatcgtcagtgtcacagccaaataagccacatcggaaacatcaagacggctttcaacgttcaggccgacgtcaaaaacgagccgcgcctcccaccgtgagtccttcggatactgggcctcctgccgtggggcctcctgccgcggggcctcctgccgcggggcctcctgccgcggggcctcctgccgcggggcctcctgccgcggggcctcctgccgcggggcctcgcatactggcgcctctttccgctgggcctcctgccgcggggcctcacatagtgacgcctccttccgcccggcctcgtataatggcgcctcccgtcgtacgtatgtttatgagggagcgacagctcccccagtccaccggccgtaaacctcagtgcttctgggaaatgcgggctggtcgtgaaattacacaaatgcaacaagaaccaagttcacacctgcagtccgccactcagcctacaacgcctcgcccatcatgggccccatcagtctgcgccctctcggtgatggatgctggtaaggcccagcccatagaaagttcacacttgagttccatgtcgcccacacagccgatatcgcacgaagaacaaccccggtatgaggatcctgacgctcctctggatttaagtttacatccagacgttgctgctcaaccagctccccaggctccataccagggataccaggagccgccggccccccaggctccataccagggataccaggagccgccgcccccccaggctccataccagggataccaggagccgccggcccacgggctccaatcatcttcatatccaggatatgcgggtccctggaccccaaggtctcaacatccatgttataggcacccctgggcaccatggtctcaagatcctgtgcatgggcacacccagggtccatgggatcccagggcaccacatctcccacctcagtgggatggatctgcaggacatggccaggatcaggtctcccagttcccacatctgcaatcggagacaggcccaccacgtcttcaactttcattggtgccactggtctcatcctctgcaccatcatggtcatctccccagccccgagcccccatacgccccattccaacaagattcccccctccccctatgccgttacaagatagcatggccgtggggtgtgactcatcaggtacagcatgcccaagcatgccctttgccagtgattacagtcaaggtgcatttaccccactggacattaatgccaccacgccaaaaaggcctcgagtagaagaaagttctcacggacctgcccggtgttcccaagctactgctgaagcacaggagattctcagtgacaattctgagatctccgtgttcccaaaggacgcgaagcagactgactacgatgcatccactgaaagtgagctagattaa

SEQ ID NO: 70 - protein, 992 aa, Homo sapiens
MESFEGQGDSRQSPDNERGDNVQTTGEHDQDPGPGPPSSGASERLVPEESYSRDQQPWGQSRGDENRGWMQRIRRRRRRRAALSGHLLDTEDNVPPWLPPHDITPYTARNIRDAACRAVKQSHLQALSNLILDSGLDTQHILCFVMAARQRLQDIRRGPLVAEGGVGWRHWLLTSPSQSWPMGYRTATLRTLTPVPNRVGADSIMLTATFGCQNAARTLNTFSATVWTPPHAGPREQERYAREAEVRFLRGKWQRRYRRIYDLIELCGSLHHIWQNLLQTEENLLDFVRFMGVMSSCNNPAVNYWFHKTIGNFKPYYPWNAPPNENPYHARRGIKEHVIQNAFRKAQIQGLSMLATGGEPRGDATSETSSDEDTGRQGSDVELESSDDELPYIDPNMEPVQQRPVMFVSRVPAKKPRKLPWPTPKTHPVKRTNVKTSDRSDKAEAQSTPERPGPSEQSSVTVEPAHPTPVEMPMVILHQPPPVPKPVPVKPTPPPSRRRRGACVVYDDDVIEVIDVETTEDSSSVSQPNKPHRKHQDGFQRSGRRQKRAAPPTVSPSDTGPPAVGPPAAGPPAAGPPAAGPPAAGPPAAGPPAAGPRILAPLSAGPPAAGPHIVTPPSARPRIMAPPVVRMFMRERQLPQSTGRKPQCFWEMRAGREITQMQQEPSSHLQSATQPTTPRPSWAPSVCALSVMDAGKAQPIESSHLSSMSPTQPISHEEQPRYEDPDAPLDLSLHPDVAAQPAPQAPYQGYQEPPAPQAPYQGYQEPPPPQAPYQGYQEPPAHGLQSSSYPGYAGPWTPRSQHPCYRHPWAPWSQDPVHGHTQGPWDPRAPHLPPQWDGSAGHGQDQVSQFPHLQSETGPPRLQLSLVPLVSSSAPSWSSPQPRAPIRPIPTRFPPPPMPLQDSMAVGCDSSGTACPSMPFASDYSQGAFTPLDINATTPKRPRVEESSHGPARCSQATAEAQEILSDNSEISVFPKDAKQTDYDASTESELD

SEQ ID NO: 71 - DNA, 672 bp, Homo sapiens
atggtttcatttaagcaggtgagggtgccattgtttaccgccatcgcacttgttattgttctactcctggcatactttttgccacccagggtaagaggaggagggcgggtggcagccgcggccatcacctgggtacccaaaccaaatgtagaggtctggccggtggatcctccaccgccggttaactttaacaagacggccgagcaggagtatggggacaaagaggtaaaactgccacattggacacccaccctgcacacatttcaggtaccccaaaactataccaaagctaactgtacatactgcaacaccagagaatacacattttcatataaaggatgctgtttttatttcaccaaaaagaagcacacctggaatgggtgtttccaagcctgtgcagagctatatccatgcacttatttttatgggccaacgcccgatattctacctgtggtaactagaaatctgaatgccattgagtccctttgggtcggggtgtacagggtgggagaagggaactggacatcattagatggggggacttttaaggtttatcaaatttttggctctcattgtacatatgtcagcaaatttagtacagttccagtctcacaccatgagtgttcattccttaaaccatgtttatgtgtcagtcaaagatcaaatagctaa

SEQ ID NO: 72 - protein, 223 aa, Homo sapiens
MVSFKQVRVPLFTAIALVIVLLLAYFLPPRVRGGGRVAAAAITWVPKPNVEVWPVDPPPPVNFNKTAEQEYGDKEVKLPHWTPTLHTFQVPQNYTKANCTYCNTREYTFSYKGCCFYFTKKKHTWNGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNS

SEQ ID NO: 73 - DNA, 946 bp, Homo sapiens
atgatggacccaaactcgacttctgaagatgtaaaatttacacctgacccataccaggtgccttttgtacaagcttttgaccaagctaccagagtctatcaggacctgggagggccatcgcaagctcctttgccttgtgtgctgtggccggtgctgccagagcctctgccacaaggccagctaactgcctatcatgtttcaaccgctccgactgggtcgtggttttctgcccctcagcctgctcctgagaatgcttatcaagcttatgcagcacctcagctgttcccagtctccgacataacccagaatcaacagactaaccaagccgggggagaagcacctcaacctggagacaattctactgttcaaacagcagcagcagtggtgtttgcttgccccggggctaaccaaggacaacagctagcagacattggtgttccacagcctgcaccagtggctgccccggcacgacgcacacggaaaccacaacagccagaatcggtaaggaggggctcaaggaggggcggcagcggacggtggggctcatggaggggcggcagcggatggtggggcttgtttcaactgactaggcaccacttgtagtaacgcattcccttgctcgcagctggaggaatgcgattctgaactagaaataaagcgatacaagaatcgggtggcttccagaaaatgccgggccaagtttaagcaactgctgcagcactaccgtgaggtcagtatatactaacacgaatgcccaggcggctagcgtcccaaacataaatgccccatctaaacgcctgatttttgtccgcaggtggctgctgccaaatcatctgaaaatgacaggctgcgcctcctgttgaagcagatgtgcccaagcctggatgttgactccattatcccccggacaccagatgttttacacgaggatctcttaaatttctaa

SEQ ID NO: 74 - protein, 245 aa, Homo sapiens
MMDPNSTSEDVKFTPDPYQVPFVQAFDQATRVYQDLGGPSQAPLPCVLWPVLPEPLPQGQLTAYHVSTAPTGSWFSAPQPAPENAYQAYAAPQLFPVSDITQNQQTNQAGGEAPQPGDNSTVQTAAAVVFACPGANQGQQLADIGVPQPAPVAAPARRTRKPQQPESLEECDSELEIKRYKNRVASRKCRAKFKQLLQHYREVAAAKSSENDRLRLLLKQMCPSLDVDSIIPRTPDVLHEDLLNF

SEQ ID NO: 75 - DNA, 1818 bp, Homo sapiens
atgaggcctaaaaaggatggcttggaagactttctgaggctaactcctgaaatcaaaaagcagctgggctctctggtctctgactactgcaacgtcctcaacaaggaatttacagccgggagtgtggagattactctgagatcctacaaaatatgcaaggcatttataaatgaggccaaggcccacgggcgagaatggggcgggctaatggccacgctcaacatctgcaatttttgggccattctccgaaacaacagggtaagaagacgggctgagaatgccggcaacgacgcatgttccatcgcgtgccctatagtgatgcgctacgtgttagaccacctgatagtggtcactgacagattcttcatccaggcccccagcaaccgggtgatgattcctgccaccataggcaccgctatgtacaagctcctaaaacacagtcgggtgcgggcctacacctacagcaaggtgctgggcgtggaccgcgcggccatcatggcctccggcaagcaggtagtggaacacctgaacaggatggagaaggaaggcctcctaagctccaagttcaaggccttttgcaagtgggtgttcacctatcccgtcctcgaggagatgttccagactatggtctcgtccaagacaggccatctgacggacgatgttaaggatgtcagggctctgattaagacactgccccgggcctcctactccagccacgccggacagaggagctacgtgagcggcgtgcttcccgcgtgcctgctgtcaaccaagtccaaggcagtggaaactcctatcctcgtgtccggagccgacaggatggacgaggagctcatggggaatgatgggggtgcctctcacaccgaggcccgctactcggagtccggacagtttcatgcttttacagatgaactcgaaagtctcccgagcccgaccatgcccctgaagcccggtgcccaaagcgccgactgcggtgacagcagttccagcagcagtgactcgggcaacagtgacaccgagcagagcgagcgggaagaggccagggccgaggccccgcgcctgcgggccccaaagtcgcgccggacatccaggcccaaccgtggtcaaactccatgtccttccaacgcggcggaacctgaacagccttggatagcagcggtccaccaagagagcgatgagagacccatattcccccacccctcaaagcccacctttcttcctcccgttaaaaggaagaagggcctcagggatagccgggaaggtatgttcctgccaaagccggaagcgggcagtgccatatctgacgtgttcgaggggcgagaggtgtgtcagccaaagaggatcaggcccttccatccacccggatccccgtgggccaaccggcccctgcctgcctctttggctcccacccccacaggacctgtccatgaaccggtcggatccctaacgccagccccggtgccccagccacttgacccggcccccgcagtaacccccgaggcaagtcatctgttggaggaccctgatgaagaaaccagtcaggccgtgaaggccctaagggagatggctgacactgttattccccagaaggaggaagcagccatatgtggacagatggacctgagccacccgccccctcgtggccatttggacgaactgaccacaacactagagtccatgacagaggatttgaatctggactcccccctgacccccgaacttaatgaaatcttggatacatttctaaatgatgaatgtctgctgcatgccatgcatatttcaactgggctgtctatttttgacaccagcttattttag

SEQ ID NO: 76 - protein, 605 aa, Homo sapiens
MRPKKDGLEDFLRLTPEIKKQLGSLVSDYCNVLNKEFTAGSVEITLRSYKICKAFINEAKAHGREWGGLMATLNICNFWAILRNNRVRRRAENAGNDACSIACPIVMRYVLDHLIVVTDRFFIQAPSNRVMIPATIGTAMYKLLKHSRVRAYTYSKVLGVDRAAIMASGKQVVEHLNRMEKEGLLSSKFKAFCKWVFTYPVLEEMFQTMVSSKTGHLTDDVKDVRALIKTLPRASYSSHAGQRSYVSGVLPACLLSTKSKAVETPILVSGADRMDEELMGNDGGASHTEARYSESGQFHAFTDELESLPSPTMPLKPGAQSADCGDSSSSSSDSGNSDTEQSEREEARAEAPRLRAPKSRRTSRPNRGQTPCPSNAAEPEQPWIAAVHQESDERPIFPHPSKPTFLPPVKRKKGLRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 77 - DNA, 933 bp, Homo sapiens
atggctagtagtaacagaggaaatgcccgaccattaaaatctttcctccatgagctttacctgaaacactatcccgaagtgggggatgtggtgcatctactgaacaccatcggggtcgactgcgacctcccacctagccacccactcctgacagcccagagggggctgttcctggcaagagtcttgcaggctgtacagcagcacaagctgctggaagacaccatcgtccccaagatcttaaagaagctggcttatttcttagagctgctaagctactactcccccaaggatgaacagcgtgacatcgccgaggttcttgaccacctcaagacgaatcgggacctggggctggacgacagactctgggccctgattaggaaactgcgccaagacagacaccatgcctctgtaaatgtcctcatgccaggaagcgactacacagccgtgtcgctgcagtactacgacggcatctccataggtatgaggaaggtaatcgcggatgtctgccgcagtggctatgcctccatgccctccatgacggccacgcacaacctctcccaccagctcttgatggcgtccgggcccagtgaggaaccgtgcgcctggcgcgggttctttaaccaggtcctcctctggactgtggccctctgcaagtttcgcagatgcatttactataactacattcagggatctatagccaccatctcccagcttctgcacctcgagatcaaggccctctgcagctggataatatcccaggatggcatgcgcctctttcaacacagcaggcctctcctcaccctctgggagagcgtggccgcaaatcaggaggtcacggatgccattaccctgcctgactgcgctgaatacatagacctactaaagcacacaaaacatgtcttagaaaactgttctgccatgcaatacaaataa

SEQ ID NO: 78 - protein, 310 aa, Homo sapiens
MASSNRGNARPLKSFLHELYLKHYPEVGDVVHLLNTIGVDCDLPPSHPLLTAQRGLFLARVLQAVQQHKLLEDTIVPKILKKLAYFLELLSYYSPKDEQRDIAEVLDHLKTNRDLGLDDRLWALIRKLRQDRHHASVNVLMPGSDYTAVSLQYYDGISIGMRKVIADVCRSGYASMPSMTATHNLSHQLLMASGPSEEPCAWRGFFNQVLLWTVALCKFRRCIYYNYIQGSIATISQLLHLEIKALCSWIISQDGMRLFQHSRPLLTLWESVAANQEVTDAITLPDCAEYIDLLKHTKHVLENCSAMQYK

SEQ ID NO: 79 - DNA, 1614 bp, Homo sapiens
atgagtgggcagcagagaggctcggttattttggttcctgaacatctggctggggcattaactaagcttatgagcgattttatcacaggacaagatgtcactctttctggaggaaatattgcagtcaaaattcgcgatgctataaaccagacccccgggggtggtgatgtagctatactttcttccctgtttgctttatggaatgccctcccaacatctggtagacaatcctccagggacgatttaatcccagccgccgtgcaggccttaaccacggcccacaacttatgtctgggtgttattccaggtgagacctcacacaaggacacacccgagtcattgctccgggctatcgtgacgggtctccaaaaattgtgggtggattcgtgcggatgtccagagtgcctacaatgtcttaagggattgaaggcaattaagcccggcctttatgaaatccctaggataataccacacactaagcagtgtagtcctgtcaatctcctgaacatgttggtccacaagcttgtggctttacgtggtcatgtgcagcttgcatacgacgcccgtgtcctgacgcctgactttcacgaaatccctgacctcgatgactccgatgctgttttcgcacgcaccttattggcagccttatttcacctcaatatgttctttattctcaaagattacataacacaagactccatgagcttgaagcaggccctcagtggtcattggatgtctgccacgggcaaccccctgcctgcagcaccggaaaccctgcgagactacttggaagctttccgaaattcggataatcacttttatctcccgacgacagggcctttaaacaccttccaatttcccgaagagcttctggggcgcgttgttgttattgattcctctttgtgtgccgccagtcacgttcaggacgttatcacccatggtgttggggcgggtgttcctcgtcctcggttttcggccctgcctccggccccatcccgcgagccccagcagacatgctctcagttaacgagcagagggaatgaaagctcacggcgaaacttgggccagcccggggggacctcccctgctgttcccccagtttgccccatcgtttccctgacggcctcaggggccaagcaaaaccgcgggggcatgggatccttgcacttagccaagcctgaggaaacctcccccgccgtctccccagtatgccccatcgcttccccagcggcctccaggtccaagcagcactgcggggtcactggatcctcacaggccgcacccagcttttcttccgttgccccagtagcatctctgtctggtgaccttgaagaggaagaggaggggtcccgagaatccccatccctaccgtccagcaaaaagggggacgaggaatttgaggcctggcttgaggctcaggacgcaaatcttgaggatgttcagcgggagttttccgggctgcgagtaattggtgatgaggacgaggatggttcggaggatggggaattttcagacctggatctgtctgacagcgaccatgaaggggatgagggtgggggggctgttggagggggcaggagtctgcactccctgtattcactgagcgtcgtctaa

SEQ ID NO: 80 - protein, 537 aa, Homo sapiens
MSGQQRGSVILVPEHLAGALTKLMSDFITGQDVTLSGGNIAVKIRDAINQTPGGGDVAILSSLFALWNALPTSGRQSSRDDLIPAAVQALTTAHNLCLGVIPGETSHKDTPESLLRAIVTGLQKLWVDSCGCPECLQCLKGLKAIKPGLYEIPRIIPHTKQCSPVNLLNMLVHKLVALRGHVQLAYDARVLTPDFHEIPDLDDSDAVFARTLLAALFHLNMFFILKDYITQDSMSLKQALSGHWMSATGNPLPAAPETLRDYLEAFRNSDNHFYLPTTGPLNTFQFPEELLGRVVVIDSSLCAASHVQDVITHGVGAGVPRPRFSALPPAPSREPQQTCSQLTSRGNESSRRNLGQPGGTSPAVPPVCPIVSLTASGAKQNRGGMGSLHLAKPEETSPAVSPVCPIASPAASRSKQHCGVTGSSQAAPSFSSVAPVASLSGDLEEEEEGSRESPSLPSSKKGDEEFEAWLEAQDANLEDVQREFSGLRVIGDEDEDGSEDGEFSDLDLSDSDHEGDEGGGAVGGGRSLHSLYSLSVV

SEQ ID NO: 81 - DNA, 1926 bp, Homo sapiens
atgtctgacgaggggccaggtacaggacctggaaatggcctaggagagaagggagacacatctggaccagaaggctccggcggcagtggacctcaaagaagagggggtgataaccatggacgaggacggggaagaggacgaggacgaggaggcggaagaccaggagccccgggcggctcaggatcagggccaagacatagagatggtgtccggagaccccaaaaacgtccaagttgcattggctgcaaagggacccacggtggaacaggagcaggagcaggagcgggaggggcaggagcaggaggggcaggagcaggaggaggggcaggagcaggaggaggggcaggaggggcaggaggggcaggaggggcaggagcaggaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggggcaggaggggcaggaggggcaggagcaggaggggcaggagcaggaggaggggcaggaggggcaggaggggcaggagcaggaggggcaggagcaggaggggcaggagcaggaggggcaggagcaggaggggcaggaggggcaggagcaggaggggcaggaggggcaggagcaggaggggcaggaggggcaggagcaggaggaggggcaggaggggcaggagcaggaggaggggcaggaggggcaggagcaggaggggcaggaggggcaggagcaggaggggcaggaggggcaggagcaggaggggcaggaggggcaggagcaggaggaggggcaggagcaggaggggcaggagcaggaggtggaggccggggtcgaggaggcagtggaggccggggtcgaggaggtagtggaggccggggtcgaggaggtagtggaggccgccggggtagaggacgtgaaagagccagggggggaagtcgtgaaagagccagggggagaggtcgtggacgtggagaaaagaggcccaggagtcccagtagtcagtcatcatcatccgggtctccaccgcgcaggccccctccaggtagaaggccatttttccaccctgtaggggaagccgattattttgaataccaccaagaaggtggcccagatggtgagcctgacgtgcccccgggagcgatagagcagggccccgcagatgacccaggagaaggcccaagcactggaccccggggtcagggtgatggaggcaggcgcaaaaaaggagggtggtttggaaagcatcgtggtcaaggaggttccaacccgaaatttgagaacattgcagaaggtttaagagctctcctggctaggagtcacgtagaaaggactaccgacgaaggaacttgggtcgccggtgtgttcgtatatggaggtagtaagacctccctttacaacctaaggcgaggaactgcccttgctattccacaatgtcgtcttacaccattgagtcgtctcccctttggaatggcccctggacccggcccacaacctggcccgctaagggagtccattgtctgttatttcatggtctttttacaaactcatatatttgctgaggttttgaaggatgcgattaaggaccttgttatgacaaagcccgctcctacctgcaatatcagggtgactgtgtgcagctttgacgatggagtagatttgcctccctggtttccacctatggtggaaggggctgccgcggagggtgatgacggagatgacggagatgaaggaggtgatggagatgagggtgaggaagggcaggagtga

SEQ ID NO: 82 - protein, 641 aa, Homo sapiens
MSDEGPGTGPGNGLGEKGDTSGPEGSGGSGPQRRGGDNHGRGRGRGRGRGGGRPGAPGGSGSGPRHRDGVRRPQKRPSCIGCKGTHGGTGAGAGAGGAGAGGAGAGGGAGAGGGAGGAGGAGGAGAGGGAGAGGGAGGAGGAGAGGGAGAGGGAGGAGAGGGAGGAGGAGAGGGAGAGGGAGGAGAGGGAGGAGGAGAGGGAGAGGAGGAGGAGAGGAGAGGGAGGAGGAGAGGAGAGGAGAGGAGAGGAGGAGAGGAGGAGAGGAGGAGAGGGAGGAGAGGGAGGAGAGGAGGAGAGGAGGAGAGGAGGAGAGGGAGAGGAGAGGGGRGRGGSGGRGRGGSGGRGRGGSGGRRGRGRERARGGSRERARGRGRGRGEKRPRSPSSQSSSSGSPPRRPPPGRRPFFHPVGEADYFEYHQEGGPDGEPDVPPGAIEQGPADDPGEGPSTGPRGQGDGGRRKKGGWFGKHRGQGGSNPKFENIAEGLRALLARSHVERTTDEGTWVAGVFVYGGSKTSLYNLRRGTALAIPQCRLTPLSRLPFGMAPGPGPQPGPLRESIVCYFMVFLQTHIFAEVLKDAIKDLVMTKPAPTCNIRVTVCSFDDGVDLPPWFPPMVEGAAAEGDDGDDGDEGGDGDEGEEGQE

SEQ ID NO: 83 - DNA, 414 bp, Homo sapiens
atgcgtgctgttggtgtatttctggccatctgtcttgtcaccattttcgtcctcccaacatggggcaattgggcatacccatgttgtcacgtcactcagctccgcgctcaacaccttctcgcgttggaaaacattagcgacatttacctggtgagcaatcagacatgcgacggctttagcctggcctccttaaattcacctaagaatgggagcaaccagctggtcatcagccgctgcgcaaacggactcaacgtggtctccttctttatctccatcctgaagcgaagcagctccgccctcacgggccatctccgtgagttgttaaccaccctggagactctttacggttcattctcagtggaagacctgtttggtgccaacttaaacagatacgcatggcatcgcgggggctag

SEQ ID NO: 84 - protein, 137 aa, Homo sapiens
MRAVGVFLAICLVTIFVLPTWGNWAYPCCHVTQLRAQHLLALENISDIYLVSNQTCDGFSLASLNSPKNGSNQLVISRCANGLNVVSFFISILKRSSSALTGHLRELLTTLETLYGSFSVEDLFGANLNRYAWHRGG

SEQ ID NO: 85 - DNA, 768 bp, Homo sapiens
atggcatcgcgggggctagacctctggctggatgagcacgtgtggaagaggaaacaggagattggtgtgaaaggagaaaatctgcttctccccgacttatggctagatttcctacaactcagccccatcttccagcgcaagcttgctgccgttattgcctgtgtccgacgcctgcggactcaggccaccgtctacccagaggaggacatgtgcatggcctgggcccgcttttgcgacccctctgatattaaggtggttattttgggccaggacccctatcacgggggtcaagcaaacggcctggcattcagcgtcgcatacggctttccagttccccccagcctgaggaacatctacgcggagctgcaccggagcctgccggagttttctcccccagatcacggctgtctagacgcgtgggcctcccagggggtgttgctactcaacaccatcctgaccgtgcaaaagggcaagcccggctcgcacgcagacattggctgggcgtggtttactgaccacgtaatttcattgctctctgagcggttaaaagcgtgcgtgtttatgctgtggggtgcgaaggcgggagacaaagcttcactaatcaactccaagaagcatctggttctgacctctcagcatccctctcccctggcccagaacagcacccgaaagagtgcccagcagaagttcctgggcaacaaccactttgtcctcgctaacaactttttgcgtgagaaggggctcggtgagatagattggaggctgtag

SEQ ID NO: 86 - protein, 255 aa, Homo sapiens
MASRGLDLWLDEHVWKRKQEIGVKGENLLLPDLWLDFLQLSPIFQRKLAAVIACVRRLRTQATVYPEEDMCMAWARFCDPSDIKVVILGQDPYHGGQANGLAFSVAYGFPVPPSLRNIYAELHRSLPEFSPPDHGCLDAWASQGVLLLNTILTVQKGKPGSHADIGWAWFTDHVISLLSERLKACVFMLWGAKAGDKASLINSKKHLVLTSQHPSPLAQNSTRKSAQQKFLGNNHFVLANNFLREKGLGEIDWRL

SEQ ID NO: 87 - DNA, 654 bp, Homo sapiens
atggccatgtttctgaagtcgcgtggggtccggtcttgcagggaccggcgcctcttgtcggacgaggaggaagagacttcacagagcagcagctacactctggggtctcaggcctcccagtctatccaggaggaggacgtgagtgacactgatgagtctgactactcagatgaagacgaggagattgatttggaggaagagtaccccagtgacgaagacccatctgagggcagtgatagcgacccctcgtggcatccttcagattcagacgagtctgactacagcgagagcgacgaggatgaagcaacccccggctctcaggcctcacgatcttcaagagtctcgccatctacccaacagtcttcaggtctgacacccacgccttcgttctcccgaccacgcacccgggcacctccgaggccgccggctcccgcgccggtcaggggacgggcctcagcacctcccaggccaccagccccagttcagcaatccaccaaagacaagggtccccatagacctacgcgacctgtacttagaggcccagctccacgccgcccccctccaccttcaagtcccaatacatacaataaacacatgatggaaaccaccccccccattaagggcaataacaactacaattggccatggctgtaa

SEQ ID NO: 88 - protein, 217 aa, Homo sapiens
MAMFLKSRGVRSCRDRRLLSDEEEETSQSSSYTLGSQASQSIQEEDVSDTDESDYSDEDEEIDLEEEYPSDEDPSEGSDSDPSWHPSDSDESDYSESDEDEATPGSQASRSSRVSPSTQQSSGLTPTPSFSRPRTRAPPRPPAPAPVRGRASAPPRPPAPVQQSTKDKGPHRPTRPVLRGPAPRRPPPPSSPNTYNKHMMETTPPIKGNNNYNWPWL

SEQ ID NO: 89 - DNA, 2430 bp, Homo sapiens
atggccgaggagccgagggcgccagaggcgctctcgtccacgttcatgttgaacatgacctcagacgcctccgtgaggcgtatagtcaggaggatagggaccctggctaggcgccgcgtccaacaactcccggatatggagacgttctcccccgagtttgacccggagctctcggagccccccttcctgcccttttcagcctatgtaattacgggaacggcgggggctggcaagagcaccagcgttagctgcctccaccacacgatggactgcctggtcacgggagccacaaccgtggccgcacagaacctctcccagacactccgagcctactgcccaaccgtctatagcgcctttgggttcaagagccgccacataaatatgacgcagcgggtcagcagtcatggtcgctctacggacgccgccctagaggaactccagcggcgggacctggccaaatactggccggtactctccgacattgccgccgagttcaggcgaaccaagcccagggggctctactcgggagtctctggccccgcctttgaggtcctgagagacatgcaccaggggcagctatggaccaccaatgtgatcgtggtggacgaagctggaacgctttccgtgcacattctaacagccgtggtcttttgctactggttcttcaacgcctggctgcgtaccccactctaccgccggggccgcattccctgcatcgtctgcgtgggctctcccacccagacagacgcctttcagtcctcctttagccacgagacccaggtaaacaagattcgcgagtgcgacaacatcctcaccttcctggtgggcaaccccagggccgcgacctacgtggacgtggcccgcaactgggccctcttcatcaacaacaagcgctgcaccgacgtccagtttggacacctcatgaagaccctcgagtacggcctcgagctctcgccggacatcctggcctacgtcgaccgcttcgtcgtccccagggcggccataatggaccccgcgcagtacgtgggctggacccggctcttcctctcccacgccgaggtcaagaccttcctcaccaccctccatgccacgctcaagactgccgggcaggggcgtgccgcgcggggaaccgggggagatgggggcggggtgaccatgtttacctgcccggtggagtgcgaggtcttcctcgatcccctggcccagtacaagaccctggtcgggcttccgggcctcacggcccacacctggctccaaaagaattatgcacgcctcggtaactactcgcagttcgccgaccaagacatggtcccggtgggcaccgagcaggacgaagaacgggtcaaggtcacctacaatgtcacctacgtcaagcacagctcggtgtcggtcaactgcaagaccaaaaagtccatctgtggctataccggcaccttcggggactttatggacacgctcgaggcggacagcttcgtggaggcccacgggcatgagcagcccgagtatgtctacagcttcctggcccgactcatctacggaggcatctacgcttttagccacggaggacattctctctgtgaaaacggggaatacgtggcggagcttggtgctgtgcccctgcccgggcgtacgtgggatccggaggtgactgctgggatggagctgggcgaactgccgctggaggttgcctgggatggggagcggagccccgccgctgtcttctatgcccgagtactggctcccccggccgcaaattctgcccccctctgctccctgttaaacatatataatgacctcagggcctatttcaggcaatgcctggatgtggccgtccgctatggcggaagggagttcagggacctccccttctgcacatttaccaacaacatgctgattcgcgacaacatagagttcacctcagacgagcctctccttcacggcctcctggactacgcctccaccaccgagaactacacgcttctgggctacacgcatctcaacgtcttctttggcatccgaggaaagcagcagcctcaggacgctggcagttcacgcatgcccaggctaatggtcaaggatgaggcaggcttcgtgtgctgcctggaacacaatactaacaaactgtatgagacgatagaggacaagtccctgaacctctgcagcatccgtgactatggcattagctcaaagctggccatgaccatagccaaggcccagggtctgtccctaaacaaagtcgccatctgcttcggcagccacagaaacatcaaacccggccatgtgtatgtggcgctgtcccgggcccggcactctaattgcgtggtcatggacaggaatcccctatccgagatgatcactggggaggggaaccccgcgagcggctacatcgtggatgccctaaagaactcacgcgcactactggtttactga

SEQ ID NO: 90 - protein, 809 aa, Homo sapiens
MAEEPRAPEALSSTFMLNMTSDASVRRIVRRIGTLARRRVQQLPDMETFSPEFDPELSEPPFLPFSAYVITGTAGAGKSTSVSCLHHTMDCLVTGATTVAAQNLSQTLRAYCPTVYSAFGFKSRHINMTQRVSSHGRSTDAALEELQRRDLAKYWPVLSDIAAEFRRTKPRGLYSGVSGPAFEVLRDMHQGQLWTTNVIVVDEAGTLSVHILTAVVFCYWFFNAWLRTPLYRRGRIPCIVCVGSPTQTDAFQSSFSHETQVNKIRECDNILTFLVGNPRAATYVDVARNWALFINNKRCTDVQFGHLMKTLEYGLELSPDILAYVDRFVVPRAAIMDPAQYVGWTRLFLSHAEVKTFLTTLHATLKTAGQGRAARGTGGDGGGVTMFTCPVECEVFLDPLAQYKTLVGLPGLTAHTWLQKNYARLGNYSQFADQDMVPVGTEQDEERVKVTYNVTYVKHSSVSVNCKTKKSICGYTGTFGDFMDTLEADSFVEAHGHEQPEYVYSFLARLIYGGIYAFSHGGHSLCENGEYVAELGAVPLPGRTWDPEVTAGMELGELPLEVAWDGERSPAAVFYARVLAPPAANSAPLCSLLNIYNDLRAYFRQCLDVAVRYGGREFRDLPFCTFTNNMLIRDNIEFTSDEPLLHGLLDYASTTENYTLLGYTHLNVFFGIRGKQQPQDAGSSRMPRLMVKDEAGFVCCLEHNTNKLYETIEDKSLNLCSIRDYGISSKLAMTIAKAQGLSLNKVAICFGSHRNIKPGHVYVALSRARHSNCVVMDRNPLSEMITGEGNPASGYIVDALKNSRALLVY

SEQ ID NO: 91 - DNA, 1842 bp, Homo sapiens
atgttcaacatgaacgtggacgagagcgcctctggcgccctcggctcctcggccattcctgttcaccccacgccggcctcggtccgactttttgagatcctgcagggaaagtacgcctacgtccagggacagaccatctacgccaacctccgcaaccccggagtcttctcgaggcaggtgtttacccatttgtttaaacgagccatctctcattgcacgtacgatgacgtgctacatgactggaacaagttcgaggcctgcatccagaagcgatggccgagcgatgactcgtgtgcgagccggtttcgtgagtccaccttcgagtcgtggtccacgaccatgaagctgaccgtgcgtgacctgctgaccaccaacatctaccgagtgctacacagccgctccgtgctctcctatgagcgttatgtggactggatctgcgccaccggcatggtgcccgccgttaagaagcccataacccaagagctccactccaagataaagagcctgagggacaggtgcgtctgtcgggaattggggcacgagaggaccatcaggagtatcgggacggaattatatgaggcaacgaaggaaataatagagtcgctcaactccacgttcatcccccagtttacggaggtgaccatcgagtaccttccgaggagcgacgagtatgtggcctactactgtggccgccgcatcaggctgcatgtgctcttccccccggccatctttgccggaacggtgaccttcgacagcccggtgcagcgcctctaccagaacattttcatgtgctaccgcacgctggagcatgccaagatctgccagctcctgaacacggcccctctcaaggccatcgtgggccacggggggcgagacatgtacaaggacatcctggcccatctggagcagaactcacagcgcaaggaccccaagaaggagctgctgaacctgctggtcaagctctcggagaacaagaccatcagcggggtcacggacgtggtggaggagttcataacggatgcctccaacaacctggtggaccgcaaccgtctatttggccagcccggggagacagctgcacagggcctaaagaaaaaggtctccaacacggtggtcaagtgtctgactgatcagataaacgagcaatttgaccagattaatggcctagagaaggagagggagctctatctaaagaagatccgctccatggagtctcagctgcaggcctccctgggtcccggcggcaacaacccagcggcgtcagcccccgccgcagttgcggcagaagccgcgtctgtagatatactgacgggcagcaccgcctccgcaatcgaaaagctgttcaactccccgtccgccagcctgggtgccagggtgtctggtcacaatgaaagcatcctaaacagtttcgtttctcaatacatccccccttcgcgggaaatgactaaggatctgactgaactttgggaaagcgagctgtttaacaccttcaagttaacacccgtggttgataatcaggggcagcgtctctacgtcagatactcgtcagacacgatctctatattattgggccccttcacctatctggtggcagagctttcaccggtggaactcgtgacagatgtctacgccaccctaggcatcgtggagatcatcgacgagctctaccggagcagtcgcctggccatctacatcgaggacctcggtcgaaaatactgccccgcgagcgcgaccgggggagatcatggcatccggcaagcaccatcagcccggggggacacggagcctgaccatgcaaaaagtaagcctgcgcgtgaccccccgcctggtgctggaagttaa

SEQ ID NO: 92 - protein, 613 aa, Homo sapiens
MFNMNVDESASGALGSSAIPVHPTPASVRLFEILQGKYAYVQGQTIYANLRNPGVFSRQVFTHLFKRAISHCTYDDVLHDWNKFEACIQKRWPSDDSCASRFRESTFESWSTTMKLTVRDLLTTNIYRVLHSRSVLSYERYVDWICATGMVPAVKKPITQELHSKIKSLRDRCVCRELGHERTIRSIGTELYEATKEIIESLNSTFIPQFTEVTIEYLPRSDEYVAYYCGRRIRLHVLFPPAIFAGTVTFDSPVQRLYQNIFMCYRTLEHAKICQLLNTAPLKAIVGHGGRDMYKDILAHLEQNSQRKDPKKELLNLLVKLSENKTISGVTDVVEEFITDASNNLVDRNRLFGQPGETAAQGLKKKVSNTVVKCLTDQINEQFDQINGLEKERELYLKKIRSMESQLQASLGPGGNNPAASAPAAVAAEAASVDILTGSTASAIEKLFNSPSASLGARVSGHNESILNSFVSQYIPPSREMTKDLTELWESELFNTFKLTPVVDNQGQRLYVRYSSDTISILLGPFTYLVAELSPVELVTDVYATLGIVEIIDELYRSSRLAIYIEDLGRKYCPASATGGDHGIRQAPSARGDTEPDHAKSKPARDPPPGAGS

SEQ ID NO: 93 - DNA, 837 bp, Homo sapiens
atggcatccggcaagcaccatcagcccggggggacacggagcctgaccatgcaaaaagtaagcctgcgcgtgaccccccgcctggtgctggaagttaaccgccataacgccatctgcgtggccaccaacgtccctgagttctacaatgccaggggggaccttaacatccgagacctccgggcccacgtcaaggcccggatgatctcgtcccagttttgcggctacgtcctcgtgagtctgctggactccgaggaccaggtcgaccacctcaacatattcccccacgtgttctccgagaggatgatcctgtacaaacccaacaatgtgaaccttatggagatgtgcgccctgctctcgatgattgagaatgccaagagcccctccataggcctctgccgggaggtgctgggtcgcctgaccctcttgcactccaagtgcaacaatctggactctctgtttctgtacaatggggccaggacgctgctgtccaccctggtcaagtaccacgacctggaggagggggctgccacccccgggccgtggaatgagggcctgagtctctttaagctgcacaaggagctgaagcgcgccccatccgaagcccgggacctcatgcagagcctctttctgacctcggggaagatggggtgcctggccaggtcacccaaggattactgcgcggatctaaacaaggaggaagatgccaactcgggcttcacatttaacctgttttatcaagattctttattgaccaagcatttccagtgccagaccgtcctccagaccttgagacgcaagtgcctcgggagtgacacggtctcaaaaataattccctag

SEQ ID NO: 94 - protein, 278 aa, Homo sapiens
MASGKHHQPGGTRSLTMQKVSLRVTPRLVLEVNRHNAICVATNVPEFYNARGDLNIRDLRAHVKARMISSQFCGYVLVSLLDSEDQVDHLNIFPHVFSERMILYKPNNVNLMEMCALLSMIENAKSPSIGLCREVLGRLTLLHSKCNNLDSLFLYNGARTLLSTLVKYHDLEEGAATPGPWNEGLSLFKLHKELKRAPSEARDLMQSLFLTSGKMGCLARSPKDYCADLNKEEDANSGFTFNLFYQDSLLTKHFQCQTVLQTLRRKCLGSDTVSKIIP

SEQ ID NO: 95 - DNA, 603 bp, Homo sapiens
gctttcctgcaaggcgtgaaagactcggaggatgccagccggctcgaccgggatgtgatgggtggagaggccaccgtggcccgcagacacattcgggtgaaggctcgacgcgggcccgggtgcctactgatggccatctttcagggggatctttacgtgggtggatgtagggagcactcggggccctttttggtttggcacgaagccttctcctggaccctggaccaactagcggcgagacccgaggcagacaaggccccgccatcccacgaccacctgttgaccctggtccgcgacctgacccggagactggcccccgggcggcgccgaaacaggttttgggctcttccgcgagcctggcttcagcggctgcggagggctgggctgcgcctctctggaagccacgtgtgtctcctggacaaggacggagcgcgcccggccccctgccagacggccactgagcatggtctcagccccaccgcctactttcgagagattatggcctttctgcttgatgtgatatcggccctccaccccggctacaccattcctatggaaatcacgcgagagacagatttactgatgactgttctcagtttattc

SEQ ID NO: 96 - protein, 201 aa, Homo sapiens
AFLQGVKDSEDASRLDRDVMGGEATVARRHIRVKARRGPGCLLMAIFQGDLYVGGCREHSGPFLVWHEAFSWTLDQLAARPEADKAPPSHDHLLTLVRDLTRRLAPGRRRNRFWALPRAWLQRLRRAGLRLSGSHVCLLDKDGARPAPCQTATEHGLSPTAYFREIMAFLLDVISALHPGYTIPMEITRETDLLMTVLSLF

SEQ ID NO: 97 - DNA, 1566 bp, Homo sapiens
ccggatgttttaaagggtccagtcctcctcagatctcaaacaatgatggaaacacccgcggagagcgtcagggcccgcgtcagctcggttaccttttataatgttacccagaccgcagggcggtggtgggcgatttgggtcgtgggcatcgtgcccatcaaaagggaggacgttgagactctgatcgtggtgcaggcctgccagccgccgcttggaggctccctggagccccccgtggtcaacgcgccctcgactaccgaactcaactttttgcgatgggagcgggagctcaggcgcagcggggggctcattgctatgctcgccgatgccgccgagaaggacctatttgacctttcatttagaacccgagaccgcagactcttgtccgccgccagggtggaggatgagcagggcctcatcttccagcctctctttccagcacaggtggtctgccaaagctgctcgggggatgatgggcgggaccaacaacccccacctgttgatggcttcgggtccgagatggagggggaacagacatgcccccatgctcagaggcactctgagtctcccggacagttggacgtatacatcaggacaccgcgtggggatgttttcacctattccaccgagactcccgacgacccctctcccgtcccctttagggacatcctgcgaccggtcacttatgaagtagatctggtgtcgtccgacggggccaccggccgtggtggggacgcccgacggcaccgcgtcagcctgaaaattcttgagccggctgggggattcgagtcctggcttgtcaactcttggagtatggccgggggcgggctctacgccttcctccgcagtatctatgcctcctgctacgccaaccacagaggcaccaagcccatcttctacctcctggaccccgaactctgcccagggggctcagatttccagccctatgtcccgggctttcccttcctgcccatccattatgtgggacgggcgaggccggccttctggcaccgggcgccacacagcgagggtctcctgctactggacctgaacctgggagtctctgggacgcccctggcagacgccctcctgggcctcgacgcgcggtcagggcaacgacgcggctcgctgctcctacaacaaatctggcccccgacccgaaaggagattaaccctcgccacgtttgcacgcgggaaggcggcgagggagggggggaggacgagacgacggtggtcgggcgcgcggaggccacagccatccttgaggctgatgccacttggtggctctacgaattggcccgctgccacctctctgccaggggcgcccccgtgggaacgcctgatggcggagggcaggcgcgggacgcgcaaacctggctgcgggcccttcaccgctatggcacctcagacacgcgacgggcactcgggggcctctacaccgccgtcacccgggttctcctacacgcggccgctgacctaggactgacctgggcttatgcagacgagttcatcctgggctttgtggcaccaacctccgcccatccttcagaggagccactagcacag

SEQ ID NO: 98 - protein, 522 aa, Homo sapiens
PDVLKGPVLLRSQTMMETPAESVRARVSSVTFYNVTQTAGRWWAIWVVGIVPIKREDVETLIVVQACQPPLGGSLEPPVVNAPSTTELNFLRWERELRRSGGLIAMLADAAEKDLFDLSFRTRDRRLLSAARVEDEQGLIFQPLFPAQVVCQSCSGDDGRDQQPPPVDGFGSEMEGEQTCPHAQRHSESPGQLDVYIRTPRGDVFTYSTETPDDPSPVPFRDILRPVTYEVDLVSSDGATGRGGDARRHRVSLKILEPAGGFESWLVNSWSMAGGGLYAFLRSIYASCYANHRGTKPIFYLLDPELCPGGSDFQPYVPGFPFLPIHYVGRARPAFWHRAPHSEGLLLLDLNLGVSGTPLADALLGLDARSGQRRGSLLLQQIWPPTRKEINPRHVCTREGGEGGGEDETTVVGRAEATAILEADATWWLYELARCHLSARGAPVGTPDGGGQARDAQTWLRALHRYGTSDTRRALGGLYTAVTRVLLHAAADLGLTWAYADEFILGFVAPTSAHPSEEPLAQ

SEQ ID NO: 99 - DNA, 1218 bp, Homo sapiens
atgaagtcctccaagaatgacacgttcgtctatagaacgtgggtcaaaacgcttgttgtgtactttgtgatgtttgtcatgtcggcggtggtccccatcaccgccatgttccccaacctggggtacccctgctactttaacgcactggttgattacggggcacttaacctgaccaattacaacctggcccaccacctgacccccacgctctatctggagccgccggagatgtttgtctacatcacactggtctttatcgcggactgcgtggctttcatctactacgcctgcggcgaggtggcgctaatcaaggcccgaaaaaaggtctcgggtcttacagacctctcggcctgggtctcggcagtgggctccccaaccgtgctgtttttggccatcctcaagctctggtccatacaggtcttcatccaggtcctttcctacaagcacgtctttctctcggcctttgtgtactttttgcactttctggcctcagttctacacgcctgcgcatgtgtgacccgcttctccccggtctgggtggtcaaggcccaggacaactctattccccaggacaccttcttgtggtgggtggtcttctacctgaagcccgtagttacaaacctgtacctggggtgccttgccctggagacgctggtcttctcgctcagcgtgttcctggccctgggcaacagcttttactttatggtgggggacatggtgctgggagccgtgaacctcttcctcatcctgcccatcttctggtacattctgacggaggtgtggctggcctccttcctgcggcacaactttggcttctactgcggcatgttcatcgcctccatcatcctgatcctgcccttggtcaggtacgaggccgtctttgtctccgccaagctgcacaccactgtggccatcaatgtggccatcatacctatcctgtgctcggtggccatgctcatcaggatatgccggattttcaaaagcatgcgccagggcactgactatgtccctgtctcggagacggtggaactggagctagagtcagagccgaggcctaggccctcgcgcacgccatcacccgggcgcaaccgccgccgctcttctacgtcctcatcttcctccaggtcaaccaggagacagaggcccgtctctacccaagccctcgtctcctccgttttaccgatgacgacggacagcgaggaggagatcttcccctaa

SEQ ID NO: 100 - protein, 405 aa, Homo sapiens
MKSSKNDTFVYRTWVKTLVVYFVMFVMSAVVPITAMFPNLGYPCYFNALVDYGALNLTNYNLAHHLTPTLYLEPPEMFVYITLVFIADCVAFIYYACGEVALIKARKKVSGLTDLSAWVSAVGSPTVLFLAILKLWSIQVFIQVLSYKHVFLSAFVYFLHFLASVLHACACVTRFSPVWVVKAQDNSIPQDTFLWWVVFYLKPVVTNLYLGCLALETLVFSLSVFLALGNSFYFMVGDMVLGAVNLFLILPIFWYILTEVWLASFLRHNFGFYCGMFIASIILILPLVRYEAVFVSAKLHTTVAINVAIIPILCSVAMLIRICRIFKSMRQGTDYVPVSETVELELESEPRPRPSRTPSPGRNRRRSSTSSSSSRSTRRQRPVSTQALVSSVLPMTTDSEEEIFP

SEQ ID NO: 101 - DNA, 228 bp, Homo sapiens
atgggtgccctctggtctctttgccgacgacgagtcaactccataggcgacgtggacgggggaataatcaacctgtataatgactatgaggagtttaacctggagactactaagctaatagcggccgaagaagggagggcctgcggggaaaccaacgaggggctcgaatatgatgaggactctgaaaatgatgaattgctgtttttgccaaataaaaaaccaaactaa

SEQ ID NO: 102 - protein, 75 aa, Homo sapiens
MGALWSLCRRRVNSIGDVDGGIINLYNDYEEFNLETTKLIAAEEGRACGETNEGLEYDEDSENDELLFLPNKKPN

SEQ ID NO: 103 - DNA, 1413 bp, Homo sapiens
atggccgacgtggatgagctcgaggatcccatggaggagatgacctcctacacgtttgcccgcttcctccgcagtccggagactgaggcctttgtccgtaaccttgaccgtccacctcagatgccggccatgcgctttgtctatctctattgcctctgtaaacaaatacaagagttctctggtgaaactggcttctgtgactttgtctcctcgttagtccaagagaatgacagcaaggacggtccctccctgaaatccatttactgggggctacaggaggccaccgacgagcagaggactgttctctgctcgtacgtggagtccatgaccagggggcagtctgagaacctgatgtgggacatattgcgaaatggcataatttcctcttccaagctgctctccaccattaagaatggacccaccaaggtgtttgagccagctcccatctccacaaatcactactttgggggacctgtggcctttggcctgcggtgtgaggacacggtcaaggacattgtctgtaagctcatctgcggggacgcatccgccaaccgtcaatttggctttatgattagtcccacggatggcatttttggggtgtctctggatctttgcgtcaatgtggagtcacagggagactttatactgttcaccgaccggagctgcatttatgagattaagtgccgcttcaagtacctcttttccaagtcagaatttgaccccatctacccatcctacactgcgctttacaagaggccatgcaagaggtcatttatcagatttatcaattctatagctcgtcctaccgtggagtacgtcccggatgggcggttgccctcggagggtgactatctgttgacgcaggatgaggcctggaatcttaaagatgtccgtaagcgcaaactgggccccggtcatgacctggtggcagacagcctagctgccaacaggggggtggagtctatgctctacgtaatgacggacccaagcgaaaatgcggggcgcattggtattaaagaccgggtcccagtcaacatcttcatcaatccacggcacaactacttctaccaggtgctcctccaatacaaaattgttggagactacgtccgccacagtgggggtggcaagcccgggagagactgctcaccccgggtgaacattgtgacggccttctttcgaaaacggtcgcctctagacccggcgacctgcacgctcggctcagacctgcttctggacgcctcggtggagattcccgtggcggtgctggtgacacccgtggtcctgccggactctgtcatccgtaagaccttgagcaccgcggctggctcctggaaagcgtacgcagacaatacttttgacaccgcgccatgggtgccctctggtctctttgccgacgacgagtcaactccatag

SEQ ID NO: 104 - protein, 470 aa, Homo sapiens
MADVDELEDPMEEMTSYTFARFLRSPETEAFVRNLDRPPQMPAMRFVYLYCLCKQIQEFSGETGFCDFVSSLVQENDSKDGPSLKSIYWGLQEATDEQRTVLCSYVESMTRGQSENLMWDILRNGIISSSKLLSTIKNGPTKVFEPAPISTNHYFGGPVAFGLRCEDTVKDIVCKLICGDASANRQFGFMISPTDGIFGVSLDLCVNVESQGDFILFTDRSCIYEIKCRFKYLFSKSEFDPIYPSYTALYKRPCKRSFIRFINSIARPTVEYVPDGRLPSEGDYLLTQDEAWNLKDVRKRKLGPGHDLVADSLAANRGVESMLYVMTDPSENAGRIGIKDRVPVNIFINPRHNYFYQVLLQYKIVGDYVRHSGGGKPGRDCSPRVNIVTAFFRKRSPLDPATCTLGSDLLLDASVEIPVAVLVTPVVLPDSVIRKTLSTAAGSWKAYADNTFDTAPWVPSGLFADDESTP

SEQ ID NO: 105 - DNA, 1365 bp, Homo sapiens
agcgggtggaggagctccgtgagcaggagcttgaggccagagacgagctgcgacaggccctcgagccatttgaggaacatggatgtgaatatggctgcggagttgagcccgacgaactcctccagcagtggcgagttgagtgtctccccagaaccccctcgagagacccaggcctttttggggaaggtgactgtcattgattacttcacctttcagcacaaacacctgaaggtgaccaacattgatgacatgacggagaccctctatgtaaagctgccggagaacatgacgcgctgtgatcacctccccattacctgcgagtatctgctggggcgggggagctacggggccgtgtatgcacatgcagataatgccacggtcaaactctatgactctgtgacggagctgtatcacgagctcatggtgtgtgacatgattcagattgggaaggccacggccgaggatgggcaggacaaggccctggtggactacctgtcggcctgcacgtcctgccacgccctgtttatgccccagttcagatgcagtctccaggattatggccactggcatgatggtagtattgagcccctggtgcggggctttcagggcctcaaagatgccgtttactttctgaatcggcactgcggcctcttccattcggacattagccccagcaacatcctggtggatttcacagacaccatgtggggcatgggtaggctggtcctgactgattatgggactgcttccctccacgaccgcaacaagatgctggatgtgcggctaaagtcttctaagggccggcagctctatcgcctctattgccagagggaaccattttctatagccaaggacacctataagcccctctgccttttgagcaagtgctacatcttgaggggggctgggcacatccctgacccctcggcgtgtggccccgtgggggcgcagacggcccttcgcctggatctgcagtcgctcggctactcgctgctctatggtatcatgcacctcgctgactccacccacaaaatcccctaccccaaccctgacatgggatttgaccgatccgacccgctctactttttgcaatttgcagccccaaaggtggtgctgttggaggtgctgtcgcagatgtggaacctgaacttagacatgggcctgacctcgtgtggcgagagtccgtgcgtggatgtcacggcggagcatatgagtcaattcttgcagtggtgccggagccttaaaaagaggttcaaggagagctacttcttcaactgtcgcccacggtttgagcaccctcatcttccaggtctggtagctgaactcttggcagacgacttctttggtccagatggccgacgtgga

SEQ ID NO: 106 - protein, 455 aa, Homo sapiens
SGWRSSVSRSLRPETSCDRPSSHLRNMDVNMAAELSPTNSSSSGELSVSPEPPRETQAFLGKVTVIDYFTFQHKHLKVTNIDDMTETLYVKLPENMTRCDHLPITCEYLLGRGSYGAVYAHADNATVKLYDSVTELYHELMVCDMIQIGKATAEDGQDKALVDYLSACTSCHALFMPQFRCSLQDYGHWHDGSIEPLVRGFQGLKDAVYFLNRHCGLFHSDISPSNILVDFTDTMWGMGRLVLTDYGTASLHDRNKMLDVRLKSSKGRQLYRLYCQREPFSIAKDTYKPLCLLSKCYILRGAGHIPDPSACGPVGAQTALRLDLQSLGYSLLYGIMHLADSTHKIPYPNPDMGFDRSDPLYFLQFAAPKVVLLEVLSQMWNLNLDMGLTSCGESPCVDVTAEHMSQFLQWCRSLKKRFKESYFFNCRPRFEHPHLPGLVAELLADDFFGPDGRRG

SEQ ID NO: 107 - DNA, 999 bp, Homo sapiens
atgttcaacgcggtcaaggccgatatgcccgatgacccgatgctcgcccgcaggtatgggcagtgtctggaactggcactggaggcctgccaggacacgcccgagcaatttaagctggtggagacgccactcaagagcttcctgctcgtgtccaatatcctcccccaggacaacaggccctggcacgaggcccggagctccggccgtgtggccgaggatgactatgacttctcgagcctggctttggagctcttgcccctcaacccccgtttaccagaagaatggcagtttggtggacaaggttggtcgagccggatggagccctctcagcctgaaatgggcatgggtctgtgttttgaggtgtttgatggggaccttatgcgaattgcgctggcctggaacaaagatgaggttataggtcaggccttgcaaatattggcccactcccaaacctggacctccctggtgcctgaagacccactcccttggatgtgggccctcttctacggccccaggtcccactgcgaagagcgtcactgcgtttacgctgcggcacggggcaaaaggggccctattctgctacccactgcggtttatacaccatgtgccaacatagaggcattcctggcacacctcaccaggtgtgtgtatgccctgtacttggacgtgcgtgactggaagggtgaggatatagctcctccctttgacgttagccgcctgaataaaatggcaaaacagctctgcctgctgcctcaggaacccttttgcatcactcgtgtgtgccttttgtgcttgctacataaacaaaacctaaacgctcagtataaaaggccggtggacacgtatgatccttgcctaattctaacgggagaggcagaaagatatatggtggacgctgtgggaaattaccgggaagcctccactggaactaccgtactctaccccacgtatgatctgggctccatagttgcggacatggtgacttatgaagatgagtag

SEQ ID NO: 108 - protein, 332 aa, Homo sapiens
MFNAVKADMPDDPMLARRYGQCLELALEACQDTPEQFKLVETPLKSFLLVSNILPQDNRPWHEARSSGRVAEDDYDFSSLALELLPLNPRLPEEWQFGGQGWSSRMEPSQPEMGMGLCFEVFDGDLMRIALAWNKDEVIGQALQILAHSQTWTSLVPEDPLPWMWALFYGPRSHCEERHCVYAAARGKRGPILLPTAVYTPCANIEAFLAHLTRCVYALYLDVRDWKGEDIAPPFDVSRLNKMAKQLCLLPQEPFCITRVCLLCLLHKQNLNAQYKRPVDTYDPCLILTGEAERYMVDAVGNYREASTGTTVLYPTYDLGSIVADMVTYEDE

SEQ ID NO: 109 - DNA, 5413 bp, Homo sapiens
atgctgtatgcctcgcagcgcggccgtctgaccgagaacctaagaaacgcccttcagcaggacagcaccacgcaaggctgcctgggtgccgagaccccgagtattatgtacacaggggccaagtcagacaggtgggctcaccctctggtgggcacaattcacgccagtaatttatattgcccaatgcttcgagcatactgccgccactatggccccaggcccgtgtttgtagcttctgatgaatcattacccatgttcggtgcgagccctgcccttcacaccccagtccaggtccagatgtgcctactaccagagctacgcgacacgttacagcgcctgctgccaccacccaatcttgaagactccgaggccttgacggaattcaagaccagcgtgtcctctgcccgtgccatccttgaggaccccaactttttggagatgagagagtttgtcaccagcctggccagcttcctgagtggtcagtacaagcacaagcccgcccgcctagaagcattccagaaacaagtagtgttacattctttttattttctgatctcaatcaaatctttagagattacagacaccatgtttgacatctttcaaagtgctttcggattggaagaaatgacgctggagaagctgcacatttttaagcaaaaagccagcgtgtttcttatccccaggcgccacggcaagacctggatagtcgtggccatcatcagcctcatcctctcgaatctctccaacgtgcaaataggctacgtggctcaccagaaacatgtcgcgtccgccgttttcactgaaattattgacaccttgaccaagagcttcgactccaagcgtgtagaggtcaacaaggagaccagcaccatcacgtttaggcacagtgggaaaatctccagcaccgtaatgtgtgccacctgcttcaataagaatgtaagacctgacgtttcagtacttggcaattgtagagcatagcccggctgtaaaggtcagaaaatcgcagcagggtccaaggttgtgctgtacatgggacctctttcccattagcaagaaccccctgcaggacacgtgacatgtccgggtgcattttgggtgggttaaatctcagtcccaccacaaagggggcatcctccggtttgaacatcagacccaacaaagcccgatgcccagttatgggtacgtagtcgttgttcagggccgtgcatggcagcagacaaggacaggtgccagatgtgcctgggctatcgtcctccgtccagccacgcaggatgttcacgtgggccccggcaccatagcatgtcacacattccccgttatcacatctggttagcaggttgataaaatgggtcagtgatggaaaggttggcatattggggcagcacatcagcatgtccatgttaacgaaaaacatgtacagggccccttctgcataccaggcaccaccccgtcccagtgggatgatctccgagggtgtgatatcttgcagttcttctactgttttaacggcggttgaggtggtaaagacgtgggccgtggtcagatctgtgcaggtgactacagggttacccctaatctccacaggcaccgcctcacccactgcatctgagaataccccaaagtacatgagagtcaggctgtgtggcccctggactgccttagtgaagagaacctcgggcctggccacggtggctagggttccattgatgtagacggtcacataggtgggcttcttcttgggcttcagcacaatgagggtaacattcatgtaggttttaggaggtccggctatctgaggcacgtacacagctgacacggcggttgtggccgtatagactttcatctggggcgtagaggcatcgctcagcacccagaggcactccttgttgaggaacttgcgaagctgttcccggctactgttcgcggcggatgccatgacgtgccagaatatatcccctctcctcgggggtgagtgccaattggcctttaataacaaagcccccaggcagcaccaaaaatgcctgcccgtccgatgtggtggccaggtggacgcagtgcccgtcagttccaagggctactagctgggaagcagccccaaccagcccacccgggggcctggagtcgatcaccttaccccaggccgaggccccttcctcatacagcgggtggctatctatccataggcaggcatccggcgtctttggtgcattggagatagtagctttcacccaacaactttcccaactaacccgtgtctggacagtgaagaacgcttccctgatcaggtctgaatttttatagatacgggagtaggaggtgggaataacaactgggatttcttgttgtgctgtccaggcctgcatggccagtttttccctgaagctagcagaaattctgagggccactgaaatgaggaagcgaaactccctctctggagctcccaaaattgaaacctcagcaagatctgttgctggggaggcatgggtgacagctgtcatcctgtgcagtctgccctgggcactcagctctggatatgtgacaacatagagagcgtgggggctaaaaatatgagcaattcccctgaccagggccctggactcacgaatggcccgacgggtcttagagaaagaaacaggcaccctcgagagtgcccccgacccgacccccacagtgccgccagtccctgctcggcctccgccgccttccccaccggcgctgccccggatgttgctggggttctcgagggctgggtggtgcttggacacagaggtctcagcagccgccttggtctcggccccggccctaagtctgagccccaggcaaagggccggactcccagcgtggcccaacctctgctcccctctattctcctcttgcgttatctccaatagaatttgcttgaggtcatacgttttagggtgctcgacctgggccgcggccaccggcatatgctctatacccgcccctccggggggcccaggatctataggtatgggctgcatagccgcagcagactcctggaccccagaggcctctctgataagatgcccgtcggtcagagcccttttggccccctcaaagagagacaggtaataaatctgtagctccccaaccagccctccttcatcgtaaaatcgaagggcggccacgtggaaggggttgtagagctctggaaggccctcatcgcagtacactggcacactggtaaacgtgccccgatggctaggccgtccgggcagcatgccccgagcagcaaacacgcggcagaccctcgtgagacccgtccggtcactgaagagagtctggcaccaggccccctcgcagtttggcacgcgattggggcaaagctctgccataaccgtgtcgggaacaaataggtgcacgaggaggggggtcccgaggccactcaacacttggttgtcaatgtggacatccatagctctctcatgcgtttggctacagcatcatagcgcttgtttctggtggatttaaataacagggccccgtagacagtcttttgtgagtaaatagagatgatgacatggatgtagagactgaggaccacatccaccaccttctcggaggaggcccccctaaacagcatcaggcagcaagggaacacaaaggaaaccagggccgggatgtgaggcctcagcgccccctcctgatcaaagagggcctcgctgaccccggagatgacattctcattcagaaagtagtgatagaggtgattgaccacagtcttaaccaggccctggacttgttcaggctcccacttgtcccgctggtcctgtgtgtcttgtcggatctcggtccagggcctcagcgccggctggaaatgcggccccatgtagttgcctgtaagggcgcacaccactccctcatgggtctcaatcagggtgcactcgctggatccatcacatacgtggtactcgccacagccccagcaggcaaacacggaggccatgctctcaggtaacgggagatggaactccagcttactatacgagcacaggtggcgaggattgggctcatccgtgcccccctccccccgcgggaggctcaatcggccttggtctgacattccaccccggccaggtccaggagggtgcaaatattctccaggcgctgcacctcagagacctcctgctcaaagagacctcccaccgccacgtagacgcgggccaccgtccggggaaggtcagtggggtcccagctcagcaattctccaaattctctctccccaatagtgcctcgcttcttatcctgtctttcagagcatccgggggcagacatttcacctcttgtttgtggacgaggctaactttatcaagaaggaggccctgccggcgatcctgggctttatgcttcagaaggatgccaagattatcttcatctcgtctgtgaactcggctgaccaggccaccagctttctttataagctgaaggatgctcaggagcggctgctgaacgtggtaagttatgtgtgtcaggagcatcggcaagattttgacatgcaggacagcatggtctcatgcccctgctttcgcctgcacatcccgtcctacatcaccatggacagcaacatccgagcaaccaccaacctctttctggacggggcctttagcaccgagctgatgggtgacacctcctcgctgagccagggtagctgagccgcactgtgcgtgacgatgccatcaaccagctggagctctgccgggttgacaccctcaacccccgagtagccggacgcctagcctcctccctctacgtgtacgttgatccggcctataccaacaacacatccgcatcaggcaccggaatcgccgccgtgactcacgacagggcggaccctaacagggtcatcgtcctgggcctggaacacttcttcctcaaggacctaacaggggacgctgccctccagatcgccacctgcgtcgtggccctcgtctcctcgatcgtcaccctgcacccccacttggaggaggtgaaggtagccgtggagggcaacagcagtcaggactctgcggtggccattgcctcaatcattggggaatcctgccccctcccctgcgccttcgtgcacaccaaggacaagacgtccagcctgcagtggcccatgtacctcctgactaatgagaagtccaaggcctttgagaggctcatctacgcagtgaacacggccagcctttctgccagtcaggtcaccgtctccaacaccatccagctctccttcgatccggtcctctatctcatctcccagatcagggccatcaagcccatccctctccgcgacggtacctacacctacaccggcaagcagcgcaacctctctgacgacgtgctggttgcgctagtcatggctcattttctcgcaacaacacagaagcacacgttcaagaaagttcattaa

SEQ ID NO: 110 - protein, 690 aa, Homo sapiens
MLYASQRGRLTENLRNALQQDSTTQGCLGAETPSIMYTGAKSDRWAHPLVGTIHASNLYCPMLRAYCRHYGPRPVFVASDESLPMFGASPALHTPVQVQMCLLPELRDTLQRLLPPPNLEDSEALTEFKTSVSSARAILEDPNFLEMREFVTSLASFLSGQYKHKPARLEAFQKQVVLHSFYFLISIKSLEITDTMFDIFQSAFGLEEMTLEKLHIFKQKASVFLIPRRHGKTWIVVAIISLILSNLSNVQIGYVAHQKHVASAVFTEIIDTLTKSFDSKRVEVNKETSTITFRHSGKISSTVMCATCFNKNSIRGQTFHLLFVDEANFIKKEALPAILGFMLQKDAKIIFISSVNSADQATSFLYKLKDAQERLLNVVSYVCQEHRQDFDMQDSMVSCPCFRLHIPSYITMDSNIRATTNLFLDGAFSTELMGDTSSLSQGSLSRTVRDDAINQLELCRVDTLNPRVAGRLASSLYVYVDPAYTNNTSASGTGIAAVTHDRADPNRVIVLGLEHFFLKDLTGDAALQIATCVVALVSSIVTLHPHLEEVKVAVEGNSSQDSAVAIASIIGESCPLPCAFVHTKDKTSSLQWPMYLLTNEKSKAFERLIYAVNTASLSASQVTVSNTIQLSFDPVLYLISQIRAIKPIPLRDGTYTYTGKQRNLSDDVLVALVMAHFLATTQKHTFKKVH

SEQ ID NO: 111 - DNA, 978 bp, Homo sapiens
atgctgtatgcctcgcagcgcggccgtctgaccgagaacctaagaaacgcccttcagcaggacagcaccacgcaaggctgcctgggtgccgagaccccgagtattatgtacacaggggccaagtcagacaggtgggctcaccctctggtgggcacaattcacgccagtaatttatattgcccaatgcttcgagcatactgccgccactatggccccaggcccgtgtttgtagcttctgatgaatcattacccatgttcggtgcgagccctgcccttcacaccccagtccaggtccagatgtgcctactaccagagctacgcgacacgttacagcgcctgctgccaccacccaatcttgaagactccgaggccttgacggaattcaagaccagcgtgtcctctgcccgtgccatccttgaggaccccaactttttggagatgagagagtttgtcaccagcctggccagcttcctgagtggtcagtacaagcacaagcccgcccgcctagaagcattccagaaacaagtagtgttacattctttttattttctgatctcaatcaaatctttagagattacagacaccatgtttgacatctttcaaagtgctttcggattggaagaaatgacgctggagaagctgcacatttttaagcaaaaagccagcgtgtttcttatccccaggcgccacggcaagacctggatagtcgtggccatcatcagcctcatcctctcgaatctctccaacgtgcaaataggctacgtggctcaccagaaacatgtcgcgtccgccgttttcactgaaattattgacaccttgaccaagagcttcgactccaagcgtgtagaggtcaacaaggagaccagcaccatcacgtttaggcacagtgggaaaatctccagcaccgtaatgtgtgccacctgcttcaataagaatgtaagacctgacgtttcagtacttggcaattgtagagcatag

SEQ ID NO: 112 - protein, 325 aa, Homo sapiens
MLYASQRGRLTENLRNALQQDSTTQGCLGAETPSIMYTGAKSDRWAHPLVGTIHASNLYCPMLRAYCRHYGPRPVFVASDESLPMFGASPALHTPVQVQMCLLPELRDTLQRLLPPPNLEDSEALTEFKTSVSSARAILEDPNFLEMREFVTSLASFLSGQYKHKPARLEAFQKQVVLHSFYFLISIKSLEITDTMFDIFQSAFGLEEMTLEKLHIFKQKASVFLIPRRHGKTWIVVAIISLILSNLSNVQIGYVAHQKHVASAVFTEIIDTLTKSFDSKRVEVNKETSTITFRHSGKISSTVMCATCFNKNVRPDVSVLGNCRA

SEQ ID NO: 113 - DNA, 1011 bp, Homo sapiens
atggcatccgccgcgaacagtagccgggaacagcttcgcaagttcctcaacaaggagtgcctctgggtgctgagcgatgcctctacgccccagatgaaagtctatacggccacaaccgccgtgtcagctgtgtacgtgcctcagatagccggacctcctaaaacctacatgaatgttaccctcattgtgctgaagcccaagaagaagcccacctatgtgaccgtctacatcaatggaaccctagccaccgtggccaggcccgaggttctcttcactaaggcagtccaggggccacacagcctgactctcatgtactttggggtattctcagatgcagtgggtgaggcggtgcctgtggagattaggggtaaccctgtagtcacctgcacagatctgaccacggcccacgtctttaccacctcaaccgccgttaaaacagtagaagaactgcaagatatcacaccctcggagatcatcccactgggacggggtggtgcctggtatgcagaaggggccctgtacatgtttttcgttaacatggacatgctgatgtgctgccccaatatgccaacctttccatcactgacccattttatcaacctgctaaccagatgtgataacggggaatgtgtgacatgctatggtgccggggcccacgtgaacatcctgcgtggctggacggaggacgatagcccaggcacatctggcacctgtccttgtctgctgccatgcacggccctgaacaacgactacgtacccataactgggcatcgggctttgttgggtctgatgttcaaaccggaggatgccccctttgtggtgggactgagatttaacccacccaaaatgcacccggacatgtcacgtgtcctgcagggggttcttgctaatgggaaagaggtcccatgtacagcacaaccttggaccctgctgcgattttctgacctttacagccgggctatgctctacaattgccaagtactgaaacgtcaggtcttacattcttattga

SEQ ID NO: 114 - protein, 336 aa, Homo sapiens
MASAANSSREQLRKFLNKECLWVLSDASTPQMKVYTATTAVSAVYVPQIAGPPKTYMNVTLIVLKPKKKPTYVTVYINGTLATVARPEVLFTKAVQGPHSLTLMYFGVFSDAVGEAVPVEIRGNPVVTCTDLTTAHVFTTSTAVKTVEELQDITPSEIIPLGRGGAWYAEGALYMFFVNMDMLMCCPNMPTFPSLTHFINLLTRCDNGECVTCYGAGAHVNILRGWTEDDSPGTSGTCPCLLPCTALNNDYVPITGHRALLGLMFKPEDAPFVVGLRFNPPKMHPDMSRVLQGVLANGKEVPCTAQPWTLLRFSDLYSRAMLYNCQVLKRQVLHSY

SEQ ID NO: 115 - DNA, 1524 bp, Homo sapiens
atggatgtccacattgacaaccaagtgttgagtggcctcgggacccccctcctcgtgcacctatttgttcccgacacggttatggcagagctttgccccaatcgcgtgccaaactgcgagggggcctggtgccagactctcttcagtgaccggacgggtctcacgagggtctgccgcgtgtttgctgctcggggcatgctgcccggacggcctagccatcggggcacgtttaccagtgtgccagtgtactgcgatgagggccttccagagctctacaaccccttccacgtggccgcccttcgattttacgatgaaggagggctggttggggagctacagatttattacctgtctctctttgagggggccaaaagggctctgaccgacgggcatcttatcagagaggcctctggggtccaggagtctgctgcggctatgcagcccatacctatagatcctgggccccccggaggggcgggtatagagcatatgccggtggccgcggcccaggtcgagcaccctaaaacgtatgacctcaagcaaattctattggagataacgcaagaggagaatagaggggagcagaggttgggccacgctgggagtccggccctttgcctggggctcagacttagggccggggccgagaccaaggcggctgctgagacctctgtgtccaagcaccacccagccctcgagaaccccagcaacatccggggcagcgccggtggggaaggcggcggaggccgagcagggactggcggcactgtgggggtcgggtcgggggcactctcgagggtgcctgtttctttctctaagacccgtcgggccattcgtgagtccagggccctggtcaggggaattgctcatatttttagcccccacgctctctatgttgtcacatatccagagctgagtgcccagggcagactgcacaggatgacagctgtcacccatgcctccccagcaacagatcttgctgaggtttcaattttgggagctccagagagggagtttcgcttcctcatttcagtggccctcagaatttctgctagcttcagggaaaaactggccatgcaggcctggacagcacaacaagaaatcccagttgttattcccacctcctactcccgtatctataaaaattcagacctgatcagggaagcgttcttcactgtccagacacgggttagttgggaaagttgttgggtgaaagctactatctccaatgcaccaaagacgccggatgcctgcctatggatagatagccacccgctgtatgaggaaggggcctcggcctggggtaaggtgatcgactccaggcccccgggtgggctggttggggctgcttcccagctagtagcccttggaactgacgggcactgcgtccacctggccaccacatcggacgggcaggcatttttggtgctgcctgggggctttgttattaaaggccaattggcactcacccccgaggagaggggatatattctggcacgtcatggcatccgccgcgaacagtag

SEQ ID NO: 116 - protein, 507 aa, Homo sapiens
MDVHIDNQVLSGLGTPLLVHLFVPDTVMAELCPNRVPNCEGAWCQTLFSDRTGLTRVCRVFAARGMLPGRPSHRGTFTSVPVYCDEGLPELYNPFHVAALRFYDEGGLVGELQIYYLSLFEGAKRALTDGHLIREASGVQESAAAMQPIPIDPGPPGGAGIEHMPVAAAQVEHPKTYDLKQILLEITQEENRGEQRLGHAGSPALCLGLRLRAGAETKAAAETSVSKHHPALENPSNIRGSAGGEGGGGRAGTGGTVGVGSGALSRVPVSFSKTRRAIRESRALVRGIAHIFSPHALYVVTYPELSAQGRLHRMTAVTHASPATDLAEVSILGAPEREFRFLISVALRISASFREKLAMQAWTAQQEIPVVIPTSYSRIYKNSDLIREAFFTVQTRVSWESCWVKATISNAPKTPDACLWIDSHPLYEEGASAWGKVIDSRPPGGLVGAASQLVALGTDGHCVHLATTSDGQAFLVLPGGFVIKGQLALTPEERGYILARHGIRREQ

SEQ ID NO: 117 - DNA, 678 bp, Homo sapiens
atgtcagaccaaggccgattgagcctcccgcggggggaggggggcacggatgagcccaatcctcgccacctgtgctcgtatagtaagctggagttccatctcccgttacctgagagcatggcctccgtgtttgcctgctggggctgtggcgagtaccacgtatgtgatggatccagcgagtgcaccctgattgagacccatgagggagtggtgtgcgcccttacaggcaactacatggggccgcatttccagccggcgctgaggccctggaccgagatccgacaagacacacaggaccagcgggacaagtgggagcctgaacaagtccagggcctggttaagactgtggtcaatcacctctatcactactttctgaatgagaatgtcatctccggggtcagcgaggccctctttgatcaggagggggcgctgaggcctcacatcccggccctggtttcctttgtgttcccttgctgcctgatgctgtttaggggggcctcctccgagaaggtggtggatgtggtcctcagtctctacatccatgtcatcatctctatttactcacaaaagactgtctacggggccctgttatttaaatccaccagaaacaagcgctatgatgctgtagccaaacgcatgagagagctatggatgtccacattgacaaccaagtgttga

SEQ ID NO: 118 - protein, 225 aa, Homo sapiens
MSDQGRLSLPRGEGGTDEPNPRHLCSYSKLEFHLPLPESMASVFACWGCGEYHVCDGSSECTLIETHEGVVCALTGNYMGPHFQPALRPWTEIRQDTQDQRDKWEPEQVQGLVKTVVNHLYHYFLNENVISGVSEALFDQEGALRPHIPALVSFVFPCCLMLFRGASSEKVVDVVLSLYIHVIISIYSQKTVYGALLFKSTRNKRYDAVAKRMRELWMSTLTTKC

SEQ ID NO: 119 - DNA, 705 bp, Homo sapiens
atggcacacgccagagacaaggcaggcgctgttatggccatgatactcatctgtgagacaagccttatttggacatctagtggcagttcgacggcctcggcggggaacgtgacaggcacaacggctgtgactacaccatcaccgtcggcatcgggtcctagcacaaaccagtctacgaccttgaccacgactagcgcccctataaccacaactgctattctgagcaccaacacaaccacggtgacattcactgggacaactgtcacaccagtcccaacaacttccaacgcatccaccatcaatgtcaccactaaggttactgcacagaacatcactgccaccgaggccggaacaggaacctccacgggtgtgactagcaatgtcaccaccaggtcctcctccaccaccagtgctactacccgtattaccaacgctaccaccttggcccccacgctgtcctccaaagggacgtccaatgccacaaaaacaactgctgagcttcccaccgttccagatgagaggcagccatctttatcttacggtcttcctctctggacactggtgtttgtggggctcacttttctgatgctaattctgatatttgcggctgggctaatgatgtccgccaagaacaagcccctggacgaagccctgctcacgaatgccgtcacgcgagacccgtcgctttacaagggactggtgtag

SEQ ID NO: 120 - protein, 234 aa, Homo sapiens
MAHARDKAGAVMAMILICETSLIWTSSGSSTASAGNVTGTTAVTTPSPSASGPSTNQSTTLTTTSAPITTTAILSTNTTTVTFTGTTVTPVPTTSNASTINVTTKVTAQNITATEAGTGTSTGVTSNVTTRSSSTTSATTRITNATTLAPTLSSKGTSNATKTTAELPTVPDERQPSLSYGLPLWTLVFVGLTFLMLILIFAAGLMMSAKNKPLDEALLTNAVTRDPSLYKGLV

SEQ ID NO: 121 - DNA, 1263 bp, Homo sapiens
atggtcgatgaacaagtggcggtggaacacggcaccgtcagccacacaatcagccgggaggaagatggcgtggtccacgagcggcgtgtcttggccagcggggagagggtggaagtcttttataaggctccggccccacgacctcgggagggacgggcctctaccttccacgacttcaccgtcccggcggctgctgccgtcccagggccggaacctgagcctgagccacacccacctatgccaatccatgccaatgggggaggagagaccaagaccaatacccaggatcagaatcaaaatcagaccacccggacccggaccaatgccaaagctgaagaacggactgcggagatggatgacaccatggcctcgtcaggagggcagagaggggcaccaatttccgcggacctactctctctctcctcgttaactggtagaatggcagccatggccccctcctggatgaagagcgaggtatgcggtgagagaatgagattcaaggaagatgtctatgatggagaggcagagaccctagctgagcctccacgctgtttcatgctgagctttgttttcatctattactgctgctatttggcgttcctggccctactcgcctttggttttaacccactctttttgcccagctttatgccggtgggggccaaggtgcttcggggtaaggggcgtgattttggggtgcccctgtcttatgggtgtccgaccaatccattctgcaaggtttacacccttatcccggccgtggtcattaacaatgtgacttattaccccaacaacacggacagccatgggggtcatggtggatttgaggcggctgcccttcatgtagctgcactttttgagtctgggtgcccaaatctacaggctgtgactaataggaacaggacatttaacgtcaccagagccagtggccgagttgaaaggcgccttgtacaagatatgcagagggtcctggcgagtgctgtggtggtgatgcatcatcactgccactatgagacatattatgtctttgatggggtgggccccgagtttggtaccattcctacgccctgcttcaaggatgtgttggcctttaggccgtcattggtgaccaactgcaccgcgccgttaaagacatccgtcaagggtcctaactggtcaggggcagctggaggcatgaaacggaagcaatgtcgtgttgaccggctcacggaccgctcattccctgcatacctcgaggaggtcatgtatgtgatggttcagtag

SEQ ID NO: 122 - protein, 420 aa, Homo sapiens
MVDEQVAVEHGTVSHTISREEDGVVHERRVLASGERVEVFYKAPAPRPREGRASTFHDFTVPAAAAVPGPEPEPEPHPPMPIHANGGGETKTNTQDQNQNQTTRTRTNAKAEERTAEMDDTMASSGGQRGAPISADLLSLSSLTGRMAAMAPSWMKSEVCGERMRFKEDVYDGEAETLAEPPRCFMLSFVFIYYCCYLAFLALLAFGFNPLFLPSFMPVGAKVLRGKGRDFGVPLSYGCPTNPFCKVYTLIPAVVINNVTYYPNNTDSHGGHGGFEAAALHVAALFESGCPNLQAVTNRNRTFNVTRASGRVERRLVQDMQRVLASAVVVMHHHCHYETYYVFDGVGPEFGTIPTPCFKDVLAFRPSLVTNCTAPLKTSVKGPNWSGAAGGMKRKQCRVDRLTDRSFPAYLEEVMYVMVQ

SEQ ID NO: 123 - DNA, 906 bp, Homo sapiens
atggatttgaaagtggtagtgtctctctcctctcgtctgtataccgatgagattgccaagatgcaacagaggattggctgcatcttacccctggcttccactcacgggacgcagaatgtgcagggattgggcttgggtcaggtgtactctctagagacggtcccggactacgtgtccatgtacaactacctgtctgattgcaccctggccgtgctggatgaggttagcgtggacagtttgatactcaccaagattgttccaggtcagacctacgccattaagaacaagtaccagccctttttccagtggcatgggaccgggagcctcagtgttatgcccccggtatttggacgagagcatgccaccgtgaagttagagtccaatgatgtggacattgttttccccatggtgctgcctacgcccatagcagaggaggtgctgcagaagattctcctgtttaacgtgtactcccgggttgtcatgcaggctcccgggaacgcagacatgctcgatgtacacatgcatcttggctctgtttcatacctggggcaccactacgagttggccctcccggaggtgccagggccccttggcttggccctgctggacaatctgagtctctacttttgcatcatggtgaccctgctgcccagggccagtatgcgcctggtccgaggccttatccggcatgagcatcacgacctcttgaatctcttccaggaaatggtgccggatgagatagcccggatagacctggatgacctgtcagtggcggatgatttatcacgcatgcgtgtgatgatgacctacctacagtctttggcatcactatttaatttggggcctcgcttggccacagcggcatactctcaagagaccctgacggccacttgctggttaagataa

SEQ ID NO: 124 - protein, 301 aa, Homo sapiens
MDLKVVVSLSSRLYTDEIAKMQQRIGCILPLASTHGTQNVQGLGLGQVYSLETVPDYVSMYNYLSDCTLAVLDEVSVDSLILTKIVPGQTYAIKNKYQPFFQWHGTGSLSVMPPVFGREHATVKLESNDVDIVFPMVLPTPIAEEVLQKILLFNVYSRVVMQAPGNADMLDVHMHLGSVSYLGHHYELALPEVPGPLGLALLDNLSLYFCIMVTLLPRASMRLVRGLIRHEHHDLLNLFQEMVPDEIARIDLDDLSVADDLSRMRVMMTYLQSLASLFNLGPRLATAAYSQETLTATCWLR

SEQ ID NO: 125 - DNA, 4146 bp, Homo sapiens
atggcctcaaatgagggtgtggaaaacagacccttcccctatctgacggtggatgccgacctgctctcgaacctgcggcagtcagcggctgaggggttgtttcatagctttgacctgctggtgggcaaggatgccagagaggcgggcatcaagtttgaggtgctactcggggtctacacgaacgccatccaatatgttcgcttcctggagacggcactggccgtgtcctgtgtgaacacggaattcaaagacctgagtcgtatgacggatggcaagattcaatttcgaatctccgtccccaccattgctcacggggacggaaggagacccagcaagcagcggacattcattgtggtcaaaaattgccacaaacaccacattagtacggaaatggaactgtccatgctggatctggagatcctgcatagtatccccgagaccccggtcgagtacgcagagtacgtgggggctgtcaagaccgtggcctcggccctacagtttggggtcgatgccctggagaggggcctcattaacaccgtcctgagtgtgaagcttcgccatgcccctcccatgtttatcctgcagaccctggcggatcccaccttcactgagagggggttctccaagactgtcaagtctgacctcattgccatgttcaagaggcatctgctggagcactccttcttcctggaccgggccgagaacatgggctccgggttttctcagtacgtgcgaagccgtctctctgagatggtagcggccgtgtccggggagagcgtgctcaagggggtcagtacctacacgaccgccaaggggggagagccagtggggggggtgtttattgtcaccgacaacgttctgcgccagctcttgaccttcctgggtgaggaggccgacaaccagattatggggccctcgagctatgcttcttttgtggtacggggggagaacctggtcactgccgtgagctacgggcgcgtgatgcgtacgtttgagcatttcatggctaggatcgtggactcaccggaaaaagccggaagcaccaagtctgacctgccggctgtggccgcaggggtcgaggatcagccccgggtgccgatctcagccgccgtcatcaagctgggcaaccacgcggtggccgtggaaagcctgcaaaagatgtacaatgacactcagtccccataccccctcaacagaagaatgcagtacagctactatttcccagtgggcctgtttatgcccaatcccaagtacacgacctccgccgccatcaaaatgctggacaaccccacacagcagctaccggtggaggcatggatcgtaaacaagaataaccttctcctcgcgtttaacctacagaatgctctcaaggttctctgtcacccccgactccacacacccgcccataccctaaacagcctcaacgcggccccggccccgcgagacaggcgcgagacctactccctgcaacacaggaggcccaatcacatgaatgtgcttgttattgtggacgagttctatgataacaagtatgcagcccccgtgacagatatagctctcaagtgcgggctgcccaccgaagacttcctccacccgtccaattatgacctgctgcggctggagctgcaccccctttatgatatttacattggcagggatgccggggagagggccaggcacagggctgtgcaccggctaatggtgggtaacctgccgactcccctggccccagctgcattccaagaggcccgggggcagcagtttgagaccgccacatctctggcccacgtggtggatcaggccgttattgagactgtgcaggatactgcctatgacactgcctatccagccttcttctacgtagtcgaggctatgatccacgggtttgaggaaaagtttgtcatgaacgtgcctttggtgtccctgtgcatcaacacctactgggaacggtcagggaggcttgcctttgtgaacagcttttccatgatcaagttcatctgccgccacctgggaaataacgccatctccaaggaggcctattccatgtatagaaaaatctatggggaacttatagccctagagcaggccctgatgcgcctggccgggtcagatgttgtgggggatgagagcgtgggtcagtatgtctgcgctctcctggaccctaacctgctccccccggtggcctacacagacattttcacccatcttctcaccgttagtgaccgggccccccagattattatcggaaatgaggtttacgctgacaccctggccgcgccccagtttattgagagggttggaaacatggatgagatggctgcccaatttgtggccttgtacggctaccgggttaacggagaccacgaccacgatttccgtctgcacctaggcccttatgtagatgaggggcatgcggatgtgctggaaaagatcttttactacgttttcctcccaacctgcaccaatgcccacatgtgcggcctcggggtggactttcagcacgtggcccagaccctggcctacaacgggccagccttcagccaccattttaccagggacgaggacatcctcgacaatttggagaatgggacgctcagggatctgctggagatctccgacctccgccccaccgtgggcatgatcagggacctcagcgcctcattcatgacctgccccactttcacccgtgccgtgcgtgtgtcggtggacaatgacgttacgcagcagctggccccgaatcccgccgacaagcggacagagcagactgttttggtgaacgggctggtggcctttgccttctccgagaggacccgggccgtcacccagtgtctctttcacgccattcctttccatatgttttacggggacccgcgagtggctgccaccatgcaccaggatgttgccacctttgttatgcgcaatcctcagcagcgggccgtggaagccttcaaccggccagagcagctctttgcagagtaccgggagtggcaccgctcgcccatgggcaaatacgcggccgaatgtcttccttccctcgtttcaatcagtggaatgaccgccatgcacatcaagatgtcccccatggcctatattgcccaggccaagctcaagatccacccaggggtggccatgaccgtggtcaggaccgatgagatcctctctgaaaacatattgtttagctccagggcctcaacatccatgttcattgggaccccaaatgttagccgccgggaggccagggtggacgcggtaacctttgaggtgcatcacgagatggcctccatcgacaccgggcttagttatagctcgaccatgactccggccagggtggcggccatcactactgacatgggtatccacacccaagacttctttagcgtctttccggccgaggcctttggcaaccagcaagtcaatgactacatcaaggccaaggtgggcgctcagcgcaatgggacgctgcttcgggaccccaggacatacctggcaggtatgactaatgttaatggagctccaggactctgccacggccagcaggccacctgtgagattatcgtaacaccggtcacggcagacgtggcttattttcaaaagtccaactctccaaggggacgggccgcctgtgtggtctcctgtgaaaactacaatcaggaggttgccgaggggctcatctatgaccattctcgcccggatgccgcctatgaataccggagcactgtgaatccctgggcatctcagctgggttctctgggtgacatcatgtacaactcctcctatcgccagacggccgtcccgggcctctacagcccctgccgggcatttttcaacaaggaggagcttctgcgcaacaacaggggactctacaacatggtcaacgagtacagccagcgacttggagggcacccagccaccagcaacacagaggtgcagtttgtagtgattgctggcactgacgtgtttctggagcagccctgcagctttctgcaggaggcattccccgcactctcagcctcctcccgggcactcatcgatgagtttatgtctgtcaaacagacccacgcccccatccattacggacactatataattgaagaggtggcgccggtacgaagaatattaaagtttggaaataaggtggttttttga

SEQ ID NO: 126 - protein, 1381 aa, Homo sapiens
MASNEGVENRPFPYLTVDADLLSNLRQSAAEGLFHSFDLLVGKDAREAGIKFEVLLGVYTNAIQYVRFLETALAVSCVNTEFKDLSRMTDGKIQFRISVPTIAHGDGRRPSKQRTFIVVKNCHKHHISTEMELSMLDLEILHSIPETPVEYAEYVGAVKTVASALQFGVDALERGLINTVLSVKLRHAPPMFILQTLADPTFTERGFSKTVKSDLIAMFKRHLLEHSFFLDRAENMGSGFSQYVRSRLSEMVAAVSGESVLKGVSTYTTAKGGEPVGGVFIVTDNVLRQLLTFLGEEADNQIMGPSSYASFVVRGENLVTAVSYGRVMRTFEHFMARIVDSPEKAGSTKSDLPAVAAGVEDQPRVPISAAVIKLGNHAVAVESLQKMYNDTQSPYPLNRRMQYSYYFPVGLFMPNPKYTTSAAIKMLDNPTQQLPVEAWIVNKNNLLLAFNLQNALKVLCHPRLHTPAHTLNSLNAAPAPRDRRETYSLQHRRPNHMNVLVIVDEFYDNKYAAPVTDIALKCGLPTEDFLHPSNYDLLRLELHPLYDIYIGRDAGERARHRAVHRLMVGNLPTPLAPAAFQEARGQQFETATSLAHVVDQAVIETVQDTAYDTAYPAFFYVVEAMIHGFEEKFVMNVPLVSLCINTYWERSGRLAFVNSFSMIKFICRHLGNNAISKEAYSMYRKIYGELIALEQALMRLAGSDVVGDESVGQYVCALLDPNLLPPVAYTDIFTHLLTVSDRAPQIIIGNEVYADTLAAPQFIERVGNMDEMAAQFVALYGYRVNGDHDHDFRLHLGPYVDEGHADVLEKIFYYVFLPTCTNAHMCGLGVDFQHVAQTLAYNGPAFSHHFTRDEDILDNLENGTLRDLLEISDLRPTVGMIRDLSASFMTCPTFTRAVRVSVDNDVTQQLAPNPADKRTEQTVLVNGLVAFAFSERTRAVTQCLFHAIPFHMFYGDPRVAATMHQDVATFVMRNPQQRAVEAFNRPEQLFAEYREWHRSPMGKYAAECLPSLVSISGMTAMHIKMSPMAYIAQAKLKIHPGVAMTVVRTDEILSENILFSSRASTSMFIGTPNVSRREARVDAVTFEVHHEMASIDTGLSYSSTMTPARVAAITTDMGIHTQDFFSVFPAEAFGNQQVNDYIKAKVGAQRNGTLLRDPRTYLAGMTNVNGAPGLCHGQQATCEIIVTPVTADVAYFQKSNSPRGRAACVVSCENYNQEVAEGLIYDHSRPDAAYEYRSTVNPWASQLGSLGDIMYNSSYRQTAVPGLYSPCRAFFNKEELLRNNRGLYNMVNEYSQRLGGHPATSNTEVQFVVIAGTDVFLEQPCSFLQEAFPALSASSRALIDEFMSVKQTHAPIHYGHYIIEEVAPVRRILKFGNKVVF

SEQ ID NO: 127 - DNA, 2253 bp, Homo sapiens
atgacacaaggtaagagggagatggggggaggtctcgagggcttctcttcacagctgggtctcttttacgccctggcctgcaaccgcagcccacccacacttcccgaggatgctacccttctaatcaaatggttggacacggccctgggcagggaggccaccttttacgcgtgtcgggctatgcgtcggcttctactcggcgttatccgaatgaatgactgccaggagctgccacccggtttaataattctgagtccgggcaccgtccctggcccccttggagtccagagtctggagcatacagactgcgaaatatggtcctctgcccaccctgaccacgctgcccacctcccggtgcccagggtcatcacatacaccgactgcccgggttccataaacacgagctcaatgtttcgccttatcatccgctacttgtctcatcaccaatttgagcgctgcttcgagcagttctgccgcgtggtcccgcgtcgcttcctagggacctgtaagcgaaactctgcaaagatgctggctcatctgaatcaggttaccaggatccccccctgtccgcccttcagcgggcgggaggccagactcaagttccacttcttctcctggagcacattcatgctgtcatggccaaacaatgccacactccgggagatcaggacgagggccgccaccaacctcacccaccacccacatctagtggatactctgtaccacgcctctccgcagaccccatttctgacacgcagcggtgctctataccgcttcgtcacctgttgcaactgcaccctgcccaatatctccatccagcagtgcaaggccggggacagaccgggggacctggagatcattctacagagtaacggcggagggaggcccgcgagcttccagttcccctcctccccaactggctccctattgcgatgcatagttgctgcgtccctgctgccggaggtgtccgtggggcaccaggagctgtctccgctgcggtccagaagccagggagggcagacggatgtcaggtcgggcccggacccggcccggagactggtggccctcctgcgaagggaagatggggcacctaaagacccccctctgggaccgtttggacacccccgggggcccggcccggccaagagcgaagacgaggagtctgagcgtcgagacgcccctccacccccgctcgattccagcttccaagcttcccggttggtgcccgtggggcctgggtttcgcctgctcgtgttcaacaccaatcgggtgatcaacactaaattggtgtgctcagagcccctggtgaagatgcgagtttgcaatgtcccccgcctcatcaacaactttgtagcccgcaagtacgtggtgaaagagacggcgttcaccgtcagtctattctttacggacggggtgggggccaacctagccatcaatgtcaatatcagtggcacctatctgagcttcctattggccatgacgtcactgcggtgcttcctgcctgtggaggctatttatcccgcggccgtgtcaaactggaactcgactctagatctccatgggctggaaaatcagagcctagtcagagagaaccgaagcggggtcttttggactaccaactttccctcggtggtgtcctgccgggacggtctcaacgtgtcctggtttaaggccgcaactgccaccatatctcgagtgcacgggcagacattggagcagcacctgatccgtgaaatcacccccatcgtgacgcatcgagaggcaaaaatctcccggattaaaaaccggctctttaccctgctagagctacgcaatcggagtcagattcaagtgctgcacaagcgtttcctggaaggcctgctagactgcgcctccctcctgcgcctggatcccagctgtatcaaccgaatcgcctccgagggcctgtttgatttctccaagagaagcatcgcccactccaaaaaccgacacgagtgcgcgcttctgggtcacagacattcggcgaacgtgacaaagctggtggtaaacgagcgcaagacccgcctggacatactgggccgtaacgctaactttttaacgaggtgtaagcatcaggttaatctaagacagtcacctattttcctgaccctcctgaggcacatccgccgacgtctgggcctgggccgtgcttccgtaaaacgagagattacccttctcctggcccacctgcgcaaaaagacagcccccatccactgccgtgatgctcaagtgtaa

SEQ ID NO: 128 - protein, 750 aa, Homo sapiens
MTQGKREMGGGLEGFSSQLGLFYALACNRSPPTLPEDATLLIKWLDTALGREATFYACRAMRRLLLGVIRMNDCQELPPGLIILSPGTVPGPLGVQSLEHTDCEIWSSAHPDHAAHLPVPRVITYTDCPGSINTSSMFRLIIRYLSHHQFERCFEQFCRVVPRRFLGTCKRNSAKMLAHLNQVTRIPPCPPFSGREARLKFHFFSWSTFMLSWPNNATLREIRTRAATNLTHHPHLVDTLYHASPQTPFLTRSGALYRFVTCCNCTLPNISIQQCKAGDRPGDLEIILQSNGGGRPASFQFPSSPTGSLLRCIVAASLLPEVSVGHQELSPLRSRSQGGQTDVRSGPDPARRLVALLRREDGAPKDPPLGPFGHPRGPGPAKSEDEESERRDAPPPPLDSSFQASRLVPVGPGFRLLVFNTNRVINTKLVCSEPLVKMRVCNVPRLINNFVARKYVVKETAFTVSLFFTDGVGANLAINVNISGTYLSFLLAMTSLRCFLPVEAIYPAAVSNWNSTLDLHGLENQSLVRENRSGVFWTTNFPSVVSCRDGLNVSWFKAATATISRVHGQTLEQHLIREITPIVTHREAKISRIKNRLFTLLELRNRSQIQVLHKRFLEGLLDCASLLRLDPSCINRIASEGLFDFSKRSIAHSKNRHECALLGHRHSANVTKLVVNERKTRLDILGRNANFLTRCKHQVNLRQSPIFLTLLRHIRRRLGLGRASVKREITLLLAHLRKKTAPIHCRDAQV

SEQ ID NO: 129 - DNA, 1275 bp, Homo sapiens
aacgagagattacccttctcctggcccacctgcgcaaaaagacagcccccatccactgccgtgatgctcaagtgtaagcagcccggggcccgcttcattcacggggccgtgcacctgccatcgggacagattgtcttccacaccatccacagccccactcttgcctcggcgctgggactgcctggggaaaatgtacccatcccggccctcttccgtgcctcgggcctcaacgtccgtgagagcctacccatgaccaacatgagggcaccgatcatctcgctggctcgcctcatcctggcccccaacccctatatcctagagggacagctgacggtgggcatgacacaggacaacggcattcccgtgctttttgccaggcctgtcattgaggtaaaaagcgggcctgagtccaacattaaagcctcctcgcaacttatgatagcagaagactcctgcctgaatcagatcgcccccttttccgcatcagagcaccccgccttctccatggttgagtccgtaaaacgagtccgggtcgatgagggagcaaacacccggcgcaccatccgggatattctggagatccccgtgactgtgctctcatccctgcaactgtctcccaccaagtccatcctgaaaaaggcaccggagcccccacctccggagccccaagccaccttcgatgccaccccctatgcccgcatcttttacgacatcgggcgacaggtgcccaagctgggcaatgcccccgccgcgcaggtcagcaacgtgctcatcgccaaccgctcccacaactctctaaggctggtgcccaatccggacttgctgcctctccagcatttgtacctcaagcacgtagtgctaaagagtctgaatctggagaatatagtgcaggactttgaggccatcttcacctccccgtctgataccatcagtgaggctgaaaccaaggcctttgagaagctggtggagcaagccaaaaacaccgtagagaacatagtcttttgcctcaacagcatctgttccacctctacactcccagatgtcgtccccgatgtcaataacccaaacattagcctggctctagagaagtattttctcatgttccctccctcaggcaccattatgagaaatgtcagattcgccacccccatcgtccggctcttgtgccaaggggctgagcttggcaccatggcacagtttctaggaaagtacatcaaggtcaagaaggaaactggaatgtacacactggtcaagctttattacctgctgcgcatc

SEQ ID NO: 130 - protein, 425 aa, Homo sapiens
NERLPFSWPTCAKRQPPSTAVMLKCKQPGARFIHGAVHLPSGQIVFHTIHSPTLASALGLPGENVPIPALFRASGLNVRESLPMTNMRAPIISLARLILAPNPYILEGQLTVGMTQDNGIPVLFARPVIEVKSGPESNIKASSQLMIAEDSCLNQIAPFSASEHPAFSMVESVKRVRVDEGANTRRTIRDILEIPVTVLSSLQLSPTKSILKKAPEPPPPEPQATFDATPYARIFYDIGRQVPKLGNAPAAQVSNVLIANRSHNSLRLVPNPDLLPLQHLYLKHVVLKSLNLENIVQDFEAIFTSPSDTISEAETKAFEKLVEQAKNTVENIVFCLNSICSTSTLPDVVPDVNNPNISLALEKYFLMFPPSGTIMRNVRFATPIVRLLCQGAELGTMAQFLGKYIKVKKETGMYTLVKLYYLLRI

SEQ ID NO: 131 - DNA, 2121 bp, Homo sapiens
atgcagttgctctgtgttttttgcctggtgttgctatgggaggtgggggctgccagcctcagcgaggttaagctgcacctggacatagaggggcatgcttcgcattacaccatcccatggaccgaactgatggcaaaggtcccaggccttagcccagaggcgctgtggagagaggcaaatgtcaccgaagatttggcgtctatgcttaaccgctacaagttaatttacaagacgtctggtacccttggtattgcgctggccgagcctgtcgatatccctgctgtctctgaaggatccatgcaagtggatgcatctaaggtccatcccggagtcattagcggcctgaattcccctgcctgcatgcttagtgccccccttgagaagcagctcttctactatattggcaccatgctgcccaacacgcggccacacagctatgtcttttatcagctgcgctgtcacttgtcttatgtggccctgtccatcaacggggacaagtttcagtacacgggggccatgacttctaaatttctgatgggcacctacaagcgagtgaccgagaagggagatgagcatgtgttgagcctggtctttggcaagacgaaggacctgccggatctgagggggccttttagttacccatccttaaccagtgcccaaagcggggactattccctggtgattgttacaacctttgtgcattatgccaactttcacaactactttgtacccaacctgaaggatatgttttcccgagccgtcaccatgacagccgccagctacgctcgctacgttctccagaaactggtcctgctggagatgaagggaggctgccgggagccggaactggacacggaaacgctgactaccatgtttgaggtttctgtggccttctttaaggtgggtcatgctgtgggtgagactggcaatggctgcgtggacctccgctggttggccaagagcttctttgagctgactgtcctgaaagacatcatcggcatatgttatggggccactgtcaagggcatgcaatcctacgggctggagcgcttggccgccatgctgatggccacggtcaagatggaggagctgggtcacctgactactgagaaacaggagtacgcgctgaggttagccaccgtcggctaccccaaggccggggtttacagtggcctcattggaggcgccacatctgtgcttctctcggcctacaaccgccacccccttttccagcccctgcataccgtgatgagagagaccctgtttatcggcagccacgtggtgctacgcgagttgcggctgaacgtgactacccaggggcccaaccttgccctataccaactgctgtccaccgccctgtgctcggccctagagattggggaggttttgcgggggctagccctggggacagagagcgggctcttctcaccgtgctacctcagcctacgatttgacctcacacgagacaagctgctgagcatggccccccaggaggcaacgctggaccaggcggccgtttcaaatgctgtggatgggtttcttgggcggctctctttggagcgagaagacagggatgcgtggcatctccccgcctacaaatgcgtggacaggctcgacaaagttctgatgattatcccgctcatcaatgtgacattcataatctctagtgaccgtgaggtccgaggctcggcgctatacgaggccagcaccacctatctcagcagctctctctttctctcccccgttataatgaataaatgttcgcagggtgctgtggctggggagccccgccagattccaaagatccagaattttaccaggacgcagaaatcctgcattttttgtggctttgccctgctcagttatgatgaaaaggaaggcctggaaactacaacctacatcacctcccaggaagtccaaaactccatcttgagctccaactactttgattttgacaacctccacgttcactatctgctgctgaccaccaacgggactgtcatggaaattgcgggcctgtatgaagaaagagcacacgttgttttggcaataatcctgtactttattgcttttgctctgggtatctttctggttcacaagattgttatgtttttcctttag

SEQ ID NO: 132 - protein, 706 aa, Homo sapiens
MQLLCVFCLVLLWEVGAASLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAHVVLAIILYFIAFALGIFLVHKIVMFFL

SEQ ID NO: 133 - DNA, 1824 bp, Homo sapiens
atggctggatttccaggaaaggaggccggcccgcccggcggctggcgaaaatgtcaggaggatgagtcccctgaaaacgaaagacacgaaaacttttacgctgagattgatgactttgccccctcagttcttaccccaaccggtagtgactcaggggcaggtgaggaagatgacgacggcctctaccaggtgcccacccactggccccccttgatggctccgaccggactgtctggggagagggtgccgtgccggacccaggctgccgtgacatctaatactggaaactctccgggtagccggcacacatcgtgcccctttaccctgcccaggggagcccagccaccagcacccgcacaccaaaagcctactgcccctaccccgaagccccgtagccgggagtgtggccccagcaagacgccagaccccttctcctggttccgcaaaacttcgtgcacagaaggaggtgcggactccacgagccggagcttcatgtaccagaaaggctttgaggagggcctggccggccttgggctcgatgacaaatctgactgtgagtctgaggatgagtctaatttccgcaggccctcttctcattccgcgttaaaacaaaagaatggtggcaagggtaagccttctggtctcttcgaacacctggccgcccatgggcgcgagtttagtaagttatccaagcatgctgcccagctgaagcggctaagtgggagtgtgatgaatgttctgaatctggatgatgcccaagacacccgccaggccaaggctcagaggaaggagagcatgcgggttccaattgtgacccacctaacaaatcatgtaccagtaatcaaacctgcctgttccctatttttggaaggtgcccctggtgtgggaaagactactatgctgaatcatttgaaggctgtctttggggacctgaccattgtagtccctgaaccgatgcggtactggactcatgtgtatgaaaatgccattaaggccatgcacaagaatgtgactcgggcgaggcatggaagggaagacacgtcggctgaggttttagcatgccagatgaaatttaccacccctttcagagtcttggcctctaggaagcggagcttgctggttaccgagtctggtgccaggtcagtcgcgcccctggattgttggattttgcatgatcgccatttgctgtcggcctccgtggttttccctctaatgctgctccggagccagcttctctcctatagtgactttattcaagttttggccacgtttaccgcagacccgggggacaccatagtctggatgaagttaaacgtggaggagaacatgcgccgcctgaagaagcgagggaggaaacatgagtcaggactggatgctggctacctgaagagtgtcaacgatgcatatcacgccgtttactgtgcctggctcctaacacagtattttgcccccgaggacattgtgaaggtatgtgccggtctaacaacaatcacgacagtatgtcatcaaagccacacccccataattcgaagtggtgtggccgagaagctgtataagaacagcatctttagtgtacttaaggaagtcatacagcctttccgagccgacgcggtgttgttggaagtttgtcttgcgttcacccgaacgctagcctaccttcagtttgtgcttgtggacctctcagagtttcaggacgatttacctggatgctggacggagatttacatgcaggcattgaagaatccggcaatacgatctcagtttttcgattgggctggtctgagcaaggtgatctctgattttgagaggggaaatcgggactag

SEQ ID NO: 134 - protein, 607 aa, Homo sapiens
MAGFPGKEAGPPGGWRKCQEDESPENERHENFYAEIDDFAPSVLTPTGSDSGAGEEDDDGLYQVPTHWPPLMAPTGLSGERVPCRTQAAVTSNTGNSPGSRHTSCPFTLPRGAQPPAPAHQKPTAPTPKPRSRECGPSKTPDPFSWFRKTSCTEGGADSTSRSFMYQKGFEEGLAGLGLDDKSDCESEDESNFRRPSSHSALKQKNGGKGKPSGLFEHLAAHGREFSKLSKHAAQLKRLSGSVMNVLNLDDAQDTRQAKAQRKESMRVPIVTHLTNHVPVIKPACSLFLEGAPGVGKTTMLNHLKAVFGDLTIVVPEPMRYWTHVYENAIKAMHKNVTRARHGREDTSAEVLACQMKFTTPFRVLASRKRSLLVTESGARSVAPLDCWILHDRHLLSASVVFPLMLLRSQLLSYSDFIQVLATFTADPGDTIVWMKLNVEENMRRLKKRGRKHESGLDAGYLKSVNDAYHAVYCAWLLTQYFAPEDIVKVCAGLTTITTVCHQSHTPIIRSGVAEKLYKNSIFSVLKEVIQPFRADAVLLEVCLAFTRTLAYLQFVLVDLSEFQDDLPGCWTEIYMQALKNPAIRSQFFDWAGLSKVISDFERGNRD

SEQ ID NO: 135 - DNA, 747 bp, Homo sapiens
atggatcccacccggggtctgtgtgccctctccacacacgacctggcaaaatttcacagtcttcccccggctagaaaggcggcaggtaagcgagcgcaccttcggtgttactccaagctgctctctcttaagagctgggagcaactggcctcttttttgtctctgcccccgggacccacgtttacagactttagactatttttcgaagtcaccctgggtcggagaatcgcagattgcgttgtggtagctctgcagccttatccccggtgttatattgtagaatttaagacggccatgagcaacacggccaacccgcaaagcgttactcgcaaggcacagaggctagagggcaccgcccagttgtgtgactgtgccaattttcttcgcacgtcctgcccccccgtgctgggcagtcagggcctggaagtcttggcggcgttggtatttaaaaaccagcgatccctgagaacgctccaggtagagtttccagccctgggccaaaagaccctccccacctccaccaccggcctgctaaacctcctctcccgctggcaggatggcgctctccgggcacgtcttgatagaccccgcccgactgcccagggacacaggccccgaactcatgtgggccccaagccttcgcaactcactgcgcgtgtcccccgaagcgctcgagctggcagagcgggaggccgaaagggccaggtcggagcggtgggacaggtgtgcccaggtgctcaaaaatag

SEQ ID NO: 136 - protein, 248 aa, Homo sapiens
MDPTRGLCALSTHDLAKFHSLPPARKAAGKRAHLRCYSKLLSLKSWEQLASFLSLPPGPTFTDFRLFFEVTLGRRIADCVVVALQPYPRCYIVEFKTAMSNTANPQSVTRKAQRLEGTAQLCDCANFLRTSCPPVLGSQGLEVLAALVFKNQRSLRTLQVEFPALGQKTLPTSTTGLLNLLSRWQDGALRARLDRPRPTAQGHRPRTHVGPKPSQLTARVPRSARAGRAGGRKGQVGAVGQVCPGAQK

SEQ ID NO: 137 - DNA, 1713 bp, Homo sapiens
atggcgctctccgggcacgtcttgatagaccccgcccgactgcccagggacacaggccccgaactcatgtgggccccaagccttcgcaactcactgcgcgtgtcccccgaagcgctcgagctggcagagcgggaggccgaaagggccaggtcggagcggtgggacaggtgtgcccaggtgctcaaaaataggctgctccgcgtggagctggacggcatcatgcgtgaccacctggccagggcggaggagatccgccaggacctggatgctgtagtggccttctctgatggcctggagagcatgcaggtcaggtccccctccacgggagggcgctctgcgccagccccgccctccccatccccagcccagccgttcactcggctcaccgggaacgcccagtatgcagtctcaatctctcccacggacccccctctgatggtggccggcagcctggctcaaacgctgcttggtaatctgtacgggaacatcaaccagtgggtaccgtccttcggaccctggtacaggaccatgtcggctaatgccatgcagcggcgcgtgttccctaagcagctgaggggcaacctgaactttaccaactccgtctccctaaagctgatgacagaagtggtggcggtgcttgagggcaccacccaggactttttctcagacgtcaggcacctgccagacctccaggctgccctgatcctctcggtggcctacctgctactccaggggggctcctcacaccagcagcgccccctccctgcctcacgggaagagctgctggagctgggcccggagagcctagagaaaatcatcgccgacctcaaggccaagtcacccggcggaaattttatgattttaacaagcggaaacaaggaagcgcgccagtcaatagcccctctcaaccgacaggcggcatatccacccggcacattcgcggacaataagatttacaacctgtttgtgggagcgggactactgcccacgacggccgcgctgaacgtgcccggggcggcgggtcgggaccgggacctggtgtaccggatcgccaaccagatctttggggaggatgtgccccccttctcatctcaccagtggaacctgcgcgtaggtttagccgcactcgaggccctgatgctcgtctacacgctctgcgagaccgccaacctggccgaggcggccacccggcgtctacacctatcgtccctgctcccccaggcaatgcagcggcgcaagcctgccatggcgtcagctggtatgccgggcgcctatccagtccagacgcttttccgccacggggagctcttccgcttcatctgggcccactacgtgaggcccacggtggcggcagacccccaggcctccatcagctctcttttccccgggctggttttgctggccctggagctgaagttgatggatgggcaggctccctcccattatgccataaacctgaccggacaaaagtttgacaccctctttgagattatcaaccagaagcttttatttcacgacccggctgccatgctggcggcgcgcacacagctgcgtctagccttcgaggacggcgtcggtgttgccctggggcgcccctcgcccatgcttgcggcgcgggagatcctggagcgtcagttctcagcctcggatgactacgaccggctgtacttcctgacgctgggctacctggcctccccggtggccccaagctga

SEQ ID NO: 138 - protein, 570 aa, Homo sapiens
MALSGHVLIDPARLPRDTGPELMWAPSLRNSLRVSPEALELAEREAERARSERWDRCAQVLKNRLLRVELDGIMRDHLARAEEIRQDLDAVVAFSDGLESMQVRSPSTGGRSAPAPPSPSPAQPFTRLTGNAQYAVSISPTDPPLMVAGSLAQTLLGNLYGNINQWVPSFGPWYRTMSANAMQRRVFPKQLRGNLNFTNSVSLKLMTEVVAVLEGTTQDFFSDVRHLPDLQAALILSVAYLLLQGGSSHQQRPLPASREELLELGPESLEKIIADLKAKSPGGNFMILTSGNKEARQSIAPLNRQAAYPPGTFADNKIYNLFVGAGLLPTTAALNVPGAAGRDRDLVYRIANQIFGEDVPPFSSHQWNLRVGLAALEALMLVYTLCETANLAEAATRRLHLSSLLPQAMQRRKPAMASAGMPGAYPVQTLFRHGELFRFIWAHYVRPTVAADPQASISSLFPGLVLLALELKLMDGQAPSHYAINLTGQKFDTLFEIINQKLLFHDPAAMLAARTQLRLAFEDGVGVALGRPSPMLAAREILERQFSASDDYDRLYFLTLGYLASPVAPS

SEQ ID NO: 139 - DNA, 366 bp, Homo sapiens
atgtgtgtgggcgcagcttggactcgcagggcgccggacctgagccaggtcaccctggctgacggaaatctgctcttcaaccttggcagtgtcctccctaaccgcctggtggtgggtgtcctctactgccttgtccactggggtgcagacgagcatgaaacccgggttcgggcacgccttcgccccctctttgtggccttcctctgcctggccggctatctgcttctggaccgtgccatcctctcggatgcgcacgactacgagggcctctggcacgccgtggccctgagcatggcggcctggcagcggccttactcccctacccgaaacaagagacgagaaggaggcaaaacccccgtgcgatga

SEQ ID NO: 140 - protein, 121 aa, Homo sapiens
MCVGAAWTRRAPDLSQVTLADGNLLFNLGSVLPNRLVVGVLYCLVHWGADEHETRVRARLRPLFVAFLCLAGYLLLDRAILSDAHDYEGLWHAVALSMAAWQRPYSPTRNKRREGGKTPVR

SEQ ID NO: 141 - DNA, 411 bp, Homo sapiens
atgggactgggggtgcgcggggatgacgcgcgaatgacacctgggattcacgggctcctcctgaagatgctaaagaactggccgctttgcgctctgcgcgaggacgagctcaggtttttgcacctatccctgaccaagctgctgaccctaagtgttaacttttacctgtggcgggaagccgtgataaatactggaaaccgcacgaatcgtgtgttggcacgcaaggtgccggatgaatactggtacctgctttaccgggccctggcccgggtgggcttcccggcagaggccctgcgccccggtaaccgctcacggtctctttgtctgtttttgcatgaccggcctgatgtcacaggggccgtctgtgcatgtgtgtgggcgcagcttggactcgcagggcgccggacctga

SEQ ID NO: 142 - protein, 136 aa, Homo sapiens
MGLGVRGDDARMTPGIHGLLLKMLKNWPLCALREDELRFLHLSLTKLLTLSVNFYLWREAVINTGNRTNRVLARKVPDEYWYLLYRALARVGFPAEALRPGNRSRSLCLFLHDRPDVTGAVCACVWAQLGLAGRRT

SEQ ID NO: 143 - DNA, 1818 bp, Homo sapiens
atggtgcaggcaccgtctgtatacgtctgcggcttcgtggagcgcccggacgccccacccaaggacgcctgccttcacctggatcccctcaccgtcaagagccagctccctctgaagaagcccttgccactcacggtggaacacctgccggatgctccggtcggctcagtctttggcctttaccagagccgagcgggtctctttagcgcagcctcgattacctctggggacttcctgtccctgctggactcaatttaccacgattgcgatattgcacagagtcagcgcctgcccctccctcgagaacccaaggtggaggctctgcacgcctggctcccctcactgtcactggcctccctccacccagacataccccaaaccaccgcagatggaggcaagctgtccttctttgaccacgtgtctatctgtgccctgggtcgtcggcgcggcaccacggcagtctacggtacagaccttgcgtgggtcctgaagcactttagtgacctggaaccgtctatcgccgcccagattgagaatgacgccaatgccgcaaagcgtgaatccggatgcccggaagaccaccctctgcccctcacgaagctcatagctaaggcaatcgatgctggatttctgagaaaccgcgtggagactctgaggcaggacaggggtgtggccaatatcccagccgagtcgtatttaaaggccagcgacgccccggacctacaaaagccggacaaggcacttcagagcccaccaccggcctccacagacccagccaccatgctatcaggtaacgcaggagaaggagcaacagcctgcggaggttcggccgccgcgggccaggacctcatcagcgtcccccgcaacacctttatgacactgcttcagaccaacctggacaacaaaccgccgaggcagaccccgctaccctacgcggccccgctgccccccttttcccaccaggcaatagccaccgcgccttcctacggtcctggggccggagcggtcgccccggccggcggctactttacctccccaggaggttactacgccgggcccgcgggcggggacccgggtgccttcttggcgatggacgctcacacctaccacccccacccacacccccctccggcctactttggcttgccgggcctctttggcccccctccacccgtgcctccttactacggatcccacttgcgggcagactacgtccccgctccctcgcgatccaacaagcggaaaagagaccccgaggaggatgaagaaggcggggggctattcccgggggaggacgccaccctctaccgcaaggacatagcgggcctctccaagagtgtgaatgagttacagcacacgctacaggccctgcgccgggagacgctgtcctacggccacaccggagtcggatactgcccccagcagggcccctgctacacccactcggggccttacggatttcagcctcatcaaagctacgaagtgcccagatacgtccctcatccgcccccaccaccaacttctcaccaggcagctcaggcgcagcctccacccccgggcacacaggcccccgaagcccactgtgtggccgagtccacgatccctgaggcgggagcagccgggaactctggaccccgggaggacaccaaccctcagcagcccaccaccgagggccaccaccgcggaaagaaactggtgcaggcctctgcgtccggagtggctcagtctaaggagcccaccacccccaaggccaagtctgtgtcagcccacctcaagtccatcttttgcgaggaattgctgaataaacgcgtggcttga

SEQ ID NO: 144 - protein, 605 aa, Homo sapiens
MVQAPSVYVCGFVERPDAPPKDACLHLDPLTVKSQLPLKKPLPLTVEHLPDAPVGSVFGLYQSRAGLFSAASITSGDFLSLLDSIYHDCDIAQSQRLPLPREPKVEALHAWLPSLSLASLHPDIPQTTADGGKLSFFDHVSICALGRRRGTTAVYGTDLAWVLKHFSDLEPSIAAQIENDANAAKRESGCPEDHPLPLTKLIAKAIDAGFLRNRVETLRQDRGVANIPAESYLKASDAPDLQKPDKALQSPPPASTDPATMLSGNAGEGATACGGSAAAGQDLISVPRNTFMTLLQTNLDNKPPRQTPLPYAAPLPPFSHQAIATAPSYGPGAGAVAPAGGYFTSPGGYYAGPAGGDPGAFLAMDAHTYHPHPHPPPAYFGLPGLFGPPPPVPPYYGSHLRADYVPAPSRSNKRKRDPEEDEEGGGLFPGEDATLYRKDIAGLSKSVNELQHTLQALRRETLSYGHTGVGYCPQQGPCYTHSGPYGFQPHQSYEVPRYVPHPPPPPTSHQAAQAQPPPPGTQAPEAHCVAESTIPEAGAAGNSGPREDTNPQQPTTEGHHRGKKLVQASASGVAQSKEPTTPKAKSVSAHLKSIFCEELLNKRVA

SEQ ID NO: 145 - DNA, 1038 bp, Homo sapiens
atgctatcaggtaacgcaggagaaggagcaacagcctgcggaggttcggccgccgcgggccaggacctcatcagcgtcccccgcaacacctttatgacactgcttcagaccaacctggacaacaaaccgccgaggcagaccccgctaccctacgcggccccgctgccccccttttcccaccaggcaatagccaccgcgccttcctacggtcctggggccggagcggtcgccccggccggcggctactttacctccccaggaggttactacgccgggcccgcgggcggggacccgggtgccttcttggcgatggacgctcacacctaccacccccacccacacccccctccggcctactttggcttgccgggcctctttggcccccctccacccgtgcctccttactacggatcccacttgcgggcagactacgtccccgctccctcgcgatccaacaagcggaaaagagaccccgaggaggatgaagaaggcggggggctattcccgggggaggacgccaccctctaccgcaaggacatagcgggcctctccaagagtgtgaatgagttacagcacacgctacaggccctgcgccgggagacgctgtcctacggccacaccggagtcggatactgcccccagcagggcccctgctacacccactcggggccttacggatttcagcctcatcaaagctacgaagtgcccagatacgtccctcatccgcccccaccaccaacttctcaccaggcagctcaggcgcagcctccacccccgggcacacaggcccccgaagcccactgtgtggccgagtccacgatccctgaggcgggagcagccgggaactctggaccccgggaggacaccaaccctcagcagcccaccaccgagggccaccaccgcggaaagaaactggtgcaggcctctgcgtccggagtggctcagtctaaggagcccaccacccccaaggccaagtctgtgtcagcccacctcaagtccatcttttgcgaggaattgctgaataaacgcgtggcttga

SEQ ID NO: 146 - protein, 345 aa, Homo sapiens
MLSGNAGEGATACGGSAAAGQDLISVPRNTFMTLLQTNLDNKPPRQTPLPYAAPLPPFSHQAIATAPSYGPGAGAVAPAGGYFTSPGGYYAGPAGGDPGAFLAMDAHTYHPHPHPPPAYFGLPGLFGPPPPVPPYYGSHLRADYVPAPSRSNKRKRDPEEDEEGGGLFPGEDATLYRKDIAGLSKSVNELQHTLQALRRETLSYGHTGVGYCPQQGPCYTHSGPYGFQPHQSYEVPRYVPHPPPPPTSHQAAQAQPPPPGTQAPEAHCVAESTIPEAGAAGNSGPREDTNPQQPTTEGHHRGKKLVQASASGVAQSKEPTTPKAKSVSAHLKSIFCEELLNKRVA

SEQ ID NO: 147 - DNA, 747 bp, Homo sapiens
atgactcatttagtgcttctattgtgctgctgtgtggggtctgtttgtgcatttttctcagatctggttaaatttgaaaatgtcaccgcccatgcaggtgctagagtgaatctcacctgcagcgttcctagcaatgagtcggtctctcggatcgagttgggccggggatacactcccggggatggacaactgcccttggccgtagctacttccaacaacggaactcatataactaacgggggttacaattatagccttaccctagaatgggtaaatgactccaacacctccgtctctctgataatccccaatgtgacccttgcgcatgcaggttactacacgtgcaatgtgacactgagaaattgcagtgtggcctccggggtccactgcaattactctgcaggggaagaagacgaccaataccatgccaaccggaccctcactcagcgtatgcatcttactgttatcccagccactaccatagcccccaccacattagtctcccacaccacaagcaccagtcacaggccccataggcggcctgtttctaaacgtcccacgcataagccagtgaccctgggcccttttccaatcgatccctggcgccctaagaccacctgggtgcactgggccctgctcctaatcacttgtgctgtggtggctccggtgctcctcatcattatcatctcctgtctggggtggctggcgggctgggggaggcggcgcaagggctggatacccctgtga

SEQ ID NO: 148 - protein, 248 aa, Homo sapiens
MTHLVLLLCCCVGSVCAFFSDLVKFENVTAHAGARVNLTCSVPSNESVSRIELGRGYTPGDGQLPLAVATSNNGTHITNGGYNYSLTLEWVNDSNTSVSLIIPNVTLAHAGYYTCNVTLRNCSVASGVHCNYSAGEEDDQYHANRTLTQRMHLTVIPATTIAPTTLVSHTTSTSHRPHRRPVSKRPTHKPVTLGPFPIDPWRPKTTWVHWALLLITCAVVAPVLLIIIISCLGWLAGWGRRRKGWIPL

SEQ ID NO: 149 - DNA, 939 bp, Homo sapiens
atgctctccaccatggcccccgggtccaccgtggggaccctggtggccaacatgacttccgtcaatgcaacggaagatgcgtgcactaaatcctacagcgccttcctcagcggcatgacaagcctgctgctcgtcctgctgattctgctaacactggctggcatcttgtttatcatctttgtaagaaaattggttcaccgaatggatgtctggctgatagctcttctcatagaactgctgctctgggtgctgggaaaaatgattcaggagttctcgtctaccggtctctgccttttgacccagaacatgatgttcttgggcctgatgtgctccgtatggactcacttgggtatggcgttggagaagaccctagcattgttcagccgaacgcccaagagaacctcccacagaaatgtgtgcctgtacctgatgggcgtgttttgtctggtactcctgctgattatcatcctcctcattaccatgggccccgacgccaatctcaacagaggccccaacatgtgcagggaaggccccaccaaaggcatgcacacggccgtccaaggactgaaagccggctgctacctactggcggcagtcctgatcgtcctcctcacagttatcatcatctggaaacttttgcgcacaaaattcggaaggaagccgcgcctgatatgcaacgtcaccttcaccggactcatttgcgccttctcctggtttatgctgtccctgccgctgctcttcctgggtgaggccgggagcctggggtttgattgcacagagtctctcgttgcccgctactacccaggccccgccgcctgtttggccctgttgctcattatactatacgcctggagcttcagccattttatggactctctcaagaaccaggtgacagtcaccgccagatacttcagaagggtgcctagccagtccacctga

SEQ ID NO: 150 - protein, 312 aa, Homo sapiens
MLSTMAPGSTVGTLVANMTSVNATEDACTKSYSAFLSGMTSLLLVLLILLTLAGILFIIFVRKLVHRMDVWLIALLIELLLWVLGKMIQEFSSTGLCLLTQNMMFLGLMCSVWTHLGMALEKTLALFSRTPKRTSHRNVCLYLMGVFCLVLLLIIILLITMGPDANLNRGPNMCREGPTKGMHTAVQGLKAGCYLLAAVLIVLLTVIIIWKLLRTKFGRKPRLICNVTFTGLICAFSWFMLSLPLLFLGEAGSLGFDCTESLVARYYPGPAACLALLLIILYAWSFSHFMDSLKNQVTVTARYFRRVPSQST

SEQ ID NO: 151 - DNA, 3048 bp, Homo sapiens
atgtctgggggactcttctataaccctttcctaagacctaataaaggccttctgaaaaagcctgacaaggagtacctgcgtctcattcccaagtgtttccagacaccaggcgccgcaggggtggtggatgtgcgggggcctcagccccccctgtgcttctaccaagactccctgacggtggtggggggtgacgaggatggaaagggcatgtggtggcgccagcgtgcccaagagggcacggcaaggccggaggcagacacccacggaagccctctggacttccatgtctacgacatactcgagacggtgtacacgcacgagaaatgcgccgtcattccatcggataaacaggggtatgtggtgccatgtggcatcgtcatcaagctactgggccggcgcaaggccgatggggccagcgtgtgtgtgaacgtgtttgggcagcaggcctacttctacgccagcgcgcctcagggtctggacgtggagtttgcagtcctcagcgccctcaaggccagcaccttcgaccgcaggaccccctgccgggtctcggtggagaaggtcacgcgccgttccattatgggctacggcaaccatgccggcgactaccacaagatcaccctctcccatcccaacagtgtgtgtcacgtggccacgtggctgcaagacaagcacgggtgtcggatctttgaggccaacgtggatgccacgcgccgctttgtcctggacaatgactttgtcacctttggctggtacagctgccgccgcgccatcccccgcctccagcaccgggactcgtacgccgagctcgagtacgactgtgaggtgggcgacctctcggtccggcgtgaagacagctcctggccctcctaccaggccctggccttcgatatcgagtgtctgggggaggagggcttccccacggccaccaacgaggctgacctgatcctgcagatatcctgcgtcctctggtcgacaggggaggaggccgggcgctataggcgcatcctgctgacgctgggcacctgcgaagacatagagggggttgaggtctacgagttcccatcggagctggacatgctctacgccttcttccagctcatcagagacctcagcgtggagattgtgaccggctacaacgtggccaactttgactggccctacattctggacagagccaggcacatctacagcatcaacccagcctctctgggcaaaattagggctgggggcgtctgcgaggtcaggcgaccccatgatgcgggcaagggcttcttgcgggccaacaccaaggtccgcatcaccggcctcatccccatcgacatgtacgccgtgtgccgggacaagctcagcctctcagactacaagctggacacagtagccaggcacctactgggggccaagaaggaggatgtgcattacaaggagattcctcgcctctttgcagcgggccccgaggggcgcaggcggctcggcatgtactgcgtgcaggactcggccctggtcatggatctgctaaaccatttcgtgatccacgtggaggtggcagagattgccaagatcgctcacatcccctgcaggcgggtgctggacgatgggcagcagatccgcgtgttctcctgcctcctggcggccgcccaaaaggaaaactttatcctgcccatgccctcggcctctgaccgggacggctaccagggggccaccgtcatccagcccctgtccggattctacaactccccggttctggtggtggactttgccagcctctacccgagcatcattcaggctcataatctctgttattctaccatgataacgccgggagaagagcacaggctagccggcctgcgcccgggagaagactatgagtccttcaggctcacggggggcgtctaccactttgtaaagaagcacgtgcacgagtccttcttggctagtctgttgacctcctggctggccaagcgcaaggccatcaagaagctgctggcggcctgcgaggatccgcgccaaaggaccatcctcgacaagcagcagctggccatcaagtgcacgtgcaacgccgtctacggcttcaccggggtggccaacggcctctttccctgcctctccatcgccgagacggtgacgctgcagggccgcacgatgttggagcgggccaaggccttcgtggaggccctgagccccgccaacctgcaggccctggccccctccccggacgcctgggcgcccctcaaccccgagggccagcttcgagtcatctacggggacacggactcgctgtttatcgagtgccgggggttttcagagagcgagaccctgcgctttgccgatgccctggccgcccacaccacccggagcctgtttgtggcccccatctccctggaggccgagaagaccttctcctgcctgatgctgattacaaagaagagatatgtgggggtgctgacggacggcaagaccctgatgaagggggtggagctcgtccggaagacggcctgcaagtttgtgcagacacgctgccggcgcgtgctcgacctggtgctggcggatgcccgggtaaaggaggcggccagcctcctctcccaccggcccttccaagagtcatttacacaagggctacctgtgggctttttgcccgtcattgacatcctaaaccaggcctacacagacctccgtgaaggcagggtccccatgggggagctctgcttttcaacggagctcagccgcaagctctcagcctacaagagcacccagatgcctcacctggccgtctaccagaagttcgtcgagcgcaacgaggaactgccccagatccacgaccgcatccagtacgtctttgtggagcccaaggggggagtgaagggggcgagaaagacggagatggccgaggacccggcctacgccgagcggcacggcgttcccgtggccgtggatcattatttcgacaagctgctccaaggagcggccaacatcctccagtgcctctttgataacaactccggggccgccctctccgtcctccagaattttacagcccggccaccattctaa

SEQ ID NO: 152 - protein, 1015 aa, Homo sapiens
MSGGLFYNPFLRPNKGLLKKPDKEYLRLIPKCFQTPGAAGVVDVRGPQPPLCFYQDSLTVVGGDEDGKGMWWRQRAQEGTARPEADTHGSPLDFHVYDILETVYTHEKCAVIPSDKQGYVVPCGIVIKLLGRRKADGASVCVNVFGQQAYFYASAPQGLDVEFAVLSALKASTFDRRTPCRVSVEKVTRRSIMGYGNHAGDYHKITLSHPNSVCHVATWLQDKHGCRIFEANVDATRRFVLDNDFVTFGWYSCRRAIPRLQHRDSYAELEYDCEVGDLSVRREDSSWPSYQALAFDIECLGEEGFPTATNEADLILQISCVLWSTGEEAGRYRRILLTLGTCEDIEGVEVYEFPSELDMLYAFFQLIRDLSVEIVTGYNVANFDWPYILDRARHIYSINPASLGKIRAGGVCEVRRPHDAGKGFLRANTKVRITGLIPIDMYAVCRDKLSLSDYKLDTVARHLLGAKKEDVHYKEIPRLFAAGPEGRRRLGMYCVQDSALVMDLLNHFVIHVEVAEIAKIAHIPCRRVLDDGQQIRVFSCLLAAAQKENFILPMPSASDRDGYQGATVIQPLSGFYNSPVLVVDFASLYPSIIQAHNLCYSTMITPGEEHRLAGLRPGEDYESFRLTGGVYHFVKKHVHESFLASLLTSWLAKRKAIKKLLAACEDPRQRTILDKQQLAIKCTCNAVYGFTGVANGLFPCLSIAETVTLQGRTMLERAKAFVEALSPANLQALAPSPDAWAPLNPEGQLRVIYGDTDSLFIECRGFSESETLRFADALAAHTTRSLFVAPISLEAEKTFSCLMLITKKRYVGVLTDGKTLMKGVELVRKTACKFVQTRCRRVLDLVLADARVKEAASLLSHRPFQESFTQGLPVGFLPVIDILNQAYTDLREGRVPMGELCFSTELSRKLSAYKSTQMPHLAVYQKFVERNEELPQIHDRIQYVFVEPKGGVKGARKTEMAEDPAYAERHGVPVAVDHYFDKLLQGAANILQCLFDNNSGAALSVLQNFTARPPF

SEQ ID NO: 153 - DNA, 2574 bp, Homo sapiens
atgactcggcgtagggtgctaagcgtggtcgtgctgctagccgccctggcgtgccgtctcggtgcgcagaccccagagcagcccgcaccccccgccaccacggtgcagcctaccgccacgcgtcagcaaaccagctttcctttccgagtctgcgagctctccagccacggcgacctgttccgcttctcctcggacatccagtgtccctcgtttggcacgcgggagaatcacacggagggcctgttgatggtgtttaaagacaacattattccctactcgtttaaggtccgctcctacaccaagatagtgaccaacattctcatctacaatggctggtacgcggactccgtgaccaaccggcacgaggagaagttctccgttgacagctacgaaactgaccagatggataccatctaccagtgctacaacgcggtcaagatgacaaaagatgggctgacgcgcgtgtatgtagaccgcgacggagttaacatcaccgtcaacctaaagcccaccgggggcctggccaacggggtgcgccgctacgccagccagacggagctctatgacgcccccgggtggttgatatggacttacagaacaagaactaccgtcaactgcctgataactgacatgatggccaagtccaacagccccttcgacttctttgtgaccaccaccgggcagactgtggaaatgtcccctttctatgacgggaaaaataaggaaaccttccatgagcgggcagactccttccacgtgagaactaactacaagatagtggactacgacaaccgagggacgaacccgcaaggcgaacgccgagccttcctggacaagggcacttacacgctatcttggaagctcgagaacaggacagcctactgcccgcttcaacactggcaaacctttgactcgaccatcgccacagaaacagggaagtcaatacattttgtgactgacgagggcacctctagcttcgtgaccaacacaaccgtgggcatagagctcccggacgccttcaagtgcatcgaagagcaggtgaacaagaccatgcatgagaagtacgaggccgtccaggatcgttacacgaagggccaggaagccattacatattttataacgagcggaggattgttattagcttggctacctctgaccccgcgctcgttggccaccgtcaagaacctgacggagcttaccactccgacttcctcaccccccagcagtccatcgcccccagccccatccgcggcccgcgggagcacccccgccgccgttctgaggcgtcggaggcgggatgcggggaacgccaccacaccggtgccccccacggcccccgggaagtccctgggcaccctcaacaatcccgccaccgtccagatccaatttgcctacgactccctgcgccgccagatcaaccgcatgctgggagaccttgcgcgggcctggtgcctggagcagaagaggcagaacatggtgctgagagaactaaccaagattaatccaaccaccgtcatgtccagcatctacggtaaggcggtggcggccaagcgcctgggggatgtcatctcagtctcccagtgcgtgcccgttaaccaggccaccgtcaccctgcgcaagagcatgagggtccctggctccgagaccatgtgctactcgcgccccctggtgtccttcagctttatcaacgacaccaagacctacgagggacagctgggcaccgacaacgagatcttcctcacaaaaaagatgacggaggtgtgccaggcgaccagccagtactacttccagtccggcaacgagatccacgtctacaacgactaccaccactttaaaaccatcgagctggacggcattgccaccctgcagaccttcatctcactaaacacctccctcatcgagaacattgactttgcctccctggagctgtactcacgggacgaacagcgtgcctccaacgtctttgacctggagggcatcttccgggagtacaacttccaggcgcaaaacatcgccggcctgcggaaggatttggacaatgcagtgtcaaacggaagaaatcaattcgtggacggcctgggggaacttatggacagtctgggtagcgtgggtcagtccatcaccaacctagtcagcacggtggggggtttgtttagcagcctggtctctggtttcatctccttcttcaaaaaccccttcggcggcatgctcattctggtcctggtggcgggggtggtgatcctggttatttccctcacgaggcgcacgcgccagatgtcgcagcagccggtgcagatgctctaccccgggatcgacgagctcgctcagcaacatgcctctggtgagggtccaggcattaatcccattagtaagacagaattacaagccatcatgttagcgctgcatgagcaaaaccaggagcaaaagagagcagctcagagggcggccggaccctcagtggccagcagagcattgcaggcagccagggaccgttttccaggcctacgcagaagacgctatcacgatccagagaccgccgccgcactgcttggggaggcagagactgagttttaa

SEQ ID NO: 154 - protein, 857 aa, Homo sapiens
MTRRRVLSVVVLLAALACRLGAQTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 155 - DNA, 2367 bp, Homo sapiens
ggacgacgcggagtacttattgggccgcttctccgtcctggcggacagcgtcctagaaaccctggcgaccattgcctccagcgggatagagtggacggcggaggccgctcgggactttctggagggagtgtggggtgggcccggggcagcccaggacaactttatcagcgtggccgagccggtcagcaccgcgtcgcaggcctcggccgggctgctgctgggtggaggagggcagggctccgggggcagacgcaagcgccgtctggccaccgttctccccggactcgaggtctagagacccctggggcggcgatgtcggggctgctggcggcggcgtacagccaggtgtacgccctggcggttgagctgagcgtgtgcacccggctggacccccggagtctggacgtggctgcggtggtgcgcaacgccggcctgctggccgagctggaggccatcctccttccccgtttgagacggcagaatgaccgtgcatgcagcgccctgtccctggagctggtgcacctgctagagaactcgagagaggcctctgccgcgctgctcgcccctggtagaaagggtacccgggtcccgcctctccgtaccccctcagtcgcgtactctgtggagttttacggggggcataaagtcgatgtaagtttgtgcctaataaatgacatagagattttaatgaagagaatcaatagcgtgttttattgcatgtctcacaccatggggctggagagcctggaacgggccctggatctgctgggccgctttcggggcgtaagtcccatcccagacccgcgcctctacatcacctctgtgccctgctggcgctgtgtgggcgagctgatggttctgcccaaccacggcaacccttccacggcagaggggacccacgtctcctgtaaccacctggcggtgccggtgaatccggagccggtctcgggactgtttgagaatgaagtccgccaggcggggctcgggcacctgttggaggctgaggagaaggcgaggccgggcggcccagaggagggcgcggtcccgggcccggggcggccggaggcagagggggcgaccagagcgctggacacctacaacgtcttctcgacagtgcccccggaggtggcggagctctcggagctcctctattggaactctggcggccatgctatcggtgcaacggggcagggggagggtggcggccattcccgcctctctgccctgtttgcccgggagcgtcgcctggccctggtgcggggggcctgcgaggaggcgctggcgggggcaaggctgactcacctgtttgacgccgtggctcccggggccacggagcggctcttctgcggcggggtctacagctcctcgggcgacgcggtggaggcgctgaaggcggactgcgccgccgccttcacggcgcacccccagtaccgggccatcctgcaaaagaggaacgagctgtacacgcggctcaaccgagccatgcagcggttgggccgaggcgaggaggaggcgtcccgggagagcccggaggtgccccggccggctggggcacgagagcccggcccgtccggcgccctctcggacgcgctcaagcgcaaggagcagtacctgcgccaggtggccaccgagggtctggccaagctgcagtcctgcctggcgcaacagagcgagaccctgaccgagaccctgtgcctgcgcgtctggggggacgtggtctactgggagctggcccgcatgcgcaaccacttcctctacagacgggccttcgtctcgggtccctgggaggacaggcgcgccggcgagggtgccgcctttgagaattccaagtacatcaaaacacacctgtttacccagaccctgagctcggagcacctgcacgcgctgacgcacagcctgtacaccttcatcacggggcccctggcggaggagagcgggctctttcccccacccagcaacgtggccctggctcgctgctgtgacgccgcaggtacgctgccccaccagaaggcattcctgacttccctgatatggccaggcatcgagccgagcgactggatagagacctccttcaacagcttctacagcgtacccgggggctcactggcatctagccagcaaattctgtgccgggcgctgcgcgaggccgtcctaaccgtgtccctctacaacaagacctgggggcggtccttgatcctgcgccgggcggacgcggtcagccccggccaggccctgcccccggatgggctttacctcacgtacgactctgaccgccccctaattcttctgtataagggcagggggtgggtatttaaggatctatatgcccttctctacctgcacctccaaatgagagatgactcggcg

SEQ ID NO: 156 - protein, 789 aa, Homo sapiens
GRRGVLIGPLLRPGGQRPRNPGDHCLQRDRVDGGGRSGLSGGSVGWARGSPGQLYQRGRAGQHRVAGLGRAAAGWRRAGLRGQTQAPSGHRSPRTRGLETPGAAMSGLLAAAYSQVYALAVELSVCTRLDPRSLDVAAVVRNAGLLAELEAILLPRLRRQNDRACSALSLELVHLLENSREASAALLAPGRKGTRVPPLRTPSVAYSVEFYGGHKVDVSLCLINDIEILMKRINSVFYCMSHTMGLESLERALDLLGRFRGVSPIPDPRLYITSVPCWRCVGELMVLPNHGNPSTAEGTHVSCNHLAVPVNPEPVSGLFENEVRQAGLGHLLEAEEKARPGGPEEGAVPGPGRPEAEGATRALDTYNVFSTVPPEVAELSELLYWNSGGHAIGATGQGEGGGHSRLSALFARERRLALVRGACEEALAGARLTHLFDAVAPGATERLFCGGVYSSSGDAVEALKADCAAAFTAHPQYRAILQKRNELYTRLNRAMQRLGRGEEEASRESPEVPRPAGAREPGPSGALSDALKRKEQYLRQVATEGLAKLQSCLAQQSETLTETLCLRVWGDVVYWELARMRNHFLYRRAFVSGPWEDRRAGEGAAFENSKYIKTHLFTQTLSSEHLHALTHSLYTFITGPLAEESGLFPPPSNVALARCCDAAGTLPHQKAFLTSLIWPGIEPSDWIETSFNSFYSVPGGSLASSQQILCRALREAVLTVSLYNKTWGRSLILRRADAVSPGQALPPDGLYLTYDSDRPLILLYKGRGWVFKDLYALLYLHLQMRDDSA

SEQ ID NO: 157 - DNA, 3387 bp, Homo sapiens
atgcagggtgcacagactagcgaggataatctgggcagccagagccagccgggtccgtgcggctacatctacttttaccccctggccacctaccctcttagggaggtggccacactggggaccggctacgcgggccacaggtgcctgacggtgccgctcctttgcggcatcaccgtggagccgggcttcagcatcaatgtcaaggctctgcacaggaggcccgaccccaactgcgggctcctacgcgctacctcctatcacagggacatctacgtgttccacaatgcccatatggttccccccatctttgaggggccgggtctcgaggccctctgtggcgagaccagggaggtgtttgggtacgacgcctacagcgccctaccgagggaaagctccaagccgggggacttcttccccgaagggctagatccctctgcctacctgggggcggtggcaataaccgaggccttcaaggagcgactctacagcggaaacctggtggccattccatcgttaaaacaggaggtagcggtggggcagtctgcgagcgttagggtcccgctctacgacaaggaggtgttcccagagggcgtgccccagctccgccagttttacaactcggacctcagccgctgcatgcacgaggcgctgtacaccgggctggcgcaggcgctgcgcgtccgacgggtgggcaagctggtggagctgctggagaagcagagcctgcaggaccaggccaaggtggccaaggtggcccccctcaaggagttcccagcctcaaccatcagtcacccggactcgggagccttaatgattgtggacagcgcggcatgcgagctggcggtgagctacgcacccgccatgctggaggcctcgcacgagaccccggccagcctcaactacgactcgtggcccctgtttgccgactgtgagggtccagaggcccgtgtggctgcgttacaccgatataatgccagcctggccccccacgtgtccacgcagatctttgccaccaattccgtcctctacgtctcgggggtctcgaagtcaaccggtcagggcaaggagagtctctttaacagtttctacatgacccacggcctggggaccctgcaggaggggacctgggacccctgccgccgaccctgcttctcgggctggggtgggccagacgtgaccggaaccaacggtccgggaaactacgctgtggagcacctggtctatgcggcctccttctcgcccaaccttcttgcccgctatgcctactacctgcagttttgccagggacagaagagctctctgaccccggtgccggagacgggcagctacgtggcgggggcggccgccagtcccatgtgctcgctctgcgagggccgggccccggccgtgtgcctgaacacgctcttctttaggctgagggaccgcttcccccccgtcatgtccacgcagcggagggacccctatgtgatctcgggggcctcgggctcctacaacgagacggactttttgggcaactttctcaacttcatcgataaggaggacgacgggcagcggccggacgacgagccccgctacacctactggcagctgaaccagaacctgctggagcggctgtctcggctgggcatagacgctgaaggaaagctagagaaggagccccatggcccgcgtgactttgtcaagatgttcaaggacgtggatgcggcggtggacgccgaagtggtccagtttatgaacagcatggccaagaacaacatcacctacaaggacctggtcaagagctgctaccacgtgatgcagtactcgtgcaacccctttgcgcagcccgcctgccccatcttcacccagctgttttaccgctcactgctgaccatcctgcaggacatctccctgcccatctgtatgtgctatgagaatgacaaccccgggcttggccagagccccccagagtggctaaagggtcactaccagacgctgtgcaccaactttaggagcctggccatcgacaagggggtcctcacggccaaggaggccaaggtggtgcatggggagcccacctgcgacctgccagacctggacgcggccctgcagggccgggtgtacggccggcggctgcctgtgcgcatgtccaaggtgctgatgctgtgccccaggaacatcaagatcaagaacagggtggtcttcacgggggagaatgccgccctccagaacagcttcatcaagtccactaccaggagggagaactacatcatcaacgggccctacatgaaattcctcaacacctaccacaagaccctattcccggacactaagctctcaagcctgtacctgtggcacaacttttccaggcggcgctcggtccctgtccccagcggggccagcgcggaggagtactctgacctggccctctttgtggacgggggctcccgggcccacgaagagagcaacgtcatagatgtggtgcctggcaacctggtcacttacgccaagcagaggctcaacaacgccatcctgaaggcgtgcggccagacccagttctacatcagcctgattcagggactggtgccgaggacgcagtcggtgcccgcccgtgactacccccacgtactgggcacgcgggcggtggagtcggcagcggcctacgcggaggccacctcctcccttactgcgaccacggtggtctgcgcggccacagactgtcttagccaggtctgcaaggcccgtccggttgtcacgctgccagtgaccatcaacaagtacacgggggtcaacggcaacaaccagatattccaggccgggaacctgggatactttatgggccggggcgtggacaggaacctgctgcaggcccccggggctgggctgcgcaagcaggccgggggctcttccatgcggaagaagtttgtctttgccacccccaccctagggttgaccgtgaagcgccggacccaagccgcgaccacatatgagattgagaacatcagggctggcctggaggccattatatcacaaaaacaggaggaagactgtgtgtttgatgtggtgtgcaaccttgtggatgccatgggcgaggcatgcgcctcgctgactagggacgacgcggagtacttattgggccgcttctccgtcctggcggacagcgtcctagaaaccctggcgaccattgcctccagcgggatagagtggacggcggaggccgctcgggactttctggagggagtgtggggtgggcccggggcagcccaggacaactttatcagcgtggccgagccggtcagcaccgcgtcgcaggcctcggccgggctgctgctgggtggaggagggcagggctccgggggcagacgcaagcgccgtctggccaccgttctccccggactcgaggtctag

SEQ ID NO: 158 - protein, 1128 aa, Homo sapiens
MQGAQTSEDNLGSQSQPGPCGYIYFYPLATYPLREVATLGTGYAGHRCLTVPLLCGITVEPGFSINVKALHRRPDPNCGLLRATSYHRDIYVFHNAHMVPPIFEGPGLEALCGETREVFGYDAYSALPRESSKPGDFFPEGLDPSAYLGAVAITEAFKERLYSGNLVAIPSLKQEVAVGQSASVRVPLYDKEVFPEGVPQLRQFYNSDLSRCMHEALYTGLAQALRVRRVGKLVELLEKQSLQDQAKVAKVAPLKEFPASTISHPDSGALMIVDSAACELAVSYAPAMLEASHETPASLNYDSWPLFADCEGPEARVAALHRYNASLAPHVSTQIFATNSVLYVSGVSKSTGQGKESLFNSFYMTHGLGTLQEGTWDPCRRPCFSGWGGPDVTGTNGPGNYAVEHLVYAASFSPNLLARYAYYLQFCQGQKSSLTPVPETGSYVAGAAASPMCSLCEGRAPAVCLNTLFFRLRDRFPPVMSTQRRDPYVISGASGSYNETDFLGNFLNFIDKEDDGQRPDDEPRYTYWQLNQNLLERLSRLGIDAEGKLEKEPHGPRDFVKMFKDVDAAVDAEVVQFMNSMAKNNITYKDLVKSCYHVMQYSCNPFAQPACPIFTQLFYRSLLTILQDISLPICMCYENDNPGLGQSPPEWLKGHYQTLCTNFRSLAIDKGVLTAKEAKVVHGEPTCDLPDLDAALQGRVYGRRLPVRMSKVLMLCPRNIKIKNRVVFTGENAALQNSFIKSTTRRENYIINGPYMKFLNTYHKTLFPDTKLSSLYLWHNFSRRRSVPVPSGASAEEYSDLALFVDGGSRAHEESNVIDVVPGNLVTYAKQRLNNAILKACGQTQFYISLIQGLVPRTQSVPARDYPHVLGTRAVESAAAYAEATSSLTATTVVCAATDCLSQVCKARPVVTLPVTINKYTGVNGNNQIFQAGNLGYFMGRGVDRNLLQAPGAGLRKQAGGSSMRKKFVFATPTLGLTVKRRTQAATTYEIENIRAGLEAIISQKQEEDCVFDVVCNLVDAMGEACASLTRDDAEYLLGRFSVLADSVLETLATIASSGIEWTAEAARDFLEGVWGGPGAAQDNFISVAEPVSTASQASAGLLLGGGGQGSGGRRKRRLATVLPGLEV

SEQ ID NO: 159 - DNA, 663 bp, Homo sapiens
atgaacctggccattgctctggactctcctcacccaggcctcgcgtcttatactattctgccacgcccattttatcatataagcctgaagcccgtgagctggcctgacgagaccatgaggccagccaagtctacagattctgtgtttgtgaggaccccggtcgaggcgtgggtcgcgccctcgccgccggacgacaaggtggctgagtccagctacctcatgttcagggccatgtacgcggtgttcacccgggatgagaaagacctgcctttgccagccctggtcctctgccggctcatcaaggcctccctgaggaaggataggaagctgtacgcggagctggcctgcaggacagccgacatcgggggcaaagacacgcacgtacggctcatcatcagcgtcctgcgcgcagtgtacaacgaccactacgactactggtcgcggctcagggtggtgctgtgctacacagtggtgtttgcggtgcgaaactacctggatgaccacaagagcgccgccttcgtgctgggggcaatcgcccactacctggccctctatcgcagactctggtttgcgaggctgggcggcatgccaagatcgctgagacgtcagttccccgtgacgtgggccctggccagcctgactgacttcctgaaatctttgtaa

SEQ ID NO: 160 - protein, 220 aa, Homo sapiens
MNLAIALDSPHPGLASYTILPRPFYHISLKPVSWPDETMRPAKSTDSVFVRTPVEAWVAPSPPDDKVAESSYLMFRAMYAVFTRDEKDLPLPALVLCRLIKASLRKDRKLYAELACRTADIGGKDTHVRLIISVLRAVYNDHYDYWSRLRVVLCYTVVFAVRNYLDDHKSAAFVLGAIAHYLALYRRLWFARLGGMPRSLRRQFPVTWALASLTDFLKSL

SEQ ID NO: 161 - DNA, 666 bp, Homo sapiens
atggccaggttcatcgctcagctcctcctgttggcctcctgtgtggccgccggccaggctgtcaccgctttcttgggtgagcgagtcaccctgacctcctactggaggagggtgagcctcggtccagagattgaggtcagctggtttaaactgggcccaggagaggagcaggtgcttattgggcgcatgcaccacgatgtcatctttatagagtggcctttcaggggcttctttgatatccacagaagtgccaacaccttctttttagtagtcaccgctgccaacatctcccatgacggcaactacctgtgccgcatgaaactgggcgagaccgaggtcaccaagcaggaacacctgagcgtggtgaagcctctaacgctgtctgtccactccgaaaggtctcagttcccagacttctctgtccttactgtgacatgcaccgtgaatgcatttccccatccccacgtccagtggctcatgcccgagggcgtggagcccgcaccaactgcggcaaatggcggtgttatgaaggaaaaggatgggagcctctctgttgctgttgacctgtcacttcccaagccctggcacctgccagtgacctgcgttgggaaaaatgacaaggaggaagcccacggggtttatgtttctggatacttgtcgcaataa

SEQ ID NO: 162 - protein, 221 aa, Homo sapiens
MARFIAQLLLLASCVAAGQAVTAFLGERVTLTSYWRRVSLGPEIEVSWFKLGPGEEQVLIGRMHHDVIFIEWPFRGFFDIHRSANTFFLVVTAANISHDGNYLCRMKLGETEVTKQEHLSVVKPLTLSVHSERSQFPDFSVLTVTCTVNAFPHPHVQWLMPEGVEPAPTAANGGVMKEKDGSLSVAVDLSLPKPWHLPVTCVGKNDKEEAHGVYVSGYLSQ

SEQ ID NO: 163 - DNA, 345 bp, Homo sapiens
atggtgccaatgggcgcgggtccccctagccccggcggggatccggatgggtacgatggcggaaacaactcccaatatccatctgcttctggctcttctgggaacacccccaccccaccgaacgatgaggaacgtgaatctaatgaagagcccccaccgccttatgaggacccatattggggcaatggcgaccgtcactcggactatcaaccactaggaacccaagatcaaagtctgtacttgggattgcaacacgacgggaatgacgggctccctccccctccctactctccacgggatgactcatctcaacacatatacgaagaagcgggcagaggaaggtaa

SEQ ID NO: 164 - protein, 114 aa, Homo sapiens
MVPMGAGPPSPGGDPDGYDGGNNSQYPSASGSSGNTPTPPNDEERESNEEPPPPYEDPYWGNGDRHSDYQPLGTQDQSLYLGLQHDGNDGLPPPPYSPRDDSSQHIYEEAGRGR

SEQ ID NO: 165 - DNA, 306 bp, Homo sapiens
gaagccaccatgcgaccgggtagaccactggctggattctacgctactctccgccgttccttcagaagaatgtccaaaaggtcaaagaacaaggccaagaaggagcgtgtccccgtggaggaccgcccaccgactccgatgcccaccagccagcgactgatccgcagaaacgcgttgggaggaggcgtccgccccgatgcggaggactgcatccaacgcttccaccccctggagccagcgctgggggtgtcaacaaagaactttgacctgttgtccctgagatgtgaattgggatggtgtggataa

SEQ ID NO: 166 - protein, 101 aa, Homo sapiens
EATMRPGRPLAGFYATLRRSFRRMSKRSKNKAKKERVPVEDRPPTPMPTSQRLIRRNALGGGVRPDAEDCIQRFHPLEPALGVSTKNFDLLSLRCELGWCG

SEQ ID NO: 167 - DNA, 183 bp, Homo sapiens
atggtacacgtcctggagcgtgctttgctagagcagcagtcctctgcctgcggcctgcccggctcttctacggagaccaggcctagccacccctgccccgaggacccagacgtcagcagactaagactactcctggtggtactctgtgtcctgtttggacttttatgcctgctcctcatctaa

SEQ ID NO: 168 - protein, 60 aa, Homo sapiens
MVHVLERALLEQQSSACGLPGSSTETRPSHPCPEDPDVSRLRLLLVVLCVLFGLLCLLLI

SEQ ID NO: 169 - DNA, 444 bp, Homo sapiens
atggaacacgaccttgagaggggcccaccgggcccgcgacggccccctcgaggaccccccctctcctcttccctaggccttgctctccttctcctcctcttggcgctactgttttggctgtacatcgttatgagtgactggactggaggagccctccttgtcctctattcctttgctctcatgcttataattataattttgatcatctttatcttcagaagagaccttctctgtccacttggagccctttgtatactcctactgatgagtaagtattacaccctttgccccacaccccctttcccttactcttccttctctaacgcactttctcctctttccccagtcaccctcctgctcatcgctctctggaatttgcacggacaggcattgttccttggaattgtgctgttcatcttcgggtgcttacttggtaagatctaa

SEQ ID NO: 170 - protein, 147 aa, Homo sapiens
MEHDLERGPPGPRRPPRGPPLSSSLGLALLLLLLALLFWLYIVMSDWTGGALLVLYSFALMLIIIILIIFIFRRDLLCPLGALCILLLMSKYYTLCPTPPFPYSSFSNALSPLSPVTLLLIALWNLHGQALFLGIVLFIFGCLLGKI

SEQ ID NO: 171 - DNA, 1158 bp, Homo sapiens
atggaacacgaccttgagaggggcccaccgggcccgcgacggccccctcgaggaccccccctctcctcttccctaggccttgctctccttctcctcctcttggcgctactgttttggctgtacatcgttatgagtgactggactggaggagccctccttgtcctctattcctttgctctcatgcttataattataattttgatcatctttatcttcagaagagaccttctctgtccacttggagccctttgtatactcctactgatgatcaccctcctgctcatcgctctctggaatttgcacggacaggcattgttccttggaattgtgctgttcatcttcgggtgcttacttgtcttaggtatctggatctacttattggagatgctctggcgacttggtgccaccatctggcagcttttggccttcttcctagccttcttcctagacctcatcctgctcattattgctctctatctacaacaaaactggtggactctattggttgatctcctttggctcctcctgtttctggcgattttaatctggatgtattaccatggacaacgacacagtgatgaacaccaccacgatgactccctcccgcaccctcaacaagctaccgatgattctggccatgaatctgactctaactccaacgagggcagacaccacctgctcgtgagtggagccggcgacggacccccactctgctctcaaaacctaggcgcacctggaggtggtcctgacaatggcccacaggaccctgacaacactgatgacaatggcccacaggaccctgacaacactgatgacaatggcccacatgacccgctgcctcaggaccctgacaacactgatgacaatggcccacaggaccctgacaacactgatgacaatggcccacatgacccgctgcctcatagccctagcgactctgctggaaatgatggaggccctccacaattgacggaagaggttgaaaacaaaggaggtgaccagggcccgcctttgatgacagacggaggcggcggtcatagtcatgattccggccatggcggcggtgatccacaccttcctacgctgcttttgggttcttctggttccggtggagatgatgacgacccccacggcccagttcagctaagctactatgac

SEQ ID NO: 172 - protein, 386 aa, Homo sapiens
MEHDLERGPPGPRRPPRGPPLSSSLGLALLLLLLALLFWLYIVMSDWTGGALLVLYSFALMLIIIILIIFIFRRDLLCPLGALCILLLMITLLLIALWNLHGQALFLGIVLFIFGCLLVLGIWIYLLEMLWRLGATIWQLLAFFLAFFLDLILLIIALYLQQNWWTLLVDLLWLLLFLAILIWMYYHGQRHSDEHHHDDSLPHPQQATDDSGHESDSNSNEGRHHLLVSGAGDGPPLCSQNLGAPGGGPDNGPQDPDNTDDNGPQDPDNTDDNGPHDPLPQDPDNTDDNGPQDPDNTDDNGPHDPLPHSPSDSAGNDGGPPQLTEEVENKGGDQGPPLMTDGGGGHSHDSGHGGGDPHLPTLLLGSSGSGGDDDDPHGPVQLSYYD

SEQ ID NO: 173 - DNA, 576 bp, Homo sapiens
atggcctattcaacaagggagatactgttagccctgtgtatacgggacagtcgtgtgcatggaaatggtaccctgcatcctgtgttggagctagcagcaagagaaacacctctccgcctttcgccagaggacactgtagttctgcgttatcatgtgttgcttgaggagataattgaacgaaattcagagacatttacagaaacttggaacagatttataacacacaccgaacatgtggatctggattttaactcagtatttttagagatatttcaccgtggagacccaagccttgggcgcgcgttggcctggatggcctggtgcatgcatgcctgcaggacattgtgttgtaaccagtctactccttactatgttgtggacctgtcagttcgtgggatgttagaagccagcgaaggcctggatggttggattcatcaacagggcggctggtctacattaattgaagacaacattcctggatccagaaggtttagctggactttgtttcttgctggactgactttgagtctgttagttatatgtagttatttatttatctccagaggaagacactaa

SEQ ID NO: 174 - protein, 191 aa, Homo sapiens
MAYSTREILLALCIRDSRVHGNGTLHPVLELAARETPLRLSPEDTVVLRYHVLLEEIIERNSETFTETWNRFITHTEHVDLDFNSVFLEIFHRGDPSLGRALAWMAWCMHACRTLCCNQSTPYYVVDLSVRGMLEASEGLDGWIHQQGGWSTLIEDNIPGSRRFSWTLFLAGLTLSLLVICSYLFISRGRH

SEQ ID NO: 175 - DNA, 2817 bp, Homo sapiens
atgaagaaagcgtggctcagcagagcacagcaagccgatgccgggggggcatctggctccgaggacccaccagattatggagatcaaggtaatgtgacacaggtgggatctgagcctatttcacctgagattggcccctttgaactctctgcggccagtgaggatgatcctcaatctgggccagtggaagagaatttagatgccgctgcaagagaggaagaggaacctcatgagcaggagcacaatggtggtgacgatcccttggatgtccatactcgccagcctagatttgtggatgtgaacccaacgcaggctccagtgatccaactagtccacgctgtctatgattccatgttgcaatcggacctccggcccctaggcagtttattccttgagcaaaacctgaacatcgaagaatttatatggatgtgcatgaccgtgcgtcacagatgtcaggccatcagaaaaaaaccattaccaattgttaagcagaggcgttggaagctcctgtcatcttgcagatcctggcgtatgggttaccgcacgcataacctcaaagtaaacagttttgagtcagggggggacaatgtccacccggtccttgtgactgctacgctaggatgtgatgagggcacgcggcatgcaacaacgtacagtgctggcattgtacagataccacgaatatcagaccaaaaccaaaagatcgaaacagccttcctgatggcacgtcgtgctaggtcactttcggcagaaagatatactttgttctttgatttagtatcctccggaaacaccctgtatgctatatggattgggctgggcacgaaaaaccgagtttcatttattgagtttgtaggatggttatgcaagaaggaccacactcatatacgcgaatggttccgccagtgcaccgggagacccaaagcagccaagccatggttaagagcgcatcctgtcgccattccttatgatgatccgttaacaaacgaggagattgatctggcctatgcccgcgggcaggccatgaatattgaggctcctagactgccagatgatcctataattgttgaggatgacgacgaaagtgaggaaattgaagctgaaagcgacgaggaggaagacaagagtggaatggaatctcttaaaaatataccgcaaacactgccgtacaatccaacagtatacggcaggcccgcggtgtttgaccgaaagtcagatgcaaaatcaaccaaaaaatgcagggccatagtaactgactttagtgtaatcaaggccattgaagaggaacacagaaagaagaaggcagccagaacagagcagccaagagccacgcctgaatcccaggcccccacagtggtcctccagcgaccacccacgcagcaagagcctggccccgtcggcccactgagtgtccaggctcggctggagccatggcaacctttgcctgggccccaagtgacagcagttctacttcacgaagaatccatgcagggtgtccaagtacatggttcgatgctagaccttcttgaaaaagacgatgaagtcatggagcagagggttatggcaaccctactgccaccagtaccacaacagccccgggctggcagaagaggcccttgtgtcttcaccggtgacctaggcatagagagtgatgagcccgcttcgacagagccggttcatgatcagctactgcctgccccaggacctgaccctcttgaaatccaaccactaacgtcccccaccacgtctcaacttagcagttcggcaccaagctgcgcacaaactccatggccggtggttcagccaagtcagactccagatgacccaacgaaacagtcccggccaccggaaacagctgccccacgccagtggccaatgcccctgcgacctatccctatgcgccccttgcggatgcagccaatcccatttaatcatccagtgggacccactccccatcagacacctcaagtggaaataacaccatataagcccacttgggctcagatagggcacattccatatcagcctacaccaacgggtcctgctaccatgctgttgcgccagtgggcacccgccaccatgcagacaccaccgagagcgcccactcccatgtcaccacctgaggtgccacccgttccccggcagaggcctcggggggcgcccactcccacgccacctcctcaggtgccgcccgttccccggcagaggcctcggggggcgcccactcccacgccacctcctcaggtgctgcccactcccatgcagctggcactaagggctcctgctggtcagcaggggccgacaaagcaaattttgcgccaattgttaacggggggcgtcaagaaagggagaccatcacttaagttacaggccgcccttgagcgtcaagccgctgcgggctggcagccttcaccagggtccggcaccagtgacaagattgtgcaggcgcctattttctacccacccgttttgcagcccatacaggttatggggcaagggggttccccaacggccatggccgcctcagcggtgacacaggcacccacggaatataccagggaaaggaggggagtggggcctatgcctcccaccgatattccgccgtctaaacgagcgaagatcgaggcctatacagagcccgagatgccgcacgggggggcctcgcactctcccgtcgttatcttggagaatgtcggccaggggcaacagcagactctggagtgcggaggaactgctaaacaggaaagggacatgttggggctgggggacattgcagtttcttccccttcctcttctgaaacatcgaacgatgagtga

SEQ ID NO: 176 - protein, 812 aa, Homo sapiens
MYIMYAMAIRQAIRDRRRNPASRRDQAKWRLQTLAAGWPMGYQAYSSWMYSYTDHQTTPTFVHLQATLGCTGGRRCHVTFSAGTFKLPRCTPGDRQWLYVQSSVGNIVQSCNPRYSIFFDYMAIHRSLTKIWEEVLTPDQRVSFMEFLGFLQRTDLSYIKSFVSDALGTTSIQTPWIDDNPSTETAQAWNAGFLRGRAYGIDLLRTEGEHVEGATGETREESEDTESDGDDEDLPCIVSRGGPKVKRPPIFIRRLHRLLLMRAGKRTEQGKEVLEKARGSTYGTPRPPVPKPRPEVPQSDETATSHGSAQVPEPPTIHLAAQGMAYPLHEQHGMAPCPVAQAPPTPLPPVSPGDQLPGVFSDGRVACAPVPAPAGPIVRPWEPSLTQAAGQAFAPVRPQHMPVEPVPVPTVALERPVYPKPVRPAPPKIAMQGPGETSGIRRARERWRPAPWTPNPPRSPSQMSVRDRLARLRAEAQVKQASVEVQPPQLTQVSPQQPMEGPLVPEQQMFPGAPFSQVADVVRAPGVPAMQPQYFDLPLIQPISQGAPVAPLRASMGPVPPVPATQPQYFDIPLTEPINQGASAAHFLPQQPMEGPLVPEQWMFPGAALSQSVRPGVAQSQYFDLPLTQPINHGAPAAHFLHQPPMEGPWVPEQWMFQGAPPSQGTDVVQHQLDALGYTLHGLNHPGVPVSPAVNQYHLSQAAFGLPIDEDESGEGSDTSEPCEALDLSIHGRPCPQAPEWPVQEEGGQDATEVLDLSIHGRPRPRTPEWPVQGEGGQNVTGPETRRVVVSAVVHMCQDDEFPDLQDPPDEA

SEQ ID NO: 177 - DNA, 1983 bp, Homo sapiens
atggggaccccctgccagtcggcgcggggtcccaggaccacgcccctccctcactgcccccccccctgccttccaggtgcaccggaccagcagacccggcggctacccccagggtggggccagcggaccgcacccacccaggtcggactagcggatgcagcatccccggatgagctccaggaccaggcaagcggggcacgccctgggggagggaaccgggtgggagcagggagggggcgtcccgggaccccagcccccagccggcaatccaggcgtaccgggccagcagagcaagcggaccacgcccattcgaaccctaccggcggatgcagcgacccccagaggagccccagaaccaggcaagccgggtacgccctgggggagggatcggcggggctggggtccaggggaccacgcccccaccctgcattccaggtgcagtggagcgccaggaaccccggctgccccaggacctggcggcggcgcagcggtgcccagcggggccaccccccacccggagcggggcagcggcccagcggacccaccggcggccgcccggctgcccccggagcgccaggaaccccggctgccccaggacctggcggcggcgcagcggtgcccagcggggccaccccccacccggagcggggcagcggcccagcggacccaccggcggccgcccggctgcccccggagcgccaggaaccccggctgccccaggacctggcggcggcgcagcggtgcccagcggggccaccccccacccggagcggggcagcggcccagcggacccaccggcggccgcccggctgcccccggagcgccaggaaccccggctgccccaggacctggcggcggcgcagcggtgcccagcggggccaccccccacccggagcggggcagcggcccagcggacccaccggcggccgcccggctgcccccggagcgccaggaaccccggctgccccaggacctggcggcggcgcagcggtgcccagcggggccaccccccacccggagcggggcagcggcccagcggacccaccggcggccgcccggctgcccccggagcgccaggaaccccggctgccccaggacctggcggcggcgcagcggtgcccagcggggccaccccccacccggagcggggcagcggcccagcggacccaccggcggccgcccggctgcccccggagcgccaggaaccccggctgccccaggacctggcggcggcgcagcggtgcccagcggggccaccccccacccggagcggggcagcggcccagcggacccaccggcggccgcccggctgcccccggagcgccaggaaccccggctgccccaggacctggcggcggcgcagcggtgcccagcggggccaccccccacccggagcggggcagcggcccagcggacccaccggcggccgcccggctgcccccggagcgccaggaaccccggctgccccaggacctggcggcggcgcagcggtgcccagcggggccaccccccacccggagcggggcagcggcccagcggacccaccggcggccgcccggctgcccccggagcgccaggaaccccggctgccccaggacctggcggcggcgcagcggtgcccagcggggccaccccccacccggagcggggcagcggcccagcggacccaccggcggccgcccggctgcccccggagcgccaggaaccccggctgccccaggacctggcggcggcgcagcggtgcccagcggggccaccccccacccggagcggggcagcggcccagcggacccaccggcggccgcccggctgcccccggagcgccaggaaccccggctgccccaggacctggcggcggcgcagcggtgcccagcggggccaccccccacccggagcggggcagcggcccagcggacccaccggcggccgcccggctgcccccggagcgccaggaaccccggctgccccaggacctggcggtag

SEQ ID NO: 178 - protein, 660 aa, Homo sapiens
MGTPCQSARGPRTTPLPHCPPPCLPGAPDQQTRRLPPGWGQRTAPTQVGLADAASPDELQDQASGARPGGGNRVGAGRGRPGTPAPSRQSRRTGPAEQADHAHSNPTGGCSDPQRSPRTRQAGYALGEGSAGLGSRGPRPHPAFQVQWSARNPGCPRTWRRRSGAQRGHPPPGAGQRPSGPTGGRPAAPGAPGTPAAPGPGGGAAVPSGATPHPERGSGPADPPAAARLPPERQEPRLPQDLAAAQRCPAGPPPTRSGAAAQRTHRRPPGCPRSARNPGCPRTWRRRSGAQRGHPPPGAGQRPSGPTGGRPAAPGAPGTPAAPGPGGGAAVPSGATPHPERGSGPADPPAAARLPPERQEPRLPQDLAAAQRCPAGPPPTRSGAAAQRTHRRPPGCPRSARNPGCPRTWRRRSGAQRGHPPPGAGQRPSGPTGGRPAAPGAPGTPAAPGPGGGAAVPSGATPHPERGSGPADPPAAARLPPERQEPRLPQDLAAAQRCPAGPPPTRSGAAAQRTHRRPPGCPRSARNPGCPRTWRRRSGAQRGHPPPGAGQRPSGPTGGRPAAPGAPGTPAAPGPGGGAAVPSGATPHPERGSGPADPPAAARLPPERQEPRLPQDLAAAQRCPAGPPPTRSGAAAQRTHRRPPGCPRSARNPGCPRTWR

SEQ ID NO: 179 - DNA, 1521 bp, Homo sapiens
atgggagaccgaagtgaaggccctggaccaacccggcccgggccccccggtatcgggccagagggtcccctcggacagctcctaagaaggcaccggtcgcccagtcctaccagagggggccaagaacccagacgagtccgtagaagggtcctcgtccagcaagaagaggaggtggtaagcggttcaccttcagggcctaggggagaccgaagtgaaggccctggaccaacccggcccgggccccccggtatcgggccagagggtcccctcggacagctcctaagaaggcaccggtcgcccagtcctaccagagggggccaagaacccagacgagtccgtagaagggtcctcgtccagcaagaagaggaggtggtaagcggttcaccttcagggcctaggggagaccgaagtgaaggccctggaccaacccggcccgggccccccggtatcgggccagagggtcccctcggacagctcctaagaaggcaccggtcgcccagtcctaccagagggggccaagaacccagacgagtccgtagaagggtcctcgtccagcaagaagaggaggtggtaagcggttcaccttcagggcctaggggagaccgaagtgaaggccctggaccaacccggcccgggccccccggtatcgggccagagggtcccctcggacagctcctaagaaggcaccggtcgcccagtcctaccagagggggccaagaacccagacgagtccgtagaagggtcctcgtccagcaagaagaggaggtggtaagcggttcaccttcagggcctaggggagaccgaagtgaaggccctggaccaacccggcccgggccccccggtatcgggccagagggtcccctcggacagctcctaagaaggcaccggtcgcccagtcctaccagagggggccaagaacccagacgagtccgtagaagggtcctcgtccagcaagaagaggaggtggtaagcggttcaccttcagggcctaggggagaccgaagtgaaggccctggaccaacccggcccgggccccccggtatcgggccagagggtcccctcggacagctcctaagaaggcaccggtcgcccagtcctaccagagggggccaagaacccagacgagtccgtagaagggtcctcgtccagcaagaagaggaggtggtaagcggttcaccttcagggcctaggggagaccgaagtgaaggccctggaccaacccggcccgggccccccggtatcgggccagagggtcccctcggacagctcctaagaaggcaccggtcgcccagtcctaccagagggggccaagaacccagacgagtccgtagaagggtcctcgtccagcaagaagaggaggtggtaagcggttcaccttcagggccactacggccacgtccccggcctcccgctcggtctcttagagagtggctgctacgcattagagaccactttgagccacccacagtaaccacccagcgccaatctgtctacatagaagaagaagaggatgaagactaa

SEQ ID NO: 180 - protein, 506 aa, Homo sapiens
MGDRSEGPGPTRPGPPGIGPEGPLGQLLRRHRSPSPTRGGQEPRRVRRRVLVQQEEEVVSGSPSGPRGDRSEGPGPTRPGPPGIGPEGPLGQLLRRHRSPSPTRGGQEPRRVRRRVLVQQEEEVVSGSPSGPRGDRSEGPGPTRPGPPGIGPEGPLGQLLRRHRSPSPTRGGQEPRRVRRRVLVQQEEEVVSGSPSGPRGDRSEGPGPTRPGPPGIGPEGPLGQLLRRHRSPSPTRGGQEPRRVRRRVLVQQEEEVVSGSPSGPRGDRSEGPGPTRPGPPGIGPEGPLGQLLRRHRSPSPTRGGQEPRRVRRRVLVQQEEEVVSGSPSGPRGDRSEGPGPTRPGPPGIGPEGPLGQLLRRHRSPSPTRGGQEPRRVRRRVLVQQEEEVVSGSPSGPRGDRSEGPGPTRPGPPGIGPEGPLGQLLRRHRSPSPTRGGQEPRRVRRRVLVQQEEEVVSGSPSGPLRPRPRPPARSLREWLLRIRDHFEPPTVTTQRQSVYIEEEEDED

SEQ ID NO: 181 - DNA, 772 bp, Homo sapiens
mtggcgacttggtgccaccatctggcagcttttggccttcttcctagccttcttcctagacctcatcctgctcattattgctctctatctacaacaaaactggtggactctattggttgatctcctttggctcctcctgtttctggcgattttaatctggatgtattaccatggacaacgacacagtgatgaacaccaccacgatgactccctcccgcaccctcaacaagctaccgatgattctggccatgaatctgactctaactccaacgagggcagacaccacctgctcgtgagtggagccggcgacggacccccactctgctctcaaaacctaggcgcacctggaggtggtcctgacaatggcccacaggaccctgacaacactgatgacaatggcccacaggaccctgacaacactgatgacaatggcccacatgacccgctgcctcaggaccctgacaacactgatgacaatggcccacaggaccctgacaacactgatgacaatggcccacatgacccgctgcctcatagccctagcgactctgctggaaatgatggaggccctccacaattgacggaagaggttgaaaacaaaggaggtgaccagggcccgcctttgatgacagacggaggcggcggtcatagtcatgattccggccatggcggcggtgatccacaccttcctacgctgcttttgggttcttctggttccggtggagatgatgacgacccccacggcccagttcagctaagctactatgactaa

SEQ ID NO: 182 - protein, 258 aa, Homo sapiens
MLWRLGATIWQLLAFFLAFFLDLILLIIALYLQQNWWTLLVDLLWLLLFLAILIWMYYHGQRHSDEHHHDDSLPHPQQATDDSGHESDSNSNEGRHHLLVSGAGDGPPLCSQNLGAPGGGPDNGPQDPDNTDDNGPQDPDNTDDNGPHDPLPQDPDNTDDNGPQDPDNTDDNGPHDPLPHSPSDSAGNDGGPPQLTEEVENKGGDQGPPLMTDGGGGHSHDSGHGGGDPHLPTLLLGSSGSGGDDDDPHGPVQLSYYD

(19) United States
(12) Patent Application Publication
(10) Pub. No.: US 2007/0196389 A1
(43) Pub. Date: Aug. 23, 2007

(54) VIRAL GENE PRODUCTS AND METHODS FOR VACCINATION TO PREVENT VIRAL ASSOCIATED DISEASES

(75) Inventors: Michael A. Caligiuri, Columbus, OH (US); Robert A. Baiocchi, Dublin, OH (US)

Correspondence Address: CALFEE HALTER & GRISWOLD, LLP, 800 SUPERIOR AVENUE, SUITE 1400, CLEVELAND, OH, 44114, US

(73) Assignee: The Ohio State University Research Foundation, Columbus, OH (US)

(21) Appl. No.: 11/561,363

(22) Filed: Nov. 17, 2006

Related U.S. Application Data
(60) Provisional application No. 60/737,944, filed on Nov. 18, 2005.

(51) Int. Cl. A61K 39/25; A61K 48/00; C12N 15/86; C12N 7/00; A61K 39/245

(52) U.S. Cl. 424/229.1; 514/44; 424/230.1; 435/5; 435/456; 435/235.1; 435/325

(57) ABSTRACT

Methods of vaccination to prevent virus-associated diseases, which methods generally result in an increase of virus-specific memory T cells that provide or restore host immunity and result in control of the viral-associated disease process. Polypeptides and DNA sequences for achieving these results are also described. In some embodiments, the virus is Epstein-Barr virus.

### CROSS REFERENCE TO RELATED APPLICATIONS

This application claims priority to and any other benefit of U.S. Provisional Application Ser. No. 60/737,944, filed on Nov. 18, 2005, the entire content of which is incorporated by reference herein

### GOVERNMENT RIGHTS

The government of the United States may have certain rights in this invention.

### FIELD

This disclosure generally relates to methods of vaccination to prevent viral-associated diseases, and in some embodiments, Epstein-Barr virus (EBV)-associated diseases. In some embodiments, the methods result in an increase of EBV-specific memory T cells that improve and/or restore host immunity and result in control of the disease. Polypeptides and DNA sequences for achieving these results are also described.

### BACKGROUND

The Epstein-Barr virus is a ubiquitous lymphotrophic human herpes virus that infects resting human memory B cells and epithelial cells. EBV gains access to the human host via primary infection of epithelial cells of the nasopharynx, and it usually does this during adolescence. This first infection of the host is termed primary EBV infection.

Primary EBV infection of healthy individuals often occurs without symptoms, however, occasionally it can result in a severe flu-like illness where the infected EBV(+) B cells in a person (the host) proliferate for a limited time, after which the host's own immune system (largely via healthy antigen-specific T cells) brings the disease under control. This self-limited B cell lymphoproliferative disease is known as infectious mononucleosis (IM) or “mono,” and can be distinguished from the more serious, uncontrollable or malignant proliferation of EBV-infected B cells that can occur following the onset of iatrogenic (immune suppressive medication), acquired (AIDS) or congenital (SCID, XLP) immune suppression.

### SUMMARY

Provided herein are methods for inducing an immune response against at least one virus-associated disease in a subject, comprising: administering to the subject at least one virus gene product. In some embodiments, the gene product is chosen from virus lytic gene products and virus latent gene products. In some embodiments, the virus is a human herpes virus chosen from HHV-1 (Herpes Simplex Virus 1), HHV-2 (Herpes Simplex Virus 2), HHV-3 (Varicella Zoster Virus), HHV-4 (Epstein-Barr virus), HHV-5 (Cytomegalovirus), HHV-6, HHV-7, and HHV-8 (Kaposi Sarcoma herpes virus: KSHV). Epstein-Barr viruses include, but are not limited to, the following strains: Type 1, Type 2, SiIIA, A4, TSB-B6, ap876, p3hr1, b95.8, cao, raji, and daudi.

Additional features and advantages will be set forth in part in the description that follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate some embodiments of the invention, and together with the description, serve to explain principles of the invention.

FIG. 1 shows EBV gene expression profiles and associated pathology. EBV infects nasopharyngeal epithelial cells and resting B lymphocytes via CD21. Primary infection (and infectious mononucleosis) involves both lytic (virion production) and latent gene programs. Antigen-specific T cells control primary (and recurrent) infection resulting in suppression of viral replication (lytic cycle) and proliferating lymphoblasts (Lat III) and establishment of resting latent state (Lat I). Patients with immune suppression (IS, organ transplant, AIDS, congenital IS) are at risk for reactivation of Lat I infection and transition to the actively replicating (lytic) or actively proliferating EBV-transformed lymphoblast with Lat III gene expression profile. The key Lat III genes are LMP-1 and EBNA2 which are capable of driving constitutive cellular bcl2 expression, activation of NFkB and autocrine survival and growth pathways, respectively.

FIG. 2 shows a Southern blot analysis of 10 EBV tumors from 10 hu-PBL-SCID mice, demonstrating Ig gene rearrangements. Lanes 1, 2 & 4 are polyclonal; Lanes 3, 5, 6, 7 & 10 are oligoclonal; Lanes 8 & 9 appear monoclonal. In Southern blots probed with a terminal repeat segment of the EBV genome, monoclonal tumors possess one copy of the EBV genome (latent, episomal) while polyclonal and oligoclonal tumors have latent episomes and multiple linear (replicative).

FIG. 3 shows combination therapy with GM-CSF+IL,-2 but not IL-2 (or GM-CSF) alone induce a robust human CD8+ T cell expansion (A) to both lytic (BZLF or RAK in B) and latent (EBNA3A not shown) EBV antigens. The response to IL-2 alone (C, D) is not significant.

FIG. 4 shows quantification of EBV-specific CTL in two HLA-B8+ patients with HLA-tetramers loaded with RAK peptide derived from the EBV lytic gene product, BZLF 1. Serial PBMC samples from two HLA-B8+ patients were analyzed by flow cytometry with APC-conjugated MHC/peptide tetramers. Representative results obtained at three time points with the HLA-B8 tetramer containing the RAKFKQLL peptide (HLA-B8/RAK) from the EBV immediate early gene BZLF-1 are shown. CD3+ events occurring in a lymphocyte gate are shown in blue. The percentage of CD8+HLA-B8/RAK+events is indicated in the upper right quadrant of each plot.

FIG. 5 shows In situ (IS) RT-PCR analysis of vTK expression in a representative PTLD tumor sample. (A) H & E stain of tumor biopsy, showing diffuse infiltration by atypical large lymphocytes. (B) IS-RT-PCR detection of EBER-1 and EBER-2 mRNA. A majority of the lymphoma cells in the field are positive for the expression of these abundant EBV transcripts, confirming the presence of EBV. (C) IS-RT-PCR analysis of the lytic EBV gene product, viral thymidine kinase (TK) mRNA (BXLF1 ORF). Viral TK expression is present in a number of lymphoma cells equivalent to those expressing EBER-1 and EBER-2. (D) RNase digestion after IS-RT-PCR analysis of vTK mRNA demonstrates that the signal present in Panel C is RNA-based. From [Porcu, 2002 #394]

FIG. 6 shows the genetic composition of the wild type AAV2 and 5 rAAV transgene vaccine constructs. All transgene constructs contain a full-length EBV-gene product cDNA (as above) constitutively driven by a standard CMV promoter, contain an internal stop codon, followed by poly A tail (pA). Other elements present within the rAAV-transgene constructs included neo resistant gene, AAV rep and AAV cap genes, each driven by an internal promoter. Additional control vectors encode β-galactosidase and green fluorescent protein (GFP).

FIG. 7 shows rAAV2 transgene replication and expression. A. Southern Blot; B. Anti-BZLF western blot. Lane 1 Hela cell lysate; Lane 2 Hela-rAAV-BZLF1 lysate.

FIG. 8 shows SYPRO orange staining of purified rAAV2 vectors.

FIG. 9 shows transduction of 293T cells with rAAV2/BZLF1. Top: Quantification of BZLF1 expression by immune fluorescence; Bottom: quantification of BZLF1 expression by western blot. DRPs=DNAse resistant particles per cell (see text).

FIG. 10 shows a Southern blot demonstrating rAAV2/EBV latent antigen replication.

FIG. 11 shows in vitro expansion of EBV-specific CD8+ T lymphocytes.

FIG. 12 shows flow cytometric analyses of the in vitro expanded EBV-specific CD8+ CTL following 14 day culture with rAAV2/BZLF1-infected human autologous APC. Y axis indicates cntl (top) or BZLF (RAK, bottom) tetramers and X axis shows CD8+ T cells.

FIG. 13 shows that full length BZLF1 polypeptide is capable of inducing a T cell response independent of HLA type or known/defined immunodominant peptides derived from BZLF1. (A) HLAB8 donor (147) peripheral blood mononuclear cells (PBMC) responding to autologous dendritic cell/antigen presenting cells (APC) pulsed with either control protein (BSA) or the BZLF1 protein. After 7 days, expansion of antigen specific T cells that recognize a single defined immunodominant peptide RAK is observed using the HLAB8 tetramer loaded with RAK peptide as a biomarker. Left panel shows a mismatched tetramer (HLAB8-FLR) as a control. Middle panel shows tetramer staining background of PBMCs stimulated with a control BSA protein. Right panel shows HLAB8-RAK tetramer specific CD8+ T cells that have expanded in vitro. (B) PBMC from HLAB8 donor 147 plated in presence of autologous dendritic cell APC pulsed with full length BZLF or control protein. After 12 days in culture, approximately 4-fold increase in amount of IFNgamma (IFNγ) signal in response to full length BZLF1 protein (compared to BSA control) as measured by IC flow was observed. There is presently one single defined immunodominant peptide (RAK) derived from BZLF for HLAB8 donors. (C) PBMC from an HLAA2 individual plated in presence of autologous DC APC pulsed with full length BZLF or BSA control protein. After 12 days in culture, we see approximately 2-fold increase in amount of IFNγ signal produced by CD8+ T cells that have expanded (flow cytometry riot shown) in response to full length BZLF1 protein (compared to BSA control) as measured by IC flow. While there is presently no described immunodominant peptide derived from BZLF for HLAA2 donors, the HLAA2 APC are capable of processing full length BZLF1 polypeptide and presenting immunogenic peptides that drive a cytokine response indicating antigen recognition that drives cellular T cell activation with effector function.

### DETAILED DESCRIPTION

The present invention will now be described by reference to some more detailed embodiments, with occasional reference to the accompanying drawings. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are incorporated by reference in their entirety.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

EBV is a ubiquitous lymphotrophic human herpes virus that infects resting human memory B cells and epithelial cells. There are two EBV strains, type A and type B, also referred to as type 1 and type 2 respectively, that are distinguished by genetic polymorphisms, whereby various genes differ in DNA sequence and/or primary amino acid sequence. Both EBV types occur worldwide, with different geographic distributions. Furthermore, both EBV types are similar in their biological activities in vivo, therefore the methods and compositions of the present invention can be adapted for use against both type A EBV, also known as type 1 EBV, and type B EBV, also known as type 2 EBV, by administering gene products from one or both types of EBV. Other strains of EBV that fall within the scope of the present invention are SiIIA, A4, TSB-B6, ap876, p3hr1, b95.8, cao, raji, and daudi. Table 1 shows nucleic acid sequences corresponding to the open reading frames of Type 1 EBV.

EBV gains access to the human host via primary infection of epithelial cells of the nasopharynx, and it usually does this during adolescence. This first infection of the adolescent is termed primary EBV infection. Within the epithelium, activation of a lytic gene program during primary EBV infection results in local virion production and infiltration of submucosal lymphoid tissue leading to infection of resting B lymphocytes. Lytic infection is initiated and driven by the EBV replicon activator gene product known as BZLF1. Approximately 80 different EBV mRNA species are expressed during the lytic phase of the primary infection and are characterized as either immediate early, delayed early or late viral lytic genes. Genes detected during immediate early lytic infection are expressed independent of new protein synthesis and are activated by the BZLF1 gene product. Examples of early lytic antigens (EA) include, but are not limited to, BRLF1, BMRF1, BMLF1 (trans activators), BALF5 (a DNA polymerase), BARF1 (a ribonucleotide reductase), BXLF1 (a viral thymidine kinase), BGLF4 (a protein kinase homologue to CMV-UL97). Late lytic gene expression encodes primarily structural proteins that are required for virion assembly. The other EBV gene program, the latent gene program, becomes activated in infected B lymphocytes during this early stage of primary EBV infection.

Primary EBV infection of healthy individuals often occurs without symptoms, however, occasionally it can result in a severe flu-like illness where the infected EBV(+) B cells in a subject (e.g. human) proliferate for a limited time, after which the subject's own immune system (largely via healthy antigen-specific T cells) brings the disease under control. This self limited B-cell lymphoproliferative disease is known as infectious mononucleosis (IM) or “mono”, and should be distinguished from the more serious, uncontrollable or malignant proliferation of EBV(+) B cells that can occur following an organ transplant, termed post-transplant lymphoproliferative disorder, which can often be fatal and will be discussed below. The inventors have found that in both of these conditions, it is clear that EBV lytic and latent gene products, expressed in the infected B cells, are the principle targets of the host's T cell immune response.

Regardless if a subject has a “silent” primary EBV infection or develops IM, the lytic phase is brought under control by the host's T cells, but the EBV is never entirely eliminated from the host's body. Rather, EBV manages to hide from the host's immune system by switching to a persistent EBV program in a limited number of resting B cells. Generally, although not always, as long as the virus only exists in the first latent form (latency type I), the lytic gene program is silenced and latent gene expression is limited to EBNA1 and LMP2A. Thus, the virus is able to persist in the human host by evading host immune surveillance networks and, because oncogenic viral proteins like LMP1 and EBNA2 are silenced, poses little threat to the infected, immune competent host. Indeed, approximately 95% of adults in the U.S.A. have a stable, latent EBV infection. However, as noted below, there are instances where a latent EBV infection can be reactivated and associated with a disease process.

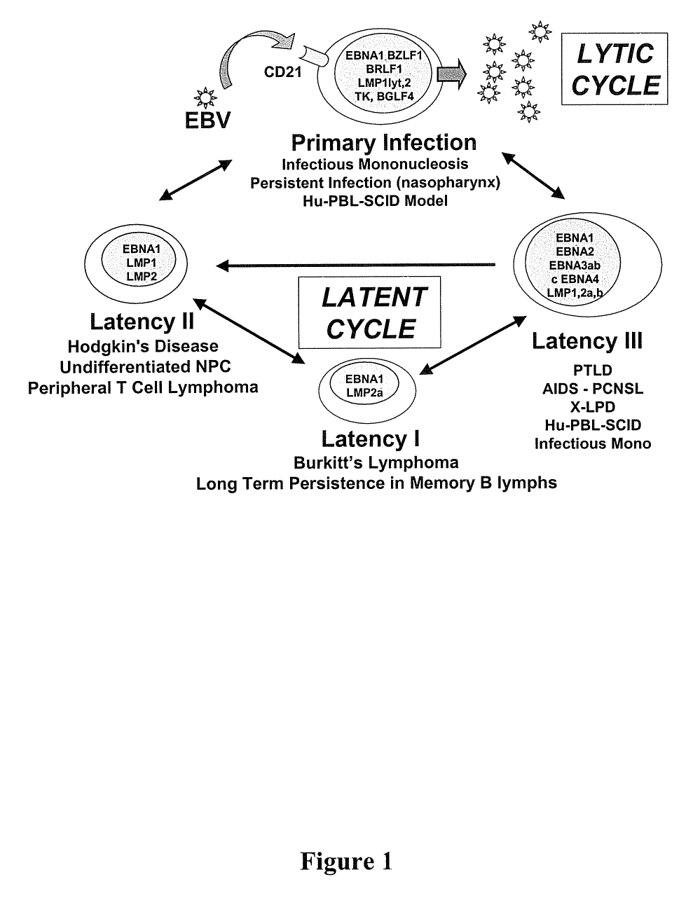

Three latent EBV gene programs exist (FIG. 1), each associated with a pattern of latent gene expression. EBV(+) resting B cells display a Latency I gene expression profile comprising, but not limited to, the Epstein-Barr nuclear antigen 1 (EBNA1) and latent membrane protein-2A (LMP2A). This is the latency program that is associated with long-term silent EBV infection of memory B cells in normal, healthy people. Latency II infected B lymphocytes show a latent gene program comprising, but not limited to, expression of EBNA1, LMP2A and LMP1. Human EBV(+) B cells that become activated and are capable of malignant transformation, i.e., proliferating indefinitely, display a Latency type III program comprising, but not limited to, expression of EBNA1, EBNA2, EBNA3A, EBNA3B, EBNA3C, EBNA-LP, LMP1, LMP2A and LMP2B.

Complications with primary EBV infection and reactivation of preexisting latent EBV infection occur with high frequency in patients with congenital, acquired and/or iatrogenic immune deficient states. The administration of intensive T-cell suppressive therapy in patients who have undergone, for example, solid organ or hematologic allograft transplantation places patients at risk for EBV-associated PTLD. This risk of PTLD is heightened with further evidence of graft rejection or graft-versus-host disease (GVHD). PTLD complicates between 2 and 20% of solid organ transplants performed annually in the United States. Reported mortality rates range from 50% to 70% and the optimal treatment approach remains highly controversial. Reduction of immune suppression is initially attempted in almost all patients and is reported to result in regression of PTLD lesions in 23% to 50% of cases, however, durable complete responses with this approach are considered rare. With the advent of novel immune suppressive regimens, solid organ transplantation is becoming the therapy of choice for end stage renal disease, type I diabetes in pediatric patients, and cardiac and hepatic failure for example. Given the increased use of organ transplantation in standard medical practice, PTLD will be encountered more frequently resulting in increased morbidity and mortality. Furthermore, management of complications related to PTLD present a major financial challenge for patients and the health care industry.

PTLD is a malignant B cell lymphoproliferative disorder that is associated with primary or reactivated EBV infection. The association between EBV viremia and PTLD has been reproducibly documented using quantitative polymerase chain reaction (Q-PCR) techniques to amplify EBV DNA from peripheral blood of transplant patients during immune suppressive therapy. The spectrum of PTLD ranges from polyclonal B-cell hyperplasia to monoclonal immunoblastic lymphoma. While polyclonal EBV-LPD is known to regress following withdrawal of immune suppressive therapy, monoclonal disease demonstrates intrinsic resistance to conventional therapy and is often fatal. In vitro data showing inhibition of EBV induced B-cell transformation in the presence of autologous T lymphocytes and the demonstration of cytotoxic T lymphocyte (CTL)-mediated reversal of EBV+ lymphoblastoid cell line (LCL) outgrowth in xenografted severe combined immunodeficiency (SCID) mice provide convincing support to the notion that T cells are critical in the control of EBV infection and EBV-transformed B cells. The delivery of in vitro-generated CTLs to patients with EBV-LPD and documentation of endogenous expansions of EBV-specific CTLs following withdrawal of immune suppression suggests that restoration of host immunity may be the most promising strategy in controlling this “opportunistic” malignancy. These observations also present a unique opportunity to devise strategies to educate host immune networks with the intent on expanding EBV-specific CTL precursor frequency prior to solid organ transplantation to prevent the onset of PTLD. Of course, similar strategies can also be applied to other patient groups at risk for reactivated EBV (or other herpes virus) infection(s) and malignant EBV (or other herpes virus)-associated disease as described above.

Due to the restricted association between different viral protein-derived peptides and specific HLA types, immunization with select peptides derived from either latent or lytic EBV gene products would provide immune dominant antigens for a relatively small group of patients. The inventors have discovered that this restriction can be circumvented by providing full-length EBV lytic and/or latent polypeptides or proteins to the antigen-processing networks, thereby allowing for optimal presentation in the context of most, if not all, types of HLA class I and II molecules. Subjects, e.g. humans, receiving these larger molecules are then able to process full-length polypeptides and present immune dominant peptides in the context of their specific HLA Class I and II molecules. Thus, provided herein are delivery of full length lytic and/or latent EBV polypeptide immunogens for the purposes of vaccination that can be accomplished via direct delivery of the purified protein with or without an excipient, with or without an immune adjuvant, or via recombinant DNA-based techniques. Consistent with this disclosure, inventive methods and compositions are provided.

The present invention is directed to methods and compositions for inducing an immune response in a subject against a viral-associated disease by administering to a subject gene products from that virus. Viruses that induce diseases, which are the target of this invention, include the human herpes viruses (HHVs) 1-8 (HHV-1, HHV-2, HHV-3, HHV-4, HHV-5, HHV-6, HHV-7, and HHV-8). Although much of the present disclosure exemplifies the invention with regard to HHV-4 (Epstein-Barr virus), it is equally applicable to the other HHVs.

In some embodiments, the virus is Epstein-Barr virus, and the at least one virus gene product is chosen from the gene products listed in Table 1. In some embodiments, the gene product is from a Type 1 Epstein-Barr virus, and is associated with the sequences listed in Table 1. In some embodiments, the Epstein-Barr virus lytic gene products are chosen from BZLF1, BHRF1, BHLF1, BALF2, BMLF1, BRLF1, BMRF1, BALF5, BARF1, BORF2, BCRF1, BKRF3, BDLF3, BILF1, BFRF1, BXLF1, BGLF4, BGLF5, gp350, gp220 and LMP1-lyt, and the Epstein-Barr virus latent gene products are chosen from EBNA1, EBNA2, EBNA3A, EBNA3B, EBNA3C, EBNA-LP, LMP1, LMP2A, and LMP2B, or, the lytic gene products are chosen from BZLF1 and BMLF1 and the latent gene products are chosen from EBNA1, EBNA3A, and EBNA3C, or, the Epstein-Barr virus lytic gene product is BZLF1 and the Epstein-Barr virus latent gene product is EBNA3C. Some embodiments of the inventive methods involve administering at least two Epstein-Barr virus gene products.

In some embodiments, the virus-associated disease is chosen from neoplastic disease, infectious mononucleosis, hemophagocytic syndrome, renal cell tubulitis, and hepatitis. Neoplastic diseases include, but are not limited to, lymphoproliferative disorder, Burkitt's lymphoma, Hodgkin's disease, non Hodgkin's lymphoma, epithelial carcinomas of gastric and nasopharyngeal mucosa, undifferentiated nasopharyngeal carcinomas, and peripheral T-cell and T/NK cell lymphomas. Lymphoproliferative disorders include, but are not limited to, those that arise as a consequence or in association with congenital immune deficiency, acquired immune deficiency, and iatrogenic immune deficiency, which generally involves immune-deficient states that arise as a consequence of or in association with a therapeutic intervention. Iatrogenic immune deficiencies include. Any of these immune deficient states can result in post-transplant lymphoproliferative disease.

In some embodiments, the subject has been diagnosed with at least one Epstein-Barr virus associated disease chosen from neoplastic disease, infectious mononucleosis, hemophagocytic syndrome, renal cell tubulitis, and hepatitis. Neoplastic diseases include, but are not limited to, lymphoproliferative disorder, Burkitt's lymphoma, Hodgkin's disease, epithelial carcinomas of gastric and nasopharyngeal mucosa, undifferentiated nasopharyngeal carcinomas, and peripheral T-cell lymphomas. Lymphoproliferative disorders include, but are not limited to, congenital immune deficiency, acquired immune deficiency, and iatrogenic immune deficiency. Iatrogenic immune deficiencies can lead to post-transplant lymphoproliferative disease.

In some embodiments, the administering is done by introducing at least one polynucleotide that encodes an Epstein-Barr virus gene product chosen from Epstein-Barr virus lytic gene products and Epstein-Barr virus latent gene products wherein the Epstein-Barr virus gene product is operably linked to a regulatory element. In some embodiments, the at least one polynucleotide that encodes an Epstein-Barr virus gene product comprises a full length Epstein-Barr virus cDNA coding sequence. Full length Epstein-Barr virus cDNA coding sequences include, but are not limited to, BZLF1, BHRF1, BHLF1, BALF2, BMLF1, BRLF1, BMRF1, BALF5, BARF1, BORF2, BCRF1, BKRF3, BDLF3, BILF1, BFRF1, BXLF1, BGLF4, BGLF5, gp350, gp220, and LMP1-lyt, EBNA1, EBNA2, EBNA3A, EBNA3B, EBNA3C, EBNA-LP, LMP 1, LMP2A, and LMP2B coding sequences.

In some embodiments, the administering is performed by introducing in a subject an immunogenic composition comprising at least one Epstein-Barr virus gene product chosen from Epstein-Barr virus lytic gene products and Epstein-Barr virus latent gene products. Gene products can be polynucleotides or polypeptides. In some embodiments, the at least one Epstein-Barr virus lytic gene product and the at least one Epstein-Barr virus latent gene products are linked. In some embodiments, the virus gene products are administered by a route chosen from subcutaneous, intramuscular, mucosal, intraperitoneal, or intradermal routes.

Also provided are pharmaceutical compositions for inducing an immune response in a subject against at least one virus-associated disease comprising: at least two virus gene products; and at least one pharmaceutically acceptable excipient. In some embodiments, the gene products are chosen from virus lytic gene products and virus latent gene products. In some embodiments, the virus is a human herpes virus chosen from HHV-1 (Herpes Simplex Virus 1), HHV-2 (Herpes Simplex Virus 2), HHV-3 (Varicella Zoster Virus), HHV-4 (Epstein-Barr virus), HHV-5 (Cytomegalovirus), HHV-6, HHV-7, and HHV-8. In some such embodiments, the virus is Epstein-Barr virus, which may be a strain chosen from Type 1, Type 2, SiIIA, A4, TSB-B6, ap876, p3hr1, b95.8, cao, raji, and daudi.

In some embodiments, the at least one virus gene product is chosen from the gene products listed in Table 1. In some such embodiments, the Epstein-Barr virus lytic gene products are chosen from BZLF1, BHRF1, BHLF1, BALF2, BMLF1, BRLF1, BMRF1, BALF5, BARF1, BORF2, BCRF1, BKRF3, BDLF3, BILF1, BFRF1, BXLF1, BGLF4, BGLF5, gp350, gp220 and LMP1-lyt, and the Epstein-Barr virus latent gene products are chosen from EBNA1, EBNA2, EBNA3A, EBNA3B, EBNA3C, EBNA-LP, LMP1, LMP2A, and LMP2B. In some such embodiments, the lytic gene products are chosen from BZLF1 and BMLF1 and the latent gene products are chosen from EBNA1, EBNA3A, and EBNA3C, or, the Epstein-Barr virus lytic gene product is BZLF1 and the Epstein-Barr virus latent gene product is EBNA3C.

The pharmaceutical composition may comprise excipients chosen from water, salts, buffers, carbohydrates, solubilizing agents, protease inhibitors, and dry powder formulating agents. In some embodiments, the pharmaceutical compositions further comprise at least one adjuvant, such as Freunds adjuvant, a water/oil emulsion, mineral oil, granulocyte/macrophage-colony stimulating factor, and/or interleukin-2. In some embodiments, the pharmaceutical composition comprises at least two Epstein-Barr virus gene products, and in some embodiments, the pharmaceutical composition comprises at least three Epstein-Barr virus gene products.

Also provided are viral vectors, such as adenoviral vectors, comprising: an Epstein-Barr virus gene product expression cassette comprising: a polynucleotide encoding at least one Epstein-Barr virus gene product; and a heterologous promoter operatively linked to the polynucleotide encoding an Epstein-Barr virus gene product. In some embodiments, the Epstein-Barr virus may be a strain chosen from Type 1, Type 2, SiIIA, A4, TSB-B6, ap876, p3hr1, b95.8, cao, raji, and daudi. In some embodiments, the at least one Epstein Barr gene product is chosen from the gene products listed in Table 1. Epstein-Barr virus lytic gene products include, but are not limited to, BZLF1, BHRF1, BHLF1, BALF2, BMLF1, BRLF1, BMRF1, BALF5, BARF1, BORF2, BCRF1, BKRF3, BDLF3, BILF1, BFRF1, BXLF1, BGLF4, BGLF5, gp350, gp220 and LMP1-lyt, and the Epstein-Barr virus latent gene products are chosen from EBNA1, EBNA2, EBNA3A, EBNA3B, EBNA3C, EBNA-LP, LMP1, LMP2A, and LMP2B. In some such embodiments, the lytic gene products are chosen from BZLF1 and BMLF1 and the latent gene products are chosen from EBNA1, EBNA3A, and EBNA3C, and in some such embodiments, the Epstein-Barr virus lytic gene product is BZLF1 and the Epstein-Barr virus latent gene product is EBNA3C.

Also provided are recombinant adeno-associated viruses comprising adenoviral vectors. Also provided are plasmids comprising: an adenoviral portion comprising an adenoviral vector; and a plasmid portion.

Also provided are mammalian cells comprising a viral vector as provided herein. Also provided are methods of culturing the mammalian cell, under conditions to produce an immune response to a virus gene product, the method comprising: growing cells under conditions favorable to the expression of a virus gene product. These cellular preparations include but are not limited to autologous dendritic cells, fibroblasts, myocytes, etc. Also provided are methods allowing for expression of a given viral gene product (from any existing EBV open reading frame, for example) that leads to ex vivo generation and expansion of EBV-specific cytotoxic T cell (or other effector cells) for use as a cellular therapy. The nature of the method described here may be applied to generate autologous or allogeneic antigen-specific or innate immune cell preparations for use as a cellular therapy. Cellular therapy may be defined as a cellular preparations that when administered to a subject, enhance a given response to a pathogen via (but not limited to) secretion of cytokines, recognition of co-stimulatory molecules, recognition of antigen, delivery of cytotoxic therapy, etc.

Also provided are vectors, further comprising at least one pharmaceutically acceptable excipient. Also provided are viral vector pharmaceutical compositions for producing an immune response against Epstein-Barr virus-associated neoplastic or non-neoplastic disease comprising: an Epstein-Barr virus gene product expression cassette comprising: a polynucleotide encoding at least one Epstein-Barr virus gene product; and a heterologous promoter operatively linked to the polynucleotide encoding said Epstein-Barr virus gene product. Also provided are adenoviral vector pharmaceutical compositions for producing an immune response against Epstein-Barr virus-associated disease comprising: an Epstein-Barr virus gene product expression cassette comprising: a polynucleotide encoding at least one Epstein-Barr virus gene product; and a heterologous promoter operatively linked to the polynucleotide encoding said Epstein-Barr virus gene product.

Also provided are methods for ascertaining a subject's response to an Epstein-Barr virus vaccine comprising: (i) assaying for the presence of at least one Epstein-Barr virus gene product chosen from Epstein-Barr virus lytic gene products and Epstein-Barr virus latent gene products (ii) assaying for the presence of immune effector cell subsets isolated from a subject using cytokines as biomarkers; (iii) assaying for the presence of antigen specific lymphocytes utilizing monoclonal antibody and/or HLA tetramer technology as biomarkers.

Thus, in one embodiment, the invention is directed to treating at least one EBV-associated disease in a subject, through the administration of a novel vaccine comprising at least one EBV gene product, which may be chosen from EBV lytic gene products and/or EBV latent gene products. Generally, EBV-associated diseases are those which result in the uncontrolled proliferation, survival, or death of human cells as a direct/indirect result of EBV-gene products expressed/encoded by any EBV ORF. EBV-associated pathology would include any condition in which EBV genome (DNA) can be detected in tissue or bodily fluids. The EBV-associated disease entity may be related to disregulation of any physiologic process as a result of expression of one or more viral gene products encoded by any open reading frame of EBV or non coding sequences resulting in polynucleotide sequences that can participate in viral or cellular regulatory mechanisms (i.e. but not limited to miRNA). The strategies outlined herein may also be applied to similar disease states that are associated or caused by other viruses with related or homologous gene products or open reading frames. These viruses include but are not limited to human herpesvirus 1 and 2 (HHV-1,-2), HHV-3 (varicella zoster), HHV-4 (EBV, as exemplified herein), HHV-5 (cytomegalovirus, or “CMV”), HHV-6, HHV-7, and HHV-8 (KSHV).

Specific EBV-associated diseases include, but are not limited to, neoplastic diseases, infectious mononucleosis (IM), hemophagocytic syndrome, renal cell tubulitis, and hepatitis. Neoplastic diseases include, but are not limited to, lymphoproliferative disorders, Burkitt's lymphoma, Hodgkin's disease, B cell non Hodgkin's lymphomas, epithelial carcinomas of gastric and nasopharyngeal mucosa, undifferentiated nasopharyngeal carcinomas, and peripheral T cell and T/NK cell lymphomas. In some embodiments, the lymphoproliferative disorder is chosen from congenital immune deficiency, acquired immune deficiency, and iatrogenic immune deficiency. Neoplasms that arise in the context of iatrogenic immune deficiencies include, but are not limited to, post-transplant lymphoproliferative disease (PTLD) and methotrexate-associated non-Hodgkin's lymphoma. EBV-associated diseases display a lytic and/or latent gene program, thus the compositions of the present invention comprising at least one EBV lytic gene product and/or at least one EBV latent gene product provide an effective means of prevention and/or treatment.

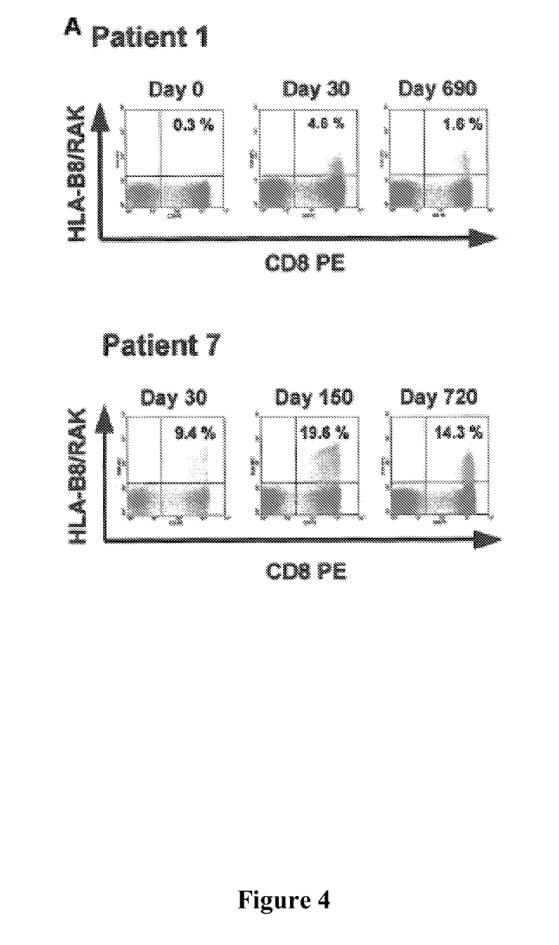

The methods and compositions can also be used to allow for expansion of T cell clones (CD4 and CD8) that are specific for lytic and/or latent EBV gene products when a subject (e.g., human) is immune competent and thus provide a quantitatively higher reserve of memory T cells. This embodiment finds use in a subject that is about to undergo immunosuppressive therapy in preparation for organ transplant. Thus, the subject is vaccinated while the immune system is fully functional, thus allowing the generation of a reserve of memory T cells against EBV. The reserve of memory T cells can be measured pre and post-vaccination by tetramer staining shown in FIG. 4 or, if tetramers are not available for a particular HLA type, by the EliSpot assay. Other methods employing flow cytometric assays can also be utilized to track absolute numbers of memory CD3/CD8 Tc and CD3/CD4 Th cells. Other methods utilizing molecular techniques to quantitatively measure EBV DNA genome copy number or determine the nature of IFN-γ-gene polymorphisms from leukocytes in an individual. Finally, cytokines/chemokines, or viral gene product (or derivations of) polypeptides can be measured by enzyme linked immunosorbent assays (ELISA).The methods and compositions of the instant invention provide greater protection against EBV-associated diseases (e.g., PTLD) once immunosuppressive therapy has been initiated or immune dysregulation triggered by mechanisms described above.

The EBV lytic gene products that can be used in accordance with the present invention include, but are not limited to, BZLF1, BHRF1, BHLF1, BALF2, BMLF1, BRLF1, BMRF1, BALF5, BARF1, BORF2, BCRF1, BKRF3, BDLF3, BILF1, BFRF1, BXLF1, BGLF4, BGLF5, gp350, gp220 and LMP1-lyt. The EBV latent gene products include, but are not limited to, EBNA1, EBNA2, EBNA3A, EBNA3B, EBNA3C, EBNA-LP, LMP1, LMP2A, and LMP2B. It is not necessary that the EBV gene products used in accordance with the present invention be isolated or purified, but it is noted that EBV gene products can be isolated and purified using standard protocols familiar to those skilled in the art.

Table 1 provides a listing of all of the open reading frames from EBV, and lists the nucleic acid sequences associated with the Type 1 strain. Any of the gene products of the open reading frames may be used in accordance with the present invention, though particular lytic and latent gene products are exemplified herein. It should be abundantly clear that the invention is not limited to the particular nucleic acid sequences set forth in Table 1, as those are only the sequences for the Type 1 strain. The open reading frames from other EBV strains may have different sequences, and these are expressly contemplated. Thus, reference is made throughout this specification to the common names of the gene products of the open reading frames (“BZLF1”, for example). It is to be understood that reference to these common names is not intended to be limited to the particular Type 1 sequences disclosed herein, but includes the sequences for other strains as well. When reference to a particular sequence is intended, reference to that sequence will be so made.

In one embodiment, EBNA1, which generates a CD8 and CD4+ T cell response, is combined with any of the other gene products listed above, all of which trigger CD8 T cell responses (but not CD4 T cell responses). This composition of the invention is therefore additionally advantageous in generating both CD4 and CD8 T cell responses. Methods of use of this particular combination are expressly contemplated.

As used herein, the term “gene product” means a polynucleotide encoding the full length coding sequence of a gene or cDNA, or polypeptide comprising the full length amino acid sequence of a given protein. However, it is noted and anticipated that mutations or modifications may be made to the EBV gene products described herein without any effect on the inventive methods or compositions. The types of mutations that are made are of various types. Deletion mutations, in which certain nucleotide bases are deleted, form open reading frame sequence resulting in deletions or changes in amino acids in the translated polypeptide. Insertion mutations occur via the addition of a nucleotide base within a given coding sequence resulting in frame shift of the polynucleotide sequence. And mutations that result in substitutions of one amino acid for another can also be made.

With regard to amino acid substitutions, a variety of amino acid substitutions can be made. As used herein, amino acids generally can be grouped as follows: (1) amino acids with nonpolar or hydrophobic side groups (A, V, L, I, P, F, W, and M); (2) amino acids with uncharged polar side groups (G, S, T, C, Y, N, and Q); (3) polar acidic amino acids, negatively charged at pH 6.0-7.0 (D and E); and (4) polar basic amino acids, positively charged at pH 6.0-7.0 (K, R, and H). Generally, “conservative” substitutions, i.e., those in which an amino acid from one group is replaced with an amino acid from the same group, can be made without an expectation of impact on activity. Further, some non-conservative substitutions may also be made without affecting activity. Those of ordinary skill in the art will understand what substitutions can be made without impacting activity.

While the naturally occurring amino acids are discussed above, non-naturally occurring amino acids, or modified amino acids, are also contemplated and may be used as substitutions in the recited gene products. Thus, as used herein, “amino acid” refers to natural amino acids, non-naturally occurring amino acids, and amino acid analogs, all in their D and L stereoisomers. Natural amino acids include alanine (A), arginine (R), asparagine (N), aspartic acid (D), cysteine (C), glutamine (Q), glutamic acid (E), glycine (G), histidine (H), isoleucine (I), leucine (L), lysine (K), methionine (M), phenylalanine (F), proline (P), serine (S), threonine (T), tryptophan (W), tyrosine (Y) and valine (V). Non-naturally occurring amino acids include, but are not limited to azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4 diaminoisobutyric acid, desmosine, 2,2′-diaminopimelic acid, 2,3-diaminopropionic acid, N-ethylglycine, N-ethylasparagine, hydroxylysine, allo-hydroxylysine, 3-hydroxyproline, 4-hydroxyproline, isodesmosine, allo-isoleucine, N-methylglycine, N-methylisoleucine, N-methylvaline, norvaline, norleucine, ornithine, and pipecolic acid.

It should be noted that EBV gene products may also comprise amino acids linked to either end, or both. These additional sequences may facilitate expression, purification, identification, solubility, membrane transport, stability, activity, localization, toxicity, and/or specificity of the resulting polypeptide, or it may be added for some other reason. The EBV gene products may be linked directly or via a spacer sequence. The spacer sequence may or may not comprise a protease recognition site to allow for the removal of amino acids. Examples of amino acids that may be linked to EBV gene products include, but are not limited to, a polyhistidine tag, maltose-binding protein (MBP), glutathione S-transferase (GST), tandem affinity purification (TAP) tag, calcium modulating protein (calmodulin) tag, covalent yet dissociable (CYD) NorpD peptide, Strep II, FLAG, heavy chain of protein C (HPC) peptide tag, green fluorescent protein (GFP), metal affinity tag (MAT), and/or a herpes simplex virus (HSV) tag. It should be further noted that EBV gene products may also comprise non-amino acid tags linked anywhere along the EBV gene product. These additional non-amino acid tags may facilitate expression, purification, identification, solubility, membrane transport, stability, activity, localization, toxicity, and/or specificity of the resulting polypeptide, or it may be added for some other reason. The EBV gene products may be linked directly or via a spacer to the non-amino acid tag. Examples of non-amino acid tags include, but are not limited to, biotin, carbohydrate moieties, lipid moieties, fluorescence groups, and/or quenching groups. The EBV gene products may or may not require chemical, biological, or some other type of modification in order to facilitate linkage to additional groups.

Additionally, while reference has been made to specific open reading frames, and the common names of the gene products resulting therefrom, variants of those are specifically contemplated as well. A “variant” as used herein, refers to a protein (or peptide or polypeptide) whose amino acid sequence is similar to a reference peptide/polypeptide/protein, but does not have 100% identity to the respective peptide/polypeptide/protein sequence. A variant peptide/polypeptide/protein has an altered sequence in which one or more of the amino acids in the reference sequence is deleted or substituted, or one or more amino acids are inserted into the sequence of the reference amino acid sequence (as described above), A variant can have any combination of deletions, substitutions, or insertions. As a result of the alterations, a variant peptide/polypeptide/protein can have an amino acid sequence which is at least about 80, 81, 82, 83, 84, 85, 86, 87, 88, 89, 90, 91, 92, 93, 94, 95, 96, 97, 98, 99, or higher percent, identical to the reference sequence.

In order to determine whether a variant polypeptide is substantially identical with the reference polypeptide, the variant polypeptide sequence can be aligned with the sequence of a first reference vertebrate polypeptide. One method of alignment is by BlastP, using the default setting for scoring matrix and gap penalties. In one embodiment, the first reference polypeptide is the one for which such an alignment results in the lowest E value, that is, the lowest probability that an alignment with an alignment score as good or better would occur through chance alone. Alternatively, it is the one for which such alignment results in the highest percentage identity.

In some embodiments, at least two EBV gene products are administered for induction of an immune response in a subject. This embodiment allows the possibility of producing multiple immune dominant peptides that can be efficiently processed by the antigen presenting cells to coordinate primary and/or memory CD4 helper and/or CD8 CTL responses against EBV-associated diseases. This approach is also viable for use in treating a subject prior to organ transplantation while the subject is immune competent and thus provide a higher reserve of memory T cells. The reserve of memory T cells can be measured pre and post-vaccination by tetramer staining shown in FIG. 4 or, if tetramers are not available for a particular HLA type, by the EliSpot assay. The at least two EBV gene products chosen can either be from the lytic gene products, latent gene products, or one from each of the lytic and latent gene products. Different combinations of lytic and latent gene products are also contemplated.

In some embodiments, the administering is performed by introducing in a subject an immunogenic composition comprising at least one EBV gene product chosen from EBV lytic gene products and/or EBV latent gene products. The at least one EBV lytic gene product and the at least one EBV latent gene product are administered by a route chosen from subcutaneous, intramuscular, mucosal, intraperitoneal, intradermal, or some other suitable route. The frequency of the administration should allow for the subject to generate sufficient cell mediated immunity that results in prevention and or treatment for EBV-associated diseases. The dose of the administration should be suitable enough to allow for the subject to generate sufficient cell mediated immunity that results in prevention and or treatment for EBV associated diseases. Both the frequency and dose can be determined by someone skilled in the art.

The invention is also directed to the administering of at least one polynucleotide that encodes an EBV gene product chosen from EBV lytic gene products and/or EBV latent gene products. The EBV gene product may be operably linked to a regulatory element in order to allow regulation in terms of expression level of the EBV gene product, localization of the EBV gene product, specificity of the EBV gene product, stability of the EBV gene product or some other reason. In some embodiments, the at least one polynucleotide that encodes an EBV gene product comprises a full length EBV cDNA coding sequence. Furthermore, the full length EBV cDNA coding sequence is chosen from BZLF1, BHRF1, BHLF1, BALF2, BMLF1, BRLF1, BMRF1, BALF5, BARF1, BORF2, BCRF1, BKRF3, BDLF3, BILF1, BFRF1, BXLF1, BGLF4, BGLF5, gp350, gp220 and LMP1-lyt, EBNA1, EBNA2, EBNA3A, EBNA3B, EBNA3C, EBNA-LP, LMP1, LMP2A, and LMP2B coding sequences. Due to the inherent variation of various EBV types, the sequences of the previously described EBV gene products may be matched to correspond to the type of EBV infection the patient has developed or is at a risk of developing. Further still, due to the degeneracy of codon usage, a differing primary nucleotide sequence may still produce the same EBV protein. Codon changes in the nucleotides may also result in additions, deletions, and substitutions of both the nucleotide sequence and/or amino acid sequence of the predicted protein. However, use of EBV gene products with changes in sequence that yield additions, deletions, and substitutions may still result in generation of sufficient cell immunity that protects the subject against EBV-associated diseases. Thus, variants in the nucleic acid sequences are also expressly contemplated.

Viral gene delivery is a method familiar to those skilled in the art for delivering polynucleotides to a subject. Viral gene delivery is a type of treatment whereby a polynucleotide is delivered to cells, allowing them to produce their own therapeutic proteins. Polynucleotides are usually transferred by using viruses that can infect cells, deposit their DNA payloads, and take over the cells' machinery to produce the desirable proteins. By replacing genes that are needed for the replication phase of the virus life cycle (the non-essential genes) with foreign genes of interest, the recombinant viral vectors can transduce the cell type it would normally infect. To produce such recombinant viral vectors the non-essential genes are provided in trans, either integrated into the genome of the packaging cell line or on a plasmid. As viruses have evolved as parasites, they all elicit a host immune system response to some extent. Examples of viral vectors include, but are not limited to, adenoviruses, retroviruses (including lentiviruses), adeno-associated viruses, and herpes simplex virus type 1.

Adeno-associated viruses are non-pathogenic human parvoviruses, dependant on a helper virus, usually adenovirus, to proliferate and assemble infectious virions. They are capable of infecting both dividing and non-dividing cells, and in the absence of a helper virus integrate into a specific point of the host genome at a high frequency. The wild type genome is a single stranded DNA molecule, consisting of two genes; rep, coding for proteins which control viral replication, structural gene expression and integration into the host genome, and cap, which codes for capsid structural proteins. At either end of the genome is a 145 base pair terminal repeat (TR), containing a promoter.

When used as a vector, the rep and cap genes are replaced by the transgene and its associated regulatory sequences. The total length of the insert cannot greatly exceed 4.7 kb, the length of the wild type genome. Production of the recombinant vector requires that rep and cap are provided in trans, along with helper virus gene products (E1a, E1b, E2a, E4 and VA RNA from the adenovirus genome). The conventional method is to cotransfect two plasmids, one for the vector and another for rep and cap, into 293T cells infected with adenovirus. More recent protocols remove all adenoviral structural genes and use rep resistant plasmids or conjugate a rep expression plasmid to the mature virus prior to infection.

cDNA clones are isolated from wild type EBV-transformed lymphoblastoid cell lines or other suitable sources. The base sequence of each full-length cDNA may be verified prior to cloning into rAAV2 vectors. HeLa producer cells are then transfected with rAAV-transgene plasmids encoding AAV-rep (for transgene replication) and cap (for viral capsid production) and an antibiotic resistant gene. Transfected HeLa producer cells are cultured under antibiotic selection and resistant colonies harvested, expanded and cryopreserved. Cells from resistant colonies are immobilized on nitrocellulose membranes (Dot Blot) and probed with cDNA probes to identify colonies with greatest replication activity. Clones with high transgene replication activity were evaluated for the presence of rAAV transgene expression (DNA) and full length protein by western blot. The resulting vectors are capable of delivering antigens into professional antigen presenting cells (APCs), i.e. dendritic cells (DCs), resulting in a cellular immune response. rAAV virions carrying EBV gene products are capable of infecting human APCs and/or DCs leading to expression of full length EBV gene products allowing the antigen processing machinery of the APC and/or DC to present peptides via class I and/or class II MHC.

The instant invention also includes pharmaceutical compositions, which contain, as an ingredient, one or more of the polypeptides and/or polynucleotides described herein. In one embodiment, the pharmaceutical composition comprises EBV polypeptides. In another embodiment, the pharmaceutical composition comprises a polynucleotide encoding such a polypeptide. In preparing the pharmaceutical compositions, the polypeptides or polynucleotides are usually mixed with an excipient, diluted by an excipient, and/or enclosed within a carrier which can be in the form of a capsule, sachet, paper or other container. Any carriers or vehicles can be used that facilitate the administration of pharmacological agents, including the polynucleotides, polypeptides, excipients, or adjuvants, to a target population of cells. Such pharmaceutical compositions may be packaged into convenient kits providing the necessary materials, instructions, and equipment. The pharmaceutical compositions can be administered in a single dose or in multiple doses through routes of inoculation and methods of delivery that are known in art.

In some embodiments, at least one EBV gene product chosen from EBV lytic gene products and/or EBV latent gene products comprise the pharmaceutical composition. The pharmaceutical composition may also comprise 2, 3, or 4 EBV gene products chosen from EBV lytic gene products and/or EBV latent gene products. The pharmaceutical composition may also comprise at least one pharmaceutically acceptable excipient. The excipient is chosen from, but not limited to, water, sterile water, salts, buffers, carbohydrates, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, syrup, and methyl cellulose. In some embodiments, the pharmaceutical composition further comprises at least one adjuvant. The adjuvant may be chosen from, but is not limited to, Freunds adjuvant, a water/oil emulsion, mineral oil, granulocyte/macrophage-colony stimulating factor, and interleukin-2.

The pharmaceutical composition may comprise, for example, the EBV lytic gene product BZLF1 and the EBV latent gene product EBNA3C and at least one pharmaceutically acceptable excipient and/or adjuvant. Furthermore, the pharmaceutical composition may comprise, for example, the EBV lytic gene products BZLF1 and BMLF1, and the EBV latent gene products EBNA3C, EBNA1, and EBNA3A, and at least one pharmaceutically acceptable excipient and/or adjuvant. The pharmaceutical composition may be administered by a route chosen from subcutaneous, intramuscular, mucosal, intraperitoneal, intradermal or other routes.

The invention is also directed to non-viral vectors comprising an EBV gene product expression cassette comprising a polynucleotide encoding at least one EBV gene product and a heterologous promoter operatively linked to the polynucleotide encoding an EBV gene product. Non-viral vectors can be divided into two broad categories, physical and chemical. Physical methods involve taking plasmids and forcing them into cells through such means as electroporation, sonoporation, or particle bombardment. Chemical methods use lipids, polymers, or proteins that may complex with DNA, condensing it into particles and directing it to the cells. The vectors described here are sometimes referred to as “naked” DNA vaccines. In some embodiments, the EBV gene products are chosen from BZLF1, BHRF1, BHLF1, BALF2, BMLF1, BRLF1, BMRF1, BALF5, BARF1, BORF2, BCRF1, BKRF3, BDLF3, BILF1, BFRF1, BXLF1, BGLF4, BGLF5, gp350, gp220 and LMP1-lyt, EBNA1, EBNA2, EBNA3A, EBNA3B, EBNA3C, EBNA-LP, LMP1, LMP2A, and LMP2B. The present invention is also directed to a cell comprising the vector as described above, and methods of culturing said cell under conditions to produce an immune response to EBV gene products comprising, growing the cell under conditions favorable to the expression of EBV gene products.

The invention is also directed to a method for ascertaining a subject's response to an EBV vaccine comprising assaying for the presence of at least one EBV gene product chosen from EBV lytic and EBV latent gene products. The reserve of memory T cells would be easily measured pre- and post-vaccination by the tetramer staining shown in FIG. 4 or, if tetramers are not available for a particular HLA type, by the EliSpot assay, or some other assay.

### EXAMPLES

### Example 1

### The hu-PBL-SCID Mouse Model of Fatal EBV(+) Malignant Lymphoproliferative Disorder (EBV-LPD) is Highly Analogous to Human PTLD

When severe combined immune deficient (SCID) mice undergo intraperitoneal injection with peripheral blood leukocytes (PBL) from normal human donors seropositive for EBV, a majority of these mice (hu-PBL-SCID mouse model) subsequently and spontaneously develop a fatal EBV-LPD of human B-cell origin. Because T cells are critical in the control of EBV infection, we hypothesized that human T-cell dysfunction accounts for EBV-LPD in the xenogenic hu-PBL-SCID mouse model and that systemic administration of T-cell-derived cytokines would reestablish protective immunity against EBV-LPD. We demonstrated that the daily subcutaneous administration of a very low dose (500 international units) of polyethylene glycol-modified recombinant human interleukin 2 (PEG-IL-2) to hu-PBL-SCID mice could prevent the development of fatal EBV-LPD and significantly improves survival (78%), compared with the survival of hu-PBL-SCID mice treated with placebo (20%, P=0.0008). Additional lymphocyte-depletion experiments showed that mouse natural killer cells and human CD8+ T cells provided cellular immunity necessary for the PEG-IL-2-mediated protective effect, whereas intraperitoneal injection of human peripheral blood lymphocytes depleted of CD4+ T cells had no adverse effect when combined with PEG-IL-2 therapy and may have been beneficial. These early data first established that very low-dose PEG-IL-2 therapy can overcome the human T cell immune deficiencies that lead to EBV-LPD in the hu-PBL-SCID mouse and pointed to the usefulness of this model for evaluating cellular responses and cytokine therapies in EBV-LPD (Baiocchi and Caligiuri, 1994).

### Example 2

### Molecular and Cellular Characterization of the Human Tumors that Spontaneous Arise from the hu-PBL,-SCID Mouse Model

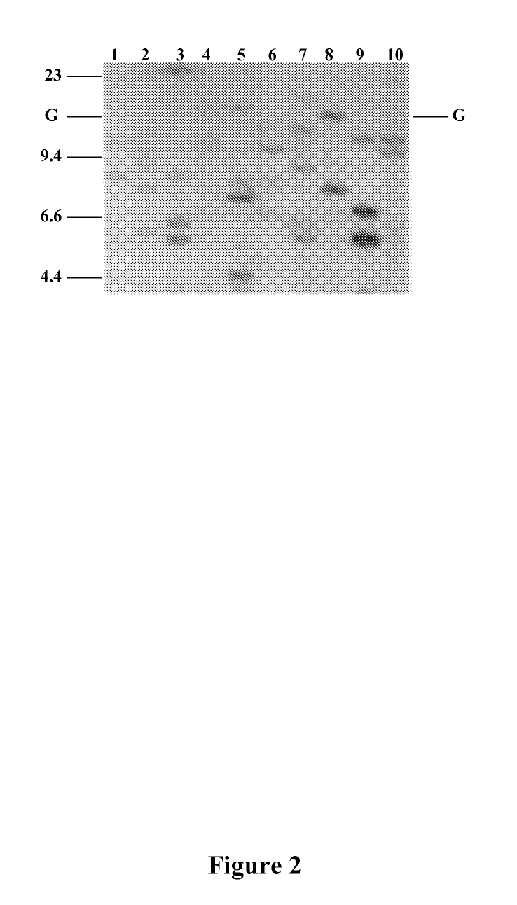

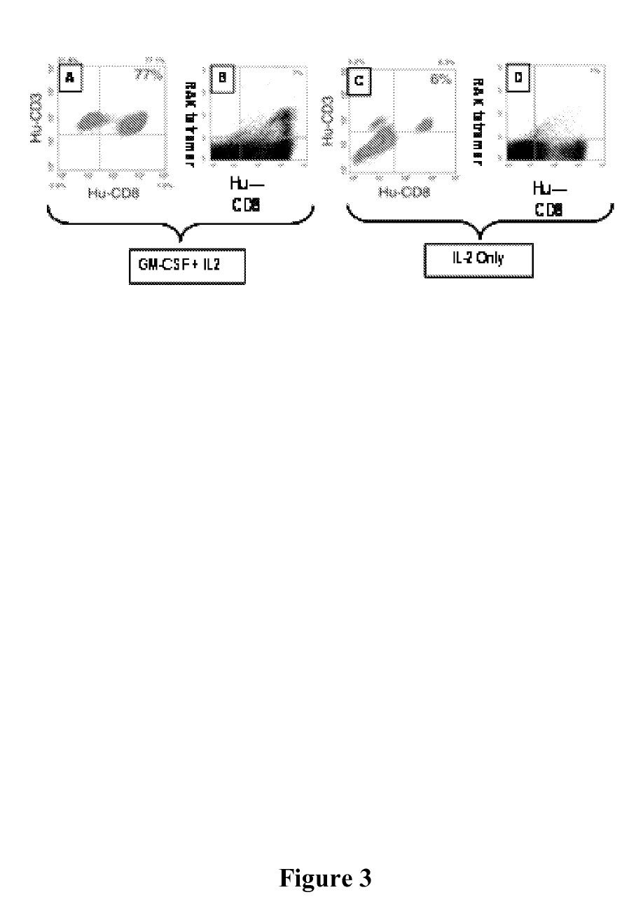

We subsequently performed an extensive molecular and cellular characterization of the human tumors that spontaneous arise from the hu-PBL-SCID mouse model (Baiocchi et al., 1995). Like PTLD, tumors can be monoclonal (FIG. 2, lanes 8,9), oligoclonal (lane 3), or polyclonal (lanes 2,7). The tumors secrete large amounts of human IL-10 and IL-6 both of which serve as survival, growth and T-cell immunosuppressive factors. All tumors integrate viral EBV DNA, and all tumors display a EBV type III pattern of viral gene expression, similar to PTLD. SCID mice lack B and T cells but do have potent natural killer cells. Because 80-100% of hu-PBL-SCID treated with low dose IL-2 succumb to EBV-LPD when mouse NK cells are depleted (Baiocchi and Caligiuri, 1994), we utilized the hu-PBL-SCID mouse model depleted of mouse NK cells to determine what combination of cytokines could replace the mouse NK deficiency and protect the chimeric mouse from fatal EBV-LPD when only engrafted with human immune cells. We discovered that a combination of low dose IL-2 and GM-CSF could restore this protection. Further, a careful characterization of the human T cell elements within the mice randomized to receive one or both of these cytokines revealed a robust human T cell presence in the chimeric mice treated with IL-2 and GM-CSF, but not in the others. This presence was associated with the absence of expanding malignant EBV(+) B cells. Characterization of the human T cells with tetramer staining for EBV lytic and latent antigens demonstrated, for the first time, that the in vivo T cell response was directed against both latent and lytic (FIG. 3) EBV antigens (Baiocchi et al., 2001). It is important to note that these human tumors developed spontaneously when unprimed human PBL were engrafted into SCID mice. Hence, we had our first evidence of how human EBV-specific T cells effectively survey against the development of fatal EBV-LPD in vivo.

### Example 3

### The hu-PBL-SCID Model of EBV-LPD Predicts the Immune Response in PTLD

We next assessed renal transplant patients with PTLD for evidence of a similar immune response. From 1997 through 2002 we treated 11 consecutive renal transplant PTLD patients at our institution with what we developed as a standardized reduction of immune suppression and standardized antiviral therapy. We reported a 91% rate of complete remission, and 82% have remained in a continuous CR with a median duration of nearly four years. One patient that relapsed has been back in remission for over 3 years, so 91% are currently alive and well (Porcu et al., 2002). We believe this to be the best outcome data for PTLD in renal transplant patients ever reported. Importantly, at the time of tumor regression, we used tetramer staining to show that PTLD patients that were successfully responding to the intervention had a quantifiable CD8+ T cell response to both latent and lytic (RAK) EBV antigens, virtually identical to that seen in the human-SCID mouse model of the disease (FIG. 4). These data provide two important points that support the effort being put forth in this application. First, a successful immune response to EBV(+) PTLD can be mounted, readily quantified and is associated with regression of disease and 2) this response is a highly specific CTL, response to both latent and lytic EBV antigens.

### Example 4

### In vivo Discoveries made in the Chimeric Mouse-Human Model and in Patients

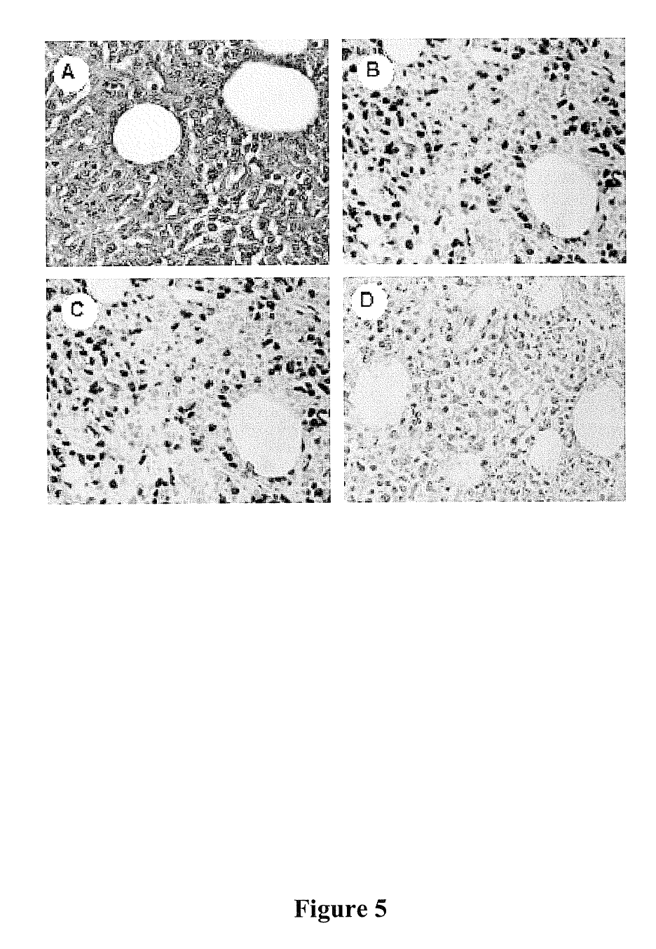

Because risk of developing PTLD correlates closely with rising EBV genome copy number in peripheral blood, it is clear that activation of lytic infection is operable and important in the pathogenesis of this disorder. Discoveries made in our laboratory have demonstrated the presence of lytic gene expression in PTLD tumors (Porcu, 2002; Roychowdhury, 2003). We evaluated 16 separate tumors collected from 8 patients with PTLD for the expression of the lytic gene product BXLF1, a viral thymidine kinase (vTK) that is positively regulated by the early lytic cycle gene product BZLF1 (Table 1). All tumors demonstrated expression of BXLF1 transcript providing evidence that lytic gene activity is operable and sustained in PTLD tumors (FIG. 5).

We have discovered several treatment regimens that have generated encouraging results in preclinical animal models of PTLD (Baiocchi, 1994; Baiocchi, 2001; Roychowdhury, 2003; Roychowdhury, 2004) and in patients with PTLD. (Roychowdhury, 2003; Khatri, 1997; Porcu, 2002) We have also characterized the in vivo T cell response to EBV-specific antigens that forms the basis for our use patent in the prevention of PTLD and other EBV-associated malignancies. Using in vivo discoveries we made with a chimeric murine-human model of human PTLD (Baiocchi, 2001) and in vivo discoveries we made while observing human immune response in PTLD patients we have treated (Porcu, 2002), we have identified several EBV antigens to which spontaneously expanding cytotoxic T lymphocytes (CTL) are reacting when successfully controlling PTLD, i.e., EBV-specific CTL,. In the chimeric mouse-human preclinical model of human PTLD, we utilized EBV antigen-specific MHC class I peptide loaded tetramers to document an expansion of EBV-specific CTL recognizing lytic BZLF1 and latent EBNA3C antigens when the mice received a combination of low dose interleukin-2 (IL-2) and GM-CSF. Animals receiving therapy with placebo or single agent IL-2 or GM-CSF showed no evidence of EBNA3C or BZLF1-specific CTLs and went on to die from human EBV(+) lymphoproliferative disorder. Animals receiving combined IL-2 and GM-CSF showed significant, measurable EBNA3C and BZLF-1-specific CTL expansion and were protected from developing fatal human EBV(+) lymphoproliferative disorder. (Baiocchi, 2001) Using the same MHC Class I tetramers loaded with specific peptides to document EBV antigen-specific T cell expansion, we discovered that the human CTL in patients with PTLD also recognize an immune-dominant peptide derived from the BZLF protein. (Porcu, 2002) BZLF is an EBV encoded gene product that is expressed by the tumor exclusively during early lytic infection (FIG. 5). (Kieff, 2001) Using tetramers loaded with peptide derived from the latent protein EBNA3C, we discovered spontaneous expansion of CTL specific for EBNA3C in these same PTLD patients (FIG. 5). (Porcu, 2002) Importantly, expansion of this BZLF1-specific CTL population occurred following the withdrawal of immune suppression in renal transplant patients with PTLD, was sustained for a significant period of time and directly correlated with regression of EBV+ tumor burden. One patient who failed to sustain their CTL response had subsequent relapse of PTLD. (Porcu, 2002) The sustained presence (up to 720 days post withdrawal of immune suppression) of BZLF1-specific CTL suggests that peptides derived from the tumor's BZLF1 protein were also either continuously or intermittently endogenously presented by antigen presenting cells in order to sustain effective T cell immune surveillance. This suspected sustained exposure to immunogen has relevance to our vaccine approach for the prevention of PTLD, described below in our use patent.

### Example 5

### Clinical Application of These in vivo Discoveries

Our published in vivo results strongly suggest that latent and lytic EBV gene products are the important proteins containing multiple immune dominant peptides that can be efficiently processed by antigen presenting cells to coordinate primary and memory antigen specific CD4 helper and CD8 CTL responses for protection against EBV diseases including malignancies such as PTLD. Given the close correlation between expansion of EBNA3C and BZLF1 specific CTLs in vivo and tumor regression and survival of PTLD patients, we believe that full length EBV latent and lytic proteins are ideal immunogens that can be utilized to vaccinate all patients who are candidates for solid organ transplantation and therefore at high risk for the development of PTLD. Such an approach would allow for expansion of T cell clones (CD4 and CD8) specific for latent and lytic EBV protein-derived endogenous peptides prior to organ transplantation when patients are immune competent and thus provide a quantitatively higher reserve of memory T cells. The latter would be easily measured pre- and post-vaccination by the tetramer staining shown in FIG. 5 above or, if tetramers are not available for a particular HLA type, by the EliSpot assay or other assay for IFNγ (intracellular flow cytometry). This approach would allow for faster mobilization and expansion of EBV-specific T cells in vivo in the event of primary (pediatric patients) or reactivated (95% of adults) EBV infection. This approach should provide protection against uncontrolled Epstein-Barr viremia and subsequent development of PTLD in most if not all patients receiving iatrogenic immune suppressive therapy. For those who may still develop PTLD following such vaccination, the vaccine prevention approach should allow for an earlier and quantitatively more robust EBV-specific CTL response with a modest lowering of immune suppressive therapy, without a comparable allograft-specific CTL response. This in turn should allow for elimination of PTLD with a much lower incidence of allograft rejection (or graft versus host disease in stem cell transplant patients) compared to that seen in our patients who did not have vaccine prior to lowering their immune suppressive therapy for elimination of life-threatening PTLD. (Porcu, 2002) It is possible that this same approach can be extended to other patient groups at risk for EBV-associated diseases including patients with acquired, congenital or iatrogenic (other than stem cell or organ transplant) immune suppression.

Because of the restricted association between different viral protein-derived peptides and specific HLA types, immunization with select peptides derived from latent and lytic EBV gene products would provide immune dominant antigens for a relatively small group of patients. For example, the RAK peptide derived from BZLF1 is immune dominant when presented in the context of class I molecules of HLA B8 patients (FIG. 5), but not HLA A2. This restriction can be circumvented by providing full-length EBV latent and lytic polypeptides or proteins to the antigen-processing networks, thereby allowing for optimal presentation in the context of most if not all types of HLA class I and II molecules. Patients receiving these larger molecules will then be able to process full length polypeptides and present immune dominant peptides in the context of their specific HLA Class I and II molecules. Delivery of full length latent and lytic EBV polypeptide immunogens for the purposes of vaccination can be accomplished via direct delivery of the purified protein with an immune adjuvant or via recombinant DNA-based techniques that we review in data presented below. As one lytic or latent polypeptide might not be sufficiently large enough to present an immune dominant peptide to all HLA types following processing by an antigen presenting cell, a minimum of two latent and two lytic EBV gene products will be developed for this vaccine, as described immediately below.

The idea to provide full length latent and lytic EBV gene products in the form of a vaccine to generate efficient cell mediated immunity with the intent of preventing PTLD in solid organ transplantation is novel and is supported by discoveries made by our laboratory and by others. In addition to the EBNA3C and BZLF1-specific CTLs detected in our in vivo studies examining the T cell response following withdrawal of immune suppression, other laboratories have demonstrated both CD4 and CD8 T cell responses specific for gene products of the EBV early antigen complex BMRF1, BHRF1, BORF2 (Pothen, 1991; Pothen, 1993) and gp340/350 (Wallace, 1991) as well as the latent gene product EBNA1 (Paludan, 2002). We therefore initially propose to deliver the following full-length polypeptides (or encoding DNA sequences): BZLF1, BMLF1, EBNA1, EBNA3A and EBNA3C. Utilizing at least two latent and two lytic immunogens in a vaccination strategy to prevent PTLD would provide optimal protection for two reasons: 1) as noted above, it ensures that each individual has an immune dominant peptide presented for both latent and lytic polypeptides, no matter what the HLA type; 2) it would minimize skewing of the CTL response and thereby prevent emergence of EBV-transformed clones expressing latent gene products only; 3) it would promote both antigen-specific Th and Tc responses. This strategy would also protect against uncontrolled lytic replication and viremia. Based on the immunogenicity of the above-mentioned polypeptides as will be determined by expansion of EBV-specific CTL post vaccination, we propose to improve the response by applying the same strategy to deliver other polypeptides coded by EBV including BZLF1, BHRF1, BHLF1, BALF2, BMLF1, BRLF1, BMRF1, BALF5, BARF1, BORF2, BCRF1, BKRF3, BDLF3, BILF1, BFRF1, BXLF1, BGLF4, BGLF5, gp350, gp220, LMP1-lyt, EBNA1, EBNA2, EBNA3A, EBNA3B, EBNA3C, EBNA-LP, LMP1, LMP2A, and LMP2B.

### Example 6

### Prevention Vaccine for Human PTLD

While 10 of the original 11 renal PTLD patients are currently alive and without PTLD, five of the patients lost their allografted kidney due to T-cell mediated organ rejection and required either re-transplantation or dialysis. Thus, the current successful approach to the management of renal transplant PTLD has two limitations: first, the loss of the allografted kidney in 50% of these PTLD patients is unacceptably high, even with the threat of a fatal malignant tumor and, 2) this approach of reduction in immune suppressive therapy with anti-viral therapy is not applicable in other solid organ transplants such as heart, lung, liver, or small bowel where allograft rejection would prove fatal. Thus, alternatives must be considered. Our preliminary chimeric mouse-human and human data, partially outlined above, along with other published data from O'Reilly's group (Lacerda et al., 1996b; Lucas et al., 1996) led us to first hypothesize that the in vivo frequency of T-cell precursors to EBV latent and lytic antigens is likely proportional to the likelihood of developing PTLD prior to the reduction of immune suppression, as well as to the robustness of an EBV-specific immune response against PTLD following the reduction of immune suppression. Second, our preliminary mouse-human and human data provide what we believe is good evidence as to what antigens need to be targeted. Collectively, we believe we have sufficient data to justify a comprehensive approach to develop an effective vaccine to prevent PTLD in immune competent patients awaiting solid organ transplantation who will subsequently undergo organ transplant and are therefore be at high risk for EBV-related complications like PTLD. This will increase the frequency of EBV-specific CTL directed against the relevant antigens and should decrease or eliminate the risk of developing PTLD. Further, in the advent of PTLD, this pre-transplant sensitization should lead to a more robust and specific anti-tumor response following reduction in immune suppression, possibly sparing the transplanted organ from rejection.

### Example 7

### Large Scale Production of rAAV2-Based Vectors

Molecular Cloning of EBV Lytic Antigen BZLF1 and BMLF1 into a rAAV2 Expression Cassette

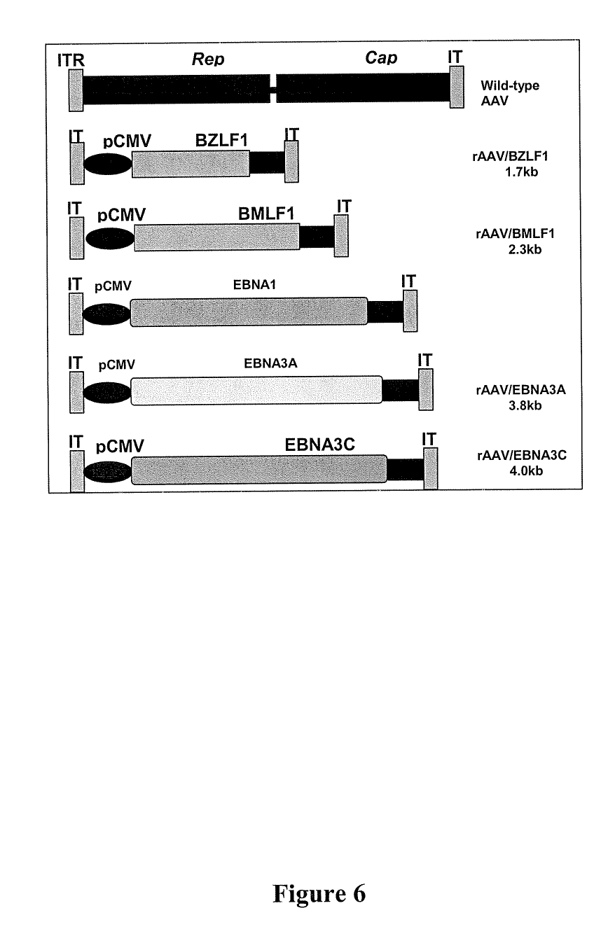

We PCR-amplified the EBV lytic BZLF1 and lytic BMLF1 full-length cDNAs from the EBV positive lymphoblast cell lines B95.8 and C7M3, respectively, and cloned these fragments into a rAAV2-based expression cassette (FIG. 6). rAAV2/BZLF1 and rAAV2/BMLF1 clones were properly identified with restriction enzyme digestion and DNA sequence analysis. Similar techniques were used to generate and validate the three latent EBV rAAV2/EBNA1, rAAV2/EBNA3A, and rAAV2/EBNA3C clones.

Analysis of rAAV2/BZLF1 and BMLF1 Viral DNA Replication and Transgene Expression

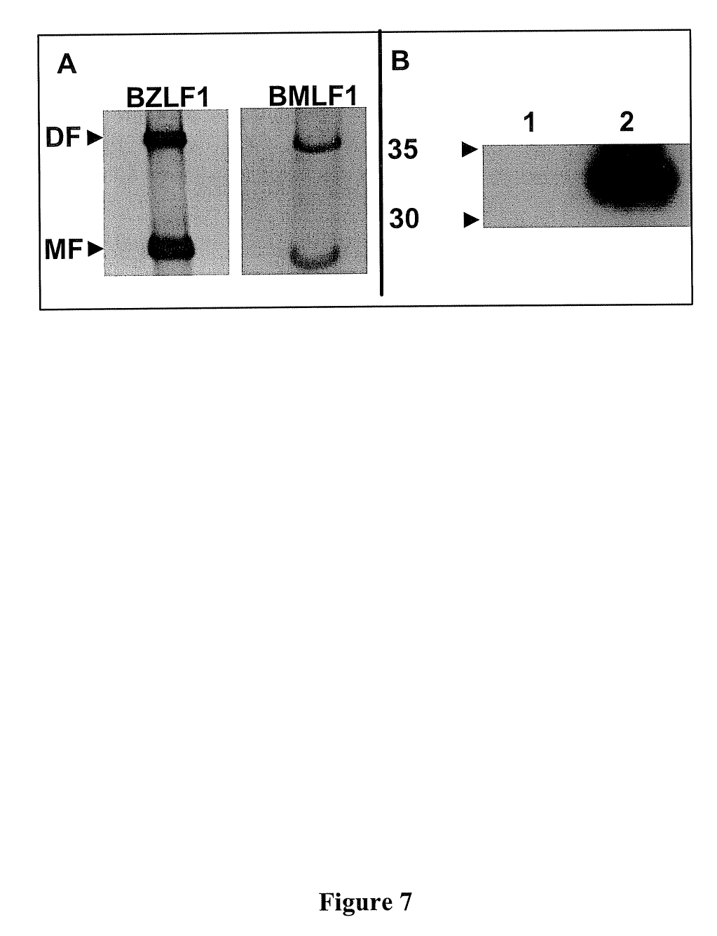

Using standard methodology, we demonstrated successful replication and packaging of rAAV2/BZLF1 and rAAV2/BMLF1 (FIG. 7A monomeric (MF) and dimeric (DF) forms). The BZLF1 transgene expression is shown in FIG. 7B lane 3 is clone 4rAAV/BZLF. Lane 1 was molecular weight standard, Lane 2 HeLa cells (negative control).

Generation of the HeLa Derived Producer Cell Lines and Large-Scale Production of rAAV2/BZLF1 and BMLF1

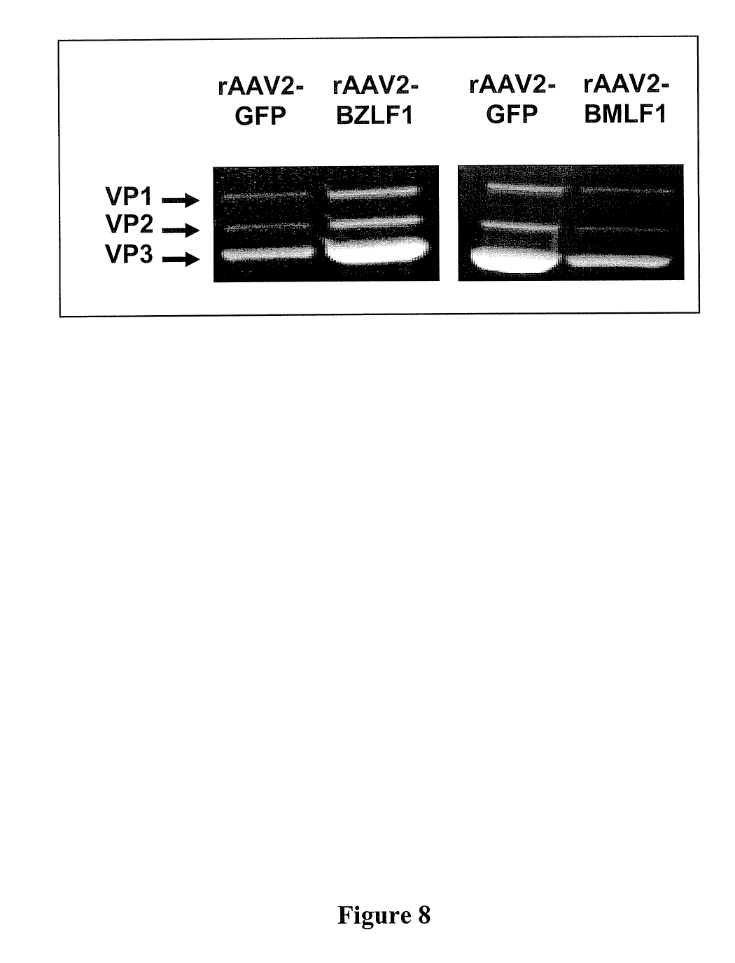

Functional plasmids were transfected, plated, picked, and transferred into 96-well plates. Individual clones were screened by a dot blot hybridization and the optimal producer cell lines (>5000 rAAV vectors/cell) were identified by quantitative real-time PCR to derive DNase-Resistant-Particles (DRPs/cell). The purity of rAAV vectors following large scale production demonstrated the presence of three viral capsid proteins (FIG. 8. VP1:VP2:VP3=1:1:10) by SDS-PAGE SYPRO orange fluorescent staining.

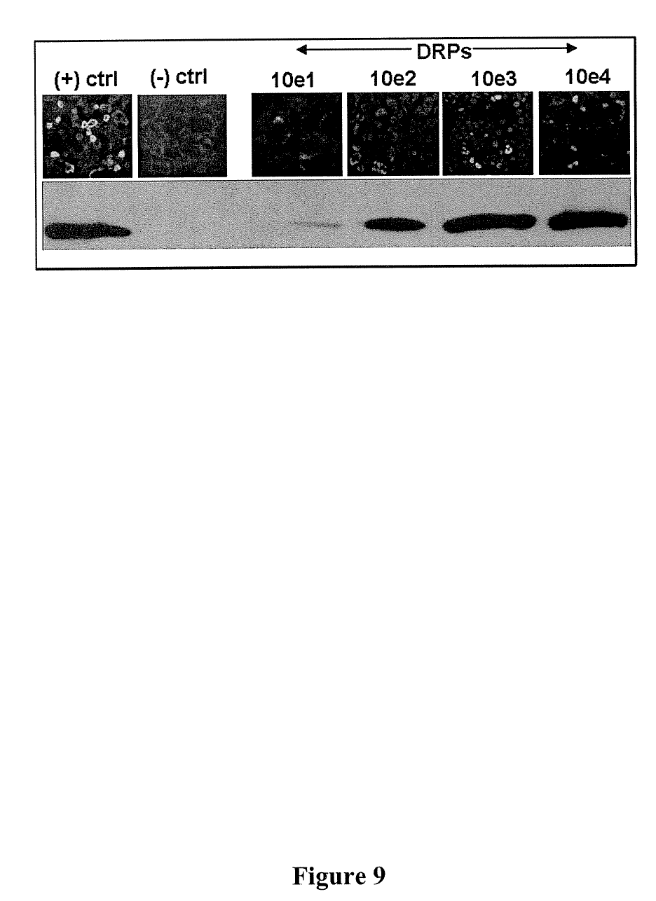

We next transduced 293T cells with different DRPs of rAAV2/BZLF1 and subsequently analyzed BZLF1 protein expression by immunofluorescence and Western blot as shown in FIG. 9. These data demonstrate that rAAV2/BZLF1 is capable of transducing the 293T cells in a dose dependent manner. All together, we have successfully generated large quantities of rAAV2 vectors carrying EBV lytic antigens BZLF1 and BMLF1 and have demonstrated that these vectors can efficiently transduce cells in vitro.

A critical issue for a successful vaccine protocol is whether the designed vectors are capable of delivering antigens into professional antigen presenting cells (APCs), i.e. dendritic cells (DC), efficiently resulting in a potent cellular immune response (Shuler et al., 2003). We next demonstrated that the peak infection of human monocyte-derived DC by rAAV2/GFP was on day 1 of in vitro differentiation (˜7%, data not shown), and showed that this infection process did not alter DC maturation compared with uninfected and mock infection control (data not shown).

### Example 8

### Molecular Cloning of EBV Latent Antigens EBNA1, EBNA3A and EBNA3C into a rAAV2 Based Expression Cassette

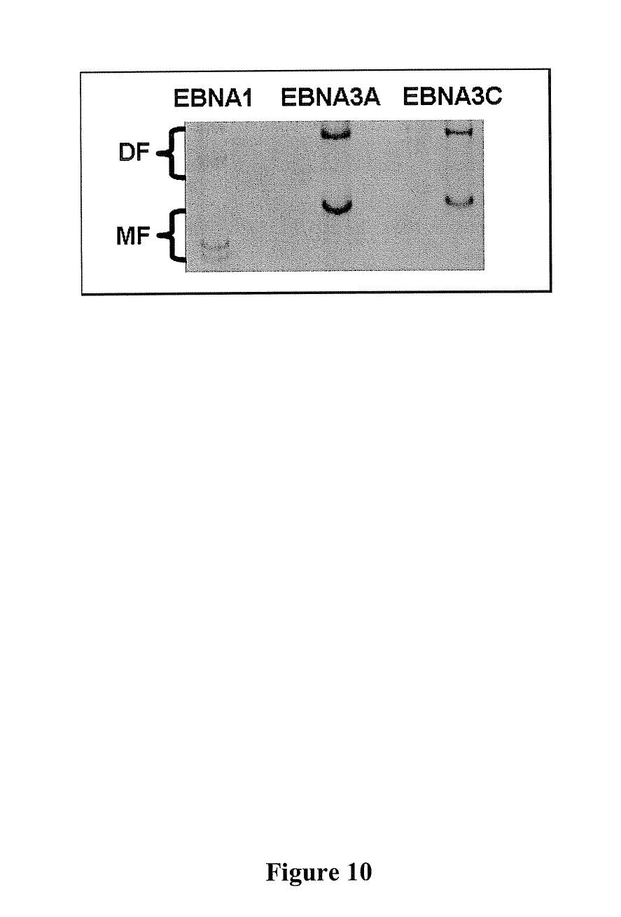

Using similar strategies we cloned full length cDNAs of EBV latent antigen EBNA1, EBNA3A and EBNA3C into rAAV2 vectors (FIG. 6). As previously shown in FIG. 7 and now shown in FIG. 10, the typical MF and DF replicative forms of rAAV2 were detected indicating a successful replication and packaging of rAAV vectors containing EBV latent antigen EBNA1, EBNA3A and EBNA3C.

### Example 9

### In vitro Expansion of EBV Specific CD8+CTL, by Co-Culturing rAAV2/BZLF1 Transduced Dendritic Cells (DC) with Autologous Human PBMC

To evaluate the immunogenicity of full length rAAV-EBV-transgene proteins, we performed experiments to test whether rAAV-BZLF1-derived protein expressed in target antigen presenting cells (APC) was capable of eliciting expansion of memory EBV-specific T cells. To test this, we utilized human peripheral blood mononuclear (PBMC)-derived DCs as our APCs and autologous PBMC from EBV-seropositive donors. EBV-specific memory T cells can reproducibly be expanded from PBMC of EBV-seropositive donors when cultured in the presence of autologous irradiated EBV-transformed lymphoblastoid cell lines (LCL). Alternatively, one can pulse professional APC (DC) with immune dominant peptides derived from EBV proteins. We hypothesized that rAAV-BZLF1 virions would be capable of infecting human DCs leading to expression of full length BZLF1 protein allowing the antigen processing machinery of the DC to present peptides via class I and II MHC. This later scenario using DCs as APCs presents a “best case scenario” to test the efficacy of rAAV-transgene vaccine preparations to activate and expand antigen-specific memory T cells.

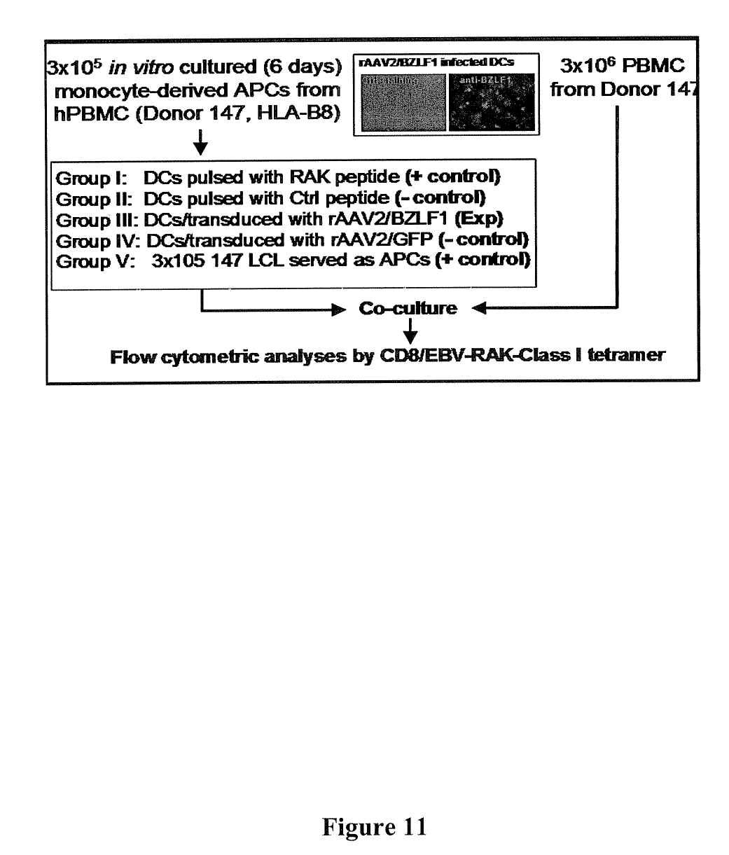

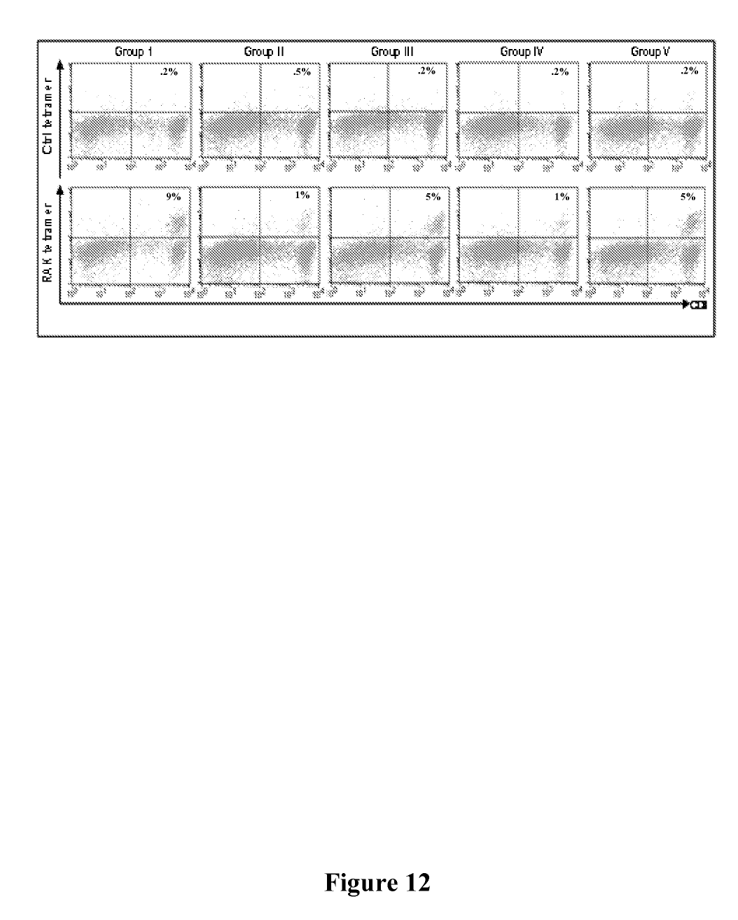

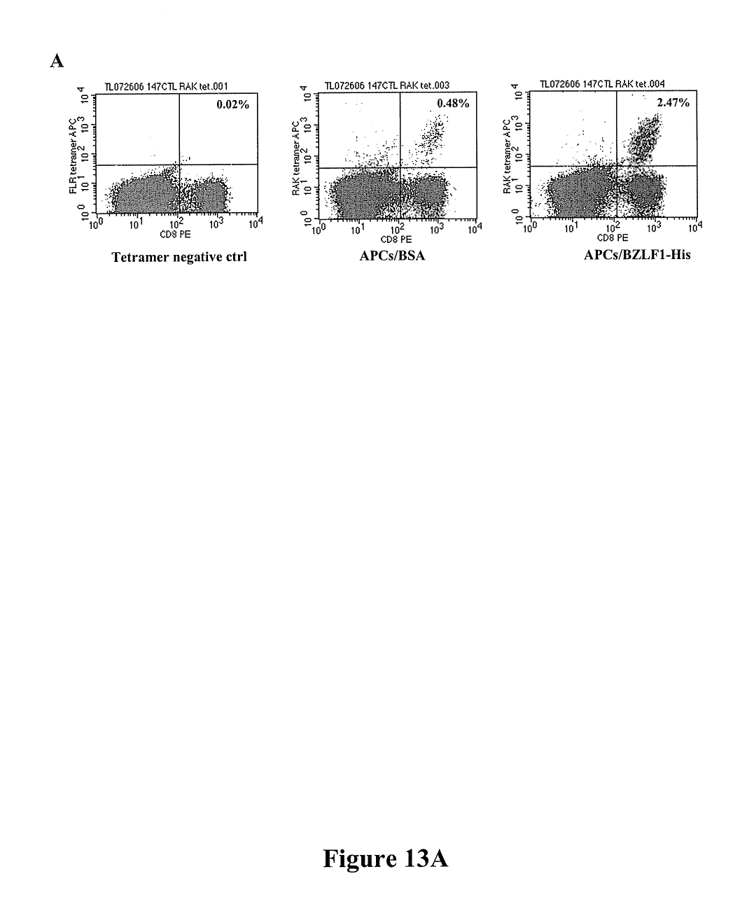

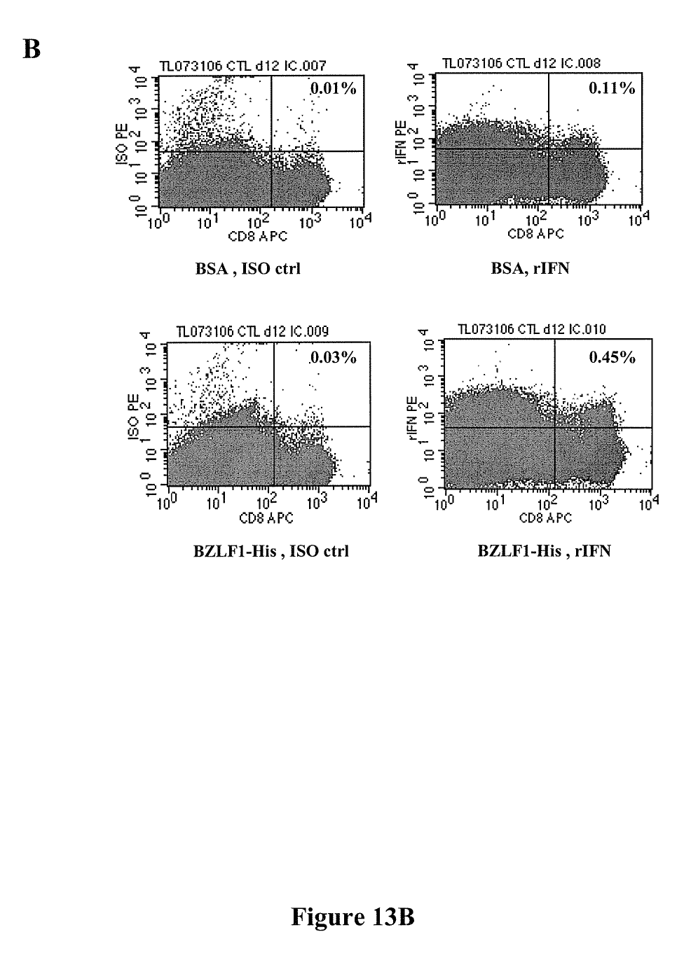

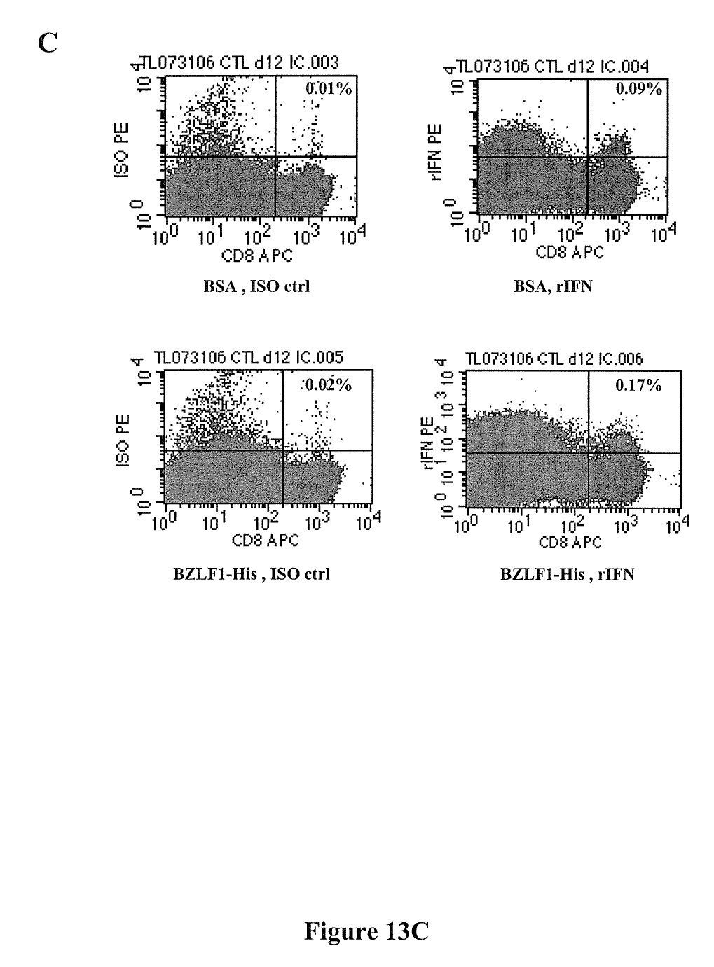

We developed a protocol for expansion of BZLF1 specific CD8+ T cells in vitro (FIG. 11). For Groups III and IV, we prepared PBMC-derived DCs (from donor 147) in the presence of rAAV-BZLF1 or control rAAV-GFP virus (10¹⁰ particles, added at day 1 of differentiation process). The other control groups were prepared using published protocols. We harvested cells at day 7 and 14 and analyzed by flow cytometry using a combination of CD3, CD8, and EBV-RAK class I tetramer staining. The RAK tetramer is specific for the TCR on CD8+ T cells that recognizes the BZLF1 lytic antigen (Baiocchi et al., 2001; Porcu et al., 2002; Rickinson and Kieff, 2001). As shown in FIG. 12, bottom row, we demonstrated that rAAV2/BZLF1 infected DC (Group III bottom row, arrow) induced a robust CD8+ T cells response against BZLF1 antigen, compared to negative controls (Groups II and IV) and positive controls (Groups I and V). The top row of FIG. 12 shows background staining with a non-reactive peptide tetramer for each group. To determine whether the full length BZLF1 polypeptide is immunogenic when processed and presented by antigen presenting cells (APC), we synthesized and purified full length BZLF1 protein for in vitro experiments. FIG. 13 shows that full length BZLF1 protein was capable of expanding CD3/CD8+ T cells that were antigen specific for an immune dominant peptide (RAK) that has been defined for HLAB8 individuals (A). To determine if expanded CD3/CD8+ T cells were capable of effector-like functions we examined this population for capacity to produce IFN-gamma (IFNγ) after stimulation with either full length BZLF1 or control BSA proteins. As seen in (B), we were able to detect 4-fold more CD8+ T cells that marked positive for IFNγ after stimulation with BZLF1 compared to control BSA protein. Our tetramer biomarkers are useful for detecting antigen specific T cells with specific donors that have defined immunodominant peptides and therefore, can not be used for testing all donors, many of whom have an HLA allele that is not associated with immune dominant peptides derived from EBV proteins. We have therefore chosen to use IFNγ as a biomarker to demonstrate expansion T cells that are capable of responding to epitopes derived from full length EBV polypeptides. As seen in (C), donor PBMC from a HLAA2 individual were capable of expanding and producing 2-fold more IFNγ in response to full length BZLF1 compared to control BSA protein. These data support the hypothesis that full length EBV polypeptides contain many unidentified immunogenic peptides that can be processed by APCs from individuals with multiple HLA types to induce a T cell response (measured by IFNγ). Full length BZLF1 polypeptide is capable of inducing a T cell response independent of HLA type or known/defined immunodominant peptides derived from BZLF1.

### Example 10

### Susceptibility to EBV-1LPD and to PTLD

While PBL, from normal individuals who are seropositive for EBV can produce spontaneous human EBV-LPD in the hu-PBL-SCID mouse model, not all donors provide the same degree of efficiency and indeed, there can be a very wide range of success between normal donors (i.e., 0% to 100% EBV-LPD following injection of 12 mice). Likewise, the majority of solid organ transplant patients do not develop PTLD, and while T cell precursor frequency appears to be one such risk factor (Lacerda et al., 1996a; Mackinnon et al., 1995), we have searched for others. We hypothesized that cytokine genotype associates with the development of EBV-LPD. With IRB approval, we developed an extensive list of HLA-typed normal, EBV seropositive donors to test our hypothesis (this list of donors will be available for the studies outlined below). We observed that the A/A (adenosine/adenosine) genotype for base+874 of the IFN-γ gene was significantly more prevalent in PBL, producing rapid, high-penetrance EBV-LPD in hu-PBL-SCID mice, compared to PBL, producing late, low-penetrance LPD or no LPD. In examining the relationship between genotype and cytolytic T-lymphocyte (CTL) function, transforming growth factor beta (TGF-beta) inhibited restimulation of CTLs in PBLs with adenosine at IFNG base+874, but not in PBL homozygous for thymidine. Importantly, neutralization of TGF-beta in hu PBL-SCID mice injected with A/A genotype PBL, resulted in reduced LPD development and expanded human CD8+ CTL. Thus, our data show that TGF-beta may promote tumor development by inhibiting CTL restimulation and expansion (Dierksheide et al., 2005). This has clinical relevance in the context of our vaccine approach as humanized neutralizing anti-TGF-beta antibodies are currently being developed. Our ongoing genotyping data in organ transplant recipients indicate that IFN-γ genotype may provide valuable information for both identifying transplant recipients at greater risk for PTLD, and for developing preventive strategies that are being addressed in this proposal.

What is claimed is:

1. A method for inducing an immune response against at least one virus-associated disease in a subject, comprising: administering to said subject at least one virus gene product.

2. The method according to claim 1, wherein the at least one virus gene product is chosen from virus lytic gene products and virus latent gene products

3. The method according to claim 2, wherein the virus is a human herpes virus chosen from HHV-1 (Herpes Simplex Virus 1), HHV-2 (Herpes Simplex Virus 2), HHV-3 (Varicella Zoster Virus), HHV-4 (Epstein-Barr virus), HHV-5 (Cytomegalovirus), HHV-6, HHV-7, and HHV-8.

4. The method according to claim 3, wherein the virus is Epstein-Barr virus.

5. The method according to claim 4, wherein the Epstein-Barr virus is a strain chosen from Type 1, Type 2, SiIIA, A4, TSB-B6, ap876, p3hr1, b95.8, cao, raji, and daudi.

6. The method according to claim 4, wherein the at least one virus gene product is chosen from the gene products listed in Table 1.

7. The method according to claim 6, wherein the Epstein-Barr virus lytic gene products are chosen from BZLF1, BHRF1, BHLF1, BALF2, BMLF1, BRLF1, BMRF1, BALF5, BARF1, BORF2, BCRF1, BKRF3, BDLF3, BILF1, BFRF1, BXLF1, BGLF4, BGLF5, gp350, gp220 and LMP1-lyt, and the Epstein-Barr virus latent gene products are chosen from EBNA1, EBNA2, EBNA3A, EBNA3B, EBNA3C, EBNA-LP, LMP1, LMP2A, and LMP2B.

8. The method according to claim 7, wherein the lytic gene products are chosen from BZLF1 and BMLF1 and the latent gene products are chosen from EBNA1, EBNA3A, and EBNA3C.

9. The method according to claim 8, wherein the Epstein-Barr virus lytic gene product is BZLF1 and the Epstein-Barr virus latent gene product is EBNA3C.

10. The method according to claim 4, comprising administering at least two Epstein-Barr virus gene products.

11. The method according to claim 4, wherein the virus-associated disease is chosen from neoplastic disease, infectious mononucleosis, hemophagocytic syndrome, renal cell tubulitis, and hepatitis.

12. The method according to claim 11, wherein the neoplastic disease is chosen from lymphoproliferative disorder, Burkitt's lymphoma, Hodgkin's disease, B cell non Hodgkin's lymphoma, epithelial carcinomas of gastric and nasopharyngeal mucosa, undifferentiated nasopharyngeal carcinomas, peripheral T-cell and T/NK cell lymphomas.

13. The method according to claim 12, wherein the lymphoproliferative disorder results from congenital immune deficiency, acquired immune deficiency, and iatrogenic immune deficiency.

14. The method according to claim 13, wherein the lymphoproliferative disorder is post-transplant lymphoproliferative disease.

15. The method according to claim 4, wherein the subject has been diagnosed with at least one Epstein-Barr virus associated disease chosen from neoplastic disease, infectious mononucleosis, hemophagocytic syndrome, renal cell tubulitis, and hepatitis.

16. The method according to claim 15, wherein the neoplastic disease the subject has been diagnosed with is chosen from lymphoproliferative disorder, Burkitt's lymphoma, Hodgkin's disease, epithelial carcinomas of gastric and nasopharyngeal mucosa, undifferentiated nasopharyngeal carcinomas, B cell non-Hodgkin's lymphoma, and peripheral T-cell lymphomas.

17. The method according to claim 16, wherein the lymphoproliferative disorder the subject has been diagnosed with is caused by congenital immune deficiency, acquired immune deficiency, and iatrogenic immune deficiency.

18. The method according to claim 17, wherein the subject has been diagnosed with post-transplant lymphoproliferative disease.

19. The method according to claim 4, wherein administering is done by introducing at least one polynucleotide that encodes an Epstein-Barr virus gene product chosen from Epstein-Barr virus lytic gene products and Epstein-Barr virus latent gene products wherein the Epstein-Barr virus gene product is operably linked to a regulatory element.

20. The method according to claim 19, wherein the at least one polynucleotide that encodes an Epstein-Barr virus gene product comprises a full-length Epstein-Barr virus cDNA coding sequence.

21. The method according to claim 20, wherein the full length Epstein-Barr virus cDNA coding sequence is chosen from BZLF1, BHRF1, BHLF1, BALF2, BMLF1, BRLF1, BMRF1, BALF5, BARF1, BORF2, BCRF1, BKRF3, BDLF3, BILF1, BFRF1, BXLF1, BGLF4, BGLF5, gp350, gp220, LMP1-ly, EBNA1, EBNA2, EBNA3A, EBNA3B, EBNA3C, EBNA-LP, LMP1, LMP2A, and LMP2B coding sequences.

22. The method according to claim 4, wherein the administering is performed by introducing in a subject an immunogenic composition comprising at least one Epstein-Barr virus gene product chosen from Epstein-Barr virus lytic gene products and Epstein-Barr virus latent gene products.

23. The method according to claim 10, wherein the at least one Epstein-Barr virus lytic gene product and the at least one Epstein-Barr virus latent gene products are linked.

24. The method according to claim 1, wherein the virus gene products are administered by a route chosen from subcutaneous, intramuscular, mucosal, intraperitoneal, or intradermal routes.

25. A pharmaceutical composition for inducing an immune response in a subject against at least one virus-associated disease comprising: at least two virus gene products; and at least one pharmaceutically acceptable excipient.

26. The pharmaceutical composition according to claim 25, wherein the virus gene products are chosen from virus lytic gene products and virus latent gene products.

27. The pharmaceutical composition according to claim 26, wherein the virus is a human herpes virus chosen from HHV-1 (Herpes Simplex Virus 1), HHV-2 (Herpes Simplex Virus 2), HHV-3 (Varicella Zoster Virus), HHV-4 (Epstein-Barr virus), HHV-5 (Cytomegalovirus), HHV-6, HHV-7, and HHV-8.

28. The pharmaceutical composition according to claim 27, wherein the virus is Epstein-Barr virus.

29. The pharmaceutical composition according to claim 28, wherein the Epstein-Barr virus is a strain chosen from Type 1, Type 2, SiIIA, A4, TSB-B6, ap876, p3hr1, b95.8, cao, raji, and daudi.

30. The pharmaceutical composition according to claim 28, wherein the at least one virus gene product is chosen from the gene products listed in Table 1.

31. The pharmaceutical composition according to claim 30, wherein the Epstein-Barr virus lytic gene products are chosen from BZLF1, BHRF1, BHLF1, BALF2, BMLF1, BRLF1, BMRF1, BALF5, BARF1, BORF2, BCRF1, BKRF3, BDLF3, BILF1, BFRF1, BXLF1, BGLF4, BGLF5, gp350, gp220 and LMP1-lyt, and the Epstein-Barr virus latent gene products are chosen from EBNA1, EBNA2, EBNA3A, EBNA3B, EBNA3C, EBNA-LP, LMP1, LMP2A, and LMP2B.

32. The pharmaceutical composition according to claim 31, wherein the lytic gene products are chosen from BZLF1 and BMLF1 and the latent gene products are chosen from EBNA1, EBNA3A, and EBNA3C.

33. The pharmaceutical composition according to claim 32, wherein the Epstein-Barr virus lytic gene product is BZLF1 and the Epstein-Barr virus latent gene product is EBNA3C.

34. The pharmaceutical composition according to claim 25, wherein the excipient is chosen from: water, salts, buffers, carbohydrates, solubilizing agents, protease inhibitors, and dry powder formulating agents.

35. The pharmaceutical composition according to claim 25, wherein the pharmaceutical composition further comprises at least one adjuvant.

36. The pharmaceutical composition according to claim 35, wherein the adjuvant is chosen from: Freunds adjuvant, a water/oil emulsion, mineral oil, granulocyte/macrophage-colony stimulating factor, and interleukin-2.

37. The pharmaceutical composition according to claim 28, wherein the pharmaceutical composition comprises at least two Epstein-Barr virus gene products.

38. The pharmaceutical composition according to claim 37, wherein the pharmaceutical composition comprises at least three Epstein-Barr virus gene products.

39. A viral vector comprising:
an Epstein-Barr virus gene product expression cassette comprising:
a polynucleotide encoding at least one Epstein-Barr virus gene product; and
a heterologous promoter operatively linked to the polynucleotide encoding an Epstein-Barr virus gene product.

40. The viral vector according to claim 39, wherein the Epstein-Barr virus is a strain chosen from Type 1, Type 2, SiIIA, A4, TSB-B6, ap876, p3hr1, b95.8, cao, raji, and daudi.

41. The viral vector according to claim 39, wherein the at least one Epstein Barr gene product is chosen from the gene products listed in Table 1.

42. The viral vector according to claim 41, wherein the Epstein-Barr virus lytic gene products are chosen from BZLF1, BHRF1, BHLF1, BALF2, BMLF1, BRLF1, BMRF1, BALF5, BARF1, BORF2, BCRF1, BKRF3, BDLF3, BILF1, BFRF1, BXLF1, BGLF4, BGLF5, gp350, gp220 and LMP1-lyt, and the Epstein-Barr virus latent gene products are chosen from EBNA1, EBNA2, EBNA3A, EBNA3B, EBNA3C, EBNA-LP, LMP1, LMP2A, and LMP2B.

43. The viral vector according to claim 42, wherein the lytic gene products are chosen from BZLF1 and BMLF1 and the latent gene products are chosen from EBNA1, EBNA3A, and EBNA3C.

44. The viral vector according to claim 43, wherein the Epstein-Barr virus lytic gene product is BZLF1 and the Epstein-Barr virus latent gene product is EBNA3C.

45. The viral vector according to claim 39, wherein the vector is an adenoviral vector.

46. A recombinant adeno-associated virus comprising an adenoviral vector according to claim 45.

47. A plasmid comprising:
an adenoviral portion comprising an adenoviral vector according to claim 45; and
a plasmid portion.

48. A mammalian cell comprising a viral vector according to claim 39.

49. A method of culturing at least one mammalian cell according to claim 48, under conditions to produce an immune response to Epstein-Barr virus gene products, the method comprising:
growing cells under conditions favorable to the expression of the Epstein-Barr virus gene product.

50. A plasmid comprising:
a viral portion comprising a viral vector according to claim 39; and
a plasmid portion.

51. A vector according to claim 39, further comprising at least one pharmaceutically acceptable excipient.

52. A viral vector pharmaceutical composition for producing an immune response against Epstein-Barr virus-associated neoplastic disease comprising:
an Epstein-Barr virus gene product expression cassette comprising:
a polynucleotide encoding at least one Epstein-Barr virus gene product; and
a heterologous promoter operatively linked to the polynucleotide encoding said Epstein-Barr virus gene product.

53. An adenoviral vector pharmaceutical composition for producing an immune response against Epstein-Barr virus-associated disease comprising:
an Epstein-Barr virus gene product expression cassette comprising:
a polynucleotide encoding at least one Epstein-Barr virus gene product; and
a heterologous promoter operatively linked to the polynucleotide encoding said Epstein-Barr virus gene product.

54. A method for ascertaining a subject's response to an Epstein-Barr virus vaccine comprising:
assaying for the presence of at least one Epstein-Barr virus gene product chosen from Epstein-Barr virus lytic gene products and Epstein-Barr virus latent gene products.

55. A method for inducing an immune response against at least one virus-associated disease in a subject, comprising: administering to said subject EBNA1 in combination with at least one Epstein-Barr virus lytic or latent gene product, chosen from BZLF1, BHRF1, BHLF1, BALF2, BMLF1, BRLF1, BMRF1, BALF5, BARF1, BORF2, BCRF1, BKRF3, BDLF3, BILF1, BFRF1, BXLF1, BGLF4, BGLF5, gp350, gp220, LMP1-lyt, EBNA2, EBNA3A, EBNA3B, EBNA3C, EBNA-LP, LMP1, LMP2A, and LMP2B.

56. A vaccine for inducing an immune response against at least one virus-associated disease in a subject, comprising: EBNA1, in combination with at least one Epstein-Barr virus lytic or latent gene product, chosen from BZLF1, BHRF1, BHLF1, BALF2, BMLF1, BRLF1, BMRF1, BALF5, BARF1, BORF2, BCRF1, BKRF3, BDLF3, BILF1, BFRF1, BXLF1, BGLF4, BGLF5, gp350, gp220, LMP1-lyt, EBNA2, EBNA3A, EBNA3B, EBNA3C, EBNA-LP, LMP1, LMP2A, and LMP2B, the vaccine further comprising at least one pharmaceutically acceptable excipient.
